(19) United States
(12) Patent Application Publication
(10) Pub. No.: US 2010/0086909 A1
(43) Pub. Date: Apr. 8, 2010

(54) Method for the prediction of individual disease course in sepsis

(75) Inventors: Stefan Russwurm, Jena (DE); Hans-Peter Deigner, Lampertheim (DE)

Correspondence Address: PATENT CENTRAL LLC;Stephan A. Pendorf, 1401 Hollywood Boulevard, Hollywood, FL, 33020, US

(73) Assignee: SIRS-LAB GMBH, JENA (DE)

(21) Appl. No.: 11/547,007

(22) PCT Filed: Jan. 14, 2005
(86) PCT No.: PCT/EP2005/000336

§ 371 Date: Jan. 8, 2007

(30) Foreign Application Priority Data
Mar. 30, 2004 (DE) 10 2004 015 605.0

(51) Int. Cl. C12Q 1/68; G06F 19/00

(52) U.S. Cl. 435/6; 702/19

(57) ABSTRACT

The invention relates to the use of gene expression profiles, obtained in vitro from a patient sample, for the generation of criteria for the prediction of an individual course of disease in sepsis. The invention is further of use for determining the probability of survival in sepsis, the assessment of the course of disease in sepsis during treatment and for the classification of sepsis patients.

### CROSS REFERENCE TO RELATED APPLICATION

This application is a national stage of PCT/EP2005/00336 filed Jan. 14, 2005 and based upon DE 10 2004 015 605.0 filed Mar. 30, 2004 under the International Convention.

### BACKGROUND OF THE INVENTION

1. Field of the Invention

The present invention relates to the use of gene expression profiles obtained from a patient sample in vitro for setting up criteria for the prognosis of the individual course of disease in sepsis, a method for in vitro measurement of such gene expression profiles as well the use of the gene expressions profiles and/or of probes used therefore for switching of and/or for changing the activity of target genes and/or for determining the gene activity for screening of active agents against sepsis and/or for evaluating the effect in the treatment of sepsis and/or the quality of the active agent and/or the integrity of the active agent in cellular and cell-free sepsis model systems and in sepsis animal models.

The present invention further relates to new possibilities of predicting the probability of surviving and the development of lethal complications in sepsis patients which can be derived from experimentally verified insights in conjunction with the occurrence of changes in gene activity (transcription) in patients with sepsis.

2. Description of Related Art

Despite advances in pathophysiological understanding and the supportive treatment of intensive care patients, generalised inflammatory conditions as SIRS and sepsis as defined according to American College of Chest Physicians/Society of Critical Care Medicine Consensus Conference (ACCP/SCCM) of 1992 [1] frequently occur in patients in intensive care facilities and contribute substantially to mortality of infections [2-3]. The mortality rate is approximately 20% in the case of SIRS, approximately 40% in the case of sepsis, and increases to up to 70-80% in the case of development of multiple organ dysfunctions [4-6]. The contribution of SIRS and sepsis to morbidity and lethality is of multidisciplinary interest, as it increasingly puts the success of the most advanced or experimental treatment methods of many medicinal fields (e.g. traumatology, neurosurgery, cardiac/pulmonary surgery, visceral surgery, transplantation medicine, hematology/oncology, etc.) at a risk, as they all, without exception, are threatened by an increased risk of the development of SIRS and sepsis. This also becomes apparent from the continuous increase of the occurrence of sepsis, namely by 139% from 73.6 to 176 cases per 100,000 hospital patients from 1979 and 1977, for example [7]. Thus, the decrease of morbidity and lethality of many seriously ill patients goes along with the concurrent progress in prevention and treatment and especially detection and monitoring of the progression of the sepsis and severe sepsis.

On a molecular basis, sepsis is defined as a clinical picture caused by pathogenic microorganisms. On the basis of an exhaustion of molecular control and regulation mechanisms near the infection site, a generalised inflammatory reaction develops that affects the whole organism. This infection is responsible for the clinical symptoms/criteria for diagnosis/SIRS-criteria according to [1] confirmed by the physician. This generalised inflammatory condition (defined as sepsis according to [1]) goes along with signs of the activation of various cell systems (endothelial cells, but also all leucocyte-like cell systems and in particular of the monocyte/macrophage system). Finally, molecular mechanisms, the normal task of which it is to protect the host against invasive microorganisms, harm the host's own organs/tissues and, thus, essentially contribute to the development of the organ dysfunctions dreaded by the physicians [8-11].

The meaning of the term sepsis has changed considerably in the course of time. An infection, or the strong suspicion of an infection, are still an essential part of the current definition of sepsis. Of particular consideration is, however, the description of organ failure functions remote from the location of infection in the framework of the inflammatory host reaction. The criteria of the American College of Chest Physicians/Society of Critical Care Medicine Consensus Conference (ACCP/SCCM) of 1992 are the ones that became most accepted in international literature as definition of the term sepsis [1]. According to these criteria [1] distinctions are made between the clinically defined degrees of severity “systemic inflammatory response syndrome” (SIRS), “sepsis”, “severe sepsis” and “septic shock”. According to this definition, SIRS is defined as the systemic response of the inflammatory system to an infectious or non-infectious stimulus. At least two of the following clinical criteria must be satisfied in this context: Fever>38° C. or hypothermia<36° C., a leukocytosis>12 G/l or a leukopenia<4 G/l or, as the case may be, a shift to the left in the differential haemogram, heart rate>90/min, tachypnoea>20 breaths/min or PaCO2 (partial pressure of carbon dioxide in arterial blood)<4.3 kPa. Those clinical conditions which satisfy the SIRS criteria and for which causatively an infection can be confirmed our at least is very probable are defined as sepsis. A severe sepsis is characterized by the additional occurrence of organ dysfunctions. Common organ dysfunctions include changes in the state of consciousness, oliguria, lactate acidosis or sepsis-induced hypotension with a systolic blood pressure lower than 90 mmHg, or drop in pressure of more than 40 mmHg from the initial value, respectively. If such a hypotension cannot be treated by the administration of crystalloids and/or colloids and the patient further needs treatment with catecholamines, then one refers to this as septic shock. Such a septic shock is detected in about 20% of all sepsis patients.

Sepsis is the clinical result of complex and highly heterogeneous molecular processes, which are characterized by the involvement of many components and their interactions on every organizational level of the human body: genes, cells, tissues, organs. The complexity of the underlying biological and immunological processes resulted in many kinds of studies comprising a wide range of clinical aspects. One of the results from these studies was that the evaluation of new sepsis therapies is rendered more difficult due to the presently used criteria which are quite unspecific and clinical based and which do not sufficiently show the molecular mechanisms [12]. Due to a lack of specificity of the currently used diagnosis of sepsis and SIRS, the clinicians are actually not sure from which point of time a patient is to be treated with a specialised therapy, for example with antibiotics, which for their part can have substantial side effects [12]. A survey carried out by the European Society of Intensive Care Medicine (ESICM) showed that 71% of the physicians questioned were unsure regarding the diagnosing of sepsis, despite many years of clinical experience [22].

Ground-breaking discoveries in molecular biology and immunology during the last two decades have allowed the development of a deepened understanding of sepsis that is oriented more towards the basic mechanisms. The knowledge resulting therefrom regarding relevant targets has formed the basis for the development of targeted and adjuvant therapeutic concepts basing primarily on the neutralisation of essential sepsis mediators [13-16]. One cause for the failure of almost all immunomodulatory approaches of therapy in clinical trials—despite effectivity in animal experiments—is considered to be the merely the poor correlation between the clinical diagnostic criteria, that are rather symptom oriented, and the basic mechanisms of a generalised immuno response [12, 17-18].

This is not surprising in retrospect, as even healthy humans may show changes of the heart rate and the respiratory rate, respectively, in the course of their daily activities, which per definition already would allow the diagnosis of SIRS. In consideration of today's biomedical possibilities, it seems anachronistic that 751 000 patients in the USA are diagnosed, classified and treated according to the above mentioned ACCP/SCCM criteria per year. Prominent authors thus have already long criticized that, at the expense of an improved sepsis diagnosis, in the past decade too much energy and financial resources have been lavished on the search for a “magic bullet” for sepsis therapy [19]. Further, recently published expert's opinions also indicate that for a better pathophysiologic understanding of sepsis a modification of the consensus criteria according to [1] is necessary [20-21]. Furthermore, many physicians agree that the consensus criteria according to [1] do not correspond to a specific definition of sepsis. A survey carried out by the European Society of Intensive Care Medicine (ESICM) showed that 71% of the physicians questioned are not sure about when to diagnose a sepsis, despite long-term experience in the clinical field [22].

Due to the above mentioned problems in the application of the consensus criteria according to [1], critical care physicians discuss various proposals for a more sensitive and specific definition of the various degrees of severity of sepsis [2, 23]. The new feature here is the fact that molecular changes should be taken into account directly in the evaluation of the severity of a sepsis as well as in the inclusion in innovative methods of treatment of sepsis (as for example the therapy with activated recombinant protein C). This consensus process [23], which currently is being carried backed by five international professional associations, is at the current time long from completion. It is the object to establish a system for the evaluation of the severity of sepsis. By means of this system it should become possible to classify the reactions of patients on the basis of their predisposed conditions, the kind and extend of the infection, the kind and severity of the host response as well as the degree of the accompanying organ dysfunction. The system described is referred to as PIRO, the abbreviation of the English terms “predisposition”, “insult infection”, “response” and “organ dysfunction”. From this, the individual chance of survival as well as the potential responding to the therapy may be derived [23]. Likewise, non-infectious conditions, which are currently subsumed under the concept of SIRS according to [1] are to be classified more precisely corresponding to the individual degree severity of SIRS.

Studies are carried out to find biomarkers which reflect the severity of the SIRS on the molecular level, as well, and render a clear discrimination of infectious conditions possible (currently classified as sepsis according to [1]). Similar classifications of the stages are already successfully used in other medical fields, for example for the classification of the different disease in oncology (TNM System, [24]).

In comparison to the consensus criteria according to [1], additional molecular parameters are to be included into the diagnosis [23] in order to render an improved correlation of the molecular inflammatory/immunologic host response with a degree of severity of the sepsis, and additionally, the making of statements on the individual prognosis possible. At the present time various scientific and commercial groups are intensively searching for such molecular biomarkers, since the conventional parameters, such as, for example, the determination of the C-reactive protein or the procalcitonin do not meet all clinical requirements and are, in particular, only insufficiently in the position to differentiate between surviving and nonsurviving sepsis patients [25]. Due to the insufficient specificity and sensitivity of the consensus criteria according to [1] and due to the faulty or delayed detection of the cause of infection, there is need for innovative diagnostic processes that are supposed to improve the capability of the person skilled in the art to prognosticate the course of disease with sepsis at an early point of time, to make it comparable in the clinical course and to make statements on the individual prognosis and the response to specific treatments.

Technological advances, in particular the development of microarray technology, make now it possible for the person skilled in the art to simultaneously compare 10 000 or more genes and their gene products. The use of such microarray technologies can now provide information regarding the status of health, regulatory mechanisms, biochemical interactions and signal transmitter networks. As the comprehension how an organism reacts to infections is improved this way, this should facilitate the development of enhanced modalities of detection, diagnosis and therapy of sepsis disorders.

Microarrays have their origin in “southern blotting” [10], which represented the first approach to immobilizing DNA-molecules so that it can be addressed three-dimensionally on a solid matrix. The first microarrays consisted of DNA-fragments, frequently with unknown sequence, and were applied dotwise onto a porous membrane (normally nylon). Routinely, cDNA, genomic DNA or plasmid libraries were employed and the hybridized material was labelled with a radioactive group [27-29].

Recently, the use of glass as substrate and fluorescence for detection together with the development of new technologies for the synthesis and for the application of nucleic acids in very high densities made it possible to miniaturize the nucleic acid arrays. At the same time, the experimental throughput and the information content were increased [30-32].

Further, it is known from WO 03/002763 that microarrays basically can be used for the diagnosis of sepsis and sepsis-like conditions.

The first explanation for the applicability of microarray technology was obtained through clinical trials in the field of cancer research. Here, expression profiles proofed to be valuable with regard to identification of activities of individual genes or groups of genes, which correlate with certain clinical phenotypes [33]. Many samples of individuals with or without acute leukaemia or diffuse B-cell lymphoma were analyzed and gene expression labels (RNA) were found and subsequently employed for the clinically relevant classification of these types of cancer [33, 34]. Golub et al. found out that an individual gene is not enough to make reliable predictions, while, however, predictions based on the change in transcription of 53 genes (selected from more than 6000 genes, which were present on the arrays) are highly accurate [33].

Alisadeh et al. [34] examined large B-cell lymphomas (DLBCL). Expression profiles were worked up by the authors with a “lympochip”, a microarray bearing 18 000 clones of complementary DNA that was developed to monitor genes that are involved in normal and abnormal development of lymphocytes. By using cluster analysis, they managed to classify DILBCL in two categories that showed great differences with regard to the survival chance of patients. The gene expression profiles of these subtypes correlated to two significant stages of the B-cell differentiation.

Yeoh E. et al. described the use of gene expression profiles established by means of microarrays for the prognosis of the course of leukaemia [35]. From various other studies with cancer patients, further examples for the use of gene expression profiles for the prediction of the chance of survival are known [36-38]. These studies show that the probability of recurrence can be predicted by the measurement of the gene expression profiles. This would be of high clinical-medicinal importance as, on the one hand, it would be possible to pay more attention to these patients, for example by means of more appointments for consultation during tumor after-treatment. This way, an early diagnosis of relapse as well as a systematic treatment of same would be possible. On the other hand, fewer appointments would be necessary for patients with a gene expression profile that does not point to an increased risk of relapse. Further, such gene expressions would also be usable in the decision whether a tumor has to be treated more or less aggressively. As a result, the authors suggested to regularly creating gene expression profiles from cancer patients for an early identification of the individual risk for therapy-related complications in the patient [35].

The use of gene expression profiles for the prediction of the individual course of disease in sepsis and in particular for the prediction of the individual risk of development of lethal complications have not yet been described.

The German Patent Applications DE 103 36 511.7, DE 103 150 31.5 and 10 2004 009 952.9 which have not yet been prepublished, describe that gene expression profiles are, in principle, usable, for example by means of microarray technology for the diagnosis of SIRS, generalised inflammatory inflammations, sepsis and severe sepsis. These applications are herein incorporated by reference.

### DETAILED DESCRIPTION OF THE INVENTION

For the creation of gene expression profiles according to the present invention, a majority of specific genes and/or gene fragments is used, selected from the group consisting of SEQ-ID No. 1 to SEQ-ID No. 247, as well as gene fragments thereof with 5-2000 or more, preferably 20-200, more preferably 20-80 nucleotides.

These sequences with the sequence ID 1 to SEQ-ID No. 247 are incorporated by the scope of the present invention and they are in detail disclosed in the enclosed sequence listing of 56 pages and 247 sequences which is, thus, part of the description of the present invention and, therefore, also part of the disclosure of the invention. In the sequence listing the single sequences are further assigned to the SEQ-ID No. 1 to SEQ-ID No. 247 to their GenBank Accession No. (website: http://www.ncbi.nlm.nih.gov/).

The present invention further relates to the use of gene expression profiles, which are obtained in vitro from a patient sample, and/or of probes used for this purpose, selected from the group consisting of SEQ-ID No. 1 to SEQ-ID No. 247 as well as gene fragments therefrom with at least 5-2000, preferably 20-200, more preferably 20-80 nucleotides, for switching off and/or for changing the activity of target genes and/or the determination of the gene activity for the screening of active agents for sepsis and/or for assessing the effect on sepsis and/or the quality of the active agent and/or the integrity of the active agent in cellular and cell-free sepsis model systems and in sepsis animal models.

In this context, also hybridisable synthetic analogues of the listed probes may be used.

Further, the gene activities in sepsis patients can be determined in a biologic fluid and from this “value” conclusions may be drawn with regard to the course of disease, the chance of survival, the course of therapy or the possibility to include or exclude the sepsis patients in clinical trials.

Another embodiment of the invention is characterized in that a specific gene and/or gene fragment is selected from the group consisting of SEQ-ID No. 1 to SEQ-ID No. 247, as well as gene fragments thereof with 5-2000 or more, preferably 20-200, more preferably 20-80 nucleotides.

Another embodiment of the present invention is characterized in that at least 2 to 100 different cDNAs are used.

Another embodiment of the present invention is characterized in that at least 200 different cDNAs are used.

Another embodiment of the present invention is characterized in that at least 200 to 500 different cDNAs are used.

Another embodiment of the present invention is characterized in that at least 500 to 1000 different cDNAs are used.

Another embodiment of the present invention is characterized in that at least 1000 to 2000 different cDNAs are used.

Another embodiment of the present invention is characterized in that the genes or gene fragments and/or the sequences derived from their RNA are replaced by synthetic analogues, aptamers, as well as peptide nucleic acids.

Another embodiment of the invention is characterized in that the synthetic analogues of the genes comprise 5-100, in particular approx. 70 base pairs.

Another embodiment of the present invention is characterized in that the gene activity is determined by means of hybridisation methods.

Another embodiment of the present invention is characterized in that the gene activity is determined by means of microarrays.

Another embodiment of the invention is characterized in that the gene activity is determined by hybridisation-independent methods, in particular by enzymatic and/or chemical hydrolysis and/or amplification methods, preferably PCR, subsequent quantification of nucleic acids and/or of derivates and/or fragments of same.

Another embodiment of the present invention is characterized in that the sample is selected from: body fluids, in particular blood, liquor, urine, ascitic fluid, seminal fluid, saliva, puncture fluid, cell content, or a mixture thereof.

Another embodiment of the present invention is characterized in that cell samples are optionally subjected a lytic treatment, in order to free their cell contents.

It is obvious to the person skilled in the art that the individual features of the present invention can be combined with each other in any desired way.

The term marker genes as used in the present invention encompasses all derived DNA-sequences, partial sequences and synthetic analogues (for example peptido-nucleic acids, PNA). The description of the invention referring to the determination of the gene expression on RNA level is not supposed to be a restriction but only an exemplary application of the present invention.

The description of the invention referring to blood is only an exemplary embodiment of the present invention. The term biological liquids as used in the present invention encompasses all human body fluids.

Further advantages and features of the present invention will become apparent from the description of an embodiment.

### Embodiment

Studies on Differential Gene Expression in Sepsis for Differentiation Between Nonsurviving and Surviving Patients.

Whole blood samples of 28 patients who were under the care of a surgical intensive care unit were examined for the measurement of the differential gene expression in connection with sepsis in order to differentiate between nonsurviving and surviving patients.

Whole blood samples of 12 surviving (9 male and 3 female patients) and 16 deceased patients (13 male and 3 female patients) were drawn during their whole stay in intensive care (patient samples). In the time period in the intensive care unit, each of these patients developed a sepsis the degree of severity being different. Gene expression profiles were analysed from those patients samples that have been drawn at the first day of treatment with the most severe degree of sepsis according to [1] during the first sceptical complications (some patients in intensive care suffer from more than one sepsis during the course of treatment).

A range of characteristics of the patients suffering from sepsis is shown in table 1. In the table, information regarding age, sex, cause of sepsis (cp. diagnosis) as well as clinical severity on the date of admission to the intensive care unit, measured according to in clinical literature well documented APACHE-II-Scores, as well as on the date of first treatment, measured according to in clinical literature well documented SOFA-Scores (both scores respectively in dots) were given. Likewise, the plasma protein level of procalcitonin (PCT), a more recent sepsis marker, from the samples of which the gene expression profiles were drawn at the date of treatment and the individual survival status are indicated.

As control samples, the total RNA from the cell lines SIG-M5 were used. All of the patient samples were co-hybridised with the control sample on one microarray each.

TABLE 1
Data of the group of patients

| | | | | | APACHE-II | SOFA | | |
|---|---|---|---|---|---|---|---|---|
| | | | | Classification | Score | Score | PCT | Survival |
| Patient | Age | Gender | Diagnosis | according to [1] | [Points] | [Punkte] | [ng/ml] | State |
| 1 | 48 | femal | acute cholezystitis (gangrenous) | severe sepsis | 17 | 8 | 0.76 | survived |
| 2 | 71 | male | other aspergillosis of the lung | sepsis | 13 | 6 | 1.69 | survived |
| 3 | 57 | male | insufficiency of the mitral valve III | septical shock | 11 | 11 | 186 | survived |
| 4 | 77 | femal | peritonitis | severe sepsis | 11 | 10 | 4.1 | survived |
| 5 | 50 | femal | infection of patchplastic prothesis AIC on both | severe sepsis | 9 | 9 | 1.71 | survived |
| 6 | 33 | male | polytrauma | septical shock | 9 | 9 | 0.65 | survived |
| 7 | 83 | male | stenosis of the aortic valve | septical shock | 33 | 14 | 1.15 | survived |
| 8 | 60 | male | prolapse of an intervertebral disc c6/7 | severe sepsis | 21 | 11 | 0.86 | survived |
| 9 | 45 | male | polytrauma at traffic accident | severe sepsis | 11 | 6 | 1.18 | survived |
| 10 | 20 | male | polytrauma after traffic accident | septical shock | 29 | 16 | 202 | survived |
| 11 | 67 | male | catheter sepsis | severe sepsis | 22 | 7 | 94.49 | survived |
| 12 | 42 | male | epidural bleeding | severe sepsis | 15 | 14 | 0.46 | survived |
| 13 | 53 | femal | acute pancreatitis (necrot.) | severe sepsis | 20 | 11 | 2.16 | died |
| 14 | 56 | male | acute posterior transmural myocardial | septical shock | 21 | 9 | 2.16 | died |
| | | | infarction | | | | | |
| 15 | 64 | femal | intracranial bleeding (non-traumatic) | septical shock | 14 | 12 | 500 | died |
| 16 | 70 | male | septical shock | septical shock | 27 | 17 | 9.42 | died |
| 17 | 78 | femal | acute peritonitis | septical shock | 27 | 10 | 29.2 | died |
| 18 | 70 | male | septical shock | septical shock | 10 | 15 | 1.02 | died |
| 19 | 72 | male | malignant neoformation of the cardia | septical shock | 10 | 12 | 4.92 | died |
| 20 | 88 | male | choledochal bile stone without cholangitis or | septical shock | 18 | 7 | 11.6 | died |
| | | | cholezystitis, bile-duct obstruction not stated | | | | | |
| 21 | 63 | male | intestinal perforation (non-traumatic) | septical shock | 29 | 10 | 9.17 | died |
| 22 | 74 | male | respiratory insufficiency, not specified | septical shock | 22 | 13 | 12 | died |
| 23 | 66 | male | acute peritonitis in intestinal | septical shock | 13 | 8 | 9.05 | died |
| | | | perforation (non-traumatic) | | | | | |
| 24 | 64 | male | cardiac disease, not specified, cardiac | severe sepsis | 35 | 9 | 405 | died |
| | | | arrest with successful reanimation | | | | | |
| 25 | 73 | male | dissection of the aorta, thoracic | septical shock | 19 | 12 | 123 | died |
| 26 | 61 | male | pleural empyema, right side | septical shock | 11 | 6 | 1.64 | died |
| 27 | 62 | female | acute peritonitis | septical shock | 27 | 16 | 16.1 | died |
| 28 | 73 | male | intestinal pneumonia, respiratory | septical shock | 32 | 11 | 10.9 | died |
| | | | insuffiency, not specified, acute | | | | | |
| | | | myocardial infarction | | | | | |

### Experimental Description:

After drawing whole blood, the total RNA of the samples was isolated using the PAXGene Blood RNA kit according to the manufacturer's (Qiagen) instructions.

### Cell Cultivation

For cell cultivation (control samples) 19 cryo cell cultures (SIGM5) (frozen in liquid nitrogen) were used. The cells were each inoculated with 2 ml Iscove's medium (Biochrom AG) supplemented with 20% fetal calf serum (FCS). Subsequently, the cell cultures were incubated in 12 well plates for 24 hours at 37° C. in 5% CO2. Subsequently, the content of the 18 wells was parted in 2 parts with the same volume so that finally 3 plates of the same format (36 wells in total) were available. Afterwards, the cultivation was continued under the same conditions for 24 hours. Afterwards, the resulting cultures of 11 wells of each plate were combined and centrifuged (1000×g, 5 min, ambient temperature). The supernatant was removed and the cell pellet was dissolved in 40 ml of the above mentioned medium. These 40 ml of dissolved cells were distributed in equal shares in two 250 ml flasks and incubated after adding 5 ml of the above-mentioned medium. 80 μl of the remaining 2 ml of the two remaining plates were placed in empty wells of the same plates that had previously been prepared with 1 ml of the above-mentioned medium. After 48 hours of incubations, only one of the 12 well plates was processed as follows: 500 μl were extracted from each well and combined. The resulting 6 ml were introduced into a 250 ml flask comprising approximately 10 ml of fresh medium. This mixture was centrifuged for 5 minutes with 1000×g at ambient temperature and dissolved in 10 ml of the above-mentioned medium. The following results were obtained by the subsequent cell counting: 1,5×107 cells per ml, 10 ml total volume, total number of cells: 1.5×108. As the number of cells was not yet sufficient, 2.5 ml of the above-mentioned cell suspension was introduced into 30 ml of the above-mentioned medium in a 250 ml (75 cm2) flask (4 flasks in total). After 72 hours of incubation 20 ml of fresh medium were added to each flask. After the subsequent incubation of 24 hours, the cells were counted as described above. The total amount of cells was 3.8×10⁸ cells. In order to obtain the desired number of cells of 2×106 cells, the cells were resuspended in 47.5 ml of the above mentioned medium in 4 flasks. After the incubation time of 24 hours, the cells were centrifuged and washed two times with phosphate buffer in absence of Ca²⁺ and Mg²⁺ (Biochrom AG).

The isolation of the total RNA is performed by means of NucleoSpin RNA L Kits (Machery&Nagel) according to the manufacturer's instructions. The above described process was repeated until the necessary number of cells was obtained. This was necessary to obtain the necessary amount of 6 mg total RNA corresponding to an efficiency of 600 μg RNA per 108 cells.

### Reverse Transcription/Labelling/Hybridisation

Subsequently, the complementary cDNA was prepared from the total RNA of the patient and control samples by means of reverse transcription under substitution of the dTTP-Fraktion by synthesized aminoallyl-deoxyuridintriphosphate (AA-dUTP). The RNA/cDNA complex was transformed into single strand cDNA by means of RNA hydrolysis. Subsequently, the cDNA samples were labelled with the fluorescent dyes Cy3 and Cy5 (Amersham) by means of chemical binding to AA-dUTP.

A microarray from the company SIRS-Lab was used for the hybridisation of the samples. On the microarray used, 5308 different polynucleotides with lengths of 55 to 70 base pairs were immobilised. Each of the polynucleotides represents a human gene. The spots were immobilised with a multitude of different control spots within 28 subarrays, each of the subarrays being arranged in a grid of 15×15 spots. The hybridisation was carried out using the hybridisation station HS 400 (Tecan) according to the manufacturer's instructions. The hybridisation solution was composed of 3.5×SSC (1×SSC comprises 150 mM NaCl and 15 mM sodiumcitrate), 0.3% SDS, 25% formamide, 0.8 μg/μl of each cot-1 DNA, yeast tRNA and polyA and the respective cDNA-samples. The arrays were hybridised for 10.5 hours at 42° C. The subsequent washing procedure is carried out as follows:

Addition of washing buffer I (2×SSC, 0.003% SDS) to the hybridisation chamber, washing at ambient temperature for 1.5 minutes, addition of washing buffer II (1×SSC) to the hybridisation chamber, washing with washing buffer for 1.5 minutes at ambient temperature, adding of washing buffer III (0.2×SSC) to the hybridisation chamber, washing with washing buffer III for 1.5 minutes at ambient temperature. Subsequently, the surfaces of the microarrays were dried with nitrogen at a pressure of 2.5 bar for 2.5 minutes at 30° C.

The hybridisation signals of the processed microarrays were subsequently read by means of the GenePix 4000B (Axon) scanner and the expression ratios of the different expressed genes were determined by means of the GenePix Pro 4.0 (Axon) software.

### Analysis

For the analysis, the average intensity of one spot was determined as median value of the corresponding spot pixel.

### Correction of Systemic Errors:

The median of the pixel of the local background was subtracted from the median of the spot pixel. For all further computations, the signal was transformed by means of arcus sinus hyperbolicus. The normalization occurred according to the approach of Huber et al. [39]. According to this approach, the additive and the multiplicative bias in a microarray was estimated on the basis of 70% of the gene samples present. For the analysis, the transformed relative ratios of the signals of the patients samples were calculated with respect to the control. This means that the calculation for the gene no. j (j=1, . . . , 5308) of the patient no. n revealed the data Gj,n=arcsin h(Scy5(j,n))−arcsin h(Scy3(j,n)), wherein [SCy3(j,n), SCy5(j,n)] is the associated signal pair. When a spot could not be analyses for a patient (no detectable signal intensity in both channels), the associated value was marked as “missing value”.

### Statistical Comparison:

For comparison the paired random student test was employed per gene. Both random samples contained the values of the patient groups of surviving and nonsurviving patients, respectively. For choosing the differentially expressed genes, the associated p-value and the number of missing values were evaluated.

### Results:

It applied for the group of the selected genes that the associated p-value was smaller than 0.05, with at least 5 detectable signals received per patient group.

The criterion for the grading of the examined genes was the level of the expression ratio of each gene. The most overexpressed or underexpressed genes, respectively, in the surviving and nonsurviving patients were the interesting ones.

Table 2 shows that 65 genes of the patient sample were found, which were significantly overexpressed in the nonsurviving patients, if compared with the surviving patients. Furthermore, Table 3 shows that 182 genes of the nonsurviving patients were significantly under-expressed, if compared with the surviving patients. From the results it is clear that the gene activities listed in Table 2 and Table 3 distinguish between surviving and nonsurviving sepsis patients (corresponding to sepsis according to [1]). Thus, the listed gene activities provide markers for the prediction of the chance of survival and the development of lethal complications in sepsis patients.

TABLE 2
Significant elevated gene activities in samples of patients with sepsis according to
[1], indicated as their relative relationship to the corresponding gene activities
of nonsurviving patients suffering from sepsis according to [1].

| | | mean normalised and | | | | | |
|---|---|---|---|---|---|---|---|
| | | transformed expression value | | Standard deviation | | | |
| | | Group of | Group of | Group of | Group of | | |
| GenBank | | surviving | nonsurviving | surviving | nonsurviving | Difference | |
| Accession No. | p-value | patients | patients | patients | patients | of mean values | Seq-Id. |
| AI560533 | 0.036 | −0.42 | −0.24 | 0.24 | 0.17 | 0.17 | 84 |
| D49410 | 0.042 | −0.40 | −0.22 | 0.14 | 0.24 | 0.18 | 125 |
| AI263527 | 0.047 | −0.49 | −0.32 | 0.12 | 0.26 | 0.18 | 67 |
| AI732958 | 0.028 | −0.25 | −0.06 | 0.10 | 0.25 | 0.18 | 102 |
| AI679923 | 0.033 | −0.45 | −0.27 | 0.19 | 0.23 | 0.19 | 97 |
| R56877 | 0.034 | −0.30 | −0.10 | 0.19 | 0.23 | 0.20 | 215 |
| AA478621 | 0.008 | −0.30 | −0.10 | 0.16 | 0.19 | 0.20 | 24 |
| R93174 | 0.038 | −0.36 | −0.15 | 0.25 | 0.24 | 0.21 | 222 |
| AA935872 | 0.030 | −0.35 | −0.14 | 0.14 | 0.28 | 0.21 | 46 |
| H22921 | 0.016 | −0.32 | −0.10 | 0.16 | 0.24 | 0.21 | 132 |
| R61687 | 0.046 | −0.67 | −0.46 | 0.13 | 0.31 | 0.22 | 218 |
| N73694 | 0.040 | −0.38 | −0.16 | 0.25 | 0.26 | 0.22 | 153 |
| H28119 | 0.040 | −0.25 | −0.03 | 0.22 | 0.27 | 0.22 | 134 |
| AI697430 | 0.046 | −0.46 | −0.24 | 0.24 | 0.29 | 0.22 | 99 |
| AI631076 | 0.043 | −0.81 | −0.58 | 0.19 | 0.31 | 0.23 | 92 |
| N63777 | 0.038 | −0.51 | −0.28 | 0.28 | 0.26 | 0.23 | 150 |
| AA889648 | 0.045 | −0.36 | −0.13 | 0.19 | 0.32 | 0.23 | 40 |
| AA679067 | 0.039 | −0.30 | −0.07 | 0.33 | 0.22 | 0.23 | 30 |
| AI865298 | 0.012 | −0.34 | −0.10 | 0.15 | 0.26 | 0.24 | 113 |
| H14986 | 0.028 | −0.06 | 0.18 | 0.19 | 0.31 | 0.24 | 129 |
| AI033361 | 0.014 | −0.36 | −0.11 | 0.17 | 0.27 | 0.24 | 54 |
| AI149817 | 0.012 | −0.42 | −0.17 | 0.30 | 0.15 | 0.25 | 60 |
| AI269981 | 0.043 | −0.22 | 0.03 | 0.27 | 0.31 | 0.25 | 68 |
| NM_003082 | 0.043 | −0.70 | −0.45 | 0.20 | 0.33 | 0.25 | 168 |
| AI620645 | 0.040 | −0.41 | −0.16 | 0.26 | 0.32 | 0.25 | 91 |
| R06585 | 0.022 | −0.48 | −0.22 | 0.22 | 0.29 | 0.25 | 204 |
| H61046 | 0.023 | −0.41 | −0.15 | 0.31 | 0.24 | 0.26 | 137 |
| AI803880 | 0.022 | −0.46 | −0.20 | 0.22 | 0.30 | 0.26 | 106 |
| AI862880 | 0.047 | −0.71 | −0.45 | 0.26 | 0.35 | 0.26 | 111 |
| AI185721 | 0.033 | −0.58 | −0.32 | 0.32 | 0.28 | 0.26 | 62 |
| AI888234 | 0.040 | −0.53 | −0.27 | 0.23 | 0.35 | 0.26 | 114 |
| AA278821 | 0.048 | −0.28 | −0.01 | 0.31 | 0.32 | 0.26 | 7 |
| AI933967 | 0.042 | −0.66 | −0.40 | 0.16 | 0.39 | 0.27 | 121 |
| AI635650 | 0.026 | −0.63 | −0.37 | 0.24 | 0.31 | 0.27 | 93 |
| R89075 | 0.032 | −0.59 | −0.32 | 0.29 | 0.31 | 0.27 | 221 |
| H14100 | 0.018 | −0.53 | −0.25 | 0.20 | 0.31 | 0.27 | 128 |
| AA699356 | 0.022 | −0.22 | 0.06 | 0.21 | 0.33 | 0.27 | 32 |
| NM_000678 | 0.012 | −0.66 | −0.39 | 0.17 | 0.29 | 0.27 | 186 |
| AI579907 | 0.003 | −0.41 | −0.13 | 0.23 | 0.21 | 0.28 | 86 |
| AA284108 | 0.009 | −0.46 | −0.17 | 0.29 | 0.24 | 0.29 | 9 |
| AA406037 | 0.030 | −0.42 | −0.13 | 0.16 | 0.33 | 0.29 | 14 |
| AA441939 | 0.007 | −0.44 | −0.14 | 0.24 | 0.25 | 0.30 | 18 |
| H11718 | 0.020 | −0.10 | 0.20 | 0.15 | 0.27 | 0.31 | 127 |
| AA621333 | 0.036 | −0.46 | −0.14 | 0.23 | 0.40 | 0.33 | 29 |
| AI864919 | 0.035 | −0.51 | −0.18 | 0.25 | 0.42 | 0.33 | 112 |
| AI184594 | 0.032 | −0.51 | −0.18 | 0.43 | 0.32 | 0.34 | 61 |
| NM-001288 | 0.048 | −0.28 | 0.07 | 0.45 | 0.37 | 0.35 | 188 |
| XM-043864 | 0.014 | −0.54 | −0.20 | 0.22 | 0.40 | 0.35 | 243 |
| AI475085 | 0.026 | −0.37 | −0.01 | 0.31 | 0.31 | 0.36 | 80 |
| H79760 | 0.027 | −0.51 | −0.15 | 0.33 | 0.34 | 0.36 | 139 |
| XM-012039 | 0.047 | −1.22 | −0.84 | 0.33 | 0.52 | 0.37 | 239 |
| AI377802 | 0.021 | −0.49 | −0.11 | 0.39 | 0.39 | 0.37 | 75 |
| NM_021972 | 0.038 | −0.94 | −0.56 | 0.46 | 0.37 | 0.38 | 183 |
| AI936462 | 0.022 | −0.61 | −0.23 | 0.33 | 0.45 | 0.38 | 123 |
| AA846117 | 0.040 | 0.00 | 0.40 | 0.45 | 0.40 | 0.40 | 39 |
| AI732878 | 0.032 | −0.54 | −0.13 | 0.61 | 0.31 | 0.41 | 101 |
| M61199 | 0.031 | −0.72 | −0.29 | 0.36 | 0.48 | 0.43 | 143 |
| NM_004513 | 0.007 | −0.54 | −0.11 | 0.35 | 0.38 | 0.43 | 176 |
| NM-012068 | 0.014 | −1.17 | −0.70 | 0.16 | 0.55 | 0.47 | 194 |
| NM_000228 | 0.032 | −1.21 | −0.68 | 0.61 | 0.55 | 0.53 | 156 |
| NM_000629 | 0.014 | −0.93 | −0.39 | 0.41 | 0.51 | 0.54 | 158 |
| AI655693 | 0.040 | −0.50 | 0.04 | 0.48 | 0.44 | 0.55 | 94 |
| AA017133 | 0.030 | −0.26 | 0.29 | 0.42 | 0.37 | 0.55 | 5 |
| H16999 | 0.027 | −0.55 | 0.07 | 0.43 | 0.45 | 0.62 | 130 |
| AI888493 | 0.024 | −0.56 | 0.25 | 0.78 | 0.42 | 0.81 | 115 |

TABLE 3
Significant reduced gene activities in samples of patients with sepsis according to
[1], indicated as their relative relationship to the corresponding gene activities
of nonsurviving patients suffering from sepsis according to [1].

| | | Mean normalised and | | | | | |
|---|---|---|---|---|---|---|---|
| | | transformed expression value | | Standard deviation | | | |
| | | Group of | Group of | Group of | Group of | | |
| GenBank | | surviving | nonsurviving | surviving | nonsurviving | Difference | |
| Accession No. | p-value | patients | patients | patients | patients | in mean values | Seq-Id. |
| AI147932 | 0.031 | −0.02 | −0.18 | 0.13 | 0.20 | −0.17 | 58 |
| R88267 | 0.036 | 0.08 | −0.12 | 0.31 | 0.18 | −0.21 | 220 |
| AI039866 | 0.039 | 0.06 | −0.15 | 0.34 | 0.14 | −0.21 | 55 |
| AI583425 | 0.023 | 0.22 | 0.00 | 0.22 | 0.22 | −0.22 | 87 |
| AA845475 | 0.032 | 0.13 | −0.10 | 0.28 | 0.22 | −0.22 | 38 |
| AA992381 | 0.049 | −0.24 | −0.47 | 0.27 | 0.29 | −0.23 | 48 |
| NM_022740 | 0.047 | 0.21 | −0.02 | 0.34 | 0.22 | −0.23 | 184 |
| R43258 | 0.025 | 0.11 | −0.12 | 0.10 | 0.28 | −0.23 | 208 |
| N32057 | 0.022 | −0.02 | −0.25 | 0.20 | 0.25 | −0.23 | 145 |
| AI277856 | 0.026 | 0.13 | −0.10 | 0.18 | 0.24 | −0.23 | 70 |
| AA454150 | 0.028 | −0.03 | −0.26 | 0.30 | 0.21 | −0.23 | 21 |
| AI597793 | 0.002 | 0.15 | −0.09 | 0.10 | 0.19 | −0.24 | 89 |
| AA012911 | 0.009 | 0.32 | 0.08 | 0.20 | 0.19 | −0.24 | 4 |
| AA960982 | 0.037 | 0.28 | 0.03 | 0.37 | 0.20 | −0.24 | 47 |
| AA906962 | 0.037 | −0.27 | −0.52 | 0.34 | 0.27 | −0.26 | 43 |
| AI933013 | 0.042 | 0.40 | 0.14 | 0.38 | 0.24 | −0.26 | 120 |
| AA704293 | 0.012 | 0.37 | 0.11 | 0.30 | 0.18 | −0.26 | 33 |
| AA682521 | 0.022 | 0.10 | −0.16 | 0.31 | 0.25 | −0.26 | 31 |
| AI142427 | 0.013 | 0.24 | −0.03 | 0.20 | 0.29 | −0.27 | 57 |
| R54393 | 0.007 | 0.11 | −0.15 | 0.32 | 0.12 | −0.27 | 214 |
| NM_015318 | 0.012 | 0.22 | −0.04 | 0.22 | 0.25 | −0.27 | 196 |
| AA528169 | 0.050 | 0.45 | 0.18 | 0.40 | 0.22 | −0.27 | 27 |
| XM-048792 | 0.049 | 0.21 | −0.06 | 0.23 | 0.36 | −0.27 | 244 |
| AI343613 | 0.039 | −0.07 | −0.34 | 0.34 | 0.27 | −0.27 | 73 |
| N91341 | 0.024 | 0.10 | −0.18 | 0.27 | 0.27 | −0.27 | 154 |
| AI023785 | 0.020 | 0.06 | −0.21 | 0.24 | 0.27 | −0.27 | 53 |
| NM_032721 | 0.021 | 0.22 | −0.05 | 0.26 | 0.25 | −0.28 | 201 |
| AA035159 | 0.044 | 0.08 | −0.20 | 0.36 | 0.21 | −0.28 | 6 |
| AI091302 | 0.012 | 0.32 | 0.04 | 0.21 | 0.26 | −0.28 | 56 |
| AA845372 | 0.040 | 0.56 | 0.28 | 0.29 | 0.35 | −0.28 | 37 |
| AI657063 | 0.005 | 0.19 | −0.09 | 0.28 | 0.16 | −0.28 | 95 |
| AI187962 | 0.005 | 0.55 | 0.27 | 0.22 | 0.23 | −0.28 | 64 |
| AA845015 | 0.018 | 0.02 | −0.27 | 0.31 | 0.27 | −0.29 | 36 |
| AA436651 | 0.042 | 0.28 | 0.00 | 0.46 | 0.23 | −0.29 | 17 |
| NM_147180 | 0.028 | 0.22 | −0.07 | 0.36 | 0.26 | −0.29 | 202 |
| AI421397 | 0.030 | 0.42 | 0.13 | 0.37 | 0.25 | −0.29 | 77 |
| W15233 | 0.034 | 0.35 | 0.05 | 0.22 | 0.31 | −0.30 | 230 |
| AA906278 | 0.009 | −0.16 | −0.46 | 0.22 | 0.22 | −0.30 | 42 |
| AI187401 | 0.044 | −0.07 | −0.37 | 0.42 | 0.31 | −0.30 | 63 |
| AI492493 | 0.038 | −0.16 | −0.46 | 0.35 | 0.36 | −0.30 | 81 |
| AA496969 | 0.001 | 0.16 | −0.15 | 0.22 | 0.18 | −0.31 | 26 |
| AI936300 | 0.049 | 0.61 | 0.30 | 0.31 | 0.36 | −0.31 | 122 |
| NM_002983 | 0.046 | 0.27 | −0.04 | 0.38 | 0.34 | −0.31 | 165 |
| N53480 | 0.016 | −0.18 | −0.49 | 0.16 | 0.29 | −0.31 | 149 |
| AI400066 | 0.009 | −0.14 | −0.45 | 0.34 | 0.24 | −0.32 | 76 |
| AI799547 | 0.022 | 0.24 | −0.08 | 0.22 | 0.35 | −0.32 | 105 |
| R94509 | 0.015 | 0.28 | −0.04 | 0.32 | 0.27 | −0.32 | 223 |
| AI539457 | 0.036 | 0.27 | −0.05 | 0.37 | 0.38 | −0.33 | 83 |
| AI921468 | 0.003 | 0.24 | −0.09 | 0.30 | 0.22 | −0.33 | 118 |
| NM_005368 | 0.016 | 0.34 | 0.00 | 0.38 | 0.23 | −0.34 | 247 |
| NM_000597 | 0.026 | 0.12 | −0.22 | 0.42 | 0.31 | −0.34 | 185 |
| NM_005658 | 0.045 | 0.29 | −0.05 | 0.59 | 0.22 | −0.34 | 178 |
| NM_014211 | 0.003 | 0.21 | −0.14 | 0.31 | 0.22 | −0.34 | 195 |
| AI342905 | 0.039 | 0.66 | 0.32 | 0.50 | 0.32 | −0.35 | 72 |
| NM_006273 | 0.013 | 0.49 | 0.14 | 0.34 | 0.29 | −0.35 | 179 |
| H28769 | 0.010 | 0.34 | −0.01 | 0.39 | 0.25 | −0.35 | 135 |
| AA460188 | 0.040 | 0.23 | −0.11 | 0.48 | 0.31 | −0.35 | 23 |
| R42778 | 0.022 | 0.43 | 0.08 | 0.48 | 0.27 | −0.35 | 206 |
| NM_004740 | 0.027 | 0.21 | −0.15 | 0.50 | 0.27 | −0.36 | 177 |
| AA432083 | 0.011 | 0.14 | −0.22 | 0.48 | 0.18 | −0.36 | 16 |
| AA397913 | 0.026 | 0.86 | 0.50 | 0.33 | 0.42 | −0.36 | 10 |
| AI473446 | 0.041 | 0.40 | 0.04 | 0.39 | 0.29 | −0.36 | 79 |
| AI289206 | 0.010 | 0.36 | 0.00 | 0.44 | 0.20 | −0.36 | 71 |
| NM_000074 | 0.018 | 0.43 | 0.06 | 0.42 | 0.30 | −0.37 | 155 |
| AA458848 | 0.012 | 0.07 | −0.30 | 0.38 | 0.32 | −0.37 | 22 |
| R61395 | 0.008 | 0.47 | 0.10 | 0.30 | 0.28 | −0.37 | 217 |
| N39164 | 0.027 | 0.22 | −0.15 | 0.41 | 0.24 | −0.37 | 147 |
| T91937 | 0.032 | 0.54 | 0.16 | 0.42 | 0.31 | −0.38 | 226 |
| AA707013 | 0.025 | 0.01 | −0.37 | 0.51 | 0.29 | −0.39 | 34 |
| H20320 | 0.024 | 0.64 | 0.26 | 0.43 | 0.40 | −0.39 | 131 |
| K03195 | 0.045 | 0.21 | −0.18 | 0.41 | 0.46 | −0.39 | 141 |
| NM_001710 | 0.049 | 0.63 | 0.24 | 0.69 | 0.23 | −0.39 | 189 |
| AA281330 | 0.030 | 0.69 | 0.30 | 0.54 | 0.35 | −0.39 | 8 |
| AI825890 | 0.049 | 0.32 | −0.07 | 0.31 | 0.58 | −0.39 | 109 |
| AI270372 | 0.016 | 0.38 | −0.01 | 0.51 | 0.24 | −0.40 | 69 |
| T97352 | 0.037 | 0.39 | −0.01 | 0.46 | 0.46 | −0.40 | 229 |
| AA002267 | 0.040 | 0.42 | 0.02 | 0.64 | 0.30 | −0.40 | 1 |
| AI561302 | 0.040 | 0.09 | −0.31 | 0.42 | 0.37 | −0.40 | 85 |
| AI741506 | 0.002 | 0.38 | −0.03 | 0.14 | 0.28 | −0.40 | 103 |
| XM-012608 | 0.009 | 0.37 | −0.03 | 0.45 | 0.29 | −0.40 | 240 |
| AA935686 | 0.012 | 0.39 | −0.02 | 0.55 | 0.21 | −0.41 | 45 |
| R53564 | 0.015 | 0.46 | 0.06 | 0.40 | 0.26 | −0.41 | 213 |
| AI744597 | 0.016 | 0.28 | −0.13 | 0.40 | 0.35 | −0.41 | 104 |
| AI148036 | 0.035 | 0.30 | −0.11 | 0.60 | 0.17 | −0.41 | 59 |
| AI925267 | 0.036 | 0.41 | 0.00 | 0.63 | 0.34 | −0.41 | 119 |
| AA731720 | 0.007 | 0.31 | −0.11 | 0.50 | 0.21 | −0.42 | 35 |
| NM_021132 | 0.006 | 0.21 | −0.21 | 0.34 | 0.34 | −0.42 | 200 |

These changes characterized in Tables 2 and 3 can be used for the inventive process.

The GenBank Accession Numbers indicated in Tables 2 and 3 (Internet-access via http://www.ncbi.nlm.nih.gov/) of the individual sequences are associated with the attached 56-page sequence listing, itemized or in detail with respectively one sequence (Sequence ID: 1 up through Sequence ID: 247).

### REFERENCES

1. Bone R C, Balk R A, Cerra F B, Dellinger E P, Fein A M, Knaus W A, Schein R M, Sibbald W J, the ACCP/SCCM Consensus Conference Committee (1992) Definitions for Sepsis and organ failure and guidelines for the use of innovative therapies in Sepsis. Chest 101, 1656-1662; and Crit Care Med 1992; 20: 864-874.
2. Marshall J C, Vincent J L, Fink M P, Cook D J, Rubenfeld G, Foster D, Fisher C J Jr, Faist E, Reinhart K (2003) Measures, markers, and mediators: toward a staging system for clinical Sepsis. A report of the Fifth Toronto Sepsis Roundtable, Toronto, Ontario, Canada, Oct. 25-26, 2000. Crit Care Med. 31:1560-7.
3. Alberti C, Brun-Buisson C, Goodman S V, Guidici D, Granton J, Moreno R, Smithies M, Thomas O, Artigas A, Le Gall J R; European Sepsis Group (2003) Influence of systemic inflammatory response syndrome and Sepsis on outcome of critically ill infected patients. Am J Respir Crit Care Med. 168:77-84.
4. Brun-Buisson C, Doyon F, Carlet J, Dellamonica P, Gouin F, Lepoutre A, Mercier J C, Offenstadt G, Regnier B: Incidence, risk factors, and outcome of severe Sepsis and septic shock in adults. A multicenter prospective study in intensive care units. French ICU Group for Severe Sepsis. JAMA 1995; 274: 968-974
5. Le-Gall J R, Lemeshow S, Leleu G, Klar J, Huillard J, Rue M, Teres D, Artigas A: Customized probability models for early severe Sepsis in adult intensive care patients. Intensive Care Unit Scoring Group. JAMA 1995; 273: 644-650
6. Brun-Buisson C, Roudot-Thoraval F, Girou E, Grenier-Sennelier C, Durand-Zaleski I. (2003) The costs of septic syndromes in the intensive care unit and influence of hospital-acquired Sepsis. Intensive Care Med. [Epub ahead of print]
7. Increase in National Hospital Discharge Survey rates for septicemia—United States, 1979-1987. MMWR Morb Mortal Wkly Rep 1990; 39: 31-34
8. Bone, R. C. Sepsis, the sepsis syndrome, multi-organ failure: a plea for comparable definitions. Ann Intern Med 1991; 114: 332-333
9. Matot, I., C. L. Sprung, et al. Definition of sepsis. Intensive Care Med 2001; 27 (suppl): S3-S9.
10. Friedland, J. S., J. C. Porter, et al. Plasma proinflammatory cytokine concentrations, Acute Physiology and Chronic Health Evaluation (APACHE) III scores and survival in patients in an intensive care unit. Crit Care Med 1996; 24: 1775-1781.
11. Beutler, B., A. Poltorak, et al. Sepsis and evolution of the innate immune response. Crit Care Med 2001; 29: S2-S6.
12. Vincent J L, Angus D, Annane D, et al. (2001) Clinical expert round table discussion (session 5) at the Margaux Conference on Critical Illness: outcomes of clinical trials in Sepsis: lessons learned. Crit Care Med 29:S136-137.
13. Abraham, E., Laterre P. F., et al. Lenercept (p55 tumor necrosis factor receptor fusion protein) in severe sepsis and early septic shock: a randomized, double-blind, placebo-controlled, multicenter phase III trial with 1,342 patients. Crit Care Med 2001; 29: 503-510
14. Abraham, E., Reinhart K., et al. Assessment of the safety of recombinant tissue factor pathway inhibitor in patients with severe sepsis: a multicenter, randomized, placebo-controlled, single-blind, dose escalation study. Crit Care Med 2001; 29: 2081-2089
15. Pittet, D., Harbarth S., et al. Impact of immunomodulating therapy on morbidity in patients with severe sepsis. Am J Respir Crit Care Med 1999; 160: 852-857
16. Abraham, E., Marshall J. C., et al. Sepsis and mediator-directed therapy: rethinking the target populations. Mediator-directed therapy in sepsis: rethinking the target populations. Toronto, Canada, 31 Oct.-1 Nov. 1998. Mol Med Today 1999; 5: 56-58.40-43
17. Abraham, E., Raffin T. A. Sepsis therapy trials. Continued disappointment or reason for hope? JAMA 1994; 271: 1876-1878.
18. Zeni F., Freeman B., et al. Anti-inflammatory therapies to treat sepsis and septic shock: a reassessment. Crit Care Med 1997; 25: 1095-1100
19. Bone, R. C. The pathogenesis of sepsis. Ann Intern Med 1991; 115: 457-469
20. Marshall J C (2000) SIRS and MODS: What is there relevance to the science and practise of intensive care?, Shock 14:586-589
21. Vincent J-L (1997) Dear SIRS, I'm sorry to say that I don't like you. Crit Car Med 25:372-374
22. Ramsay G, Gerlach H, Levy M M et al (2003) An international sepsis survey: A study of doctor's knowledge and perception about sepsis. Crit Care Med 31
23. Levy M M, Fink M P, Marshall J C et al. (2003) 2001 SCCM/ESICM/ACCP/ATS/SIS International Sepsis Definitions Conference. Cri Car Med Vol 31, No 4
24. http://www.krebsinformation.de/tnm-system.html (Stand 1. March 2004)
25. Rulβwurm S. (2002) Procalcitonin als Marker bakterieller Infektionen and Sepsis: Einfluss sepsisrelevanter Bedingungen auf die Expression von Procalcitonin, Habilitationsschrift eingereicht bei der Medizinischen Fakultät der Friedrich-Schiller-Universität Jena
26. Southern E M (1974) An improved method for transferring nucleotides from electrophoresis strips to thin layers of ion-exchange cellulose. Anal Biochem 62:317-318
27. Gillespie D, Spiegelman S (1965) A quantitative assay for DNA-RNA hybrids with DNA immobilized on a membrane. J Mol Biol 12:829-842
28. Lennon G G, Lehrach H (1991) Hybridization analyses of arrayed cDNA libraries. Trends Genet 7: 314-317
29. Kafatos F C, Jones C W, Efstratiadis A (1979) Determination of nucleic acid sequence homologies and relative concentrations by a dot hybridization procedure. Nucl Acid Res 7:1541-1552
30. Fodor S P, Read J L, Pirrung M C, Stryer L, Lu A T, Solas D (1991) Light-directed, spatially addressable parallel chemical synthesis. Science 251:767-773
31. Pease A C, Solas D, Sullivan E J, Cronin M T, Holmes C P, Fodor S P (1994) Light-generated oligonucleotide arrays for rapid DNA sequence analysis. Proc Natl Acad Sci USA 91:5022-5026
32. Schena M, Shalon D, Davis R W, Brown P O (1995) Quantitative monitoring of gene expression patterns with a complementary DNA microarray. Science 270:467-470
33. Golub T R, Slonim D K, Tamayo P, et al. (1999) Molecular classification of cancer: class discovery and class prediction by gene expression monitoring. Science 286:531-537
34. Alizadeh A A, Eisen M B, Davis R E, et al. (2000) Distinct types of diffuse large B-cell lymphoma identified by gene expression profiling. Nature 403:503-511
35. Yeoh E, Ross M E, Shurtleff S A, et al. (2002) Classification, subtype discovery, and prediction of outcome in pediatric acute lymphoblastic leukemia by gene expression profiling. Cancer Cell 1(2):109-10.
36. Kari L, et al., (2003) Classification and prediction of survival in patients with the leukemic phase of cutaneous T cell lymphoma. Journal of Experimental Medicine, 197[11]:1477-88
37. van't Veer L J, et al., (2002) Gene expression profiling predicts clinical outcome of breast cancer. Nature 415:530-6,
38. Beer D G, et al., (2002) Gene-expression profiles predict survival of patients with lung adenocarcinoma. Nature Medicine 8:816-24
39. Huber W, Heydebreck A, Sueltmann H, et al. (2003) Parameter estimation for the calibration and variance stabilization of microarray data. Stat. Appl. in Gen. and Mol. Biol. V

1-24. (canceled)

25. A method for generating criteria for the prediction of an individual course of disease in sepsis, comprising
obtaining a biological sample from a patient,
preparing a gene expression profile from the sample in vitro,
correlating data from the gene expression profile with observed or measured data regarding the course of disease in sepsis for the individual from which the sample was obtained, and
using the gene expression profile to generate criteria for the prediction of the course of disease in sepsis for said individual.

26. The method as in claim 25, wherein prediction of the course of disease in sepsis includes determining the probability of survival in sepsis.

27. The method as in claim 25, wherein the course of disease in sepsis is determined during therapy.

28. The method as in claim 25, wherein said gene expression profile is used to generate criteria for classification of sepsis patients.

29. The method as in claim 25, wherein said gene expression profile is used to generate inclusion criterion or exclusion criterion of patients with sepsis in clinical trials of stages 2-4.

30. The method as in claim 25, further comprising using said gene expression profile to generate gene activity data for further electronic processing.

31. The method as in claim 30, wherein said further electronic processing comprises using the gene activity data for the production of software for the description of the individual prognosis of a sepsis patient, for diagnostic purposes and/or for patient data management systems.

32. The method as in claim 30, wherein the gene activity data is used for the production of expert systems and/or for modelling of cellular signal transmission paths.

33. The method as in claim 25, including
using a specific gene and/or gene fragment for the generation of gene expression profiles, the gene and/or gene fragment being selected from a group consisting of SEQ-ID No. 1 to SEQ-ID No. 247 as well as gene fragments therefrom with at least 5-2000 nucleotides.

34. The method as in claim 33, wherein said gene fragments comprise 20-200 nucleotides.

35. The method as in claim 33, wherein said gene fragments comprise 20-80 nucleotides.

36. A method for in vitro measurement of gene expression profiles for generating criteria for the prediction of an individual course of disease in sepsis, comprising
(a) determining the gene activity of various certain genes associated with sepsis in a patient sample, wherein the sepsis-specific genes and/or gene fragments are selected from the group consisting of: SEQUENCE-ID No. 1 to SEQ-ID No. 247, as well as gene fragments thereof with 5-2000 nucleotides, and
(b) using the results of step (a) to produce a gene expression profile, and
(c) using the results of (b) to generate criteria or the prediction of an individual course of disease in sepsis.

37. The method as in claim 36, wherein said gene fragments comprise 20-200 nucleotides.

38. The method as in claim 36, wherein said gene fragments comprise 20-80 nucleotides.

39. The method according to claim 36, wherein at least 2 to 100 different cDNAs are used.

40. The method according to claim 36, wherein at least 200 different cDNAs are used.

41. The method according to claim 36, wherein 200 to 500 different cDNAs are used.

42. The method according to claim 36, wherein at least 500 to 1000 different cDNAs are used.

43. The method according to claim 36, wherein at least 1000 to 2000 different cDNAs are used.

44. The method according to claim 36, wherein the genes or gene fragments and/or the sequences derived from their RNA listed in claim 10 are replaced by synthetic analogues, aptamers, mirrormeres as well as peptide- and morpholine nucleic acids.

45. The method according to claim 44, wherein the synthetic analogues of the genes comprise 5-100 base pairs.

46. The method according to claim 36, wherein the gene activities are determined by means of hybridisation methods.

47. The method according to claim 46, characterized in that the gene activity is determined by means of microarrays.

48. The method according to claim 36, wherein the gene activity is determined by hybridisation-independent methods, in particular by enzymatic and/or chemical hydrolysis and/or amplification methods, preferably PCR, subsequent quantification of nucleic acids and/or of derivates and/or of fragments of same.

49. The method according to claim 36, wherein the sample is selected from the group consisting of body fluids, in particular blood, liquor, urine, ascitic fluid, seminal fluid, saliva, puncture fluid, cell content, or a mixture thereof.

50. The method according to claim 36, wherein cell samples are optionally subjected to lytic treatment, in order to free their cell contents.

51. A method for switching off and/or for changing the activity of target genes and/or the determination of the gene activity for the screening of active agents for sepsis and/or for assessing the effect on sepsis and/or the quality of the active agent and/or the integrity of the active agent in cellular and cell-free sepsis model systems and in sepsis animal models, said method comprising
obtaining a sample from a patient
optionally determining the gene expression profile in vitro from the patient sample, and
using the gene expression profile and/or the probes, selected from the group consisting of SEQ-ID No. 1 to SEQ-ID No. 247, that were used for the determination of the gene expression profile, with at least 5-2000, preferably 20-80 nucleotides for switching off and/or changing the activity of target genes and/or determining the gene activity for the screening of active agents for sepsis and/or for assessing the effect on sepsis and/or the quality of the active agent and/or the integrity of the active agent in cellular and cell-free sepsis model systems and in sepsis animal models on the basis of said gene expression profile.

52. The method according to claim 52, wherein hybridisable synthetic analogues of the probes listed in claim 23 are used.


SEQUENCE LISTING (247 sequences)

SEQ ID NO: 1 - DNA, 435 bp, Homo sapiens
  misc_feature (2)..(2): n is a, c, g or t.
tntttagaaaagctgttctttaatgaagaggaaatgatataccatgggtatcaaatgttcttttctgataaaggaagcaactacagaaagcttttatttatttgttcaaagtaaccagtgctatggatgcaaaaaacccccaacaactcttccaacgctactttcaaaacaaacatatcaaacttgattttcattaggatgtagttattttgtcacactagtaaatcaaaatctaagacccaatttttatatatatatataaatatatataaaagaacaatgtgaacaattattgagcttcatcttctcggacaagaatgccaagttagtctctcttcataaaaagcaattacaatttcgaggacacttcatatttgcctctttcgccagcaccaagtctgcctcatctgaatctttccatttcatgagaaacat

SEQ ID NO: 2 - DNA, 293 bp, Homo sapiens
  misc_feature (18)..(18): n is a, c, g or t.
atagccagcaactttttnttaatgttacatatactattctcaaggcacatctgatgatatattttataacacagtaggtgtcaaagtatgtttaacatatgatttcttcaggatccctcccctttttgaattccaaatggaggaactgaaagtgcgatgtaaagactggcgatccatattctatctttggcaagcttgtacaagcactattgtaaatgtaatgtaaaagaactgtaaactaggaacttcttcagtttatgaaacaccattcaggactgctgcttcttgagngt

SEQ ID NO: 3 - DNA, 440 bp, Homo sapiens
  misc_feature (351)..(351): n is a, c, g or t.
caaactaatggctttttttttcagattatattaacttcaacttacattgtgataagaacagttctcgttaagtgatataataacacttcttgtttgagccagtttgaattgcttctaaaacaactgccactaaaagcatcttaagtgacagttatgtttagatatggaaacatagttaaatattactttaatttttctacctactttggtttaggacagatgcctctgacttcaacagaccatcagagatgtgtaacattaattttagaaaaccactcagacagagagtgctgagctcccttaggaaacaacacaagtctctgctttcccatcaccaatgctctgggcctngcgctcatgggtngaagtcctctgttatcaggctgggcttcataagtcagtcccagcatctttgaaggcatattggatggctgaaatgn

SEQ ID NO: 4 - DNA, 497 bp, Homo sapiens
  misc_feature (369)..(369): n is a, c, g or t.
tctcatcggtgttagaaagaaatgatattgaatgaaacgatgtaattcaaagacctgctgtgtatttatcccaaatgctccttatcacacacacaaagagaaagctattgtaggctttccttaagtggtagccttcctccctttctgcctgatgaacacaaatacatgtcctctcagcagcacagttacaaaggcaagcctgagatgtattctcagcacctggccttgtggtcatcccatgctctgtgtgagtgcaggggacagaacttgcactgtgcagacagcattctgcctgggaacaggaataaaaagatgggagagtaaagctggagctcagcagggtgtggtgccaatgcactatagtgagtnagggggaaaccaaatcagattcaaaccatttttatttcctagctaccggagtgaaaacagagcccataatttgttatgaacccaaaaattaataaagaaacatgccactttaccnaatatggacatga

SEQ ID NO: 5 - DNA, 428 bp, Homo sapiens
  misc_feature (362)..(362): n is a, c, g or t.
ttatggaacatggacattttaatgaatttcatattgcaatgaagacagtcaacacttagagacaattatatcgatacaataaaaactgattccatggaacacttataaatgcatacattttcctccaaagaaatatgtttttcacagacttaagtaacaaatgatttacataggattcttcagattccaaaatatgtacagagtttttgtctctacagaaactgatgagcagtggttgaaaaagagttgctttgcccaacagcatggctcttatgaaattcctgtatcatatacagttttcagtaaccacacattttcccgtctatcattaaatggaattctgggacacatccagcataccnggaccaaactggttcccactttnccaaacactggtttcagacgagattcacgtggtnggtttgaac

SEQ ID NO: 6 - DNA, 426 bp, Homo sapiens
  AA035159
ggtaataagatttaattttccagtagcctgcatgaattgttccccacataaaactgtacagttagtgactgaattgtatacttaagtcccagtattttacattagtgagactgaaattagaggtaaatttctttaacaagtgtaaggcttacctatttataaagaattattctgttagtgtttaagaaaaacagatctagagacaatccagtaggctgcattgtaaacattatgattataaatctcttagtactgccattattattgacagttttgtaaagacttgtaaaaagtccagtttctcaggaatatgaaaattatcttcagaaacctggttggggcctttctccaattcctccagccagctgaaatactgccaagctcacttcatgtggcaagggtgatctggcaccttcctgtgaggct

SEQ ID NO: 7 - DNA, 435 bp, Homo sapiens
  AA278821
catttatttttatttttagatggtgtttcactcttgttgcccaggctggaagagcaaaactcggtctcaaaaaaaaaaaaagctatcaatcatctaggcctttagcaagtcatattctttgctagttggagggtctttgctccttgttaatggctgctgactgatctggatggtggctgctgaaggttggggtggctgtggcaattccttaaaataagacaacaatgacatttgctatattgattgacttgttttcatgaaagagttcctgatggcgtgtgatgctggttgataatattttacccacagtggaccttttcaaattggagtcaatccagttttctcaaaacctgcctctggctttatcaactaagtttatgggatattcaaatcctttggtgccattttcacagtgttcacagcgtcttcaaaagg

SEQ ID NO: 8 - DNA, 434 bp, Homo sapiens
  AA281330
gagtatgatgtttaatgtaagatccagtggcttggcagagttggtgcaaaactgtaaagtaccagattaagttctagtcagtgagttagcagcctaggcacttgggcaaaaaaattggccctgctgtcccaagtcctccaaagccctctccgtatcaccagctaggagctccaagactgatatgccactacttggaggcttgagtagaatccacgcgtcctcagtttccaaagtgacaagagactggccccagccatcaaagcagtagaattttcaggacagggttgaggacagtggggacagaactggacatgggggcctgagctgctgaacatttgctacatgcagtagtctaagaggctcacagctggagaggttagcctgtccaccttcccacccatgtgtggtagggaagaaggtgaccatctagttaa

SEQ ID NO: 9 - DNA, 494 bp, Homo sapiens
  AA284108
gcaggatggagatcctgtgcctgtgcagccgtcttggcgccgcgcctcggcccgttgcccggactttcggcgcccggacgcctctttgaccagcgcttcgggaggggctgctggaggccgagctggctgcgctctgccccaccacgctcgccccctacagcctgcgcgcacccagcgtggcctgcccgtcgcccaggtgccgacggaccccggccacttttcggtgctgctagacgtgaagcacttctcgccggagacaattgctgtcaaggtggtgggcgaacacgtggaggtgcacgcgcgccacgaggagcgccggcatgagcacggattcgtcgcgcgcgagttccaccgtcgctaccgcctgccgcctggcgtggatccggctgccgtgacgtccgcgctgtcccccgagggcgtcctgtccatccaggccgcaccagcgtcggcccaggccccaccgccagccgcagccaagtaggagggggctgggc

SEQ ID NO: 10 - DNA, 433 bp, Homo sapiens
  AA397913
gcgaccgcgcctccggtccgctccgtagccgacccctaaagatccgggcacaaaacacactaaaagcaatcagcaaagcgtcctctgcagactgtttcgcctagcaaagtcctgaccctcccaacctggccagcagctcacagcctccttagggctctagcttctcccttttgccttgccccacccccactgggtgacagcgaggaccccaacttcagacctcctataaggtgggacctgtgtgtcccaaagaaaggtgtgtggtgggggggggggcgggggggcgggcaaaaggcaaacagaaagctgcaagtctaaaagcagatttttgaaaaatcccagataatggggatgagggcccctttctcaaggactgggctcccccatctccccaagcatcctagttccttggaaacttctccccacttatctg

SEQ ID NO: 11 - DNA, 443 bp, Homo sapiens
  AA398611
tttgaatctttaatgaatatttaattaggaccttttcagacagtgtaatagttgggggaaaaaagatgcctttttccaacacaccactgagttgaagaggatggaaaaagtcaacagatgagtttcctgtctttcccgccagttcccttaaaccatttctgcttttcatttcttctccttttcttacatctctgcatctgtggactctcctgtcgtctccaggagcaaggcagagacacattcttgtctgtgagcagatgacttgtctagaaatcatggacagcctgcaagtccttccagtcctcccaagaatctgacatggttttaggcagagaagaggctcagaggcctgctgactcaacatctcttacaagcgcctttgagtttctccaggaagcgctgaggttttctcagaagtcaaaaaactttttctgtattgtttg

SEQ ID NO: 12 - DNA, 374 bp, Homo sapiens
  AA400488
tttttttttggtttttagtaaaaggtttatagaggtgagcagccatcaccacacacaacctaagaacattttcatcatccccaaaacaaacccacacactggccactgtgtccccagccccaacccccaccaactcccggccccggcacccttcaatctctggctgtgctcgggacactccctatagccggagccttccttcagttagcgtcaggttccgggttcatccacggggtgcctatgctggtgcctcactcctttttctctcctggtgtggatttcccacgttttattctccattcatcagctgacggacatgtggtttctagtttttggctactgtgaacagtgctgctgagagatttgtgtacgtt

SEQ ID NO: 13 - DNA, 421 bp, Homo sapiens
  AA405180
tttttttttttttggcattaaaatgtattttgcagtacaagatacataccaataaaatataaacaaaaagcaaacaaacgcattttttaaaacttagctattaagaacatttcaattgtcaaaagatacatttgagtagaatataccaggtcaatttttctatttaacttctactccagcaatacaacatacatcagtgacatcaaataaggcagtcagacttctgtttattttatttcttacaattctcactaagatctctaaagagagtcttattcttttgtttaagtagcataagagagtcttattctttaaaaccattatgtttacatgagaaagtatcttttcctcttcaaatatgaagacagatatagggattacatgaaattaatagcaaagttaattgaatgtggtagccagc

SEQ ID NO: 14 - DNA, 454 bp, Homo sapiens
  misc_feature (283)..(283): n is a, c, g or t.
tttgaatattgacataaacactgctaattaatctgcacagcttattgaagttctgtcagttgtcctaaagtccttgtcctggagcaggaaccaatccaagatggcactaacgtgacctcgtcctcctgtctccttactccccaccagcctgtgacggtcctcagtgtttcctcatcacctctgaagagctgaggccagttattctgtagaatctcactcactttgggtttgtctggtggccccagggtttgggaacttgcaggcccccaggcctcagccgccntgagcaggagggtttccgtggagcagaggggtgggagcctgacaggagtgagctgaagtacaggnnggagatggtgggtgtgggcaacacccaagggaacagagaaagnggaccccaagcttgagaagcgatatgggggtccagggaagctcagacaagggccacgtgtgc

SEQ ID NO: 15 - DNA, 469 bp, Homo sapiens
  AA427522
tttttttctaacaacagaattgtggttttatttaacagtccttatcttttagacatacatattgaaatatttacaaatgaaatgatttgctatctgggatttgcttcaaaataaataggggttcaaagatgaatggcatttagataaatcaagattggccatggattgatcatgttaacatgagttcattatgcaattgtccttttacatatacctgacatacgccattaattttttaaaaagcactcactccgatgtctcctatgtataagaacacaggcagctggcagcaactaaatggtatcaactaaatttttataaattccaagagtcgttaattctcagctatctcaaaagtaaaatctctgggaaacttaatattaagacagtaactatctgcacacagtaatctgtacaaagtttaattaccaccaccagtcagtcataggctagtcgagtagctgtatca

SEQ ID NO: 16 - DNA, 389 bp, Homo sapiens
  AA432083
ttttttccagtagtaaacgtctttattcttccgtgacgtcgcacacccaagccacctcccgcggactctccgctggcagtcccacccagtgagggtagaggcggtggagggcccacccaaggccggctcttccaagcaccacaaatcctctcctcgcccaggtccctttccccctttctggccttcgtggggacaccttcagagtccccaaggaaggctgtgctgatagcctcactcaacccagcttttcaaagttctgcaggaggcaaagggcaagcccagagattagggttagagaccaaagacaagcggtcgttacccctaatatccaaggggaggggacgggagggggctttttgtaaaaatgcccctgaggtatagaaccggca

SEQ ID NO: 17 - DNA, 345 bp, Homo sapiens
  AA436651
tttgctatacaataaaatgctttattcagccacagctttctaaaaatagatgtggcatcaaaatgcgcattcatcaaatcttgaaggagtttcaaatctttagagtgactctgaactctcgttttggttgacacaataatccataaaaacaaataaagcacccattctgatctggaaggggtaacgaggtgaaaatattgataagccaaagtgacagcctcttttgtagacatgatcctgcctaaatttctcatgcctcacaaaagagggccctgaattccctgcttctccagagctccacgcagtccttgaagcactctctggctgtctctgcaagtggctgaa

SEQ ID NO: 18 - DNA, 423 bp, Homo sapiens
  AA441939
gaataaaaaaaataaccgtatttattaaaatgaaacaaattatgtgtatgactgcaagagttctggcatgatgcatcttggactcaacaaatctttgtttggggaatcccatgtggttggagaacagcaggatagttatttactctggtgagtctgtcaccaggcagttgtagtaccatgaggttttacatgcaccatttaaacaatgtactaggccgggcactgtggctcacgcctgtcatcccagcactttgggaggctgaagtgagtggatcacttgaggttgggagtttgagaccagccaggtcaacatgatgaaaccccatcttgggccgggtgcagtggctcacacctgtaatcccagcactttgggaggccaaggcgggtggatcacgaggtcaagagatcaagaccattctggcc

SEQ ID NO: 19 - DNA, 309 bp, Homo sapiens
  AA443712
atttatttttaatttagtttcatactttagggcatatccactaaaaatgcatagacaagtgactgtgttttaaaaatacttgaagtaattctcctctgtgtgtttaggacaccaacacaattagtttcatttatttcatttagcttttgctttggatatatagttacgcacctcataacattgtttcagtcaacaatggaccacaatgaaccacgtatgcaccagtggttccataaggttgtaatggagctggacaatttctatcacctagtgacactgtaactgcagtgacatcatagctgtcttaat

SEQ ID NO: 20 - DNA, 430 bp, Homo sapiens
  AA452134
ttttttgttttgtcttcctttattacatttttgcttttgagatacaaaccaacctctcataccacacaaagaacatccattcttgggatttttaaagtgcatttccccttgaactctgtgtacaaaaatatttatcttttaaaacatgcaaaaatttcttgacaaggcacttttaggtataaaatgaagatgagtccttggttctacattcacactgaagtaatagtgaaacatcatcacagctgcactctcaaagccctcagaggtccagcagtctctaaaaactcgtcaacaagactaaaaacattcacggctttacaatgtgggttacagagctttacaaccatgaccaggaaaaactgctcgtaacaacagctgtccttcccagttccacatgtgttgtctcactgcaggaaattaagataagctg

SEQ ID NO: 21 - DNA, 397 bp, Homo sapiens
  AA454150
acagaatccggcggggtcctcgcaggggcgagctccagctccgcggcgtcactacccggggcattatccagctcctccagaagctgcggtccggctccgcggttccaccaccacggccgccatggaggcagcagcgcccggcctcaccgcggctcagcagccgctcgaaggcagctttttcgcccggcgccacgctccacagagtcccgataggccgccgggcgccgggccggagctgaggcctgcttccccaggctcgtcctcggtctcgcgttgctgacgcagccggcgtagggcgcttgctaggcggctgggaggagcgctgcaaccgcggactctcccagcacctggtcacggtagaagttttctgcgcaggtgccatgcgtccggtagcagc

SEQ ID NO: 22 - DNA, 396 bp, Homo sapiens
  AA458848
acacacaataagagtgccaagggatacaagaagccacagggggaaaaacaagtgacatatctgcatggagcctaaatttcgcttggtgattttcagggtagaagcattacatattaaaaacactgctattttaggggaatacagaatttaaaaatccacacagattcgcaacgtaaaacatgccgcccaaagacacgtccgcggtgtcccgacagttccggctccgccgcgggccacgaggcggggagcgacggtcactcccctcggttacccggagtacttgggcgggaacgcgtactcgcgggagagacgggctcctgggcgagggctgcgtcttagtcgccctgcttcttagctctcgaggtcccgcgtgttacgtgttagcaccgcccgggg

SEQ ID NO: 23 - DNA, 335 bp, Homo sapiens
  AA460188
cgaggagaagagtaatgtcttgctctccgccctgacaacttttatgtattataatttgaccgcgctgggccacgctgcgcagtcgcacgtcggaaccgctgcgttaacccagccccggaaataacgtaccgatcttcggagtccaagtcggaagttgactagagagtcacctgctttcgtccgctctaggacaccagacacaccaggccgccatgttgtgtaaggatccccagccagtgagcctaaaatgggtagaaggaaaagatatttcttaccctacgcacccaggtccaagtcatcattattttgtctgaaatttatcttcctaactgatt

SEQ ID NO: 24 - DNA, 399 bp, Homo sapiens
  AA478621
aattttgagagatgttgctcaggctggtctcaaactccaggcctcaagtgatcctcccaccttagactcctaaagcaccgagattagaggcatgagccactctctacttctatgagatcttttttaccttccacataagtacagtacatggagtatttatctttctgtgcctggcttacttaacataatgtcttctaagatcatacatgttgctgagaataacagaattttgtttttttaatggccaaatagtattccacggggtatatataccacattttatctgttcatttgctgatagacacaggttgattccaatcttgggtactgtgaatagtgctggaacgagcatggcaggtgcagatgtctccatatatggattttcttttggatatatac

SEQ ID NO: 25 - DNA, 372 bp, Homo sapiens
  AA479964
ggatatatggatgggattctttattattacttaaagtttactgttctcataattacaggctacaacttagaggtttgaaactttaagagaaaattctataaagagagctcaaataaacatgaatcagtatgtagaagacatttaaaactttgtaagtagtgccacttagctctgggcaatacttatctgtccttttcaagcacaggccctggagccagagtcaacaacataaaataccacaaacgactctaagacattcatcctacacaagcgagcacatatacatatatatttatataatatatacatactttaatattatgttggtccacggggtatacacattttaacaaaagataaaaatctatgtaa

SEQ ID NO: 26 - DNA, 550 bp, Homo sapiens
  AA496969
cgattaattacaaaacatatatttatgtgtaatgtgttcttaaagctgtaatttacaaatattcataatgatttccaagcttcagtctgcctcttgatataggtccatttcatttatataatcataaataataataagttagtatttattttgctgatttaaggtgttcatccaaaagttcagcaatggcttcttcatctgctgattgaagcagtctctgaattagctgatccaggcccttcccaccacataaaaattccttaatatcacgaacttcatatgctatcctgtggtcactgactctatcctgggtgaaattatatgtccgaattcgctctgactgggctcttgttcccacctgcagttttctagcactttgttgctgacgcttgtctttctcaataatctgctggtagagtctagctctcaacacacgaaaggctatttctttattttttatctgtggatcttcttgttggcattctactactagccctgtggggatgtggacaagtctgacggcactatcagttttattaacatgctgccc

SEQ ID NO: 27 - DNA, 611 bp, Homo sapiens
  AA528169
ttagtgttggttaagcatttatttttgcatcctaatcctaatatccactctatgaatttaggacctactattactcccattttaaagataagtacaccgaggtttaccaagattaactgacttgccaagggtcacacactggagtaagggcagaaccttagactcatgtctgacccctcgccccatgctcttaaccacataatgccatcttttatttcagtgacaagacaaaagcatttaggtcacactcttgttgttaaacaggaaaattatataaatgcaaattgtttagaataggttttggatttccttttgagcaaacccccagtcctttaaaggatatacttaaaattttttttaagcagaggaaaatagactactatttaccaatgacaagtttatagagttttaaaacgagaggttttttaaaaatctgcatccaaggtgaagacgttatctgtggtttctgccataactgcaaaacgctgatactctgaaactcgtttctcaaagaaatttgtttttccttctaaagaaatgttttcccataaatcaaaaggattttctgcctgaaaaacctttgagaatccaagttccacaagtaatctgtc

SEQ ID NO: 28 - DNA, 443 bp, Homo sapiens
  AA535107
tatctttacgtatttaatgtaactgcttgtatacactgccaaaagaaaagctactttccataaacttgagatcatagggaaactggatattatacttttagcttaggcgttaactaatggtacaaagtatgtaaacccctatcacttctaaatggcagttcactgaatgaaaatcagtttacttccaatttttgttttgaggattagaggtttagaaaacatctttaaatattaaacagtcattaaaacaagggaagcaaattaaggttcaaaattaagttactgtggttatccttgggccactagttggtcaagccagttacaaatgtgctcccatttctctagaaccctaggctcctaagtccggaagcctggtaacataaccttaaatttttcacattcctaggcccagggttcagcgggctacatggcacctccgtacc

SEQ ID NO: 29 - DNA, 498 bp, Homo sapiens
  AA621333
taaagtgaaagcaagtttattaagaaagtaaaagaataaaagaatggctactccatggctctagacagagcagccccgaggctgggagttgcccatttttatcgttatttcttgattatatgctaaacaaggggtggattattcaggcctcccctttatagaccatatagggtaacttctgacgttgccgtggcatttgtaaacttatggcacaggtaggagtgtagcagtgtggactcccagaggtcactctcctagtcatctcggttttggtgggttttggccagtttctttactgcaacctgttttattagcaagctctttatgacctgtatcttgtgccgatatcctgtcttattctgtgacctagaatgctttaaccatctgggaatgcagcccagcaggtctcatcctcatttttacccagcctttattcaagatggagttgctctggttcaaatacctctgacaattcctccccccaaccgttgttcaggc

SEQ ID NO: 30 - DNA, 449 bp, Homo sapiens
  AA679067
gagacagggtctcactttgtcccaggctccaggctggagtgcagtggcacaatgtcagctcaccacagcctcagcctcccaggctcaagtggatcctcccatctcagctcccaagtagctggggccacatacgcacatcaccatgcctggctaatttttgtatttttttgtagagacaggtttttgccatgctgcttaggctggtcttgaactcccaggctcaagcgacccacctgtctcggcctcccaaagtgctgggattataggcatgacccaccacacctgacccaacagtggttatctttgagtgatattattttcttttcttttccctataattcctaattttctacgatgaatgtcatcattttcttataagaaaaaacaaagttttgaaaactcaggagagttgtgataaataaaagtcctactataatactttacttttt

SEQ ID NO: 31 - DNA, 556 bp, Homo sapiens
  AA682521
aaaagggccttcctagaaaaaaagacaaagactaccaaggatgtatgaagggaagaaagaagtgtgaggaactgtggcactaaggatggcctggtgagcaccaaatgatgtgtagctagaggaatgcaagaggagtgagagaactcagggccgcccacccctaatcagaccaccacccatggccacacaatgttcctttgttgtttcaattacagtgcatagaatttgttttaaaataacacttctgataccaaattttaaatatggtatcatctcaatggtgtaaataatgcattaaaaatgggctggcaggaaatatgccaacctggttactgcctctaggaagagcgggtaggaataatcatctttttgcttcttcattcttttccattttccagttttctgtaactatgtattatcaggggaaaaagcatacttttcaaaatgaatttcatattataggagaaataaagccagttatttcccccaaataatccctggccgaggaggaccctcgagtcaaataaacagatatataaaggattctctgttactg

SEQ ID NO: 32 - DNA, 401 bp, Homo sapiens
  AA699356
tttttttttttttttagagagtacaagtgcaggtttcacattcattgtgtcgtggtgaagtctgggcttttagcatacccatcttctgaatagcgaacgttgtacccaacaggtaatttttttgagcctgaccctcctcttaccctcccaccttctgtaatcgccagtgtctattatttcactctgtaggaccacatgtacccattgtttagctcccacctataagtggaaacatgaagtatttgactttctgtttctgagatatttcagttaggataatggcctccagttccattaactaaaatatttgtatatatgttcatatgtttgacctcgctaactccttgggggaaattttgactgttttataatatattagaagttttttaacatctgaattt

SEQ ID NO: 33 - DNA, 418 bp, Homo sapiens
  AA704293
tctctacgttatcttttgtaacctcaatcccaaggtggaactagaaagacaaccactagctccacatgtaattactatttacttcctttggatgtgttttttcccattatcagatggggaagttcaggtcttaaccaaggaagcctaagtgggcttgtgttcaggctctgtgggactccctaccccactgggtcacattccagctgtgttcttggctcagttcacttttgtggtttcaagaatgtaaataatttgcacttttaccctcttctttttatctatttctgcacaaggtattggtgacctgaatatatttccttgaattcatcgacttcaaatattccatttctgtaactgacctcaatatgttttcctcctttgaaacaataaactcgctaacagatcaacacagtctact

SEQ ID NO: 34 - DNA, 352 bp, Homo sapiens
  AA707013
gaggacctttttctttttgatctaaactttttgaggcttcgtttgcctaaggagttgtctccttggccacattaaaacaggtctagcagtggctattacggatcccccaggattgaggcaaagacagagtgaagctgtcaggtactgcaaggtgtaaacaactgactccaggtcagcccagagaagggacaatgaagtctgattgaccgcacagaggtattcactcacgtctgggggctgctgattgaaaagcaccatgctggatgtctggcaggttttggctagcttgtgcttgaatctccttcccagatatgctgaggcaccatctgtatgagtcttgcaggaaaggtct

SEQ ID NO: 35 - DNA, 491 bp, Homo sapiens
  AA731720
tttttttttactaattttggatttggttttttcttcttttggtagatttgtatctttgtgcatcatcaggctgtttatttgaaatatttctagtgtcttcatgtaggcattgattgctataaatttgccatttaacacttcttttgctgtgccccataggttttggtatgttgtgtttctattttcatttgtttcaaggaatttttaaattcccttcctaatttattccttcactcattggtcattcagaagcatgttttctaactcccatgtatttgcatagttttaaatgttcctcttgtaattgatgtctagtttattccactgtggtcacataagatatttgacataattttgatgttttagtgttttgagacttggaatttccttgtctcaaggaaattccttgtgggcaaaggacagaattcaaagtcatccctctgaggttgacctgagacaaatggatatctgattgtttcctctgtcttatt

SEQ ID NO: 36 - DNA, 484 bp, Homo sapiens
  AA845015
tttttttgcatagttgtcactttattatattattttccttctgtgacctttccaagtctgaaacagtcccagggacttcttttggttagttatttgcaatcaccgaattgatccagtcgatgtaattggagacccgcgtgaagacggagggcttgtggtagtagttgcagccgaggcgagacccgaagctgacgatgccgtgcacctgccaccggccgtcagacgcctgacagttcagtggcccgccagagtctccgttgcagctggagatcacgccatcacccccagcacagatcatactggttttcacgctgctgccccaccaggcagagctggagcaggtggcatagtccacaaccagcaaccggccctgctgcaggacatcaggaacagccccgttggtctgcagccttccccagcccgtgacgtagcaggggtagttgttgggtagaatggtgccggcagaggggaggcaggccagctg

SEQ ID NO: 37 - DNA, 493 bp, Homo sapiens
  AA845372
ttttttttttttttttttggggaattaaagaaatgtttattgaaacattattataatcatgattttacacaattatattatattgtataaattatatcacaatatgatggcatttatattttcaaaaggatccccagatttttgtgatactttcaagcagtgaaattggtaaaccagctctctctaaaaaaaaaaaaaagcgaaactgttgctttatagcatctgtgaatttctgtggtgtaaatactcccactatggccaatttcaacatgagggctcatgaagtagctcatggctaccaacgcaaggtcactgagtgcagagttaggaagaaattctcacagttgtcccttgtgagtgggcatgagctggcttcacacaccactgctttcaagtatacttttgaggaggaatcaatcattcaataagcctcatctgaggattctattcaccaggaggtgaacaggaggttacacaaaagcaaagaagccaa

SEQ ID NO: 38 - DNA, 457 bp, Homo sapiens
  AA845475
gcctgccttcccgtggtttaagacaaaaacacataaacaagttcagacaactgattgtatgattctgggaattctttgctttcctttccttctccctcggcaccacctcctctccccaggcctccctgtcgggcatggggaggaggttggagctcagcatcttgaggaatgtgtcaagacagcccctccgctccgcgctgcacggccagccgcctttgtccgggaggacagacagaaacgcagcaaggcacacaccaaggctgccggggaggcccgggcaccggacagatgcctgttgggaagccaggagctgccgagccacggaccacctaacagcctctccgcccggaacgtgacatagagagtgttggcattaactcggggggtgtccgatttcacttcagacaccaagtttttctttgtttgtttaaacatggaattcagacttcattgtaca

SEQ ID NO: 39 - DNA, 543 bp, Homo sapiens
  misc_feature (223)..(223): n is a, c, g or t.
ttttaatcttttgttattatttgtgtccagcttcctcacctgatagagcaggcactcaacaaatacatgatgacatatttttaacaaccaatatgtgcgtccctgcccctggctccatagatttgcaagatttgtgtgtccattcataagccttccaaacgcagggcctgcttgctgccctctttgctctgtggggcccagatggcagccccggaggcgctgngtgggaggctgtgtcctggggtccaccactgctccccggtaagccaagcccgactgcccctacatggaaaattcatccctcctccagatgggggattcagggcaccgccacttcccacaaccccgggaatctccctggccacagtcctggcgggaccccccccatccccagctcactgtgacagagtcccctgcacagtcctgggacggctccaggtcgaagtcctggaagacgagcctcacagcaaagccctctggagccttgatgtccgtgctgctctcctggcctttggcataaggctctgggtatccgggggatgt

SEQ ID NO: 40 - DNA, 380 bp, Homo sapiens
  AA889648
tttaatttttttttttttaattctcagcaaggcaatgtaattctatagaagggtgcccccttacatttggagcaatagtgagcacacacctggacaagggaggagaggaggttcttattcccgatccacgtggaccctgctgctgtgttcccctattggctagggttagatgcacaggccaaactaattccaattggctaatttaaagatagtgtacgggtgagtggtttggctggaaaaatggctatggcagagcaggaaatcagaaagagtcagggtggaaaatgagtcagggcagagcaggtaatcggaatgagtcagggtggagcaggtaatcgaaaaacgttgtgccacaaggatgttaagtttaaaagtagaaa

SEQ ID NO: 41 - DNA, 404 bp, Homo sapiens
  AA889842
cgttttattgtgcttcactttattgcactttgctgatattgcattttttacaaattaaaagtttgtggcaaccctgtgaccaacaagtctattggcaccatttttccaacagcatgtgctcacttcgtgtttgtgccagtaacatgttagcaataaagtattttaaaataaggtatgtacattgttttttagacataatgctattgcacatttaccagactacaggatagtgtaaacataaaattttatatgcactgggaaaccaaaaaaatttgtgtgacttgctttattgtaatatttgcttcattgcagtgatctggaaccaaatctgcattgtctccaaagtatgcctgtatgatgtgatgaaaatggcacttcttctccgtggtcctactttcaaaaac

SEQ ID NO: 42 - DNA, 475 bp, Homo sapiens
  AA906278
ttttgttttttaaaaaaatattttttaatgtacaagtcagtagtaaaatatggacatgtgaattttttagacatcaccttttccatgtatagaataaatgaacatcactgtgttaacaatgcctcaaattagagtccaagaaagaagttgtttgcttgttcctattggactcattttatatgtcttcatggatggacaaagtaggaaatcaagatgaaactagatttgatttttttttcagtgattttatccatttataatgtaacatcaaattatttttagtaattttatgaaaataattgtcttaaaacaaggtcattaaggactctaaacataggaaggtgtatgcatatatctaatatgttttctttaatcacattgtggcctctggaggaaacagttctttgtgtctacttgttttctcacctctcacgaaactactacaaggatgaggagagttatggatgtggtttgt

SEQ ID NO: 43 - DNA, 336 bp, Homo sapiens
  AA906962
tattgacttatgtctgttcaaataactggtgtggtttctctctcctgactcaaccccgaatgacacattcccttgaaggggcagaacatcccgagacaagatgcaacggtacacctaaataaagacataaaatcttcaaagaaagataatgtccttcagaaaagggtttgtcagccataggcgcctggcccagggagggtcagagaataaaccccaggatgacatcctcagcagtgacaagagaacagaccaccaccatccatagcctggatgactgtaaagactcctgctatagactggataactgcttcatgtgccccctaccctccaccccac

SEQ ID NO: 44 - DNA, 313 bp, Homo sapiens
  AA910846
aattgtgcatttctgattattttctcaaaaacatttaaagacaagtcaatgtgaactaaggagtgggaggaggtttcaaggaaaaggtagaacattttaagggtttttctgcagaggaaaaagtgagcaaaaaaaaaaaaaaggaaaaggaatttaagttgaaaaatactccaacaaacgaaccaacatgttttcagaactttttgaagttccagtatggtgctgtcatggttggtgagctgtctgtcccttgatcagtagacaatgcgagtttcttccgtcattcgccacgctgaccacttcccctctgctg

SEQ ID NO: 45 - DNA, 479 bp, Homo sapiens
  AA935686
taattaaaaaaaaaaaggacatagaaaacatcagtcttgaacttctcttcaagaacccgggctgcaaaggaaatctcttttttttttttttttatgtgctgtcaagttttgaagtggtgatctttagacagtgactgagtatggatcatttgaacgaggcaactcaggggaaaagaacattcagaaatgtctaacaatgtgagtgatccgaaggtccaccagccaagattgcccgcctggagcaaaacgggagcccgctaggaagaggaatgcttgggaatacaggtgcaaaaatgcagggagtgcccttaaaacactcgggccatctgatgaaaaccaacctttggaaaggaaccatgctgccagttttctgcgtggtggaacttatgaaaacgcattgaatatgcttgccaggaggagcctgcaaaattttttcctcggaaaaaaggaattgcttttcaacaggttaatccaaatgg

SEQ ID NO: 46 - DNA, 431 bp, Homo sapiens
  AA935872
tgcatcttgagaagatcgtgtggttttggtcttcttttaatatggtgtattacattaattgattttataaagcatgttgaaccagtcttggattgctgggataaatcttacttggtcatggtgtataatccttttagtatgttgctgattctgtcaatggcttttatttctatagtcatatgaaatattgctgtacaatcttcttttctcgtaatgtattttacttgttttagtatcaggtaatactggccacatagaatgagttggtaaatatttctctcccattttttgctagagttaatgaaagattaatgcaaattcttctgtaaatttttggtagaattcaccagtgaagccatttgatcatgaggctgagtagctgggattacaggcagaatggcacttttatcattataaaatgttaattgttt

SEQ ID NO: 47 - DNA, 366 bp, Homo sapiens
  AA960982
caatcaacttagaaagtttattttgccaaggttgagcacacactcatgacatgatacagcctcaggaggttctgacaacacgtgctaaaggtggtcggtgcacagcttggttttatacattttggggagacatgagacatcaatcaatacgtgtaaggtgtacactggttcagtctggaaaggcgggacaactcgaagtgggggcttccaggtcataggtagataagagacaaaaggctgcatttttgggggtctttgatcagcctttcactgaatacacaatttacatggagtggggaataaaggaatagtcacactgatgccttagtctggctcagtggatctgcatttttgcataaatagggc

SEQ ID NO: 48 - DNA, 432 bp, Homo sapiens
  AA992381
tttttcttttcgagagctgtttatttttctttatcagccaaaaagtatgattactatgttaatgcaggttttgcttcatcaagagattttccagttattgaacttaaataacattgttctaaaccccaaatcctctaaattcctgttgtcagtgaaagaggtaccttctaatttggaaagaatatctagttcagttctgatttcaaaccagcagagaaaaataagaaaaatgttgctcctgccatatggagaatgaccatcatttaaggaaagaaaagttggttgagagtttttgggaagtaactgttttgtacttcctttaatgtactcagtgtccagagcaggaactgggtggttagaacagctgcaatatgattccactggaaagcagtttgcagagaggcaatcaaaagctgcaacacttagattaag

SEQ ID NO: 49 - DNA, 361 bp, Homo sapiens
  AA995114
ttttgatgtttaacctcattagtatgtaaagtttattacataatatctgacactaagtgttcaatagtgtcagctatgatttttgtagaaagtgctcaataagttatcatcatcatcactaatatcatgaacgctgttctttgatgcttttctcttccctccatccatcttcatcatggcaattccacatggtctgtattcactcctctcttctcttcctaataaaatttctcctttagcaagagcacccatcgcaatagcatcaagacactgctctgcccgtcattcccctttgttaactcatgctatggtagtatgccactgttaaattattgatgatgaattggttgatcccccttga

SEQ ID NO: 50 - DNA, 3558 bp, Homo sapiens
  AB007950
cagaagccgaaagaactgttcacatggagctgtttattttccggcctgaggttgccgagacaattggcgagctgtcttgaatatatctctatcaattaaaacagcagctgagataaataatgcacctttgccggaactgccacagggactgcaggctcaggcttctcaagccagctcaccgtccagctgagcgagatgtcagcccaaggaaggaacttagatgccttggaaattgatgcctcacagttattttctccagaggaggtgcagggtctgggctagggaaacggaaaggactctgttgcatttaataaagcctgtatcctatggcagcagccactaaggagctcaccagaataagccaatgccattcctcatttggcctgagcagctcagagtcaggaagtcagagcgcagaaaatccagcagctgtcagagggctccatgtttggccacggtctgaagcacctgttccacagccgccgtcggtctcgggaaagggagcaccagacgtctcaggattcccagcagcatcagcagcagcagggtatgtccgaccatgactccccagatgagaaggagcgctctccggagatgcatcgcgtctcctacgccatgtccctgcacgacctgcccgcccggcccaccgccttcaaccgcgtgctgcagcagatccgctcccggccctccatcaagcggggcgccagcctgcacagcagcagtgggggcggcagcagcgggagcagcagccggcgcaccaagagtagctccctggagccccagcgtggcagccctcacctgctgcgcaaggccccccaggacagcagcctggccgccatcctgcaccagcaccagtgccgtccccgctcttcctccaccaccgacactgctctgctgctggccgacggcagcaacgtgtacctcctggctgaggaggccgaaggcatcggggacaaggtcgataagggagacctggtggccctgagcctccccgccggccatggtgacaccgacggccccatcagcctggacgtgcccgatggggcaccggacccccagcggaccaaggccgccattgaccacctgcaccagaagatcctgaagatcaccgagcagatcaagattgagcaggaggctcgcgacgacaatgtggcggagtatctgaaactggccaacaacgcggacaagcagcaggtgtcacgcatcaagcaagtgttcgagaagaagaaccagaagtcagcccagaccatcgcccagctgcacaagaagctggagcactaccgccggcgcctgaaggagattgagcagaacgggccctcgcggcagcccaaggacgtgctgcgggacatgcagcaggggctgaaggacgtgggcgccaacgtgcgcgcaggcatcagcggctttgggggtggcgtggtggagggcgtcaagggcagcctctctggcctctcacaggccacccacaccgccgtggtgtccaagccccgggagtttgccagcctcatccgcaacaagtttggcagtgctgacaacatcgcccacctgaaggaccccctggaagatgggccccctgaggaggcagcccgggcactgagcggcagtgccacactcgtctccagccccaagtatggcagcgatgatgagtgctccagcgccagcgccagctcagccggggcaggcagcaactctggggctgggcctggtggggcgctggggagccctaagtccaatgcactgtatggtgctcctggaaacctggatgctctgctggaagagctacgggagatcaaggagggacagtctcacctggaggactccatggaagacctgaagactcagctgcagagggactacacctacatgacccagtgcctgcaggaggagcgctacaggtatgagcggctggaggagcagctcaacgacctgactgagcttcatcagaacgagatgacgaacctgaagcaggagctggccagcatggaggagaaggtggcctaccagtcctatgagagggcacgggacatccaggaggccgtggagtcctgcctgacccgggtcaccaagctggagctgcagcagcaacagcagcaggtggtacagctggagggcgtggagaatgccaacgcgcgggcgctgctgggcaagttcatcaacgtgatcctggcgctcatggccgtgctgctggtgttcgtgtccaccatcgccaacttcatcacgcccctcatgaagacacgcctgcgcatcaccagcaccaccctcctggtcctcgtcctgttcctcctctggaagcactgggactccctcacctacctcctggagcacgtgttgctgcccagctgagtggccagccacaccaaccctgtgctctctggcccccagctggccacacttctccaggagggacccttggacttctttgtgtgtccagtttggcctcctgcccaaactgtccattccagcagctcctgcccccttctctgtacttgcttctgtctgacaccttctccctgttggcctgaagggagcttagaatgcagccctacctggagatagtgcgggcacctgtggccaagtggagcagaggtggacatggggttggattgttttgattatttatagttacacaaggacttctcccagctgaccctcaggatgccccaagtcaggaagaccattaagaataggaggagagggctctgcctcaactttcctaggaaagagcccacctcggagatagctacggtttcctctggtggagatggtgaggatgaaggctggagagtgagggaggaggctctgctggccgcagagaacacagggatgggagggtccctagccttcgggcacctccagggccagagagcaggctcagagcagctagtgtggagctcagcatccccaccccacccctcctccctgtagagctgatttgaggcctccttctggggctgggctctgcaggccaggtgggtgtggcctgtgttttcccttctgttctttctgcctgtactggatctgttattttcagggaaacaggccccagggcccccctgagcctcaccctaagcccttaggcctctgagagtgctgttgggttctatttatttatttatttgttcctttgttccctacccgtgcccccagtgtcttccctgctgagtaccaggagaggtcctgccccatcctctctctgaagccagggcccttccattccatttagcctttggatcatcctggctgggagaagtgggaccgagccacccagccccactatccccaagcagccctacagccgggatgggaggcacgtggcctctcttttatccgtctatttattttgtaagtgtattcgtgtggaggaggttgttgctttatttttttaaggctctggagtgttgtgtatggtttcttttcacatcccagcctcccatgggcacttctaagaagagaggggatttcttggaaaaggagagaggaatcccctagagcagggaaagcagtgcctgccagctgttgtgcaccttcctgagaaataaatatcctctaaattttcaaacc

SEQ ID NO: 51 - DNA, 486 bp, Homo sapiens
  AF458059
atgtaccaggtggttgcattcttggcaatggtcatgggaacccacacctacagccactggcccagctgctgccccagcaaagggcaggacacctctgaggagctgctgaggtggagcactgtgcctgtgcctcccctagagcctgctaggcccaaccgccacccagagtcctgtagggccagtgaagatggacccctcaacagcagggccatctccccctggagatatgagttggacagagacttgaaccggctcccccaggacctgtaccacgcccgttgcctgtgcccgcactgcgtcagcctacagacaggctcccacatggacccccggggcaactcggagctgctctaccacaaccagactgtcttctaccggcggccatgccatggcgagaagggcacccacaagggctactgcctggagcgcaggctgtaccgtgtttccttggcttgtgtgtgtgtgcggccccgggtcatggcttag

SEQ ID NO: 52 - DNA, 391 bp, Homo sapiens
  AI016806
tttgcctgtggtgagttgtttattttgttgaagtttgcggtgggcccgggggacgtggggggcgagcggcaggcagcggccgggactgttttgtttaggtctccatccacctgtgtggtataaatctgccagcctcaaggcggcgggggaccggagtgagaaagctctgacgctcccaaaagccccttttcgcaacttacagatctaggaccacatttgattctccacgcctggaaccggctagatcctagtttacgctcaccccgccctccagcctcccatcaacgcttcccgcacccgctgtgtgcagggctggcgctccgcagagcaaacagcgcccacgacccagggacagctggccaccgaagtccctccccggcgcccttccccg

SEQ ID NO: 53 - DNA, 478 bp, Homo sapiens
  misc_feature (459)..(459): n is a, c, g or t.
tttcctggctgccttccttgtcggttgactttcccagataatcttgaacattccctttcccttcacacttgccagaagcattacccactgtagagaaaacagggataacaaatccagttttccatttatgaactgggtcctcagctctgctgcatccccttgatgggaaaccatcacctgtaacaatctatatgcccttgactaatgatgtgggcacagctccgacctgtggcctccccccattcaagagaacagtctggaaagtagaagtagccgctgcatcgaagtagaggcttcagtggcctgacttttcccacttcctgctcccctgtgagctgaacgtcagtccaaggatgcggtcttgggctgcccatggtgggagacagggcagtttgggggaaggaagttccctttattgctttttctgcatggccctaaaagaatgcagatgagcatccncacctcggcaaaaaggctg

SEQ ID NO: 54 - DNA, 415 bp, Homo sapiens
  AI033361
tgagacaaggtcactctgtcgccggaggagatggcatgatcataggtcactgtaacctggaactcctgggttcaggcaatcttcccgcttcagcttcctgagtagctggaattacaggtgtgtgctaccattctggcagttttttttttttagagactatatctctatgggatataaatttctctctaagcaactcactttattccttcaagtgttaataactgtattttcactattattcacttataaataattcacttttgtatttaaaaagaataagtattctttcttgtttgggagtatagagttccatataatgcctattcaaatttgctgattatattgtttaaatcttttaaatcaaaggtattttatctacttattctattagttattgatagtatgttgaaatatt

SEQ ID NO: 55 - DNA, 336 bp, Homo sapiens
  AI039866
ttttttttccgagcttgtgctaagcctttattgagcactactgagggtcaagctctgggcttgactttgtaatcccaagaggggcaaaaaccggctgctttcacgacactcaggaaaacacgggcggagaaagctcactgatcacttggacctaccaagcgagcgccccctggagaaaacgctgggatcaacacgcatttgccaagcccaaacggtatagggggcactgctggaaaggccgggaaccttccctcttaacctcccacccaaacccatccctcttttgggaacttggcaggcaatgaggagtagtggccctcttaaaatttttaaaaa

SEQ ID NO: 56 - DNA, 480 bp, Homo sapiens
  AI091302
tttaagtggcccacttatttaatgagtattggataaaaacaaaattctagtacaactgaaggctgtaaatcctgagagttggttataataggatctggcctggatttttaaatggcacaccattcaaaaagtggttgggaacataaaaggttctccacaaaagccttttgctgcagaaggtggcttggatggctaaagtagtatttactcttagcaattaaccaccaaagcatttagttatttgcatcccagcaaaacaacaatcccacactttactccatcacacctggaatccaagaggtctacaagtccaaagacaactgtgttatcagctttgcatgggaaagacaggaggtagctaaactgacctgtaggtgccctttcagtgtggctagggtcatctgagcagtcagggagtctctcattcatcaacagtttagcctcctttagtgttgattatttgacttatctgcaaaatgg

SEQ ID NO: 57 - DNA, 489 bp, Homo sapiens
  AI142427
cttctccccaaggcacaaatatttctggtttaacttatttttgtgatgtacccaactaaccacaaagccaggcctgctgggatgaggataaaaatcaaataaacaatcctctcaacgtccaggaaatggctgccaggccttctgacttcatgaaacgccggggccagctgtgctcttctatgctgggtcctcttcgtccattgccacattgctgtttgtattcttcttctgctcagcctctggctctgcaggcagaatgctggtgacggtgtggagaagagcttcaatctgctcctccgtgagccgctcttcactctcttccaccatcagctggatctccacagcagtcacaggccggtggttcagcagctggagcttctcagctttggtcaacttgtggcttttcaatgctgtgagaaattctctgacaatttcaggactctggtgcctgcatggtgtttttgatatgtattttaacgtttcataggt

SEQ ID NO: 58 - DNA, 428 bp, Homo sapiens
  AI147932
aaattcagatgaaaattttattcactaattctgcatttcacttgagatagccacaaacaaccaacccttccatctgcagaaatgggtaataaaagcacaggtgtcacattaacctttttggctgagattaggtgccattggttttagattccagctttgaggagtctgtctcaataaaaatgtgccgtgttcttgatatgtgccaggctctgtgcaaggttctgggacagacagtgaaatgcactgacagacaaggtctgtcctcagggggctgagagtctagttcctgccctgctatcatgtacagataatcagtaaataggtgctgtgggggatgggagaggttgagagagcttagggtgcatttctaatggagctcacatgattgtcggccaaattcatttctgtgaagctgcagaactcacagc

SEQ ID NO: 59 - DNA, 502 bp, Homo sapiens
  AI148036
tgggagaataatgtcagtttactgaatgaatacatggctcgagtccattcagggtggagtgaggcctggcaggtgccctcagcagtgggaccccaggccactggacctggcagggcttctgggctaagggggcagcaccaggccagcctgccaggggctccacagtcccacgctgcttgacctgtgctgtctgggactagactggtcgctgtggctgcccatagatgctcaagccagaccccaaaagagagtggcacaaggtgaaaatctaactgtgaacgctttggtttcagtttcagtaaagaggaggagggagggaggcacttggggacgtagaaagaagggctggcgggagaggtctccgccgagctccacagttggcttccttgctagacgacctggctttgagaaatgctgccttcatttccatctcccaactccgaatctccggcaacccatattaacaaagtgctgccaaccaggcgagtcatgccaggatgcc

SEQ ID NO: 60 - DNA, 509 bp, Homo sapiens
  AI149817
tttttgcgacagggtctcactctgtcacccaggctggactgcagtgttgtgatcctaactcactgaagccgtaaccctctgggctcaggtgatcctcccatctcagcctgggcaacatagtgagacgctgccttgacaaagaatttaaaaattagccaggagtggtggctcatgaccttagtcccacctactcaggagactgcagaaggactgcttgagcacgggaggttgaggctgcagtgagccttgactaggccattgcactccagtctaggcactagagtgaaatcctatttaaaaaaaaaaatctgcacacttttacattagcaaggccatttctaggaaagtatcctgaggaaacatttcagattctaaggaaggactttcctgtaggtgtgctgtttagaagagctgaacgctgaaaactgtaatgcccacaagcagaggactgggtcaaaatgagcgtgctaaaaccatgccaaggaatgccacacagtggtccaaattac

SEQ ID NO: 61 - DNA, 388 bp, Homo sapiens
  AI184594
tttttctgagtcaacacatctttattaaacacctgaagttactgggaggaggccatgatgctggacacacctgtggggaaggagagaccagactgaaggtgagaagggcgggagacaaacctccagccacctgctacagcacccacaattctttagggggagatcataaactccttccaggccccagttaccatcacaaaggcagtttggtgcagtattttaaaaagatggactctggagcaaggagggctaggttcaaatcccagctctgccaattaacagctgggtcacttggacaagttactgaacatctctgagcatcacctaccttcctatctttaagaagcacctaccttataggattgtaggaccaaacgaggtgcttaga

SEQ ID NO: 62 - DNA, 472 bp, Homo sapiens
  AI185721
tggtaagactttattttttagtcagttttagattcacagcaaaattgagaggaatgtaccaagatttcccagatactccttttctccacacatgcataactttcccccattatcaatacccctaccaaagtagtacatttgttacaatcaatgagcccacaatgacatcattattacccaaagtctatagtttacattagagttcagttttgtattgtacattttacgggtttggacaaatgtgtaatgagctgtatccaccattatagtatcacatagaatagttccactgccttcaaagtcctctctgctctgtctattcgccacttcttcccccagccttctcgcaactagtgatcttattactgtctacatagttttttactttcccagaatgtcatatagttgatatcagccttccccaactttgttcttctccttcaatactgtgatagctacctattctaggtct

SEQ ID NO: 63 - DNA, 455 bp, Homo sapiens
  AI187401
gcgggagactgggagggagagggcctgtcggcggcgctgcgcgtggtggcgatggccaacagcgctctcaatcccttcgtctacctcttcttccaggcgggcgactgccggctccggcgacagctgcggaagcggctgggctctctgtgctgcgcgccgcagggaggcgcggaggacgaggaggggccccggggccaccaggcgctctaccgccaacgctggccccaccctcattatcaccatgctcggcgggaaccgctggacgagggcggcttgcgcccaccccctccgcgccccagacccctgccttgctcctgcgaaagtgccttctaggtgcttggtggtcagagacgggtcatctgtcgctaaggcgcaacctccagggaactcgaggcctgccagggtctgtccagatcacaaggggcaagagagtctgtgagagagtgacactggag

SEQ ID NO: 64 - DNA, 170 bp, Homo sapiens
  AI187962
cggaaattctcacttcttggagcactaaagggttgttagtctatctgggcaagcctcgtttccctggagaaaatggagtctcagagattagtgactggtcctagagctaatgagtgaccaaagtgtgaccagagcccaggtctgcttctcaacccagggcactttgattc

SEQ ID NO: 65 - DNA, 399 bp, Homo sapiens
  AI220247
tttggagtgacagcatttaacctttaatgtgggtctctcagccctgagtacagctgcagtctaagggtggggttggggggatctaccgttttttcctcttagtgtgtgctttggattagtaacctgatggaaagcacgctggagttggacggcctggtttgcatctgcttctaccacctttgttatttcggcagtcctcagtctcggactctccctctgtgaaatgggggaacatgaacttcctcagatcgtgacagcgcagctccacctggcacagggtgaatgcccacaggtggcagctgagtgacgcagggagggccggccagccctgcagcaactgaagcatccactggcacctcaagtgctggtcataggaccagacaccaccacaccagctag

SEQ ID NO: 66 - DNA, 472 bp, Homo sapiens
  misc_feature (433)..(433): n is a, c, g or t.
atctgcttgtattgattcaaaagaatggtctttgagttctgagattctttccttagcttggtctattctgatattaatgcttccaattacattatgaagttcctgtgctaatttttcatttcagaagttcagtttggttctctcttaaaatgaatatgtcctctctcaactcttgaaccattttactgttttctgtggattgagtttcaaccttctgtatctcaatgagcttccttgacattgagattctgaattctatgttatttcagtcatttcagtctggttgagaaccatggctggggagctagtgtgatcatttggaggtatgaagacactcttgctttcagagtggctggagttattgcactcgcactttctcacctgtgtaggctgatgttcctttaatctttaaagttattgtcttttgaatggngctntttgcctttatattcttcaccatcatgcccagact

SEQ ID NO: 67 - DNA, 337 bp, Homo sapiens
  AI263527
ttttttttttttttatttttagacaaagtcttgctctattgcccaggctgtagtgcagtggcacaatcatagctcactataaccctcgacctcccgggctcaagcaatcctcccacctcagcctcccgaatagctgggactacaggcatgcaccaccaagcctggctaatttgctatttttgtttttcatagagacagagtctggccatgttgcttaggcaggtttcgaattccttgcctcagcctctcaaggaatttgcattgtttttaatgaaaaaacacacatatggtgaacagtaaaagtgggagaattgaacagccctaaaatcaagtagtc

SEQ ID NO: 68 - DNA, 450 bp, Homo sapiens
  AI269981
attttaatcacatttatttcattttaagtgaagctaaacttttttttaacatatttgttgcctatttgtggttctgtccttttctcctttatatcttttactgattcttttttttttgagatggagtctcgaacaaacaaaaacaacaacaaaaacacacacacggactttccagagccctgcagttttgcgcttcagggacaacagtgaacatttatcgatcccatggttctcctacaattgccacgtggccagcgtctacacagccccctatcctgcccctgctgtcaaacacagcgtttccagaacgcagtcgtgctgggctccaggtgagctttgggaggagccccggctgagcccctgcactgggccccgcgcttcgggaagcatctcgctcgttcgtctcgcgcgcccgcgtgaccggagccctcacgctggcagcgggcaccg

SEQ ID NO: 69 - DNA, 425 bp, Homo sapiens
  AI270372
tttttttttttttttgcagggggagggccagcggcaggaaagccgctacaatttatttccgagtcccctccagaccaccaggctgagcctgtcacgacctcctcaagggaccagccattcctgtccccagtgctgctagccacaggcctgggatccggccagagtcagcccagccccacccctggagcgagcatgtgacgtgggctctgcatctgcatcgtccctgcggccacctggccttggaatctgctcctccaagacccagccaggctgggccagggcaggcttcccacacgagggcccgtcgacaggccctcagcgtccaggtggcgggatccagatgagaccagggcgggctcccaccagcctggccagttcctgccgacactgtccctgtagtcgacgtagcaccgcctccaggcagg

SEQ ID NO: 70 - DNA, 463 bp, Homo sapiens
  AI277856
ttcttcttctcctttttttccagactcatttgcaacaattctttgattttcttcattttcactggattatttaatacctctcgttttttctcctcctccttcttcctgatgatgaagagtgggtcctcccggatcttgctggccatgtcaagaagggaattggcacctgatggggcaaagatagagcctgggagaagtcctgtttcagaagagcagcctgcctccttctcctccatcttctcaaaaacatatttgtcaatggggcgccccagcaggtactcgtcacggttcaccatcccaccaggaccctggtacatccagtacaacttttcttcttttttcctggttttgacggccccaacatcctccgcatagcgctgcatctcttcccgggctctctcttctcgcagctcccgctgaagctcctcaatcttcttccgctcagcctcatgcttctgctcgg

SEQ ID NO: 71 - DNA, 396 bp, Homo sapiens
  AI289206
ttttttggcttaaacaatagaaatttattttctcctagttctggaggctggaggtcaaagatgaagtgctggcagtctggggttcttctgaggcctctcctccttggcttgcagatggtcgtccctttatgcacttgtgctcctgttgtctttcttatgacaccagacatattgaagtagggccccaccctaatgacatcattttaatgtaatcacctctttcgaaaccttatcttcaattatggttacatttggaggtactgggagttatggcttgaatatatgaatttgagcgtggggaccaggggtgcacaatttagtccatagcagaatcctaactgtaaaacaaacatggatccaataaaaagctaattttaaaaaatcacagttgctgct

SEQ ID NO: 72 - DNA, 424 bp, Homo sapiens
  AI342905
cttaccaagacgtaataatgtctgaaaagtttcagctttgctaagttaatagcagctagagagaagaaagaacaaaatgaagagctcgttaacttctcatttttaaaagtaagcctcattcagataccttctcagtcaagaggccacctctaatgcagatgaggaccactcagaggcacatccatctatctggaatcaagtccgctgggcggtctctcctccaatcaccaggaaaaaatgtgggagcagcacagccccagtgagccccatgagctggagttggcttgcacaggcgattcccgccccagggtcagcagctgtaacctggctatggtgggagtatactgacatcacaggaattggcagatgctacaaatcaggaattttttccccccagagagccagcattaaacatttcagctct

SEQ ID NO: 73 - DNA, 382 bp, Homo sapiens
  AI343613
ttttttaaagattttcctagcattttattaatagcatccgccagaaaaggtagaaataaacaaggagtcagggagcatagggagtccacacatgattcactttaaatgttctcattcagcacagttcctcttcagctcccacagggatcggcgctggtctggtgggacgctgggggccggtacgtggagcacatgctttctgctgcagagacctctggaagaggccccggtgagcacaggtgaagacaggcctgtccctgtgcacacagtgtggctggaccagctgctcttgtcctgacctctgcatgctcatagttctgcaggctgtacagaagcctcaggaaacttacaatcctggcaaagggtgaaggggaagcaagcc

SEQ ID NO: 74 - DNA, 439 bp, Homo sapiens
  misc_feature (436)..(436): n is a, c, g or t.
ccatttttttttactgtgataaaatatgattaccattttagccatggttaagtgtacagttcagtggcatgaaacacattaatgtttagttttaataaaatatttgttagcttcattttttttttacatttcacatacatgaaataagccaatcacaaaaagataaatactacacaattccacttaaatgaggttatctacagtgatcaaattcttagaaacaaaaagtagaatggtggttactccggactggaggaagcaggaaatgagaaattgttcagtggatacagattgagtatcctctctgaagatttgtatatttaaaatttttttctttatctgattgagttgatttgaagaatcagtttttgagctctgagattccacagcttgatctattcctctgttaatgcttccaacactattatgaaaattntgg

SEQ ID NO: 75 - DNA, 483 bp, Homo sapiens
  AI377802
ttttttttaaggattataaacagaattttattataatggaacagatggaacaaaaaagatgagacttttagcaaagagggaatgatcactaataaactgctgattttattagaaagtttcaaaagaagaaaaaaatgaaattgtaatttaatgaataaacattacatgtacaatataccatttggaggttacttttgtttggtgagtctacaaataaacttcattgctattgatattcatctgttaataaaattctcttttgattatatttaaattacatatctataagtagacctcaaaagtaaaacaaacaaaaaccccaaaagctttctactctatggtaagggattgattaccctttgttgtttagtaactcctggtttttaccatttgggaaaactgggggaaaaaaaggagacagaaaggcaaaggcagaaatcacgagagatataataataagagatgaagacagaaacagagata

SEQ ID NO: 76 - DNA, 301 bp, Homo sapiens
  AI400066
ggctttcaattatagcgacttgttttcttgtttatttaatctttgtgttgtgctcgttgcctgattttgattgttggggctcacactagggactgggaaagctcctgaggagagaattgctcctgtcctttcctatatcacagggcattgctggcctcgagggtccctctccatgtggggtgctgggttcaccttttcactctgctgctagccaagctcacagcagccctcaggagccctgtctctcccagctgctcatgagcttgtttctttggcctgacaccacctccttgctccagcc

SEQ ID NO: 77 - DNA, 501 bp, Homo sapiens
  AI421397
tcttcgcttggaagaaagcgtgagatcacgctttgttttcttttgttgtttgttttgttttttttttctgagggaagccttagcttaatgtatgccggtccttcaaaagacatgatagcaatttgagagtctgaactgtaagtttttgttactttattcgttcaccaaatgtttttatggaagccttgccacgtggaacatgtgtcccgggttttaaaaaatgaatttgactcaccagctaacactgaaaaaataatgtacagcctagaaggaaagatcaaagtgtaaaagtggttagtcctgtcgcctcctgagcatttttgtattatatcacttggtttttcgtctgaagtggctgtaaataatcgtattaaaaatcttctggtccagacgcaggaaaaccttttccggtataaagcccaccttcctctaatgttgcgcatgcccactgtgcccagctgctccgctttcgtgcatcccagacttctgtcaccaggtaag

SEQ ID NO: 78 - DNA, 436 bp, Homo sapiens
  AI435179
ttttttttttccagctataaccttttgtttattaaagaattgatgaaaataaaaacaacgtatctcacagcatgtgtgataatatttccatacgtatgtgataatggatcaacgtttcctagagatgaaataaaaagttctaatttggacaaaagcaaacaaggaaaattataccgtagaaaagcccggctgacggaagtgaggccctgtctcagggagaaacatcggagacgtttaaaagcagggagtgaaacagaggatagcgtgccatttttaatactggcccttgtttcagtgggaaaaggccaaaataagccgtgcatatcctggattctcgcatcgatcgttcaggatcggagatgtgttctccatttccagttacgcattgtatggtggaattgcagttagcacatttggtgccaaaattgtaatgctg

SEQ ID NO: 79 - DNA, 206 bp, Homo sapiens
  AI473446
aggtggattactaacagtagaagtcatctggggatgacctagtaattgaaaatctgactgatctatcccagccattgtggcaagctgcccaagcccagcatctttaaggagatccaagaaagcatgaaacttctgaaaagaattctcaatgctgcccaaaacaccttcatcatcaggatcatatttgtcatgactgtgcatgaagg

SEQ ID NO: 80 - DNA, 330 bp, Homo sapiens
  AI475085
cagctctcaagcgtgtgtttaattatcttcactgcttactaaggcatgagcataaacttgtaaatatttacaaagactttaaacaagagattgcacttaatattcccgtgagaaagcgtgtccttgagggttttgctggtgaaggcacccacctgtgtgcgtgcacaccgctccgctccacctgtgtaaccaggcgtctctctgcaggcctccccaccgtctgcatcggtgcatggactgcggccaggctctacttagaagacaccgggtgagtccatgaccagactgcaacctcgaaaacgggcccggcaacatctggcacatgaagtc

SEQ ID NO: 81 - DNA, 462 bp, Homo sapiens
  misc_feature (288)..(288): n is a, c, g or t.
gcggccgcctctgctctcagtgccccggatcggaggccgtccatcgcccctcgggccgacgccatgaagatcaaagatgccaagaaaccctctttcccatggtttggcatggacattgggggaactctagtaaagctctcgtactttgaacctattgatatcacagcagaggaagagcaagaagaagttgagagtttaaaaagtattcggaaatatttgacttctaacgtggcatatggatccaccggcattcgggatgtacaccttgaactgaaagatttaacactntttggccgaagagggaacttgcactttatcaggtttccaacccaggacctgcctacttttatccaaatgggaagagataaaaacttctcaacattgcagacggtgctatgtgctacaggaggtggtgcttacaagtttgaaaaagattttcgcacaattggaaacctccacctg

SEQ ID NO: 82 - DNA, 458 bp, Homo sapiens
  misc_feature (446)..(446): n is a, c, g or t.
cgggcgccgggccttagtgctggcgcgggcccgggagagcgccgagctgccgctgcccgccggctgggaggaggcgcgagactacgacggtcgcgtcttttacattgaccacaacacgcgccagacgtcgtggatcgacccccgcgaccggataacaaagccattgacctttgccgattgtgttggggacgaacttcctttaggatgggaaaccgtatatgataaacaaattggagtttattacatggaccacataaataaacttacccagattgaggatccaagagaacagtggaggcgagagcaagagcgcatgttgaaggaatatttaattgtagctcaggaggctctcaatgccaagaaagaaatctaccagattaagcagcagcggttcgagctggcccaggaggaataccagcagctgcacaaaatgtgtgaggatgacnagccgtcgtacg

SEQ ID NO: 83 - DNA, 468 bp, Homo sapiens
  AI539457
tccattttcaacttttattttaggtttggaggtacatgtgcaggtttgttacttgggtagattgcatgtcattggggtctggagtacaaatgattccttcacccaggtcgtgagcacagtactcaataggtagtttttcagcccttgccttcctcctaaggtactactaaactaaaacaaaataacaagaaataaaatttttaaaaatcgaagtcttagtgaagaagatatcttgtgggctttctagaggaatgtagtcgggaatgagtgtgcagttagcatgatacagccacttcaacattcctatttgtctgtcttcagattccctcctcctcctcatgccttggaagctgtggcatctcaacctcattaaatacgggcagtagtgtgctcatgccagctcactccagctcacaagagcatggtgttaaatttgcagaagtttcatgagccatttgttgaatacag

SEQ ID NO: 84 - DNA, 284 bp, Homo sapiens
  AI560533
ttcagctattttttttttattaaaattgagtgcctatacctatagagaatcaaggtggtagatacaaaggtgttcactatacaatttcttcaatgtttcagtatttttgaaatttttcacaacaaaatgttaggggagaagcaagtcctgcactccaaccctcctcccccacagtcccagctgcacacactccttcaccacagtggctgtttcagaactccacctccacctgggcttcctcggtaactgccagctctgctcacattagaaggctctttgtagcc

SEQ ID NO: 85 - DNA, 229 bp, Homo sapiens
  misc_feature (208)..(208): n is a, c, g or t.
ttttaaaccaaccaaacatatgtcttattttggatgttctatatgttcttacattctctcaggtcttttgtgtcattatgaacacaattctaacaagcttgattattttatttccattcacatattacaggcaacaagctgaaaaagtagaacggggtgtagagagacaggaccaaagtacagataagggcttggaagtgcccctganccagtcgacagcaaccacatg

SEQ ID NO: 86 - DNA, 341 bp, Homo sapiens
  AI579907
ttttttttttttttttttttttttttttttttttttttttttttttagagccaattgatttaagggaataagacacagtcattgaaaggctaaaaaaaacaatagcaacacaaaaaccacttgggagggaacattaaggggagcagcgggctaggccttttgggataatatcccgccctgaattctttaacactactggtaatttagggtcaaacggggttctggtctgaccataaaggtttctacattggatgaaaaagggtggctcaaacccaaagggaatttcttaatagcaaatctcatttattactaatcatttctttaatttagcatacccattt

SEQ ID NO: 87 - DNA, 329 bp, Homo sapiens
  AI583425
tttttttttttttttttttttgtagagatgggattttgccatgttgcccaggctggtctcgaactcctgagctcatgcgatctgactgcctcagctttccaaagtgctgggattacaggtgcgaaccactgcactcggcctcgtggctttaattcacatttctctgatgataagtgatactcacacttacatttctgtgtataaactttacattcagcaggtgcagaataatggaacgcttaagaaaaccgaaatttccttctttgaagactaagaagcaagaaagcaccttgcaagactgattttttaaacacgagagaaaaaaatac

SEQ ID NO: 88 - DNA, 398 bp, Homo sapiens
  AI591136
agtgggggaagggtgagaggagcttttattagcaaaaagagattacataagctgcttggaaacagagttcatcacttccagaggttcaaagccagagttgtcagttccttggtggagatgcaagtgttctttcaagaacatcttaaccgaattactgaaatcctaaagaatgtctagtgataaaccttatcaaagcaggagatttgtgaaggatatgaaaggatttttagaaagtccttggaaacagttcttatttcagatatgcaagcatgagcctcctctccttcagaccttcctggtcctactttgcctgggtctgagaaaagtgatttcttcctggtatctctaactttcacaagtctatgataaaattggttttgttaaaaaccaatttaact

SEQ ID NO: 89 - DNA, 368 bp, Homo sapiens
  misc_feature (164)..(164): n is a, c, g or t.
caaagcattaatattttaattataccacattcaaacggcacctaagttaacccacaaaacgccaagtggcaagttgcttcaaagggcccggaagagaggacagaagctggctcttcctgctttcctgccccctacccactgggtctgggttgctcaggacccanagactggagacagcctgaggctggaatgaaggttttcagacttccttcctgtgcaaccacgggcaaggacatcaagagagaactccctgcctgccacatcttcgttgaagtcaattgctatggccttctaagatctcctcaaaagccctttcctacagagagtggatgacccaccctccaggagcggggtgaacactcaagggg

SEQ ID NO: 90 - DNA, 207 bp, Homo sapiens
  AI612918
gatattgtgaacacaattttctttatttcatttttggagttttctgaacagaaaaatacaattgattttctgtatattgatctagcctgtgaccttgctgaacttgattaattctattacactatgattttttgttggtggttagacccttacacaatcaaatgaggttaaaaaaaattgtccagagtggccccagaccaacaacag

SEQ ID NO: 91 - DNA, 391 bp, Homo sapiens
  AI620645
ttttttttttttttttttttttttttttttttttttttttttttcccagggggaactttatttcagcgaatagaggacaaaaatcagcgccggctcctgggggattcgggaaatttgccacccccgtaggcataaaaaaggggaaaaggggacccacttttttcccctttggggccaccctggcaatttgggtgcaaagcccaggaattccacacaaggaatttggcgtaataaaagtaattagggctgggcgtgctgcctcatgcctgtaattccacccctttgggaggccaatgcaggaggattgcttgaccccaggagttgaaaaccagcctgggtcccatagcaggatcctatctttcccaaaacaaaacaaaacaaaacaaattag

SEQ ID NO: 92 - DNA, 223 bp, Homo sapiens
  AI631076
tttttttttttttttttttttttttttttttttttttttttttttttttttttttattgggaaaggtttatttaaaggtttgggggccccttttacccccccccaaaaagaaaaaaaaaaattttttttttgccgtcccaaacagggaagtcccttcaaagaggagctggcgggtgggttcaaaccccaaaagggggacccctgccagggggtttttcccggc

SEQ ID NO: 93 - DNA, 458 bp, Homo sapiens
  AI635650
ttctcagcaaggcaatttacttctgcagaagggtgctgcctgcatcaatcatgatcacaggagcacactgaacaaaggaaggaaaaggtttttatccctaacacagtccctacctctgtgtcactcccccaggggctggggttggaccgctcattctaagttgacccgattggctatttgtgaatacttttccaaataaggaagggaagggggatgtgagttacagtggtgggaagtgcagtttcagtgggaagaatgggtgcagagtgggtaaccaagggagcagatgtgagttattgattagaactggtgggaaggttgttcacagtaactagggacaaggaggcatggacaacaaggaagtcgagtttgagaacaaagaataaggaagtcaacaggctaaatctttgaagaggaatttactgtatcttataatttccccctttcaattgttatgg

SEQ ID NO: 94 - DNA, 449 bp, Homo sapiens
  AI655693
aagagtgaaagcttttagtttcctagttaagacttttcttgagaatggattcactggtgcagacataattttgtagtacaatgagatttttgttacagagctcaagttgttagaacttctatctactagtggtgtctgatataacctgggctctccagtgttacagtgggcttcccaatctcatcacaatcatggggttgaggagctgccatgactataactaaagactttcccttatatcatacacacaaaacatttctccatcaggtatgttatccctgaaaacttcaaacaactgctacaatgacaacactgtttaagtcaaaattctgttccaaggcaagaaaacctaattcaggtgtgccaaagccaaaaccaaccaaacaaaaatggaatttattggctgctgcaattcaaaaacccgtatatatttttagcttcaggcatgg

SEQ ID NO: 95 - DNA, 450 bp, Homo sapiens
  misc_feature (324)..(324): n is a, c, g or t.
tttttttttttttttttgtgttttaagctacatttattatagaagagtcaaaatgtttaaaaaaacaaaattataaagtaaaaagttacagtaagctaaggtaaatttattattaaagaaaaagaaatattttcaataattgtagggtattctaagtgtacagtatttataaagtctgcagtagtatacaatagtgttttaggccttcacgttcattcaccactaattgactcacccacagcaactttttggcctgaaaactccattcatggtaatttccctataagggaccattttttatcttttacactggatttttgcagnatcttctctatatttagagacacaaatacttaccattgngttacagttgcctacactattcagtacagtaacctgccatacaggtttgtagcctgaggacaaaaggctccaccatggagccttagg

SEQ ID NO: 96 - DNA, 564 bp, Homo sapiens
  misc_feature (549)..(549): n is a, c, g or t.
taaaggcgagttcgggtattttattttgccttaagagtcaacacaatttctaacattctgccattagaagtaatacaatttctaatatcctgcaatgagcattgctaaaagagtcaggagaaccatgagaaatatgcatatgaaaataaaccatgtgacaggggacatagaatccaaagagattccagcagagaatcaaagtgcacgctcacctactctgacctctccccataaaagatgagagtcatatacaaatagatgcttgtattctgtacagtcctatttaaaaatgcaccacataaaataagaataagacaggaacacactcatgaccacataaaatacagagaatgtgagacgtgcttcttgctgagatacacaacaatcaaatttagtatcaaagtgagctggactgataagaggctaagacacaactgaggatcgatggctgaaagaaagctatagacaaagaaaaaaatgttcactcgctgttgaagttataccaagaaccatgaactgtcatcagtctcatatattagcctttcattntgactntcagcagat

SEQ ID NO: 97 - DNA, 434 bp, Homo sapiens
  AI679923
gttttgtttttgagatggagtctcgctctgccacccaggttggagtagagtggtgcacgcaatctcggctcactgcaacctccgcctcccaggttcaagtgattcttctgcctcagcctcccgagtagctgggattacaggcgtgtagcaccacgcccagctaatttttgtatttttagtagagacggggtttcaccatgttggtcaggctggtctcgaactcctgacctcatgatctacccgctttggcctcccaaagtgctgggattataggtgtaagccacagcgcccagccagggcccatgtttttaaattttttaaggagcttgttgaggtctgcaaaagcttctgctaaacctttcactgtgaattttcttggaggttcttcctttttgtctcctgcagtttccttttctcttgcaccttcttcagct

SEQ ID NO: 98 - DNA, 445 bp, Homo sapiens
  misc_feature (428)..(428): n is a, c, g or t.
ttatttcattatttttaaatatattaatgaaatgggaaatagataaaattcattttaaaaaacaagcctacaggtgaggccaggcagggtgcctcacccctgtagaccaagcacttttggagcctaggcaagtggatcgcctgagcccaggagttcgcgatcagactgggcaaaaaggtaaaaccctctctacagaaaatacaaaagttatccagttgtggtggtgcatacctgtatttccagctactctggaggctgaggtgggcagagtgcttgagcccaggaagcagaggttgcaatgagctgagattgtgccacggtactctagcctgggagacagagcgagactttgtctcaaaacaaaaaaaatgcaaaacataaaccactgactcaaaagttataaccacttattaaaataataaacaatntcaaatactaaccatac

SEQ ID NO: 99 - DNA, 467 bp, Homo sapiens
  AI697430
taaagacagagtctcattctgtcacccaggctggagcccagtggcaggatctcagctcagtgcaatctccacctcccgggttcaagcgattctcatgtctcagcctcctgagcagctgggaccacaggtgcccaccatcacgcccggccaatttttgtattcttagtagactgggttccgtcacgctggccaggctggtctcgaactcctgacctcaggtgatccgcccaccttggcctcccaaaatgctgggattacaggtgtgagccactgcgcccggcctattgtgatatttctttacttaataactagcagatgaatctcttaacagtattctgttttcctctaagcatgcataaaaagtggtcaaataagtccatgatgactcatcaccttttccctgcaaggagtgtactgtagattaccaaacctgcagaagccgtctgtgtggagacaggaggaggaca

SEQ ID NO: 100 - DNA, 612 bp, Homo sapiens
  AI732314
acacttgttttttctttattaaaaataaaaaattataataacatatggcctatgaaaatatggtataggtatacacagagaacactttcatagaacagcaaagggctacttccccaatcctacaaaaggaaggatcatgaacaatttgctaaagaagagaaagtcttacctaaatcataatcggaaagtagaaattatcctactggaaatgaagaggtagaggaaggttaatcaaccttggcaatattgacatttgggccagataattctttgttatggaagggtgacattcttggactctatgcactatgtgccagtagcacccctccccaagttatgacaaacaaaaatgtctgcagatgttgccaactgtctcttcaagggacaaaattgtccctggttgaaaatcactggggtagagttttataggtagaggaaaaaattgtaaccaaaggcaccaggtatggaacagcatgatatttctgaaaactacacatagttctatattactcaagtacaaagtgcaatgccgggcataatgggagatagcaatgacaaatatggaagcctgataatggaacatctggtcgggattgcagatgttatggaatg

SEQ ID NO: 101 - DNA, 572 bp, Homo sapiens
  misc_feature (368)..(368): n is a, c, g or t.
tttttttttttaagaatatatctgcattcccatgtttattgaaacactagtcacaatagccaagatttggaagcaacctaagtgtccaccaacagacaaatcaataaggaaaatgtagtatatatacacaatgaataattatttatccataaaaaagaatgggattctgtcatttgtaacaacatggctgaaactggaggtcatattgttaagtgaaataagccaggcatagaaaggcaaacgttgcatgttttcacctatttctgggagctaaaagttaaaacaattgaattcatagagactaaaaggatggataccggggcctgggaagggtagtgggggttgggagaagtgggatggttaatggnaacaaaaatataattagacagaatgaataagttctagtatttgatatcacaacagtgtgactgtagtcaacaataatatattgtacattttaaaataactaaaagagtgtaattggattatctgtaacacaggaaaggataaatgcttgaggtgatgtataccccagttaccttcatgtgatcattaggcattgcatacctgta

SEQ ID NO: 102 - DNA, 445 bp, Homo sapiens
  AI732958
ttttttttttgagacaaggtctcactctgttacccgggccggaatggagtggctcaatcatagctcactgaagcttcaaattcctgggctcaaacgatactcctgcctcagcctcccaagtagctgggaccacaggcatgagccaccatgcgtggccaatttatgtatttatttatttttaagaaatggggtttcaccatgctgcccaggctgatcctgaactcctggcctcaagcaatcctcccgcctcagcctcccaaagtgttgggatgacagatgtgagccaccacactcacaattctttttttttaaattatttcttgtatctgtccctttcttttccacccccattgctacttcttcagttgagacctcacaaatgtcacctggatcactgtaaaacattaaacttgatcacttgatcactagttcccactttagaata

SEQ ID NO: 103 - DNA, 592 bp, Homo sapiens
  AI741506
ttttttttttttttgtagatacaggatctccttatgttgcccaggctagacttgaactcctgggctcaagggatcctcctgccttggcctccaaaagtgctgggattataggtgtaaaccagtgtgcctagcctacagttttttaattttataaaatgttatttctaatttttctccaaaagtaaaagtggcattccaatggcaatattaattcaggtatccagaactcttaacctaaatttgggtgagatgaggaaaagtgtattgttaattttatgtgtcaacttggaaggtgtttctggatgggaataacattgacactgatgtcctctgagtaaagaagattgccctccatgatgtatgtgcactttatccaatcaatagaagacctgagtagaatgaaaagaccaccttctccaagccagaggaaattctccagcagacttctgtgagactggaactgcaccatcagctctcttgggtcttgagcctgccagaccacattgcagattcttggacttcccagttgccataatcacatatgccaattccttataataaatgtgtgtgtgtgtgtgcacgcacgtgtgtt

SEQ ID NO: 104 - DNA, 491 bp, Homo sapiens
  AI744597
tttttttttttttttattcttactcaatgccaacatttattcagggtgcatggtggaccaggcatggggctaagcaccttatactcaatatcccatttactcctcacatcatgcctatgaaccatgcattatcattatccgcattttacagatgaagaaactgattccggagcctgagcgcttaaccactgcaacacactggctcgtagtatagtgttcagggtggggcacaaagaaagcatgagctctctgccttcatctttgccatacaacatccacattgattcactagaggaaggaatcaatatttcatcgttttgcagccgtggagcacaaatgagggtagaaaacggagacacaaagttattaattgtgatctagatgctgaacaccgtattcagggccctcgtctctccaacattcctcgttctcctctgttccctctcagtccacctgctgtacctgaggatcccttgaaacatgacagtcca

SEQ ID NO: 105 - DNA, 433 bp, Homo sapiens
  AI799547
tggtggtatgaataaatgccgtttattctctttctgacgggataattttcaaagcagtgatttttataaaaactcaggaagaaaacaattttgtttcatttccataacagaatccagaccttgagcttcaccctgcgatcagtgattacacacacactgcaccgtttcggctgcagcggctcctgctgtccaccggctcccgtcctccctgcctgtaaccgtctgtggtgattcgcggcactgcaggaccatgatggcccggatctgctcggacgctccgcctcagcttctcatggccgccctcgctattgccacccctgcccaggctgcatcaaggaggtcaggggccaggggagaactcttttgttattgagaagtttgattgcaagacctgacccttcctgtgtggacggatgggcagatgcccagactg

SEQ ID NO: 106 - DNA, 468 bp, Homo sapiens
  AI803880
tctaagagatttttacatattgaagctccttatgtcagatacataattagcaaataattttcccattatgtagattttctttctgttttcttgatagtgccttttgaggcacaaaagtattttattttgatgaagtccaatttatatacatatatatatatatatatatatatatatatatatacacatttttttaacttgagcttttggcatcatacttaagaaaccattttcaattccgagattatgaagatgtatgtctatgctttcttctaatggtttcatagttttagctcttacgtttatgtctttgatccagtttgagttcattttgtatatggtgtgaagtagagctccaaattcatactttttcatgtggatatccagttgtttagcaccatttgttgaagagactattccttattccttcccccattaaattcttttgacattcttgtcaaaagtcaa

SEQ ID NO: 107 - DNA, 535 bp, Homo sapiens
  AI804425
ttttttcatttccttccagtttaatcctccgtgctaaaacaatcaaacaaacaaaactagttctcagtttggtgtcagctgctaatgagctcagtttacaaaaagaaggggaataagtttgaagtatatgcatttttcagtaaaaggcttttagcggaaaatgcaggatgagttgctgattgatggcacaagcacatttttcgtaaacagcagagaatatagaaatgctaactctggggatcttgactgtgtcagtgctgagtcatctgcatttcccatattattttataccagcaggcctaagaaggcccagacagaaatcacgcatacaaacacctccacacagacatctcacccccacacagacatctgcctccttatgtccattacactacatagaaaagtccctccattcccacctggggctgctacacgcgccactttgtgagcaggttcctcaagatgtgtgactgatgcttgccaaaggtgaagaaagaacaggggcaaaaaggggaagagtccgctgtgagaatca

SEQ ID NO: 108 - DNA, 267 bp, Homo sapiens
  AI807985
tttgagaattgaaactttgatttattggctaggggagagagatgaaaagaggatcactctggtagcaggagttttgggcatgtccctctgagatccatttgatgaaaacattttttaaaacatggtagggcctcattttgcacattttcttcacggtgtcatcaaataaacatcatggtaaatataaacttgttcgcccgtcagaaagcggcgccgggagggagcgagcgtgagaaagtgagcgtgtgcgctgccggagagcacgac

SEQ ID NO: 109 - DNA, 534 bp, Homo sapiens
  AI825890
aagtttaaactacagtctagtggatttagttcttcatgcttttgcctaagtgctacaatggcagggaaagaggctttggaatttcagatttttttttctttcttttttgtaaagaaaacctctcaagagggtatccaaccactttttggagacataaacatatgtaaagcaaagtgacccaaataaaggaagaatgaaaaaaaatatatatttattcttcaatttaggccaattctcttttacgggctccttgcaattagctggcaatcttgtcctgtgggtctgaaggattaaagcaggagcaaagaacaaaaggagtgtctgctacactcctccatcggtcctttctaaacaaatcagacttcatctcctccagtgcctccttaccacctcatcttctttcctctacccataatcccttaggggaaggactgttaacatgtgaaaatgctacgtaaatgaaattccttcagaatcaacacagatttgaaatggctccagaaactagagggtggtttttatggttctctcatt

SEQ ID NO: 110 - DNA, 579 bp, Homo sapiens
  misc_feature (578)..(578): n is a, c, g or t.
tttttgacaaattaagcaaatcatttaattaaaaggaatttccagagtatacagccaaatcattcacattttatatttacaattaaatactacacagcaagttcagaatgatcctgaggcattaaaagataatgaatttaaaaattatagtcacttactgaaatgtagcaatataattttaagttttctccttgattttcacttgaccaaaagttcaagtgacaagactttacattttgattattattctgtagatactgtctgttctaatattcatttatattattcttattcttagaaatctaaatagctaccagaaggttgtattgggaatgaccattaccattctatgcaagtatcttcccatgtgggtaatatcaagaccagggaataatcgaaatttcttgtcaagttcccatcgttcttgagagaccaaaggactcgacattttcaaatctattagtttacgccccctctttcagggaagcgtggttgggagcgtggactacttataagttaagtccagctttatgggcaaatcactgccaatgccacggaccggatggtttatagatganc

SEQ ID NO: 111 - DNA, 337 bp, Homo sapiens
  AI862880
ttttttttttttttttttttttttttttttttttttttttttttttttttttttttttttttttttttttttttttttttttttttaaaaaaaaaaccccccccttttttgggggaaaattaccccgggggaaaaaaaccaaaggggggggggtaaaaaaaggaaccctgggaagggggaacccccgggaggggctctttaaaaagggcggcaacgggggggggggggtccccccccccccccaaggggccacccccttttaaaaaaaaaaaaaggggggggggggggggttttttttccccggggggaaaaaccgcccccaaaaccctttttggga

SEQ ID NO: 112 - DNA, 399 bp, Homo sapiens
  AI864919
agcggccgccctttttttttttttttttttttttttcctgtcatcagagggctgtttccctttataaaaggggaaaacaattgctttttaaatttcttttcttcttggagtctaagattttaaaagttgcagcccacatgtgaatccaaatttcctttgaaccatcaaatttgaatccctctaattcggatttcccattcaaatcatgtcagggcagttacttgttgaactttgtcctgaaatcctgtggttctgtggttccggctactcaggaggctgaggtgggagaactgctcgagcctgggacatggaggctgtagcgagccacgatcgtgtcacggtattccagcctgggtgactgacagagcatgatcttgcctcaaaaaataaacacataaa

SEQ ID NO: 113 - DNA, 257 bp, Homo sapiens
  AI865298
tttttttttttttttttgaagacagagctccacttttgtttcccgggctgcagtgcagggtggcgatctcggctcactgcaacctccacctcccaagttcaagtgatcctcccgcctcagcctcctgagtagctggaatcacaggcgcctgccactgcacgcggccaacttttgcatttttagtagagacggggcggtctcaccatgttggccaggctggtcttgaactcctgacctcaggtcatccgcccgcctca

SEQ ID NO: 114 - DNA, 471 bp, Homo sapiens
  AI888234
tgcattagtaaatttttaattataaaaattcaatgctatcataaaacatacatgagaaaagtgtgatattaatcatctaacttgagaactagaagtaatttgggtgccaggtgcagtggctcgtgcctataattccagcaatgcaggagactaaggtgggaggattacttgagtccgggagttggagtccagcttgaacaacacagcaagatactaactatataaatgaaaaagaattagccaggtttgttggtgcatggctgtagtcctacctactcagcagatggaagcaggaggattgcttgaacccaggagttcgaggatgcagtgagttacaatcatgccactgcacttcagcctgggcaacagtgcgagaccccatctcttaaaaaaaaaaagagaaagggaattttgcaatactaagttaagcttttcctaagaggtagtctgaagtgtacaaatactgcagca

SEQ ID NO: 115 - DNA, 473 bp, Homo sapiens
  AI888493
aagtttttaaagtcatgtttattgaagtatgattttacattcagcaaaattcatcctgttaaggtttgacaaacgcacagtcataccataatcaagatgcagaactgtagaatcacactaacaattccttcctgcccttgtaatcaacccctttttgatcccaaggaactcactgacctgtttttctgtcaaaagtatcaaatttcatcactaaaatgatctacagtactatggaaaaatgtgtaagcacataaaatacacaaacctatttttttaaaagtgagatacttaaaatttctttagcataccaacaaaaaatgaaggtagcattgcattttactacagatgtgcggacattcaaagtccaccattgtcgtatatatggctcaacagtaatatttaagaatatactgctgggtatagtggctgacgcctgtaatcccagccctttgggaggctgaggcaggctgatc

SEQ ID NO: 116 - DNA, 503 bp, Homo sapiens
  AI910882
tttttttttttttttttcttttttcatgctctttaccaggttaagaaagttaccttctatatctagtttgcaagagttcttatcataaatggatgctgaattctgtcaaatctggtaatgtgttagtattaagtgataacttccatactgcagacaaagaaagtcagagctggaaggagattcaggatcagccagttcagtcacctaatttatagatgagaagactgaggtccagagagataaaatgacatgttcaaagcaacagctagataagaagtggaatcaggtctataatttaaacctttatatgtctaaatttttttaatataattatagtgtttatatggtctagtagaaatgagctaaagcaccataaaagcattgagaaaaaagaaaatatagagatcaaaatctcttagcagtttctgagaaagggtaaagggtaacaatggatttttaactgaggctcactaggtttccagtttaagtggtaatatctaccg

SEQ ID NO: 117 - DNA, 238 bp, Homo sapiens
  AI915031
tttcacagtcagctccattttattatattcataaaatgaccccacacctcctactgctccagtcaccccccgaaaggtggctaaaactgtgacccccaaagttaaaagattgatgtacccaatgagaagtggacatctgaaaacggtgcctgtgtggctggccggtgaccccagggcctccggcctcctaaccctgctgccctagtttccaaagagctgctctggccccctccagcca

SEQ ID NO: 118 - DNA, 359 bp, Homo sapiens
  AI921468
cttggaggcataatttagtcatctcacctaaagcacttttcactttatctctggcaaccaagggttacagaaaactcagcaccaaaggatgaaaggggaacttgtccccttcggtccccagccctgccctcccctgcagcctaaaataccctttctatgatcacagaacaaagttcacactcaccacacagccattctcacacacactcgcacaaaaagaaaaccaaagcccactaaagcacatggggaaaaaaagattacaaaacatcttcctccccatccggccttgagaccagactccctggctggagaggtcgtgacttccgccgtgcccagcaggggctcacccagcctttgtc

SEQ ID NO: 119 - DNA, 425 bp, Homo sapiens
  AI925267
tttttttttttagaacacaacactgactttaatggggcagccctgagccgtaaccttcaaaccttccgcctcagtaccccgtgcgcgaggagggaggggcgactgctacgggcacatcgtcgatgtcctccctactcctggcgatcccacgcctcaccccttcctctccagctgctgcggtctccgtcgccgaggtgggtcccaggtaagctcccaggaggggcaggggagcccctcagcggcccgggacagattcccccatggctgagggcatggggaactagcctggatggagacgccgccgtcctcggagctgggcggggacctgattctggggtgtggacagagccaagggaccgccccccaaggcccagcgccgggagatgcaaggccgggcccaaggtgttggacactgctttggggga

SEQ ID NO: 120 - DNA, 441 bp, Homo sapiens
  AI933013
gcaaaattccactttatttattcattcctcctccacattaaccttattaaaacccaaggagaagaagggggggaagggaaaaaataacctgaagaccccaattagggttggttccctttggaaaaaaggtccaaagcccaaagattggaaggggtcaagcttgaacttaagaaccagtgggggcacttcttccttttacccttttccctgttgaaactttttgggacgggccaacctcagcccttattggggaattccccagccttaacttttggtttctccgaatctgctttgctcaacctcaaggtgcttctgaggcttgaagtgctggccttgctttcctgctttttgacacttttcttggaatttccccattggaaggcgtttttcacctttcactgtaccttttgcctttctgggtaaaaagttatttcaccaggc

SEQ ID NO: 121 - DNA, 306 bp, Homo sapiens
  misc_feature (231)..(231): n is a, c, g or t.
ttttttttttttgagacagtctcactttgtcgccaggctggagtgtggagtgcagtggcacaatcttggctcactgcaacctctgcctcctgggttcaagcgattctcctgcttcagcctcctaagtagctgggattacaggcacaccccaccacacccggctaatttttatatttttagtagaaagggggtttacccatgttggccagctgggtcttgaactcctacctntntggatccacccgcctcggcttccaaaagggggaagtcactgcacccagccttgctggatttttctaaaacctt

SEQ ID NO: 122 - DNA, 289 bp, Homo sapiens
  AI936300
agcaaaaacagcttttttattgtggtagtttgtggtatgtgctcctggatcatgcagaaaaaaggctgggccctcagttagctccgggagccattcttaggaccctccggctgcacacagagaggggctgggtagctggctgggctggggcacgcattcactgggctggcacaggctgaggggtctctcgcccactatcattaggcccctccagcccgttatgttcagcccccggctcaggatgctccagggcgtgccgggtatcagcctgccagagctgcaccaggtc

SEQ ID NO: 123 - DNA, 516 bp, Homo sapiens
  AI936462
tcagcttctcagtcatttttaataaatgcttacggtttttcttccaaagcaggtgaaaggagaatatataagtaacgatatatttgcggtggctatgcctattccacagcaatcacaaggggtgtccttggggtgggagtgcgcctgggaaagagttggcaatcagtgagtgacagtcctagacttcaaggaccagaaagactaggcagccgtgggggaccagaataagtaggaccacgagagagagtagagagaagcactgtggcaaaagatggagggtctcactagctctttatttcccagaagaaatatcagacacgcagggtagacttctgcattaagcaccgcaggcccagtgccagggaccatagtcttttggggcccatgaaaatgttttgatttgttttaaaatcagaaaaaaataagtaaagctttaggctgaagaacatgttttaatatgcaatattaatatacttttccttatgccaacgcagttgcaaaatcttatttttaata

SEQ ID NO: 124 - DNA, 944 bp, Homo sapiens
  BC018761
gcaagatggtgttgcagacccaggtcttcatttctctgttgctctggatctctggtgtctacggggacatcgtgatgacccagtctccagactccctggctgtgtctctgggcgagagggccaccatcaactgcaagtccagccagactgttttatacagctccaacaataagaactacttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatattatactactcctatcactttcggccctgggacccaagtggatatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagagggagaagtgcccccacctgctcctcagttccagcctgaccccctcccatcctttggcctctgaccctttttccacaggggacctacccctattgcggtcctccagctcatctttcacctcacccccctcctcctccttggctttaattatgctaatgttggaggagaatgaataaataaagtgaatctttgcaaaaaaaaaaaaaaaaaaaaaa

SEQ ID NO: 125 - DNA, 1021 bp, Homo sapiens
  D49410
aaatgaaaaaaaataatagttcactcaaacacaacttccgggttgaaggttcaacgattctcctcctcacctccaagtactgggactacagacatgcaccacacacccagctaattctgcatttttagtagagaaggggtctcaccatgttgcccaggatggtctggatctcctgaccttatggtccgctcgcctcggcctcccaaagtcctgggattacaggtgtgacccaccgcgcctggcccaaagtgctgggatgacaggcgtgagacaccatcctgccccacagaaaagatctgagatgggacagcccccgcagatcaggacgtgggctctgttatctggggggtgaccgactcaccctgcctcctctcgtctctgcaggtggtctgggaggcgggcaaagccggcctggaggagtgtctggtgactgaagtacaggtcgtgcagaaaacttgagactggggttcagggcttgtgggggtctgcctcaatctccctggccgggccaggcgcctgcacagactggctgctggacctgcgcacgcagcccaggaatggacattcctaacgggtggtgggcatgggagatgcctgtgtaatttcgtccgaagctgccaggaagaagaacagaactttgtgtgtttatttcatgataaagtgatttttttttttttaacccactcactggtcccggtctctggattcagccccattcctccaacactactagagagactgtttccccggttttttttttggggagatggagtcacgatctgtctcccaggttggagtgcagtgatgcaatctcagctcactgcaaccgctgcctcccgggctcaagcaattctcctgcctcagcctcccaagtaggtgggattacaggcacctgccaccacccctggctaatttttatattagcggtctcgaactcctgaccttgtgatctgcccgcctctgcctcccaagtgctgggattacaggggtgagccaccacacctggccttttttctttaaa

SEQ ID NO: 126 - DNA, 446 bp, Homo sapiens
  misc_feature (68)..(68): n is a, c, g or t.
ttttttttaattttatataaagtggttttttattaatgaaacatttcattgcattaagggtagaaatnaacatgtcaagaaccgcatgaagtgaaccaactgtataagctacatgaagagcattctggtcagtattcttgttaacaggcacaagcaatttagtgataatgtccagagggccaaggatgcggaccaccttttgcagaactcatatctcgagcagtttaaattaaaaattaggcctcccttaagttaaaaaccccaatagggttnaggtcgtgtagtgcttgttccacttgaccattaggtgatctttctaggaacatgagggaaatcaagtctgggttagaaaagcctggggagcttaaaagtgggccaaagcccatttttcctgttcctgtggcttcaactccttaatttttccctgaggaggangttgagtttaa

SEQ ID NO: 127 - DNA, 376 bp, Homo sapiens
  misc_feature (309)..(309): n is a, c, g or t.
ttttttttttctattgaaatatttatactttattcaaggatatattcattttgaggctcatgagcaagagttggtaaaaattacagttcagtctatagtcttgagaaatttgctaatctcccagatcttaagcaaaaagaaaaaacaagcaaaccttttgcctggccctaaacacatctaaagctgcttgggacaatcatcctagctgttgttctcatatgacttatagcagataagcagattaggtatataaaccataccactttttcaaaaatttccaatcatttaggaaatagttactcatggganataaattttgacttacaaatttcttgggtgggtggggnaaggcaaaattaagaggaaggaaggtgtg

SEQ ID NO: 128 - DNA, 351 bp, Homo sapiens
  misc_feature (70)..(70): n is a, c, g or t.
cacctaggctggagcgcaatggcacgatctcagctcactgtagcctccacctcccaggttcaagcgatanccctgccttagcctcccgagtagctggaattacaggtgtgcgccaccatgcctggctaatttttgtatttttagtagagatggggtttcaccatgttgggaaggctggtttcgaactcctgacctcaggngattcacccgccttggcctcccaaagtgctgggattatagggcgtgacanncttcacctgctgaaaaataaactttaaacatctctcttcgttttcataaaaaaaatctgataccttttgacatttcagctttaacacagatgaatctctt

SEQ ID NO: 129 - DNA, 454 bp, Homo sapiens
  misc_feature (419)..(419): n is a, c, g or t.
tttttttttcaaggagagtcattttattatgaatcattgtgcaaagaacataaactaaattgtaagctcttctagggcaacacccaaatatcctaattttgaattctgtacttcaaagtgattataaaggaataagactttttataaaacaaacgtatcatatagctcatatatgcaaaggagttttatcctttcaaataagtccctgtatcgcttaaaacatttttgggaagttctatctaaaattttgtttcatgtgagggcaggtgtcaggcaccgaaaaggtactagggtccacttcacactattccacctgggttaggaccatacttcatggatgggccaggtcagttatgggggaccgttaaacaaaatggattactgggactaggcttaggaacaagtttcaacttgttccnggaatggatggaggggantggaanttcaaaggtnt

SEQ ID NO: 130 - DNA, 426 bp, Homo sapiens
  misc_feature (301)..(301): n is a, c, g or t.
tttctggttttaaaataatttatttcataggtctgttttaatttcatgtctcacagcatacattatttaaccaagaggtgctggacagagattgtaaaaagggaggaaacatttaggataagaaaccggctcaattccattagaatttgatcctatcttgtaaagtcttgctaggataaatttctataatacttcttttgtgcttacaaaaaaacattaacttctgaatttccaatgttgtaaataaaaaaattttacaattaggtatgcattcaattaacacacaagggcaaccaaatanattctgtcatcaacattaggcataatgtgtatttcccgtcactggcactaacattaaactaaactttcaaaccacaanaacattgtggactaacntttaaaagggcaggctgttagggtactcaa

SEQ ID NO: 131 - DNA, 485 bp, Homo sapiens
  misc_feature (342)..(342): n is a, c, g or t.
atgttttctattttatatttctcaagccaaatatctgcaacaataacatgtattgaaaggtatagaaataaacagatggatagacagaaaaagactcagactgtctaagtagtgaagtttgtgcagaactagaaacaaaaatccacctgggtcagaggtatagggttaatttctcttgctgaggctcagatttctccttcatgcatgtttaatggtccacctaagttcaactggaggagcatctttcttctttgttaaccaaacatgcccagggtaggagctgcttctcttttctatgggtaggctgctgagttctgactgtgtcagcctgggagggtaccnccggacgttaggcatctttagggctgcctattcacgggcagccnggggagggtagtgggcngggctgggggcatagtncccgccagatgggnaaaagggncacacccggcccgnttacaagggccttggggattttttcagaa

SEQ ID NO: 132 - DNA, 441 bp, Homo sapiens
  misc_feature (282)..(282): n is a, c, g or t.
tttttttttttttgagacagggtctcactctgtcacccaggctagagtacagtggcacaatctcggcttactgcaacctctgcctcccaggttcaagcgattctcctgcctcagcctcccgagtaactaggaccacaggcacacaccaccatgcccggctaatttttgcatttttagtagagacagggcttcaccatgttggccagggctggtctcaatttcttgacctcatgatccaccagcatcggcctcatgatgtgctggggattacagggcatgagncaacgcactcgggcctagtattcaattttacagggcagggccacctcttacctattttcacaggaaaaccagtntttacacaggancagtnaggaaccactggaattcagtnggtcttttcnggggggttntaggttcacantggatttaaantacagg

SEQ ID NO: 133 - DNA, 405 bp, Homo sapiens
  misc_feature (1)..(1): n is a, c, g or t.
ntattgaataggttcaaatatgaagacattattggtgaaaaaacaaaaaacaaatagtggacaagaaataatagtcaccacaatcatctttttcaacctaacacaattcagaataatacataagcaatttttacatttttaatcttacaatcatcttttattctaccatactgtcaaatacttgtgaaaataaataaaaataaaatacatgatgcataggaagattttttttaaaactgaactattgcacaaatcaatgtgagantatgcaagtaatataaaagtgcatttaaatatattgtcacaacatatttcggtaagtcataaatctatttcacctactgtttataatctctggggctctgggnactaggtggtttcttaccnactcatctccngtgggga

SEQ ID NO: 134 - DNA, 369 bp, Homo sapiens
  misc_feature (269)..(269): n is a, c, g or t.
ttagtgatatcaatacagcagaggtgcccatggagcagggggaagggacccatgcctggaccagtcccttcctccttctgcccctgctgggtccaggaggatgaggcctggcctggaccagttcctcctgttccacctgaaacacatggggatggagaaccagggctggccttgcttcctgctcctcttgttaccactccagtgttatcacttccttgaagggattagatctctccctccccagctcctaggcagggaaccgtagctantggggaggggcatatttgggaggcctgaggctaagggaatagcctctagacccctccctgacccacaacctttagttccctggggnccaanaaaagnagg

SEQ ID NO: 135 - DNA, 462 bp, Homo sapiens
  misc_feature (236)..(236): n is a, c, g or t.
aagtaagtttattaacctgccaggcttctccaccagtcggaggtggcagccctgagcttacaaaatgaggggtttatatgggggagagagaccctggggttgttagtcaattaactttaccacatataatctcatgaccggcttacaatatattatcttgtgaaaataggattttacaagggggtgtaatccacgtttctcatgacctcccccgtgccacccagagggcttcggtntagcaagtctggtgaccttgctatagcgcctagataacggttcaggaatgcagctgcagagtnttcagggtaagggtcagctgcattgagttagcgggggcggantggtcctgggggcagcctgtccctaacagantccacctccgagttgtgacaattaattngaaaaggttgaaaaggtgaacatgaagcacctgctacactgccctnttggccaccagctttt

SEQ ID NO: 136 - DNA, 230 bp, Homo sapiens
  misc_feature (194)..(194): n is a, c, g or t.
catctccttgcccaagggtgtggtggaggtgacccatgacctgcagaaacacctggctgggctgggctgactgaggccattgacaagaacaaggccgacttgtcacgcatgtcaggcaagaaggacctgtacctggccagcgtgttccacgcacccgcctttgagttggacacagatggcaacccctttgaccngggccatntacgggcgcgaggagntgcgnagcccca

SEQ ID NO: 137 - DNA, 198 bp, Homo sapiens
  misc_feature (1)..(3): n is a, c, g or t.
nnnaanctgatagaaggtcttgctctgtcacccaggctggagtgcagtggtgccagatcatggctcatgacagtctcaacctcccaggctgaagtgatcctcccaccttacctccccatccccccaatccccagtagctagctgggactacaggagtatgctactgtgtccagataattaaaaaaaatttttttcctt

SEQ ID NO: 138 - DNA, 420 bp, Homo sapiens
  misc_feature (1)..(1): n is a, c, g or t.
natcagtaanattacatctgcaaaacagctgggtttagcctaatattcctgagctggcttaaagagaaatctgagtctttcaggtaaagatgagcatgaaaattaatttgtatttattttttgtcagctactattttacccaaaaggatcaaacgttatagatatactacaaatcactttaaaacaagaggttcctcagtcagtcccaatccaaggatatgcattaaatctgaattactgttatatctatatactttaacccagcatgaggtctgggttaaccaaatgccaaagcaggttttgtgggtaagggcccatcatgaaaaaactaaggtcattattctacaaatctttaaggntttgtcttaaataaattcactgttcacactaaatttgtcttaaattaaattccaatggtaa

SEQ ID NO: 139 - DNA, 417 bp, Homo sapiens
  misc_feature (300)..(300): n is a, c, g or t.
aaaacaatcaatttttaggtttattttttacttttaataagccagtactataagagcaccatttcagcacaacacaatcatttgtgaagtaaggggttttagttgagatctgtgaaaatagtttatatgggaaagaggtatgatggctcagagtgctgtcaagaacagctttctttctaaaatgaacctgtaactttccaacaaccttcaaatgcacagaagtcttagttatcattctctaggcattagaagtgggagctggggagagggctcaggaataccagtgggtaggcccggggnccagccggggaaaggcgaggaggggtgggcctttactatttgaatacagtttataactgtgaagggcaatattgctgtaagagagggnttgggagcnttgtaaatctactctgtggc

SEQ ID NO: 140 - DNA, 572 bp, Homo sapiens
  misc_feature (427)..(427): n is a, c, g or t.
cacttttagtcagagggtttttattaggagtaaattttcctaggttctcgataataaaggttaatatatttttttcaaaggcacaacgaagcatcctttcaggtcaccactgatttcaaaacaaaacaaaacaaaacaaaacagaaatttcctgatttgatgtgtaaaaggtcaacatctgctgatgctcagatatggggaatgtggtggaggcggccgaatgggatgtcgagtgagcagtacagttagaggggccatgcaacatacaactgacactccaaacagaaagacaccccaacgccaaagaactacttttccatccatccatttctgaaactttctgacaggccagatcactatagcagcctcaataatctgtgtcagcttgcttctacttgcttccctgttgactaaaatggcacgtgcnaatgcaattgtcaagggcctgaaaggagcacattgtgggggatatataccgttagggcaatgccaagggggatactaaagccgaaaattagactattagagctgaaggntttcanttgcaagttttaacctggtgcnggttccan

SEQ ID NO: 141 - DNA, 2856 bp, Homo sapiens
  K03195
tagtcgcgggtccccgagtgagcacgccagggagcaggagaccaaacgacgggggtcggagtcagagtcgcagtgggagtccccggaccggagcacgagcctgagcgggagagcgccgctcgcacgcccgtcgccacccgcgtacccggcgcagccagagccaccagcgcagcgctgccatggagcccagcagcaagaagctgacgggtcgcctcatgctggctgtgggaggagcagtgcttggctccctgcagtttggctacaacactggagtcatcaatgccccccagaaggtgatcgaggagttctacaaccagacatgggtccaccgctatggggagagcatcctgcccaccacgctcaccacgctctggtccctctcagtggccatcttttctgttgggggcatgattggctccttctctgtgggccttttcgttaaccgctttggccggcggaattcaatgctgatgatgaacctgctggccttcgtgtccgccgtgctcatgggcttctcgaaactgggcaagtcctttgagatgctgatcctgggccgcttcatcatcggtgtgtactgcggcctgaccacaggcttcgtgcccatgtatgtgggtgaagtgtcacccacagcctttcgtggggccctgggcaccctgcaccagctgggcatcgtcgtcggcatcctcatcgcccaggtgttcggcctggactccatcatgggcaacaaggacctgtggcccctgctgctgagcatcatcttcatcccggccctgctgcagtgcatcgtgctgcccttctgccccgagagtccccgcttcctgctcatcaaccgcaacgaggagaaccgggccaagagtgtgctaaagaagctgcgcgggacagctgacgtgacccatgacctgcaggagatgaaggaagagagtcggcagatgatgcgggagaagaaggtcaccatcctggagctgttccgctcccccgcctaccgccagcccatcctcatcgctgtggtgctgcagctgtcccagcagctgtctggcatcaacgctgtcttctattactccacgagcatcttcgagaaggcgggggtgcagcagcctgtgtatgccaccattggctccggtatcgtcaacacggccttcactgtcgtgtcgctgtttgtggtggagcgagcaggccggcggaccctgcacctcataggcctcgctggcatggcgggttgtgccatactcatgaccatcgcgctagcactgctggagcagctaccctggatgtcctatctgagcatcgtggccatctttggctttgtggccttctttgaagtgggtcctggccccatcccatggttcatcgtggctgaactcttcagccagggtccacgtccagctgccattgccgttgcaggcttctccaactggacctcaaatttcattgtgggcatgtgcttccagtatgtggagcaactgtgtggtccctacgtcttcatcatcttcactgtgctcctggttctgttcttcatcttcacctacttcaaagttcctgagactaaaggccggaccttcgatgagatcgcttccggcttccggcaggggggagccagccaaagtgataagacacccgaggagctgttccatcccctgggggctgattcccaagtgtgagtcgccccagatcaccagcccggcctgctcccagcagccctaaggatctctcaggagcacaggcagctggatgagacttccaaacctgacagatgtcagccgagccgggcctggggctcctttctccagccagcaatgatgtccagaagaatattcaggacttaacggctccaggattttaacaaaagcaagactgttgctcaaatctattcagacaagcaacaggttttataatttttttattactgattttgttatttttatatcagcctgagtctcctgtgcccacatcccaggcttcaccctgaatggttccatgcctgagggtggagactaagccctgtcgagacacttgccttcttcacccagctaatctgtagggctggacctatgtcctaaggacacactaatcgaactatgaactacaaagcttctatcccaggaggtggctatggccacccgttctgctggcctggatctccccactctaggggtcaggctccattaggatttgccccttcccatctcttcctacccaaccactcaaattaatctttctttacctgagaccagttgggagcactggagtgcagggaggagaggggaagggccagtctgggctgccgggttctagtctcctttgcactgagggccacactattaccatgagaagagggcctgtgggagcctgcaaactcactgctcaagaagacatggagactcctgccctgttgtgtatagatgcaagatatttatatatatttttggttgtcaatattaaatacagacactaagttatagtatatctggacaagccaacttgtaaatacaccacctcactcctgttacttacctaaacagatataaatggctggtttttagaaacatggttttgaaatgcttgtggattgagggtaggaggtttggatgggagtgagacagaagtaagtggggttgcaaccactgcaacggcttagacttcgactcaggatccagtcccttacacgtacctctcatcagtgtcctcttgctcaaaaatctgtttgatccctgttacccagagaatatatacattctttatcttgacattcaaggcatttctatcacatatttgatagttggtgttcaaaaaaacactagttttgtgccagccgtgatgctcaggcttgaaatcgcattattttgaatgtgaagggaa

SEQ ID NO: 142 - DNA, 13649 bp, Homo sapiens
  L39062
aagctttttcctgccccacacatgcttttaccgccgccagttttaacacatatttacacttccttttttgctgcattagtgtctcatctttgggaatattcttgctgcagttgtttttttttttttttgagacagggtttcactgtcacctaggttggagtaaagtggcgcaataacgactcaccacaacctgaaattgccgggctcaggagatcctcccacctcagcctcccaaggagctgggactataggcgtgggccaccctgcccagctaagggagggtgaatggctgagcttgcctgaagaccctcccgtgggtgtgcagagcctgtggtctgagggaggggtctagctgcagggcacgcacagcttcacagtggcccagggaaccagggcaggcaggccgtgaggcccagtggtcatctgggccaggccttgatgctgaggccatggaacaggtgaggctctaagaagggcccagagtgaccatgggctggaggctatgtccacagacccagtgcaggagagggtgtcctgggcagtgatgtcagccaggctccctagcaggactgggggtgtcggggcagctctttcccaggtggcagacactggcttcccctcttgctctcacaactgtcctgtgacctggtgtctgagctgtggtgaggcctccctggtgacacagaggagcacggagcatgtgggtgggggtgtgtgcacctgccctgacttcctggtcctgtggtcaaggatgggggaaggtagatctgcctgtagctccaccctatttgtaaagcactgtggtgccttctgctggggcatgtgctgagtggtgcctcgcaggcactgccctcgggaagttcacaggcttgtgtagaagctgatgggaatgtgccaagaacagaggtgtcaggagccaggtattgggcaggtcctaggtctctgagcctcagtctccttatctgtaggagggtggtaaccctgccctgctcagcttaccaggtgcagctgtgagttttcagtgcagaggaaaagcagagaccttcccctcagatggccatacccccttgtcgctatacccaacactctaatgctcccctagggagtgtcctgggctcctcctttgtagctctcaggtgtcacatgtgggtcttgccctaccatcccctctccctttctagaaatgaggactgctgagcttggagccgccccactccctgctctcaagccgtctgctcctgggttagcctgtggctggcctggcctgattctacaccgacgtgggtgtttctccactgctggggcagcggtcggccatcctggggcctgggtcagctctcagctgtggctgttgtgcctgtgcttccccaggtcctggtggtcactccaaccccgccctcagatatcccaagagcaggctgactgtcttccccattcccacctttccagtagctgctgcaagaagggacagacgttgctgcagagaccttgccacggtgtttcatgttgtggccggtggtgccagactgcacgctccattctagggaagggtgagtctggccgatcatcagtcttaacaggggactgtcctgtgtgtacctgggtaggctgcaggagtggggttagagtagtgcctactgcctgttgagagtgtgtgagttccttaagagcatgcatgctgggccaggcatggtggctcatgcctgtaatcccagcactgtgggagactgaggcgggcggatcatgaggtcaggagttcaagaccagcctgaccaacatggtgaaaccctgtctctactaaaaatacaaaaattagctgggagtggtggtgcgcacctgtaatcccagctactcaggaggctgaggcaggagaatcactggaacccaggaggcagagtttgcagtgagccgagatcgtgccactgcactctagcctctgtctcaaaaaaaaagaaaaaacaaggcgtgcatgctgtagtctgtgtgtatgtctgtttttgttctcctccttaacactagtcagtcttgtgtaaccaggctggccctgcttcctgcttagaagctattggtgtatcatctggagctgcaaatagggtgatagggcgtcagtagccttcccacactcaagagcttcctgacacccagcccctcatctccagccagcctctggctaccccaggaatcttggggctcaggcctaacactccagcatagaggctttgcccttctggtgtactctcgcttggaggcatcttggaacaggggtacaacagagctctggaagtgctgtctgttgcatcgataccccactgccactttcgtccaccttcctggggcagaatcctgggatgggtctttatttaagacaaaagggagagagaaggcatagaggctaggcatggtggctcactcctgtaatcccaggactttgggaggccgaggctggtggatcacttgaggtcacgggtttgagaccagcctggccaacacggtgaaaccgtctctactaaaaatacaaaaaaaattaactgggtggcagtgtgcacctgtaatcccaactacttgagaggctgaggcagaagaatcacctgaacccaggagtcggaggttgcagtgagccgagatcacgccactgcactccagcctccagcctgggcaacagagggagactccgtctcaaaaaaaaaaaaaaaaaagagagagacagagaagaaaaaaagaaagagagagagaagaaaaaaagaatgcatagaggccatctgtgtgaccctgggcaagtcattttccctgcttcactccttgggactcagcctgtgaacggggacagtgcttcttccttacagcgctgttgtgagaattttcattgaaaatgcacctgtggtggttgtaaggagtgactggttcccccaatcctgacccctcctgcagctgggtcaatcggcctttctgagcttcagttccttatcagtaaaccctgggcagtggtgcccagcgtctttcacaggacactgtgtgagtgcagatggagacccgctgagcactccgctggggagcaattcatggggagcacccctccagagagggatggctcgcacaggccctcagcccagccccttgcaggctggaccttggagagtgaggccctgaggcaagacatgggcacctggctcctggcctgcacctgcgtctgcacctgtgtctgctcgggagtctctgtctcgggggatggacgaggtgagggctgggcactagtgtctgtatgaggtgtgtggagaactagggcatgtttgggggactggtttgtccgatgttgagaccctagggaaaggtttggcccaaactgtgctggggcatgtcctctagggttcagcctggacctcagtctctattcttgcttcctttacctccctatcttcagtccctggacagaccttagtctcccttccttcaccctcttgacgcctctctgggtctgacttgcctgtgtaccacgggtcagagcccatcacttcccaggcctctcagtgctttcttggacagattctgggatcattcactggtgactgccctgctagggtgtcagctgtcagatccccccgcaaccccccaactcagctctgccctcgagcactcactgtgggctcccgaccgtcactgtctccagggccaagggctggaacctccacctgcctcaccaacaacattctcaggatcgattgccactggtctgccccagagctgggacagggctccagccccgggctcctcttcaccaggtgaccttggaggccatgcccacctggacagggatgagggtgaggtccccaggatggcagcagatatgccaggatagtgtagcagccccgtggtgctgacacatgccctttccagcaaccaggctcctggtggcacacgtaagtgcaccttgcgaggcagtgagtgcaccgtcgtcgtgccgcccgaggcagtgctcctgccgtccgacagttgcatcatcactttccaaatgcgtgtctgggagggagcaggtcagcctggtggacccggagtacctgccctggagacacggtgagcagcggctataggcctggggcagggccctttggcaagaacatcctggctgcctgggggctgggagcagggccctgcagcctgtgacccctgaggcccagtgagtgttctcagtcccagccaagtgagatccagggctgggggcaggcttggcccctgggaggggagggccgacgtggtgacggcaggggcagggttttgtagaaaataaagatgcagggctgctagttggggcaggggctggcacttgagtcatgtgaaatgcacttcggtcataccaggaaggactgccagcagcagactgtgaaggaaaggggaagcaagatttagaaaccacctagtctagctgcagaggccagaggaagtcattgctgtcctgtccctcccgaaggttttctggaccagtctcccagtgaggtgcctggtctgagagggccttgaccatgccccttgggaatctttcagatccccggtcttggatgtgctgactgacacacccagacctatggggcttcagccttacgtagactcactctgttccagtgaagctggacccactctgacttgcagggcaacatcagctctagccactgcatcctgacctggagcatcggtcctgccttggagtcgaggaccacacttctcagctatgacctatggccttcgagaggcaggaagaggcctgggaggtaacgctttggctggcttcccctggggcctccctcctgggagcagcagtccagggcaggctccccactctaaataaggagaggtcaacttggggtgaagaggaggggaggagctagagccagacgtgtgtgtgcacatgctgacatgcacatgtgtgtgcctcgtgtgtgtatgtgagtagggtgagtgctcctgtgtttatgtgtgtgcacatgtaagtgtgcccgtgtttgtgtttgtacatgagtgtgtgcctcgtggttgtatacgtgagtgcacttgggcatgcctacaggtatgagtgtgtgtgtgagtgtggtgagttgcttctgtgcacacacttgtgtttatgagtgtgatgcaagtgtgatgagtgtgaaagtgcctgtagacacgtttgccctgtgtgtgcctatgtgtatttgtgggcaaacgcagctgtgtctgcgtgtgactgtgagtgtgccttctccgtgtgtgtgtgcacatgaacctgctgagtgtgcttgtgtgaacacagatgtgttcctgtgtgttatgtgagcgtgtgcgtgtgtgtattctcgagggggaccagccccaccttcagcacctgctaactgtcgccacccccacagtgggcccagcacagggatcacatcgttggggtgacctggctcatacttggagcctttgagctggaccctggctttatccatgaggccgggctgcgtgtccagatggccatgctggaggatgacggggcacaggaggagcgttacacaggccagtggagcgagtgtagccagcccgtgtgcttccaggctccccagagacaaggtggtcgctgctacggctgctgcacttccagagtctgggctgggcatcctctcccctgatcacctcacccctgcaccctttcagcctcctggaaggccctccctgaggcagccatgccccactttgacccccttcctcggaagctctgaggtctgcagggaggaaacaaacacctgccaactctggggcttcctgggaacccatagtcagtggctcctcttagcagtgagggtggcagggctgcacaccagggctggtgctcctgcctggaggctggacatgacctcagtgtccttagtgagggctgggctgacccttgcgcactgcagtgctgagacggcccagggaccttatgacccaccatgtggcagatgggaagagtgaggcccaggagtggggctcacacaaggtccttcagcaggtgacacaaacctccaaggcccatcacaaggtccttcagcaggtgacacaaacctccaaggcccatcacaaggtccttcagcagatgacacaaacctccaaggcccatcacaaggtccttcagcagatgacacaaacctccaaggctcatcacaaaccttccactttggccccagggcactaaagggcgcacctttgccaggtgggtttggggggagcctcctggcactgaggctgctcacagccctgggcccttcctgcccacgggccctctgatcccaccctgggggtggccaggcaacacccttgttgctgtgtccatctttctcctgctgactggcccgacctacctcctgttcaagctgtcgcccaggtaggtggctgatgtgtgcgtgtgtgtacatgtgtgagcgggcaagagtgtgcatgttagtgtatgtgtgcagatgtgtgactgtgtgcatgtgtgagtgtgtggtgggtgggccgtcaagggccgcccttgtctggttcctcccctcccctctccactgcctggacctggacggggtgggcttttcgagtctccaccctggtccagaagagggttctcaacttgccaggggagagcagggaaggggggtctgaggcagaggctgaagataagggcagcttggtcccgaccagacccagagtcaccgagatcaagagccggagtcctagtctcctgcctctggaggagcttggtttgttcatttgttcacacgttatatgaaaggtcagttggggcatctggtgtacacaggaccctcttctcaccctggccgatgccttagagaacaagatggacaaggaccctccttaaataaaccgtcacgcctcatgcaccacatgtcaggttacaagagggagtgcaggggccatgtcaggccgaggatgtgaggaagagggtgtggggagaggggtgttgggtggggctaagtgggagggtgcttcgcttctccttggagcaaaggaaagtgtaaaactgtgtgacacaacctagtttatgttttgaggaatagattgctgggacaaggggacatgggggacctgctggaggccaatgggggccagtgaggtgcccacagggagacgatttggcatgagccagtgaaagaagtggagatagagaaaagtggagagaattgcagggtctcatgaggggaaatagaagggacatgtgggtggatgagatatgagtggtgaaccctgaggtggggaaatggggcacagccttgcggggaggttggcgaggtctatttgcacctgttgggttggagccccaggtgacatccttgtgggagggtcagtggatccttggcagcaagtctggtgctcagggagacactgggtggggtgggagccacagaccttctgctgatggcaaagacagggttcctggaggtgctggctccctctgtgatctgaggacccagctagtaactccccgttctgaacccgccatcgcagctcacgctgtaaaggacgcgcgcctcagtataaatcagttctatgcggccgttaggcaaggaggcccagttgggtcctgccctgagagtgggttggaatgtgatgagatgggagagaggcagtggcagggacgaggtgggcctcctgctgatggaaggaagctcagcctctgcagtgacctcaggccacctgggtcgccataggcctctgactggcctctcttggcctcagggtgaagagaatcttctaccagaacgtgccctctccagcgatgttcttccagcccctctacagtgtacacaatgggaacttccaggtgtgtgcagagaccacagcaggacatggggggcaggggttgcccagagctctggcctgcccaagatgttggctttcatgagggttggcggccagtatgggaggcttgtcagtgcttggagccttttttgtatattcagtgaatttcaatttatacgcggatctcaaatggggaaaaattagctttattttccattgctgatttctttttgttctcaggtctctagttccattgttgattgaaaaaatgtaaatttctcatgacttatctctgtcccctctggtttgcagctttctgcacccaccatgtgcctcacctcctccttctgcgaaggtgtctgtcctgtggccatggggaagggtctgtggtgtgtgtgctgcccttggggctctcactgcctctgggctcctgctctgcctggtcccctggtctccccggatcacatgatggcaccacagctgaggagtgggctctgcacttcccccccttccactcatgttgggctcctacagcccaggcaccagtgagcaacttgggggttgcatcagcccctcccctccctgctgggctgttggttcatgccccctgggtgggaggagggggagagggagagctccagtgagtggtctctggtttttcccctcagactcctcactttgggcaaaggacaagaggcagtgagggcccctccctggggtctgggccaagctgaccactcttctccagaatcttccctccctgtccccttcacactgtggctccagcttactatgcagaaaaatccttttctctctcaatgaggagcctagttttcaagatttttgtccaaaaatataatttgaaccatgaaccgggcatctggctcttggcagagtctccctctttccccaaggtggtagatgtgactgtcaggagtctgggcagctgacgacaaggctgagcaggtcagattgtgactgttccctggactgtcatcctgttgcgggcaccagctgttccctagagaactaggacacctgccacgggttatttagacccgcgggtgaggatctggtgccataggttggtctccagggagcactgcagtgatggagggtgttgtgtgtgtgatgcatgggatggaggctcctggtcccaccaagggaacagcttccttttggaggcgggggcctcctgtggccccacagaaggatccaggtctgctggccatagccgagtgctttgaaagtcaccagtcctgacagcgattcgtgtgtgtgtctgtgtgtgtgtgtgtgtgtgtttatgtgtctgtgtgtgttcgtgtgtgtgtctgtgtgtgtgtttgtgtgtgtatgtctgtgtgtgtgtgtttatgtgtgtgtgtctgtgtgtgtttatgtctgtgtgtgtttgtgtgtgtgtgtctctgtgtgtgtgtctgtatctgtgtgtgtttgtgtctgtgtgtgtttgtgtgtgtgtctcgtgtgtgtgtgtctgtgtgtatctgtgtgtgtttgtgtgtgtgtgtgtctctgtgtgtgtgtgtgtttgtgtatgtttgtgtgtgtgtgtgtgtgtgttgggaatgcccagtctctgcagctgctgaaaggccctgaggcacatgctgtcaggagctggctctgtcctgggcagatatcaccatctgtaccttggttcaggctgccgtgggcaccaggccctgtgctgggggagtgctgaggagcctgaagggactcagggtcccgtgatgaggctgggctggcacatggaggaaagacagaatgtccaagacacaggcgctgcttggcctctgggtgtggacctcaggagggcttcctggaggaggagggatgctgggcttgccagaaaggaggcagctgctcccaggatgagttctaaacatgctgcctgagcctttccctcctcccgtgctctgttccagacttggatgggggcccacagggccggtgtgctgttgagccaggactgtgctggcaccccacagggagccttggagccctgcgtccaggaggccactgcactgctcacttgtggcccagcgcatccttggaaatctgtggccctggaggaggaacaggagggccctgggaccaggctcccggggaacctgagctcagaggatgtgctgccagcagggtgtacggagtggagggtacagacgcttgcctatctgccacaggaggactgggcccccacgtccctgactaggccggctcccccagactcagagggcagcaggagcaggagcagcagcagcagcagcagcaacaacaacaactactgtgccttgggctgctatgggggatggcacctctcagccctcccaggaaacacacagagctctgggcccatcccagccctggcctgtggcctttcttgtgaccatcagggcctggagacccagcaaggagttgcctgggtgctggctggtcactgccagaggcctgggctgcatgaggacctccagggcatgttgctcccttctgtcctcagcaaggctcggtcctggacattctaggtccctgactcgccagatgcatcatgtccattttgggaaaatggactgaagtttctggagcccttgtctgagactgaacctcctgagaaggggcccctagcagcggtcagaggtcctgtctggatggaggctggaggctcccccctcaacccctctgctcagtgcctgtggggagcagcctctaccctcagcatcctggccacaagttcttccttccattgtcccttttctttatccctgacctctctgagaagtggggtgtggtctctcagctgttctgccctcatacccttaaagggccagcctgggcccagtggacacaggtaaggcaccatgaccacctggtgtgacctctctgtgccttactgaggcacctttctagagattaaaaggggcttgatggctgttccaaagtgttgatggctgggagaaggggccagaggaggagtgaggggtggggtttgtccagccctgggctttccgggctctagagatagcatggtgtaggctcaatgacagttctggggacagcaagttggaggttcaggggcagcttcaggacagcaggatggaggctcagggacaattcctgggaggccagtgccctcgttcctccttgtcctcatcctcccacttgctccaggaaactgagagctgagcctggagcttccagacagtcagtgctgggggtgaccatccagcagtgatggtggcctgtgaagggtcctgcttctgtcctcagcctctcatggggtgggcttgtggaggagctgtggtctggagagagtggcagttggagcagaacgtgcctgcgtttgtttcctagggctgtcgtaacaaagtgccacaaaatggttagcttagaaccacagagatttgttgtctcacaattctgaagtccagaagttggaaataaagatgttggcagtgttcccaaccacatgttcttgggctcccatgaaacagaagttgatatgaggccaaggaagcttcccagacaagactttattaagtcttatgccccgaaagtttgggcagaagagagacggtgcaggaggaagaattcttggctgactccccaaggggaatgcattgtggtgtcttaaggagggtgacatacataatttatgagctacatgagtgtcattgcacgtatggggtggagtgaagggtgctcagacgcatgctaacacatacgttgcatgatcagaaaatggcagataagcccctccctgggtgaggactttagtattatcataagaccagggtcattctcctggccttgtgcacaagcaggtgatggagtcaactcccgtcagtaagacttatggcgggatgctgcttatcttagtttatttcagacagttggcaaggtctggccagcgagtatggcacctggagggtggtgctgcaaggtctagtggtcagcgggcacgtatggaacaatacgttagtgggggtgggccgagtcccatttatactctctcagcagggccatgctccctctgaaggcactagggaaggattagtttcaggcctctcttcagcttctgttagtttcttggcttgtgacaccaaagctgtaatctttcttttgttttgtttttgtgacggagttttgctcttgttgcccaggctggagtgcaatggcacaatctcggctcactgcaacctcctcctcccaggttcaagcaattcttctgcctcagcctttggagtagctgtgattacaggcgcctgctaccacgcctggctagtttttgtatttttagtagagacggtgtttcgctatgttggccaggctggtctcaaactcctgatatcaagtgatccgtctgtctctgcctcccaaagtgttgggattacaggcatgagccaccgtgcctggccaaagttccagtctttacagggatttttccttgtgtgcatttctgtgtccaaatttcccctttttaaaatcacaataatagtgaattaaggctggccctaacgatttaatcttaacttgatcatctgcaaagacactatttccatataaggtcacattcacagctactggggttaggacttcaacctagagggcctgacttctggcccaacacatcatggcccatcccagcatgccccatccccttcctgggtgccccaggcagatcacaggagggcctgactgctgggctttgggctgacattgggatcatctgcctagttagggctgtgaccagactgagataggaggtgggacctgactcctgaggcagggcttgaactctggaccagattacagactagctgaaacaggcaaaagcacccctccataagacacacccactggtgccaagtgagtttgccgttctcatggtaacagctggaaattactgcccctttccatggcaatgacctgaaagttaccaccccttttctagaaatttctaaataacctcctccttaatttgtatatagttacaagtgggtataaatatgtgtgcagaactgcctctgagctgctactctgggctcactgcctatggggcatccctgctccataaggagcagtacctctgctgccactgtgcacagctgcttaaataaaagttgctctctaataccaccgactcgcccttgaattctttcctgggtgaagccaaaagccctcctgtgctaaaccccaatttctgggctcacctttcctgcatcaagagtgttttggggctgggagcgctgaagttttgaaggcaggatgaaatggagactgttagctccctgggtcctgagctacagggagcaactctcctgggtgctctcatgcttcctgcaacctgacgcagaaagaagtacctgtagcctgatgcccccaaaggcaaaagccccttctggctcagataggcagccctcccaccctgcatgcctgcagagcttctggggttttgtggggagcctcagcagagctggatcctggtctgtgggggcgaggcaggccccagaatgtatctttagcatccagagagtgggaggggtgtggacggaagcgaggtacaggcatcctcagctctaaccaagtataaaaatactagtgtgaatccacttcctggagctgggctctggggagccgggctagtgtatttataacaggggctgtgtgctcatgaggctccatgtgtgacctggctgacctcagtgatcacataaacgaacccacaattgggatgcctctgcctgtgtggcctggacacgtcttgccattcaactcataaactttgccctgagtgggccctggagagctggttctgggctggaaccgtgagagtgaagcca

SEQ ID NO: 143 - DNA, 1828 bp, Homo sapiens
  M61199
ggggctgacgcagcattgccaattctaaatccatcatttgactgaggaggagaggtttgaagttgatcagctccagggtttgagaaattcagtccgaatggaacttcaggacctggaactgcagctggaggagcgcctgctgggcctggaggagcagcttcgtgctgtgcgcatgccttcacccttccgctcctccgcactcatgggaatgtgtggcagtagaagcactgataacttgtcatgcccttctccattgaatgtaatggaaccagtcactgaactgatgcaggagcagtcatacctgaagtctgaattgggcctgggacttggagaaatgggatttgaaattcctcctggagaaagctcagaatctgttttttccaagcaacgatcagaatcatcttctatatgttctggtccctctcatgctaacagaagaactggagtaccttctactgcctcagtgggcaaatccaaaaccccattagtggcaaggaagaaagtgttccgagcatcggtggctctaacgccaacagctccttctagaacaggctctgtgcagacacctccagatttggaaagttctgaggaagttgatgcagctgaaggagccccagaagttgtaggacctaaatctgaatctgaagtggaagaagggcatggaaaactcccatcaatgccagctgctgaggaaatgcataaaaatgtggagcaagatgagttgcagcaagtcatacgggagattaaagagtctattgttggggaaatcagacgggaaattgtaagtggacttttggcagcagtatcttcaagtaaagcgtctaattctaagcaagattatcattaaacagaaattataggttggcatggatcctattagctgtgtaatactggaattatcaatgatatgcactggtggaggtgttatttgtgctttagaagatacttgctgttgagctgggctactgtatacagtgtacaatgtgtatttcttcaaccatatattttaaaaagacgtacatagaaacttaggcactttgctatttcttttctaaactatcaaaaactctagcagtttgaaaagcctaatatttatttgtatgtcaatatttttcatttgattccctattagaattaattttaaaacttgaagacttccagacttatccaacttataaataacatatttcttcagactaacatcttaaaacactgacctctatgaggtatttactgtgcaataactgattcatttttttcagagcttgaagcatccaatgatttttccctccactgctgttaattaatgtcacttccaagaagaaaaactgttctgttgtaaaaaatataattgctcttaattcttggggaggttactaatagcagtaggatagaattttatgaggttacctacaactacttaatgtacttacactgtaagccttgttgctttacccaagacaaatgtaattttatcattgcttatgtagtatttttcttttggaaatgtgccttatgttaaacactatgtacttttactttttgcattgtccagacttctttattagatggagatgtttctttttctgtcttctagactaaatagagtatcatccaaataatggggcctatgacttgaatgaatagaaatgaataagctggtgtttgttttttcaaaatggaagtaatttagatttgttctcctcatacataaaatgattttagttcagttttaaccagtgaaaactttgtttttatgaaaaaaaaggaaaatggtttcccatttggttttatatgtgttaaataaatgtgtaaagtaaccaccccc

SEQ ID NO: 144 - DNA, 1406 bp, Homo sapiens
  M90746
tttttcatctctccacttctcctaataggtttggcagtgtcaaccatctcatcattctctccacctgggtaccaagtctctttctgcttggtgatggtactcctttttgcagtggacacaggactatatttctctgtgaagacaaacatttgaagctcaacaagagactggaaggaccataaacttaaatggagaaaggaccctcaagacaaatgacccccatcccatgggagtaataagagcagtggcagcagcatctctgaacatttctctggatttgcaaccccatcatcctcaggcctctctacaagcagcaggaaacatagaactcagagccagatcctttatccaactctcgatttttccttggtctccagtggaagggaaaagcccatgatcttcaagcagggaagccccagtgagtagctgcattcctagaaattgaagtttcagagctacacaaacactttttctgtcccaaccattccctcacagtaaaacaacaatacaggctagggatggtaatcctttaaacatacaaaaattgctcgtattataaattacccagtttagaccggaaaaaagaaaataattattcctaaacaaatggataagtagaattaatgattgaggcaggaccctacagagtgtgggaactgctggggatctagagaattcagtgggaccaatgaaagcatggctgagaaatagcagggtagtccaggagagtctaagggaggtgttcccatctgagcccagagataagggtgtcttcctagaacattagccgtagtggaattaacaggaaatcatgagggtgacgtagaattgagtcttccaggggactctatcagaactggaccatttccaagtatataacgatgagccctctaatgctaggagtagcaaatggtcctaggaaggggactgaggattggggtgggggtggggtggaaaagaaagtacagaacaaaccctgtgtcactgtcccaagttaagctaagtgaacagaactatctcagcatcagaatgagaaagcctgagaagaaagaaccaaccacaagcacacaggaaggaaagcgcaggaggtgaaaatgctttcttggccagggtagtaagaattagaggttaatgcagggactgtaaaaccaccttttctgcttcaatgtctagttcctgtatagctttgttcattgcatttattaaacaaatgttgtataaccaatactaaatgtactactgagcttcactgagttacgctgtgaaactttcaaatccttcttcatgtcagttccaatgaggtggggatggagaagacaattgttgcttatgaaaaaaagctttagctgtctctgttttgtaagctttcagtgcaacatttcttggttccaataaagcattttaca

SEQ ID NO: 145 - DNA, 530 bp, Homo sapiens
  misc_feature (3)..(3): n is a, c, g or t.
ctngaactgcagcgggtggcagtagagaagagcatcacagaagttgctcaggagctgacagagctggtnggaacatcttgtagacattgtcagaagcctgcagaatcagaggcccctatcagaatctggaccagacaacgaactgagcatcctgggcaaggctttttcttctnggggtggatctttccatggcttctccaaagctggtgacttccgacacagctgcagggaaaatcagagatattctccatgacctagaagaaattcaggaaaaattacaagaaagcgtcacctggaaagaggctcctgaagcacaaatgcagggagaactcctgggaagcccccgtcttcctcctcatggcccatttgccttggaccaagagccacccttgnactcaagggagagcttctcgggttttgctcagttgggccatccatccaacctttaactttgaagagcctttcctgcagccttggaggtgtccaaggattcccggtacagtgactgngttttcaccggcccaaaaccc

SEQ ID NO: 146 - DNA, 670 bp, Homo sapiens
  misc_feature (503)..(503): n is a, c, g or t.
gttattggacgaggtgtattaaaataaaatcaaaaatagaaatattaggtatatacacaacagcataagtgaaacacatcatgtgatacagaagtttccattcaaagcttacaggagaaaaatttagtgaacagaaccgcttgaacctcctgccaccgtggcttattagagccttgtagtcacttatgttccttcatatccttgggtataaatagtctcatacacatgctacacaatttagaaaagattgctgaatcataaaaatagccccgaatgtggcactttagctttctagagtacaactaaaattaatacttgatctttaacataatgttcaaatatttgaagcaacaaaggaaactcttatgttttagaaggccctataaggttcttcagagattagaatcctagttcttccaaactacattgtcaaaacaaatctggatataggaaattgtggaaatggaaaagctggactcctaaaaatattccctctaacaccngagcagtttctggaggctcttctcccnttcctgggaggtccaggtgagcccggtgccttcngggtgagcagacagggcagctgtggtgctcacgggctcccctgctggaagagagagagtgtgccgccactcctatgggatgtctagcaggttgagcagggtcaana

SEQ ID NO: 147 - DNA, 547 bp, Homo sapiens
  misc_feature (449)..(449): n is a, c, g or t.
gaaatgcagaaatatttattttggtttccttcattgtttttggaaattttgttttggtttataaaacatagaaatagccaacacttaaagcaaacattcaaaaccccaaggtgacaaattattgactttttgtgcaattaagaatacatatatgaagttaggctaccaagtagtgttattacaatgacaattctttagtgcaagccctgttgtgcttgtatataatacatgtactctcacagaccccaaaacagctgctttaaatgtacaaatgacagctcaattcagtcaaatgtggcaaagactcacaataaagtgaactgctgttaatttcccaaattaactttaaaaaatccctggagaagtgaatcccaggaaatgaactttccagatttctacatcctagaattttgggctggtcaaacacatttcatagaaatcaggtagcnttataaggacctttgcctattactgnccatttccataagtactccagtatatactcccttatagccttaaaggaatgcctggccngagagtttanttt

SEQ ID NO: 148 - DNA, 475 bp, Homo sapiens
  N49899
ttccaacaacatttggtttataaaggaatacaaacaggcacaaaacatggttcagaagatttattaagtaaacttgctaaaatatggacagatacacttagcagtcaaacagttgaatattaattgctacctcattaaagtttttgtatctgtattaccaggtccaaacataaaaaccacctctgttcaaaaaataaatgttcagagagctgtatgttctttgttcttgtatgtacattttaaaaaaacacctctttccagtcttgctaaccaagaatattagtcatataaaagaacttagaatttttttccccaagtacaagctatcttttggctccaaaacagttctgaaggttttatttatattttatcttatcccgagggaccaacagcagggcatacctttggccaggccttcttggcagaaagacacagagccgtaaagggaaaaaataaaattgccataaaggtatag

SEQ ID NO: 149 - DNA, 506 bp, Homo sapiens
  misc_feature (454)..(454): n is a, c, g or t.
ttgaggttataaacattaacttgatttaatcattttacaacataaacatatatcaaaatattacactgtagacagatatatacaactattatttttcaattaaaaatagaatttttttaaaatttaatttttctttttcaatgaatctttgatttcttcactttaaactcttaggtattaagcatacaaatgtttaatggcttagaatagttacctaacacattatatgagccaaatactaactactcctccaaactatcttttttcatagcattcttcaaatacaagattattctgtatatcttattaaaatttatttgtctgccttatctaaatattgataaaaatagaaggtaggggcatttgcctggcttcatacattccaaaatcaaagggcagttttactaattatttttacattcagtaactcatttggtctcacacagtaaacacnaatattacgtaacaggactatattcntattccncatccnaaaaatgggaaaa

SEQ ID NO: 150 - DNA, 451 bp, Homo sapiens
  misc_feature (369)..(369): n is a, c, g or t.
accattttgaaatgatgtatggtgtgattgttagagatggatcagcatgattggtcatcttggtttttaaaggaagccagtgcttggcttcaaataatgctatgtggaattttatttatgtggtataccttgcttaaaaattgtaaaaattcgttaaaattgtgaaaaccaaaataaattttctttccacttctattaaaattttctttccaactctttacacattcatttgaggtttcctgggaaaagtaggaggcaaaccatccaaaggtactgtgggttttctaaaaaaattttaaattcttgctattaaagttcgtttatcataattctatacacagtactccccccttatccatagtgtttgcntttcctgcagttcaggttactggagggtcaactggttggtcaaaagtagggtgcaggggccaggcatagtgggctcacacct

SEQ ID NO: 151 - DNA, 430 bp, Homo sapiens
  N64374
cagttcattgcaatttatttaatttaaaaataaaaacagaaacaaaaaccaaaatgaaacaaaaatcagtttccaacgaaaatacaaacactttgggtggttatccagcttgtttttcccctgtaggctgttctgggctcaaaccaatcaaatgagtgaaaaccaattttgacaggagccataaagcatgttgcaccaattaaattacaccaatatattattataatacctttgaaatgcctttcagaccaataaataaaaaagacaaattcaaataaaaaagtcaactttttattaccaaaaaaaatagaaattaaataaaagtcacaatgtggatttttttttttttttaatgtgcagtcaagtttctctctttttttcttctaggaatgataccatgccagtaaatccctacagaacatttccagtt

SEQ ID NO: 152 - DNA, 444 bp, Homo sapiens
  N66205
taaaacaatccatattatttatttaatcctaagtatttttaaacactgctatcatgctacttcacctaattctggctgctgttcatttggggaatgctatattggactattcttgaaacaatatatataaaatcaagcaaggcacgtgacagtcaggctttaggttggagccaggcctcggaaaatgtttttcaaacaattttgtgtctatcctttcatcctgagctggaactattgagaatgtgatagaaaaatgcatcgagtggcttgttgggtcttgtaagggtcctacgactctcatactctgtaaatggaatgtgtggagagggttaggagagccttcaggtagtactagagatgctggagcctctgaggacctctgcactgagattcctaatgttaatctggcctctgccatcactgagggtacccggggcttggctt

SEQ ID NO: 153 - DNA, 179 bp, Homo sapiens
  N73694
gtatttttttatttttatgtatttatgcatttaatttttatgtattcatatgtatttaattttttaaatttatttattgagacagagtctcactctgacacccaggctgcaatgcagtggggcaacagagtgagactgtctcaatcaatcaatcaattaatcaataccttcataaaaac

SEQ ID NO: 154 - DNA, 300 bp, Homo sapiens
  misc_feature (266)..(266): n is a, c, g or t.
agagagcttaggattttatttttcgtttacacagggaatccaagaaaggagacagacaaagtgagtctcaaggatgagagcagagaaaagctcgagataaagagctgtgcagcccgggtcaggaagtgatcggtctaggtcggaacaggagaatcagttagacacaacggatgtgtttgagcctttggaaaacggtatcgttaggcatgtggcaaaaacgttggggtacttgaaaaaaaggctggccatgggttagtaaaaaccangatatgtggaaanattagggtaattattaacccc

SEQ ID NO: 155 - DNA, 1816 bp, Homo sapiens
  NM_000074
cttctctgccagaagataccatttcaactttaacacagcatgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctgaacagtgtcaccttgcaggctgtggtggagctgacgctgggagtcttcataatacagcacagcggttaagcccaccccctgttaactgcctatttataaccctaggatcctccttatggagaactatttattatacactccaaggcatgtagaactgtaataagtgaattacaggtcacatgaaaccaaaacgggccctgctccataagagcttatatatctgaagcagcaaccccactgatgcagacatccagagagtcctatgaaaagacaaggccattatgcacaggttgaattctgagtaaacagcagataacttgccaagttcagttttgtttctttgcgtgcagtgtctttccatggataatgcatttgatttatcagtgaagatgcagaagggaaatggggagcctcagctcacattcagttatggttgactctgggttcctatggccttgttggagggggccaggctctagaacgtctaacacagtggagaaccgaaacccccccccccccccccgccaccctctcggacagttattcattctctttcaatctctctctctccatctctctctttcagtctctctctctcaacctctttcttccaatctctctttctcaatctctctgtttccctttgtcagtctcttccctcccccagtctctcttctcaatccccctttctaacacacacacacacacacacacacacacacacacacacacacacacacacacacacacacacacagagtcaggccgttgctagtcagttctcttctttccaccctgtccctatctctaccactatagatgagggtgaggagtagggagtgcagccctgagcctgcccactcctcattacgaaatgactgtatttaaaggaaatctattgtatctacctgcagtctccattgtttccagagtgaacttgtaattatcttgttatttattttttgaataataaagacctcttaacattaaaa

SEQ ID NO: 156 - DNA, 3971 bp, Homo sapiens
  NM_000228
gctttcaggcgatctggagaaagaacggcagaacacacagcaaggaaaggtcctttctggggatcaccccattggctgaagatgagaccattcttcctcttgtgttttgccctgcctggcctcctgcatgcccaacaagcctgctcccgtggggcctgctatccacctgttggggacctgcttgttgggaggacccggtttctccgagcttcatctacctgtggactgaccaagcctgagacctactgcacccagtatggcgagtggcagatgaaatgctgcaagtgtgactccaggcagcctcacaactactacagtcaccgagtagagaatgtggcttcatcctccggccccatgcgctggtggcagtcccagaatgatgtgaaccctgtctctctgcagctggacctggacaggagattccagcttcaagaagtcatgatggagttccaggggcccatgcccgccggcatgctgattgagcgctcctcagacttcggtaagacctggcgagtgtaccagtacctggctgccgactgcacctccaccttccctcgggtccgccagggtcggcctcagagctggcaggatgttcggtgccagtccctgcctcagaggcctaatgcacgcctaaatggggggaaggtccaacttaaccttatggatttagtgtctgggattccagcaactcaaagtcaaaaaattcaagaggtgggggagatcacaaacttgagagtcaatttcaccaggctggcccctgtgccccaaaggggctaccaccctcccagcgcctactatgctgtgtcccagctccgtctgcaggggagctgcttctgtcacggccatgctgatcgctgcgcacccaagcctggggcctctgcaggcccctccaccgctgtgcaggtccacgatgtctgtgtctgccagcacaacactgccggcccaaattgtgagcgctgtgcacccttctacaacaaccggccctggagaccggcggagggccaggacgcccatgaatgccaaaggtgcgactgcaatgggcactcagagacatgtcactttgaccccgctgtgtttgccgccagccagggggcatatggaggtgtgtgtgacaattgccgggaccacaccgaaggcaagaactgtgagcggtgtcagctgcactatttccggaaccggcgcccgggagcttccattcaggagacctgcatctcctgcgagtgtgatccggatggggcagtgccaggggctccctgtgacccagtgaccgggcagtgtgtgtgcaaggagcatgtgcagggagagcgctgtgacctatgcaagccgggcttcactggactcacctacgccaacccgcagggctgccaccgctgtgactgcaacatcctggggtcccggagggacatgccgtgtgacgaggagagtgggcgctgcctttgtctgcccaacgtggtgggtcccaaatgtgaccagtgtgctccctaccactggaagctggccagtggccagggctgtgaaccgtgtgcctgcgacccgcacaactcccctcagcccacagtgcaaccagttcacagggcagtgccctgtcgggaaggctttggtggcctgatgtgcagcgctgcagccatccgccagtgtccagaccggacctatggagacgtggccacaggatgccgagcctgtgactgtgatttccggggaacagagggcccgggctgcgacaaggcatcaggccgctgcctctgccgccctggcttgaccgggccccgctgtgaccagtgccagcgaggctactgcaatcgctacccggtgtgcgtggcctgccacccttgcttccagacctatgatgcggacctccgggagcaggccctgcgctttggtagactccgcaatgccaccgccagcctgtggtcagggcctgggctggaggaccgtggcctggcctcccggatcctagatgcaaagagtaagattgagcagatccgagcagttctcagcagccccgcagtcacagagcaggaggtggctcaggtggccagtgccatcctctccctcaggcgaactctccagggcctgcagctggatctgcccctggaggaggagacgttgtcccttccgagagacctggagagtcttgacagaagcttcaatggtctccttactatgtatcagaggaagagggagcagtttgaaaaaataagcagtgctgatccttcaggagccttccggatgctgagcacagcctacgagcagtcagcccaggctgctcagcaggtctccgacagctcgcgccttttggaccagctcagggacagccggagagaggcagagaggctggtgcggcaggcgggaggaggaggaggcaccggcagccccaagcttgtggccctgaggctggagatgtcttcgttgcctgacctgacacccaccttcaacaagctctgtggcaactccaggcagatggcttgcaccccaatatcatgccctggtgagctatgtccccaagacaatggcacagcctgtggctcccgctgcaggggtgtccttcccagggccggtggggccttcttgatggcggggcaggtggctgagcagctgcggggcttcaatgcccagctccagcggaccaggcagatgattagggcagccgaggaatctgcctcacagattcaatccagtgcccagcgcttggagacccaggtgagcgccagccgctcccagatggaggaagatgtcagacgcacacggctcctaatccagcaggtccgggacttcctaacagaccccgacactgatgcagccactatccaggaggtcagcgaggccgtgctggccctgtggctgcccacagactcagctactgttctgcagaagatgaatgagatccaggccattgcagccaggctccccaacgtggacttggtgctgtcccagaccaagcaggacattgcgcgtgcccgccggttgcaggctgaggctgaggaagccaggagccgagcccatgcagtggagggccaggtggaagatgtggttgggaacctgcggcaggggacagtggcactgcaggaagctcaggacaccatgcaaggcaccagccgctcccttcggcttatccaggacagggttgctgaggttcagcaggtactgcggccagcagaaaagctggtgacaagcatgaccaagcagctgggtgacttctggacacggatggaggagctccgccaccaagcccggcagcagggggcagaggcagtccaggcccagcagcttgcggaaggtgccagcgagcaggcattgagtgcccaagagggatttgagagaataaaacaaaagtatgctgagttgaaggaccggttgggtcagagttccatgctgggtgagcagggtgcccggatccagagtgtgaagacagaggcagaggagctgtttggggagaccatggagatgatggacaggatgaaagacatggagttggagctgctgcggggcagccaggccatcatgctgcgctcggcggacctgacaggactggagaagcgtgtggagcagatccgtgaccacatcaatgggcgcgtgctctactatgccacctgcaagtgatgctacagcttccagcccgttgccccactcatctgccgcctttgcttttggttgggggcagattgggttggaatgctttccatctccaggagactttcatgcagcctaaagtacagcctggaccacccctggtgtgtagctagtaagattaccctgagctgcagctgagcctgagccaatgggacagttacacttgacagacaaagatggtggagattggcatgccattgaaactaagagctctcaagtcaaggaagctgggctgggcagtatcccccgcctttagttctccactggggaggaatcctggaccaagcacaaaaacttaacaaaagtgatgtaaaaatgaaaagccaaataaaaatctttgg

SEQ ID NO: 157 - DNA, 591 bp, Homo sapiens
  NM_000590
ccgctgtcaagatgcttctggccatggtccttacctctgccctgctcctgtgctccgtggcaggccaggggtgtccaaccttggcggggatcctggacatcaacttcctcatcaacaagatgcaggaagatccagcttccaagtgccactgcagtgctaatgtgaccagttgtctctgtttgggcattccctctgacaactgcaccagaccatgcttcagtgagagactgtctcagatgaccaataccaccatgcaaacaagatacccactgattttcagtcgggtgaaaaaatcagttgaagtactaaagaacaacaagtgtccatatttttcctgtgaacagccatgcaaccaaaccacggcaggcaacgcgctgacatttctgaagagtcttctggaaattttccagaaagaaaagatgagagggatgagaggcaagatatgaagatgaaatattatttatcctatttattaaatttaaaaagctttctctttaagttgctacaatttaaaaatcaagtaagctactctaaatcagtatcagttgtgattatttgtttaacattgtatgtctttattttgaaataaat

SEQ ID NO: 158 - DNA, 2755 bp, Homo sapiens
  NM_000629
ttaggacggggcgatggcggctgagaggagctgcgcgtgcgcgaacatgtaactggtgggatctgcggcggctcccagatgatggtcgtcctcctgggcgcgacgaccctagtgctcgtcgccgtgggcccatgggtgttgtccgcagccgcaggtggaaaaaatctaaaatctcctcaaaaagtagaggtcgacatcatagatgacaactttatcctgaggtggaacaggagcgatgagtctgtcgggaatgtgactttttcattcgattatcaaaaaactgggatggataattggataaaattgtctgggtgtcagaatattactagtaccaaatgcaacttttcttcactcaagctgaatgtttatgaagaaattaaattgcgtataagagcagaaaaagaaaacacttcttcatggtatgaggttgactcatttacaccatttcgcaaagctcagattggtcctccagaagtacatttagaagctgaagataaggcaatagtgatacacatctctcctggaacaaaagatagtgttatgtgggctttggatggtttaagctttacatatagcttacttatctggaaaaactcttcaggtgtagaagaaaggattgaaaatatttattccagacataaaatttataaactctcaccagagactacttattgtctaaaagttaaagcagcactacttacgtcatggaaaattggtgtctatagtccagtacattgtataaagaccacagttgaaaatgaactacctccaccagaaaatatagaagtcagtgtccaaaatcagaactatgttcttaaatgggattatacatatgcaaacatgacctttcaagttcagtggctccacgcctttttaaaaaggaatcctggaaaccatttgtataaatggaaacaaatacctgactgtgaaaatgtcaaaactacccagtgtgtctttcctcaaaacgttttccaaaaaggaatttaccttctccgcgtacaagcatctgatggaaataacacatctttttggtctgaagagataaagtttgatactgaaatacaagctttcctacttcctccagtctttaacattagatcccttagtgattcattccatatctatatcggtgctccaaaacagtctggaaacacgcctgtgatccaggattatccactgatttatgaaattattttttgggaaaacacttcaaatgctgagagaaaaattatcgagaaaaaaactgatgttacagttcctaatttgaaaccactgactgtatattgtgtgaaagccagagcacacaccatggatgaaaagctgaataaaagcagtgtttttagtgacgctgtatgtgagaaaacaaaaccaggaaatacctctaaaatttggcttatagttggaatttgtattgcattatttgctctcccgtttgtcatttatgctgcgaaagtcttcttgagatgcatcaattatgtcttctttccatcacttaaaccttcttccagtatagatgagtatttctctgaacagccattgaagaatcttctgctttcaacttctgaggaacaaatcgaaaaatgtttcataattgaaaatataagcacaattgctacagtagaagaaactaatcaaactgatgaagatcataaaaaatacagttcccaaactagccaagattcaggaaattattctaatgaagatgaaagcgaaagtaaaacaagtgaagaactacagcaggactttgtatgaccagaaatgaactgtgtcaagtataaggtttttcagcaggagttacactgggagcctgaggtcctcaccttcctctcagtaactacagagaggacgtttcctgtttagggaaagaaaaaacatcttcagatcataggtcctaaaaatacgggcaagctcttaactatttaaaaatgaaattacaggcccgggcacggtggctcacacctgtaatcccagcactttgggaggctgaggcaggcagatcatgaggtcaagagatcgagaccagcctggccaacgtggtgaaaccccatctctactaaaaatacaaaaattagccgggtagtaggtaggcgcgcgcctgttgtcttagctactcaggaggctgaggcaggagaatcgcttgaaaacaggaggtggaggttgcagtgagccgagatcacgccactgcactccagcctggtgacagcgtgagactctttaaaaaaagaaattaaaagagttgagacaaacgtttcctacattcttttccatgtgtaaaatcatgaaaaagcctgtcaccggacttgcattggatgagatgagtcagaccaaaacagtggccacccgtcttcctcctgtgagcctaagtgcagccgtgctagctgcgcaccgtggctaaggatgacgtctgtgttcctgtccatcactgatgctgctggctactgcatgtgccacacctgtctgttcgccattcctaacattctgtttcattcttcctcgggagatatttcaaacatttggtcttttcttttaacactgagggtaggcccttaggaaatttatttaggaaagtctgaacacgttatcacttggttttctggaaagtagcttaccctagaaaacagctgcaaatgccagaaagatgatccctaaaaatgttgagggacttctgttcattcatcccgagaacattggcttccacatcacagtatctacccttacatggtttaggattaaagccaggcaatcttttactatg

SEQ ID NO: 159 - DNA, 781 bp, Homo sapiens
  NM_000758
acacagagagaaaggctaaagttctctggaggatgtggctgcagagcctgctgctcttgggcactgtggcctgcagcatctctgcacccgcccgctcgcccagccccagcacgcagccctgggagcatgtgaatgccatccaggaggcccggcgtctcctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggccagccactacaagcagcactgccctccaaccccggaaacttcctgtgcaacccagattatcacctttgaaagtttcaaagagaacctgaaggactttctgcttgtcatcccctttgactgctgggagccagtccaggagtgagaccggccagatgaggctggccaagccggggagctgctctctcatgaaacaagagctagaaactcaggatggtcatcttggagggaccaaggggtgggccacagccatggtgggagtggcctggacctgccctgggccacactgaccctgatacaggcatggcagaagaatgggaatattttatactgacagaaatcagtaatatttatatatttatatttttaaaatatttatttatttatttatttaagttcatattccatatttattcaagatgttttaccgtaataattattattaaaaatatgcttcta

SEQ ID NO: 160 - DNA, 1285 bp, Homo sapiens
  NM_001671
ggggcccagggccctcctatggaccctgcccgctcccctcccattgtccacggctgtccgcccacccccattctccaagcttcagccccctccttagttcggcatctgcacagcactgaagaacctgggaatcagaccctgagaccctgagcaatcccaggtccagcgccagccctatcatgaccaaggagtatcaagaccttcagcatctggacaatgaggagagtgaccaccatcagctcagaaaagggccacctcctccccagcccctcctgcagcgtctctgctccggacctcgcctcctcctgctctccctgggcctcagcctcctgctgcttgtggttgtctgtgtgatcggatcccaaaactcccagctgcaggaggagctgcggggcctgagagagacgttcagcaacttcacagcgagcacggaggcccaggtcaagggcttgagcacccagggaggcaatgtgggaagaaagatgaagtcgctagagtcccagctggagaaacagcagaaggacctgagtgaagatcactccagcctgctgctccacgtgaagcagttcgtgtctgacctgcggagcctgagctgtcagatggcggcgctccagggcaatggctcagaaaggacctgctgcccggtcaactgggtggagcacgagcgcagctgctactggttctctcgctccgggaaggcctgggctgacgccgacaactactgccggctggaggacgcgcacctggtggtggtcacgtcctgggaggagcagaaatttgtccagcaccacataggccctgtgaacacctggatgggcctccacgaccaaaacgggccctggaagtgggtggacgggacggactacgagacgggcttcaagaactggaggccggagcagccggacgactggtacggccacgggctcggaggaggcgaggactgtgcccacttcaccgacgacggccgctggaacgacgacgtctgccagaggccctaccgctgggtctgcgagacagagctggacaaggccagccaggagccacctctcctttaatttatttcttcaatgcctcgacctgccgcaggggtccgggattgggaatccgcccatctgggggcctcttctgctttctcgggaattttcatctaggattttaagggaaggggaaggatagggtgatgttccgaaggtgaggagcttgaaacccgtggcgctttctgcagtttgcaggttatcattgtgaactttttttttttttaagagtaaaaagaaatatacctaaa

SEQ ID NO: 161 - DNA, 1809 bp, Homo sapiens
  NM_002185
gtcttcctccctccctcccttcctcttactctcattcatttcatacacactggctcacacatctactctctctctctatctctctcagaatgacaattctaggtacaacttttggcatggttttttctttacttcaagtcgtttctggagaaagtggctatgctcaaaatggagacttggaagatgcagaactggatgactactcattctcatgctatagccagttggaagtgaatggatcgcagcactcactgacctgtgcttttgaggacccagatgtcaacatcaccaatctggaatttgaaatatgtggggccctcgtggaggtaaagtgcctgaatttcaggaaactacaagagatatatttcatcgagacaaagaaattcttactgattggaaagagcaatatatgtgtgaaggttggagaaaagagtctaacctgcaaaaaaatagacctaaccactatagttaaacctgaggctccttttgacctgagtgtcgtctatcgggaaggagccaatgactttgtggtgacatttaatacatcacacttgcaaaagaagtatgtaaaagttttaatgcacgatgtagcttaccgccaggaaaaggatgaaaacaaatggacgcatgtgaatttatccagcacaaagctgacactcctgcagagaaagctccaaccggcagcaatgtatgagattaaagttcgatccatccctgatcactattttaaaggcttctggagtgaatggagtccaagttattacttcagaactccagagatcaataatagctcaggggagatggatcctatcttactaaccatcagcattttgagttttttctctgtcgctctgttggtcatcttggcctgtgtgttatggaaaaaaaggattaagcctatcgtatggcccagtctccccgatcataagaagactctggaacatctttgtaagaaaccaagaaaaaatttaaatgtgagtttcaatcctgaaagtttcctggactgccagattcatagggtggatgacattcaagctagagatgaagtggaaggttttctgcaagatacgtttcctcagcaactagaagaatctgagaagcagaggcttggaggggatgtgcagagccccaactgcccatctgaggatgtagtcatcactccagaaagctttggaagagattcatccctcacatgcctggctgggaatgtcagtgcatgtgacgcccctattctctcctcttccaggtccctagactgcagggagagtggcaagaatgggcctcatgtgtaccaggacctcctgcttagccttgggactacaaacagcacgctgccccctccattttctctccaatctggaatcctgacattgaacccagttgctcagggtcagcccattcttacttccctgggatcaaatcaagaagaagcatatgtcaccatgtccagcttctaccaaaaccagtgaagtgtaagaaacccagactgaacttaccgtgagcgacaaagatgatttaaaagggaagtctagagttcctagtctccctcacagcacagagaagacaaaattagcaaaaccccactacacagtctgcaagattctgaaacattgctttgaccactcttcctgagttcagtggcactcaacatgagtcaagagcatcctgcttctaccatgtggatttggtcacaaggtttaaggtgacccaatgattcagctatttaaaaaaaaaagaggaaagaatgaaagagtaaaggaaatgattgaggagtgaggaaggcaggaagagagcatgagaggaaaaaaa

SEQ ID NO: 162 - DNA, 2545 bp, Homo sapiens
  NM_002416
atccaatacaggagtgacttggaactccattctatcactatgaagaaaagtggtgttcttttcctcttgggcatcatcttgctggttctgattggagtgcaaggaaccccagtagtgagaaagggtcgctgttcctgcatcagcaccaaccaagggactatccacctacaatccttgaaagaccttaaacaatttgccccaagcccttcctgcgagaaaattgaaatcattgctacactgaagaatggagttcaaacatgtctaaacccagattcagcagatgtgaaggaactgattaaaaagtgggagaaacaggtcagccaaaagaaaaagcaaaagaatgggaaaaaacatcaaaaaaagaaagttctgaaagttcgaaaatctcaacgttctcgtcaaaagaagactacataagagaccacttcaccaataagtattctgtgttaaaaatgttctattttaattataccgctatcattccaaaggaggatggcatataatacaaaggcttattaatttgactagaaaatttaaaacattactctgaaattgtaactaaagttagaaagttgattttaagaatccaaacgttaagaattgttaaaggctatgattgtctttgttcttctaccacccaccagttgaatttcatcatgcttaaggccatgattttagcaatacccatgtctacacagatgttcacccaaccacatcccactcacaacagctgcctggaagagcagccctaggcttccacgtactgcagcctccagagagtatctgaggcacatgtcagcaagtcctaagcctgttagcatgctggtgagccaagcagtttgaaattgagctggacctcaccaagctgctgtggccatcaacctctgtatttgaatcagcctacaggcctcacacacaatgtgtctgagagattcatgctgattgttattgggtatcaccactggagatcaccagtgtgtggctttcagagcctcctttctggctttggaagccatgtgattccatcttgcccgctcaggctgaccactttatttctttttgttcccctttgcttcattcaagtcagctcttctccatcctaccacaatgcagtgcctttcttctctccagtgcacctgtcatatgctctgatttatctgagtcaactcctttctcatcttgtccccaacaccccacagaagtgctttcttctcccaattcatcctcactcagtccagcttagttcaagtcctgcctcttaaataaacctttttggacacacaaattatcttaaaactcctgtttcacttggttcagtaccacatgggtgaacactcaatggttaactaattcttgggtgtttatcctatctctccaaccagattgtcagctccttgagggcaagagccacagtatatttccctgtttcttccacagtgcctaataatactgtggaactaggttttaataattttttaattgatgttgttatgggcaggatggcaaccagaccattgtctcagagcaggtgctggctctttcctggctactccatgttggctagcctctggtaacctcttacttattatcttcaggacactcactacagggaccagggatgatgcaacatccttgtctttttatgacaggatgtttgctcagcttctccaacaataagaagcacgtggtaaaacacttgcggatattctggactgtttttaaaaaatatacagtttaccgaaaatcatataatcttacaatgaaaaggactttatagatcagccagtgaccaaccttttcccaaccatacaaaaattccttttcccgaaggaaaagggctttctcaataagcctcagctttctaagatctaacaagatagccaccgagatccttatcgaaactcattttaggcaaatatgagttttattgtccgtttacttgtttcagagtttgtattgtgattatcaattaccacaccatctcccatgaagaaagggaacggtgaagtactaagcgctagaggaagcagccaagtcggttagtggaagcatgattggtgcccagttagcctctgcaggatgtggaaacctccttccaggggaggttcagtgaattgtgtaggagaggttgtctgtggccagaatttaaacctatactcactttcccaaattgaatcactgctcacactgctgatgatttagagtgctgtccggtggagatcccacccgaacgtcttatctaatcatgaaactccctagttccttcatgtaacttccctgaaaaatctaagtgtttcataaatttgagagtctgtgacccacttaccttgcatctcacaggtagacagtatataactaacaaccaaagactacatattgtcactgacacacacgttataatcatttatcatatatatacatacatgcatacactctcaaagcaaataatttttcacttcaaaacagtattgacttgtataccttgtaatttgaaatattttctttgttaaaatagaatggtatcaataaatagaccattaatcag

SEQ ID NO: 163 - DNA, 715 bp, Homo sapiens
  NM_002704
tgcagacttgtaggcagcaactcaccctcactcagaggtcttctggttctggaaacaactctagctcagccttctccaccatgagcctcagacttgataccaccccttcctgtaacagtgcgagaccacttcatgccttgcaggtgctgctgcttctgtcattgctgctgactgctctggcttcctccaccaaaggacaaactaagagaaacttggcgaaaggcaaagaggaaagtctagacagtgacttgtatgctgaactccgctgcatgtgtataaagacaacctctggaattcatcccaaaaacatccaaagtttggaagtgatcgggaaaggaacccattgcaaccaagtcgaagtgatagccacactgaaggatgggaggaaaatctgcctggacccagatgctcccagaatcaagaaaattgtacagaaaaaattggcaggtgatgaatctgctgattaatttgttctgtttctgccaaacttctttaactcccaggaagggtagaattttgaaaccttgattttctagagttctcatttattcaggatacctattcttactgtattaaaatttggatatgtgtttcattctgtctcaaaaatcacattttattctgagaaggttggttaaaagatggcagaaagaagatgaaaataaataagcctggtttcaaccctctaattcttgcctaaaaaaaaaaaaaaaaa

SEQ ID NO: 164 - DNA, 1345 bp, Homo sapiens
  NM_002923
aaaacagccggggctccagcgggagaacgataatgcaaagtgctatgttcttggctgttcaacacgactgcagacccatggacaagagcgcaggcagtggccacaagagcgaggagaagcgagaaaagatgaaacggacccttttaaaagattggaagacccgtttgagctacttcttacaaaattcctctactcctgggaagcccaaaaccggcaaaaaaagcaaacagcaagctttcatcaagccttctcctgaggaagcacagctgtggtcagaagcatttgacgagctgctagccagcaaatatggtcttgctgcattcagggcttttttaaagtcggaattctgtgaagaaaatattgaattctggctggcctgtgaagacttcaaaaaaaccaaatcaccccaaaagctgtcctcaaaagcaaggaaaatatatactgacttcatagaaaaggaagctccaaaagagataaacatagattttcaaaccaaaactctgattgcccagaatatacaagaagctacaagtggctgctttacaactgcccagaaaagggtatacagcttgatggagaacaactcttatcctcgtttcttggagtcagaattctaccaggacttgtgtaaaaagccacaaatcaccacagagcctcatgctacatgaaatgtaaaagggagcccagaaatggaggacatttcattctttttcctgaggggaaggactgtgacctgccataaagactgaccttgaattcagcctgggtgttcaggaaacatcactcagaactattgattcaaagttgggtagtgaatcaggaagccagtaactgactaggagaagctggtatcagaacagcttccctcactgtgtacagaacgcaagaagggaataggtggtctgaacgtggtgtctcactctgaaaagcaggaatgtaagatgatgaaagagacaatgtaatactgttggtccaaaagcatttaaaatcaatagatctgggattatgtggccttaggtagctggttgtacatctttccctaaatcgatccatgttaccacatagtagttttagtttaggattcagtaacagtgaagtgtttactatgtgcaagggtattgaagttcttatgaccacagatcatcagtactgttgtctcatgtaatgctaaaactgaaatggtccgtgtttgcattgttaaaaatgatgtgtgaaatagaatgagtgctatggtgttgaaaactgcagtgtccgttatgagtgccaaaaatctgtcttgaaggcagctacactttgaagtggtctttgaatacttttaataaatttattttgataaataatattg

SEQ ID NO: 165 - DNA, 776 bp, Homo sapiens
  NM_002983
aaggacacgggcagcagacagtggtcagtcctttcttggctctgctgacactcgagcccacattccgtcacctgctcagaatcatgcaggtctccactgctgcccttgctgtcctcctctgcaccatggctctctgcaaccagttctctgcatcacttgctgctgacacgccgaccgcctgctgcttcagctacacctcccggcagattccacagaatttcatagctgactactttgagacgagcagccagtgctccaagcccggtgtcatcttcctaaccaagcgaagccggcaggtctgtgctgaccccagtgaggagtgggtccagaaatatgtcagcgacctagagctgagtgcctgaggggtccagaagcttcgaggcccagcgacctcggtgggccagtggggaggagcaggagcctgagccttgggaaacatgcgtgtgacctccacagctacctcttctatggactggttgttgccaaacagccacactgtgggactcttcttaacttaaattttaatttatttatactatttagtttttgtaatttattttcgatttcacagtgtgtttgtgattgtttgctctgagagttcccctgtcccctcccccttccctcacaccgcgtctggtgacaaccgagtggctgtcatcagcctgtgtaggcagtcatggcaccaaagccaccagactgacaaatgtgtatcggatgcttttgttcagggctgtgatcggcctggggaaataataaagcacgctcttttaaaaggt

SEQ ID NO: 166 - DNA, 696 bp, Homo sapiens
  NM_002984
ttccccccccccccccccccccccgcccgagcacaggacacagctgggttctgaagcttctgagttctgcagcctcacctctgagaaaacctcttttccaccaataccatgaagctctgcgtgactgtcctgtctctcctcatgctagtagctgccttctgctctccagcgctctcagcaccaatgggctcagaccctcccaccgcctgctgcttttcttacaccgcgaggaagcttcctcgcaactttgtggtagattactatgagaccagcagcctctgctcccagccagctgtggtattccaaaccaaaagaagcaagcaagtctgtgctgatcccagtgaatcctgggtccaggagtacgtgtatgacctggaactgaactgagctgctcagagacaggaagtcttcagggaaggtcacctgagcccggatgcttctccatgagacacatctcctccatactcaggactcctctccgcagttcctgtcccttctcttaatttaatcttttttatgtgccgtgttattgtattaggtgtcatttccattatttatattagtttagccaaaggataagtgtcctatggggatggtccactgtcactgtttctctgctgttgcaaatacatggataacacatttgattctgtgtgttttccataataaaactttaaaataaaatgcagacagtta

SEQ ID NO: 167 - DNA, 3752 bp, Homo sapiens
  NM_003010
ggccgtgcgagaggccgagcttgctgcattgcagccgccgcggcgccgctcggctcttcactcccaacaatggcggctccgagcccgagcggcggcggcggctccgggggcggcagcggcagcggcacccccggccccgtagggtccccggcgccaggccacccggccgtcagcagcatgcagggtaaacgcaaagcactgaagttgaattttgcaaatccacctttcaaatctacagcaaggtttactctgaatcccaatcctacaggagttcaaaacccacacatagagagactgagaacacacagcattgagtcatcaggaaaactgaagatctcccctgaacaacactgggatttcactgcagaggacttgaaagaccttggagaaattggacgaggagcttatggttctgtcaacaaaatggtccacaaaccaagtgggcaaataatggcagttaaaagaattcggtcaacagtggatgaaaaagaacaaaaacaacttcttatggatttggatgtagtaatgcggagtagtgattgcccatacattgttcagttttatggtgcactcttcagagagggtgactgttggatctgtatggaactcatgtctacctcgtttgataagttttacaaatatgtatatagtgtattagatgatgttattccagaagaaattttaggcaaaatcactttagcaactgtgaaagcactaaaccacttaaaagaaaacttgaaaattattcacagagatatcaaaccttccaatattcttctggacagaagtggaaatattaagctctgtgacttcggcatcagtggacagcttgtggactctattgccaagacaagagatgctggctgtaggccatacatggcacctgaaagaatagacccaagcgcatcacgacaaggatatgatgtccgctctgatgtctggagtttggggatcacattgtatgagttggccacaggccgatttccttatccaaagtggaatagtgtatttgatcaactaacacaagtcgtgaaaggagatcctccgcagctgagtaattctgaggaaagggaattctccccgagtttcatcaactttgtcaacttgtgccttacgaaggatgaatccaaaaggccaaagtataaagagcttctgaaacatccctttattttgatgtatgaagaacgtgccgttgaggtcgcatgctatgtttgtaaaatcctggatcaaatgccagctactcccagctctcccatgtatgtcgattgatatcgctgctacatcagactctagaaaaaagggctgagaggaagcaagacgtaaagaattttcatcccgtatcacagtgtttttattgctcgcccagacaccatgtgcaataagattggtgttcgtttccatcatgtctgtatactcctgtcacctagaacgtgcatccttgtaatacctgattgatcacacagtgttagtgctggtcagagagacctcatcctgctcttttgtgatgaacatattcatgaaatgtggaagtcagtacgatcaagttgttgactgtgattagatcacatcttaaattcatttctagactcaaaacctggagatgcagctactggaatggtgttttgtcagacttccaaatcctggaaggacacagtgatgaatgtactatgtctgaacatagaaactcgggcttgagtgagaagagcttgcacagccaacgagacacattgccttctggagctgggagacaaaggaggaatttactttcttcaccaagtgcaatagattactgatgtgatattctgttgctttacagttacagttgatgtttggggatcgatgtgctcagccaaatttcctgtttgaaatatcatgttaaattagaatgaatttatctttaccaaaaaccatgttgcgttcaaagaggtgaacattaaaatatagagacaggacagaatgtgttcttttctcctttaccagtcctatttttcaatgggaagactcaggagtctgccacttgtcaaagaaggtgctgatcctaagaatttttcattctcagaattcggtgtgctgccaacttgatgttccacctgccacaaaccaccaggactgaaagaagaaaacagtacagaaggcaaagtttacagatgtttttaattctagtattttatctggaacaacttgtagcagctatatatttccccttggtcccaagcctgatactttagccatcataactcactaacagggagaagtagctagtagcaatgtgccttgattgattagataaagatttctagtaggcagcaaaagaccaaatctcagttgtttgcttcttgccatcactggtccaggtcttcagtttccgaatctctttcccttcccctgtggtctattgtcgctatgtgacttgcgcttaatccaatattttgccttttttctatatcaaaaaacctttacagttagcagggatgttccttaccaaggatttttagccccaaatctctcatattcgctagtgtttaaaaggctaagaatagtggggcccagccgatgtggtaggtgataaagaggcatcttttctagagacacattggaccagatgaggatccgaaacggcagcctttacgttcatcacctgctagaacctctcgtagtccatcaccatttcttggcattggaattctactggaaaaaaatacaaaaagcaaaacaaaaccctcagcactgttacaagaggccatttaagtatcttgtgcttcttcacttacccattagccaggttctcattaggttttgcttgggcctccctggcactgaaccttaggctttgtatgacagtgaagcagcactgtgagtggttcaagcacactggaatataaaacagtcatggcctgagatgcaggtgatgccattacagaaccaaatcgtggcacgtattgctgtgtctcctctcagagtgacagtcataaatactgtcaaacaataaagggagaatggtgctgtttaaagtcacatccctgtaaattgcagaattcaaaagtgattatctctttgatctacttgcctcatttccctatcttctcccccacggtatcctaaactttagacttcccactgttctgaaaggagacattgctctatgtctgccttcgaccacagcaagccatcatcctccattgctcccggggactcaagaggaatctgtttctctgctgtcaacttcccatctggctcagcatagggtcactttgccattatgcaaatggagataaaagcaattctgactgtccaggagctaatctgaccgttctattgtgtggatgaccacataagaaggcaattttagtgtattaatcatagattattataaactataaacttaagggcaaggagtttattacaatgtatctttattaaaacaaaagggtgtatagtgttcacaaactgtgaaaatagtgtaagaactgtacattgtgagctctggttatttttctcttgtaccatagaaaaatgtataaaaattatcaaaaagctaatgtgcagggatattgccttatttgtctgtaaaaaatggagctcagtaacataactgcttcttggagctttggaatattttatcctgtattcttgtttgaattcctcctctatttaagatatatacatggaatcgaagtgtttatgtaatagttctatccttttgcctgcaggtcagttgtaataaatctaggatgtgatgatgaaaaaaaaaa

SEQ ID NO: 168 - DNA, 2594 bp, Homo sapiens
  NM_003082
gcttcgggtgccatggggactcctcccggcctgcagaccgactgcgaggcgctgctcagccgcttccaggagacggacagtgtacgcttcgaggacttcacggagctctggagaaacatgaagttcgggactatcttctgtggcagaatgagaaatttagaaaagaacatgtttacaaaagaagctttagctttggcttggcgatattttttacctccatacaccttccagatcagagttggtgctttgtatctgctatatggattatataatacccaactgtgtcaaccaaaacaaaagatcagagttgccctgaaggattgggatgaagttttaaaatttcagcaagatttagtaaatgcacagcattttgatgcagcttatatttttaggaagctacgactagacagagcatttcactttacagcaatgcccaaattgctgtcatataggatgaagaaaaaaattcaccgagctgaagttacagaagaatttaaggacccaagtgatcgtgtgatgaaacttatcacttctgatgtattagaggaaatgctgaatgttcatgatcattatcagaacatgaaacatgtaatttcagttgataagtccaagccagataaagccctcagcttgataaaggatgatttttttgacaatattaagaacatagttttggagcatcagcagtggcacaaagacagaaagaatccatccttaaagtcaaaaactaatgatggagaagaaaaaatggaaggaaattcacaagaaacggagagatgtgaaagggcagaatcattagcgaaaataaaatcaaaggccttttcagttgtcatacaggcatccaaatcaagaaggcatcgtcaagtcaaactcgactcttctgactctgattctgcatctggtcaagggcaagtcaaagcaactaggaaaaaagagaagaaagaaagattgaaaccagcaggaaggaagatgtctctcagaaacaaaggcaatgtgcagaatatacacaaggaagataaacctttaagtctgagtatgcctgtaattacagaagaagaagagaatgaaagtttgagtggaacagagttcactgcatccaagaagaggagaaaacactgaacaaagagcctggtgtagtttttaattttgagttttctgacagaagaaaagattgatattttgtgtattgaacaggaagactgccagtattaaaaaaatccttctgggaatctgtaggttatttcttggaaattgcaatacgtagttctagaataaaagtacaaaaaattagaataagaattctttaacattttctttaatgatttgcataaatggagataaaacttgtatttagtatgtaatagaaaaaattctgttattcgcagattgttactatttcctataaggttttgtgatactatactgtcctaatacagtctggtaatactattctattttatttaaaatattttttattgaaatattaatgtttattacatgcaaataactattttgtatctacagtcggataatggattttttattttgtatatttattctattttgtatattgttaagtgcaataaagtttttgccttgctttattttttaatacataaaacttacattctcataacgtgattgataacttaggaagttcacaatgtattttctacttctgcaattaaatattctttagtgcttgtttattattactaaatactaattaagtactaacaagtacttaaatactaatgtattaagtatttaagtactttctaataaaatctttaacaataataatgtaaatttcagaatgtgtctctggtacagaatagttgatattaacagaaaaaaaaaaatctgtagcttcatgaatatgccactctgttaatttcttgttccagacattttaatagagattgcttgagccatgttgtttgaattgctgccaatagcagaccatatccctatcatgttgttggctcaactgttttttttttttccctaatagagatggagtatcgctatgttgctcaggctggtcttgaactcctgggctcaagctatcctcctgcctcagcctcccaaagtactgggattataggtgtgagctactgtacccagccttaacctgtttcacagttgattatacttcatgctgttttccagcatggtattattaagggatttaaagtttgggttgcatgcctgtaatcccagcattttgggaggccgaggtgggcggatcacgaggtcaggagatcgagaccatcgtgactaacacagtgaaaccccgtctctaataaaaatacgaaaaattagccaggcgtggtggcgggcgcctgtaatcccagctactcgggaagctgaggcaggagaatggtgtgaacccagtgagccgagatcgtgccactgcactccagcctgggcaacagagtgagacttcgtctcaaaaaaaaaaaaaaaagtttgggttgaagatcaaattcgtgatatctctatatctaatctttaaaaatcagaatgctaatgctgacgcaaataaaattttcatttattagcaaaaaaaaaaaaaaaaaaaaaaaaaaaaa

SEQ ID NO: 169 - DNA, 1931 bp, Homo sapiens
  NM_003651
gcccaagagcgagcgcgcagcacgaagctcgagccgcctccgccgcgcgaccccacctcggccgccgccgcctgcgccgcgagatccgccccggcctccccgagagcgagccccggccgccgcgaccaccagccgcgctaaccgccgaccaaccgccaccgaggcgcctgagcgagagcagaggaggaggaggcatgagtgaggcgggcgaggccaccaccaccaccaccaccaccctcccgcaggctccgacggaggcggccgccgcggctccccaggaccccgcgcccaagagcccggtgggcagcggtgcgccccaggccgcggccccggcgcccgccgcccacgtcgcaggaaaccccggtggggacgcggcccccgcagccacgggcaccgcggccgccgcctctttagccgccgccgccggcagcgaagacgcggagaaaaaagttctcgccaccaaagtccttggcactgtcaaatggttcaacgtcagaaatggatatggatttataaatcgaaatgacaccaaagaagatgtatttgtacatcagactgccatcaagaagaataacccacggaaatatctgcgcagtgtaggagatggagaaactgtagagtttgatgtggttgaaggagagaagggtgcagaagctgccaatgtgactggcccggatggagttcctgtggaagggagtcgttacgctgcagatcggcgccgttacagacgtggctactatggaaggcgccgtggccctccccggaattacgctggggaggaggaggaggaagggagcggcagcagtgaaggatttgacccccctgccactgataggcagttctctggggcccggaatcagctgcgccgcccccagtatcgccctcagtaccggcagcggcggttcccgccttaccacgtgggacagacctttgaccgtcgctcacgggtcttaccccatcccaacagaatacaggctggtgagattggagagatgaaggatggagtcccagagggagcacaacttcagggaccggttcatcgaaatccaacttaccgcccaaggtaccgtagcaggggacctcctcgcccacgacctgccccagcagttggagaggctgaagataaagaaaatcagcaagccaccagtggtccaaaccagccgtctgttcgccgtggatatcggcgtccctacaattaccggcgtcgcccgcgtcctcctaacgctccttcacaagatggcaaagaggccaaggcaggtgaagcaccaactgagaaccctgctccacccacccagcagagcagtgctgagtaacaccaggctcctcaggcaccttcaccatcggcaggtgacctaaagaattaatgaccattcagaaataaagcaaaaagcaggccacaaccttaaccaacaccaaagaaacatccaagcaataaagtggaagactaaccaagatttggacattggaatgtttactgttattctttaagaaacatctacaaaaagaaaatgtcaacaaatttttccagcaagctgagaacctgggaattcctgcacggaagacaagagagtagcctctccagtttcagcaaccgctaggtttctattttttttcctggtttttactgttttggtaatatatatattgaaacaagaaatattaataccacatggggagaaccccaaccaaagaaatctgaaatatatagtaaatgcttttttttccgtttttgttcattttggatgctggtgctaaacctccaagtgtcatgatttaaaaaaaaaaaaaatttatgtccttcttatttatttctaggatgaggggaggataacatttttgctttcttatgtgactctctttgaaaatgtgcagtaagaaattcctcaaaaataaaatttttacccttcagaggaaaaaaaaaaaaaaaaaaa

SEQ ID NO: 170 - DNA, 716 bp, Homo sapiens
  NM_003806
gaaacttggtgtccaggggaggcccccggcggctggagcgcggcggcagcgggcgcagaggccggagggagaggaggcgaggggcggcccgagcgcggggcgggagcgaggccagcggtcatgtgcccgtgccccctgcaccgcggccgcggccccccggccgtgtgcgcctgcagcgcgggtcgcctggggctgcgctcgtccgccgcgcagctcaccgccgcccggctcaaggcgctaggcgacgagctgcaccagcgcaccatgtggcggcgccgcgcgcggagccggagggcgccggcgcccggcgcgctccccacctactggccttggctgtgcgcggccgcgcaggtggcggcgctggcggcctggctgctcggcaggcggaacttgtaggaacgcggggcttcttggtggggccggagccgagacccagccggagcgagcaacaggttggtgaaaaccctgtgtccttggagaaagctggttcccgttttccagagggggagcccagagcttgaaaggccgcggttggcacttcgagaaggaagtggagagtaaagacagcgcctggagcgatcgtagaaacacagaatgggactggggaagccctttggaaatccagctgcagaaacagacaccccaatgctatttacatacagctctatatatataaaaaaagaaaatatgaatattaaaaaaaaaaaaaaaaaaaa

SEQ ID NO: 171 - DNA, 1645 bp, Homo sapiens
  NM_003811
agtctctcgtcatggaatacgcctctgacgcttcactggaccccgaagccccgtggcctcccgcgccccgcgctcgcgcctgccgcgtactgccttgggccctggtcgcggggctgctgctgctgctgctgctcgctgccgcctgcgccgtcttcctcgcctgcccctgggccgtgtccggggctcgcgcctcgcccggctccgcggccagcccgagactccgcgagggtcccgagctttcgcccgacgatcccgccggcctcttggacctgcggcagggcatgtttgcgcagctggtggcccaaaatgttctgctgatcgatgggcccctgagctggtacagtgacccaggcctggcaggcgtgtccctgacggggggcctgagctacaaagaggacacgaaggagctggtggtggccaaggctggagtctactatgtcttctttcaactagagctgcggcgcgtggtggccggcgagggctcaggctccgtttcacttgcgctgcacctgcagccactgcgctctgctgctggggccgccgccctggctttgaccgtggacctgccacccgcctcctccgaggctcggaactcggccttcggtttccagggccgcttgctgcacctgagtgccggccagcgcctgggcgtccatcttcacactgaggccagggcacgccatgcctggcagcttacccagggcgccacagtcttgggactcttccgggtgacccccgaaatcccagccggactcccttcaccgaggtcggaataacgcccagcctgggtgcagcccacctggacagagtccgaatcctactccatccttcatggagacccctggtgctgggtccctgctgctttctctacctcaaggggcttggcaggggtccctgctgctgacctccccttgaggaccctcctcacccactccttccccaagttggaccttgatatttattctgagcctgagctcagataatatattatatatattatatatatatatatatttctatttaaagaggatcctgagtttgtgaatggacttttttagaggagttgttttggggggggggtcttcgacattgccgaggctggtcttgaactcctggacttagacgatcctcctgcctcagcctcccaagcaactgggattcatcctttctattaattcattgtacttatttgcctatttgtgtgtattgagcatctgtaatgtgccagcattgtgcccaggctagggggctatagaaacatctagaaatagactgaaagaaaatctgagttatggtaatacgtgaggaatttaaagactcatccccagcctccacctcctgtgtgatacttgggggctagcttttttctttctttcttttttttgagatggtcttgttctgtcaaccaggctagaatgcagcggtgcaatcatgagtcaatgcagcctccagcctcgacctcccgaggctcaggtgatcctcccatctcagcctctcgagtagctgggaccacagttgtgtgccaccacacttggctaactttttaatttttttgcggagacggtattgctatgttgccaaggttgtttacatgccagtacaatttataataaacactcatttttcctcaaaaaaaaaaaaaaaa

SEQ ID NO: 172 - DNA, 1402 bp, Homo sapiens
  NM_003946
gagaggcaggaggacaccgagttccccgtgttggcctccaggtcctgtgcttgcggagccgtccggcggctgggatcgagccccgacaatgggcaacgcgcaggagcggccgtcagagactatcgaccgcgagcggaaacgcctggtcgagacgctgcaggcggactcgggactgctgttggacgcgctgctggcgcggggcgtgctcaccgggccagagtacgaggcattggatgcactgcctgatgccgagcgcagggtgcgccgcctactgctgctggtgcagggcaagggcgaggccgcctgccaggagctgctacgctgtgcccagcgtaccgcgggcgcgccggaccccgcttgggactggcagcacgtgggtccgggctaccgggaccgcagctatgaccctccatgcccaggccactggacgccggaggcacccggctcggggaccacatgccccgggttgcccagagcttcagaccctgacgaggccgggggccctgagggctccgaggcggtgcaatccgggaccccggaggagccagagccagagctggaagctgaggcctctaaagaggctgaaccggagccggagccagagccagagctggaacccgaggctgaagcagaaccagagccggaactggagccagaaccggacccagagcccgagcccgacttcgaggaaagggacgagtccgaagattcctgaaggccagagctctgacaggcggtgccccgcccatgctggataggacctgggatgctgctggagctgaatcggatgccaccaaggctcggtccagcccagtaccgctggaagtgaataaactccggagggtcggacgggacctgggctctctccacgattctggctgtttgcccaggaacttagggtgggtacctctgagtcccagggacctgggcaggcccaagcccaccacgagcatcatccagtcctcagccctaatctgcccttaggagtccaggctgcaccctggagatcccaaacctagccccctagtgggacaaggacctgaccctcctgcccgcatacacaacccatttcccctggtgagccacttggcagcatatgtaggtaccagctcaaccccacgcaagttcctgagctgaacatggagcaaggggagggtgacttctctccacatagggagggcttagagctcacagccttgggaagtgagactagaagaggggagcagaaagggaccttgagtagacaaaggccacacacatcattgtcattactgttttaattgtctggcttctctctggactgggagctcagtgaggattctgaccagtgacttacacaaaaggcgctctatacatattataatatattcgcttactaaatgaaaaaaaaaaaaaaaaaaaa

SEQ ID NO: 173 - DNA, 1159 bp, Homo sapiens
  NM_004221
ccccagccagctgtcccgagtctggactttccctctgcccctccccactctcaggctggtggggtggggaaagcagcccattcctgggctcagagactcccaccccagctcagagggagcaggggcccagccagggacggaccctcattcctcccagggaccccagacctctgtctctctcgggccttggctccttgaacttttggccgccatgtgcttcccgaaggtcctctctgatgacatgaagaagctgaaggcccgaatggtaatgctcctccctacttctgctcaggggttgggggcctgggtctcagcgtgtgacactgaggacactgtgggacacctgggaccctggagggacaaggatccggccctttggtgccaactctgcctctcttcacagcaccaggccatagaaagattttatgataaaatgcaaaatgcagaatcaggacgtggacaggtgatgtcgagcctggcagagctggaggacgacttcaaagagggctacctggagacagtggcggcttattatgaggagcagcacccagagctcactcctctacttgaaaaagaaagagatggattacggtgccgaggcaacagatcccctgtcccggatgttgaggatcccgcaaccgaggagcctggggagagcttttgtgacaaggtcatgagatggttccaggccatgctgcagcggctgcagacctggtggcacggggttctggcctgggtgaaggagaaggtggtggccctggtccatgcagtgcaggccctctggaaacagttccagagtttctgctgctctctgtcagagctcttcatgtcctctttccagtcctacggagccccacggggggacaaggaggagctgacaccccagaagtgctctgaaccccaatcctcaaaatgaagatactgacaccacctttgccctccccgtcaccgcgcacccaccctgacccctccctcagctgtcctgtgccccgccctctcccgcacactcagtccccctgcctggcgttcctgccgcagctctgacctggtgctgtcgccctggcatcttaataaaacctgcttatacttccctggcaggggagataccctgaaaaaaaaaaaaaaaaaaaaaaaaaaaaaaaaaaaaaaaaaaaaaaaa

SEQ ID NO: 174 - DNA, 891 bp, Homo sapiens
  NM_004324
tcacgtgacccgggcgcgctgcggccgcccgcgcggacccggcgagaggcggcggcgggagcggcggtgatggacgggtccggggagcagcccagaggcggggggcccaccagctctgagcagatcatgaagacaggggcccttttgcttcagggtttcatccaggatcgagcagggcgaatggggggggaggcacccgagctggccctggacccggtgcctcaggatgcgtccaccaagaagctgagcgagtgtctcaagcgcatcggggacgaactggacagtaacatggagctgcagaggatgattgccgccgtggacacagactccccccgagaggtctttttccgagtggcagctgacatgttttctgacggcaacttcaactggggccgggttgtcgcccttttctactttgccagcaaactggtgctcaaggccctgtgcaccaaggtgccggaactgatcagaaccatcatgggctggacattggacttcctccgggagcggctgttgggctggatccaagaccagggtggttgggtgagactcctcaagcctcctcacccccaccaccgcgccctcaccaccgcccctgccccaccgtccctgccccccgccactcctctgggaccctgggccttctggagcaggtcacagtggtgccctctccccatcttcagatcatcagatgtggtctataatgcgttttccttacgtgtctgatcaatccccgattcatctaccctgctgacctcccagtgacccctgacctcactgtgaccttgacttgattagtgccttctgccctccctggagcctccactgcctctggaattgctcaagttcattgatgaccctctgaccctagctctttcctttttttttttt

SEQ ID NO: 175 - DNA, 6193 bp, Homo sapiens
  NM_004437
gccccggcggggcaaagtggcaggaacctcttaaagggcgagagcggcgcggagccagaacgcggtcggcccggtccccgccgcacccagcccagagaagagtttagtgactgaggccgaaaattcacagcaccaacagaaggaagagggtgaggaagccataaactcaggccaacaagaacctcagcaggaggaatcttgtcaaacagcagctgaaggagataattggtgtgaacagaagctgaaagcttctaatggagacactcctacacatgaagacttgaccaagaacaaggagcggacatcagaaagcagaggactttcacgactattctcctcgtttctcaaaaggcccaaatctcaggtgtccgaggaagaaggcaaagaagtagagtcagataaagaaaaaggtgaaggaggtcagaaagagatagaatttggaaccagtcttgatgaagagatcattttaaaggccccaattgcagctcctgaaccggaactcaaaacagacccatctttggatcttcattcattaagcagtgcagaaacacagcctcaccattacaattaagaattattttcagagtcttcttatttctgaagcatgtgaatattatctgcatcgttaaaagtcctgctcaggaagaactcagagaagatccagattttgaaattaaggaaggagaaggacttgaagagtgctccaaaatagaagtaaaagaagaaagccctcaatcaaaagcagaaacagaattaaaagcttcccaaaaaccaatcagaaaacacaggaacatgcactgcaaggtttctttgttggatgacacagtttatgaatgtgttgtggagaaacatgctaagggacaagatttgcttaaacgagtatgtgagcatctcaatcttttggaagaagactattttggtctagccatttgggataacgcaacctctaagacatggctggattccgccaaagaaataaaaaagcaggttcgtggtgtcccttggaattttacatttaatgtaaagttttatccacctgacccagcacagttaacagaagacataacaagatattatttatgtcttcagcttcggcaggacatagttgcaggacgtctgccctgttcctttgcaaccttagcattattaggttcttacaccatccagtctgaactgggagactacgacccagaactccatggcgtggattatgttagtgattttaaactggccccgaatcagaccaaggaacttgaagagaaggtcatggaactgcataagtcatacaggtccatgactccagctcaggctgacttggagtttcttgagaatgccaaaaagttgtctatgtatggagttgatcttcataaagcaaaggacttggaaggagtagatatcatcctaggtgtctgctctagtggccttctggtttacaaagataagctgagaattaaccgcttcccttggcccaaagtgctgaagatttcttataaacgtagtagctttttcatcaagattcggcctggagagcaagagcagtatgaaagtaccatcggattcaaacttcccagttaccgagcagctaagaaattatggaaagtctgtgtagaacatcacacgtttttcagattgacatctacagacaccattcccaaaagcaaatttcttgcgctaggatccaaatttcgatacagtggccggactcaagctcagaccaggcaagctagtgctctaattgacaggcctgccccacacttcgagcgtacagcaagtaaacgggcgtcccggagcctcgatggagcagcagctgtcgattcggcagaccgaagtcctcggcccacttctgcacctgccattactcagggtcaggttgcagaaggtggcgtcctagatgcctctgctaaaaaaacagtggtccctaaagcacagaaggaaacagtgaaggctgaagtgaaaaaggaagacgagccacctgagcaagctgagccagagcccacagaagcatggaagaaaaagagagaaagactagatggtgaaaacatttatatcagacatagcaatttaatgttggaggatttagacaagagtcaagaggagatcaaaaaacatcatgccagcatcagtgagctgaaaaagaacttcatggagtctgtaccagaaccacggcctagtgaatgggataaacgcttatccactcactcacccttccgaactcttaacatcaatgggcaaatccccacaggagaaggacctcccctggtgaagacacaaactgtcaccatctcagataatgccaatgctgtgaaaagtgaaatcccaaccaaagacgtccctattgtccacactgagaccaagaccatcacttatgaggctgcccagactgtaaaaggtgggatttcagagacacgtattgaaaagagaattgtgatcacaggagatgctgatattgaccatgatcaggtccttgtacaagccatcaaggaggcaaaggagcagcacccagacatgtcagtgaccaaggtggtcgtccaccaggagaccgagattgctgatgagtgagctcaggaactaacctaccccaactctgcccttctcccatccaagagaaaccagcaaaatgataaagaagctaacctgccatagtcagacttcagactttcaagattattctaaatcaccagaaaattaatttcagtttctattgggagtttataccaagagattcttctagatctcattgatccttttgaagagctttttctatattaggatatcagaattgttcaacttttcactctatagactgttttaagagttttggggtttttttaattgggtggtttgtaaccccttcagcctagcctctctgcccatttatttccaaccccaacagacactgacagggtccatggaattcttcgggaaatcctccaaggactcttgtcagctgtgttggaagccaaagccagcttagtgggacttccgcgtctctccctagtcttatcccctttggatgatggcagaaacttcatgaaccagccctttctcagagccagtgatgtgagtgtatcagaatgccagggagggcaccagccctgatccacagacctcggaaagatgcccctgttcctttgttgcgggtggttttggtaaggcagagccctctgctgagaatgtagtattgtttttcccctctccctcctgctttctttttggagcttctttgggtcaaagacatggaagttgcttcagatatctgatactgtgaatgtttgaacatatccgtggccttcacctctccagctacccttttacctcatcagaagcagtggctcagctaagtgctccccctagctcccatctcaggagaccaaatctcacagaaaaataggcactttgggccaaaagctctaatggaacatttttagtggtgatttggggaaggaaagttaatgaggtttttaaaataaggttttctagttttgagagtgtgcacttcacacaggggaatggggttacttctgtctgatcctgggcctttctttcatcccaaatgacaaggaatgtggctcagagaagggtttttcttttttgacctttcttctctcaacaggaacctgcctgaggacacccttctagagcaaggaattgacttttaggagccgttctccccacaagacaccacatgacaaggggtataagccccagccctgctcattcccactcaccagctgaggtctgtcaggttttgaaggcttgattttgtggtgggtttggggcttagttttcctttttttcattttgatttttgaaagtgaagatgatgccctaattcctggtaaggatttggggcatagttttttgtttttttgagacggagtttcgctcttgttgcccaagctggagtgcagtggcgcgatctcggctcactgcaacctccgcctcctgggttcaagcagttctcttgcctcagcctcccaagtagctgggatgacaggcgcacaccaccacgcccagcaaattttttgtatttttagtagaaacgggatttcaccttgttaggctggtctcgaactcctgacctcaggtgatccacccaccttggcctcccaaagtgctgggattacaggtgtgagccaccacgcccggccgatttggggcatttttatttaacagaacttctctaaccttccaactgcttcccacaaacacattggcctcaaggctccttagaatcccagttccagcttcctaaaatagacagtgggtatcgggcagcagtcactggggctcaagggcagtgagcaagagaaatgtctaaagctgcttctcccaacaccgtccaaagtctccactgcctgagttttgtttcggctggtttgaactcatttcgggtgtgtgcatttttcttttggtacccatgtgagacatgaacaacaggagggagggaaagagcccaggtgggacgtgggacaggcttaggggaaagagcttgtcctatctcaggaacaaaattataggctgtgggcagagggtctgaaaggtgggctttggggtagtgcccaagcctggtcgtgttgccaggagtggtgacaagaaatgcagcttacatcaaacgaacatgtagtgcatgcccactgcctgatggccagatggcctgtaggaagagctaccagggcttccagacctgtggaacgaagaggatggggaaaaggcagagggcactgagtgtccctttaaaaactaacccactgaatattccgtgtgatctagaacagtgtggcagctttcacagcacaggaccgttcatcgggggcctaaacgtttccctcagctctgtcaccaactcacttctctcggcttcgttgtctgtaaattggatgaaaagagctctaatgcctttcaggctcttagaagccatagatttggacaagcccagcaagatgggtgtccttccaggcctcttcccctttcctccatctctggcaacagttcttggggtttggcaattgtttggattttttttctttctgcagttgtgtgtatgtgtgtttgtgtgaagaaaaacagactctgtccaggtagaaatggtgaggagggggaagagaattacatttccagggtcagaaacttggcaacagttttcctagagtgactcagacacaccacagtaacaactctcgctgcaattttattttaatttgagaaataaagatttcctccaagccacatgaggactctggcacccacccacaaagcaagacctgtatttataagccgagggctcagggagcctaactgcgggacccgtcagggccccgtgacccatccccgtccccacccccccctccaccgctgggcccatcagtgtgtgttggggggatgcttggcagctgggggtgaggagacaacaaacctcgggaactggagccagagctgcggcctgactgacgccttttgatgctcacgggaaatttctgcccaggatctcagccccaggctggttgtttctacaaatctctctcaaatgtattattttggtgacaaaaatgaaggagctttgtaaattttttttaaaattatgaatcatatcaagtagttgtttacatttcttgaaaaaataggaactcgggcagcagaatcagattggcagaatctttagactacacaggcaataatcaagtctgctgttttggcctttcgtagtagaagtggttgtagtgtttagatatctgtttggtcttgcttcttgtattgcatttttttcaataaacaacaacaaaaagaactctctctgtgaggattgatccacttttaaatttctcttctaccagcaacttgggaaaaattaaatatgggtgggggagacctaaactcaagtcattttctaaagtaagttacccacattgaccaaaatgcagcttcaacgttgagtaaagggatttctgagagctggccaatgccttttgccagctgcagtgagattctgcagcataggccacgataaaggaaggagagaaggggcttctcagacttatttgcagaagggcccagaactcagtatgaaggcattggcagtagtgtagctctagagggatataccccagatggctgagggaagaaagggattgaggtggtaggagttcaaggctcagtccccgtcccagatggcagtggagagtctcatcccgtg

SEQ ID NO: 176 - DNA, 6193 bp, Homo sapiens
  NM_004513
tgcttaaaaaaacacaacaggattttcgaagaatcctttcttagaaaacaaacaaaaaaaccaaacaaaaacgtactttctccccactagtttacaccacaggaagcgagagagctgctgccactgctgctaccacaggaagacacagcagggagaagccctagtgcctctgccggctgcccaggacctggtatcggcccacagaccaagtcctccacagagggcgagccagggtggagaagagccagcccagtgacccaaacatccccgataaaacacccactgcttaagaggcaggctcggatggactatagctttgataccacagccgaagacccttgggttaggatttctgactgcatcaaaaacttatttagccccatcatgagtgagaaccatggccacatgcctctacagcccaatgccagcctgaatgaagaagaagggacacagggccacccagatgggaccccaccaaagctggacaccgccaatggcactcccaaagtttacaagtcagcagacagcagcactgtgaagaaaggtcctcctgtggctcccaagccagcctggtttcgccaaagcttgaaaggtttgaggaatcgtgcttcagacccaagagggctccctgatcctgccttgtccacccagccagcacctgcttccagggagcacctaggatcacacatccgggcctcctcctcctcctcctccatcaggcagagaatcagctcctttgaaacctttggctcctctcaactgcctgacaaaggagcccagagactgagcctccagccctcctctggggaggcagcaaaacctcttgggaagcatgaggaaggacggttttctggactcttggggcgaggggctgcacccactcttgtgccccagcagcctgagcaagtactgtcctcggggtcccctgcagcctccgaggccagagacccaggtgtgtctgagtcccctcccccagggcggcagcccaatcagaaaactctcccccctggcccggacccgctcctaaggctgctgtcaacacaggctgaggaatctcaaggcccagtgctcaagatgcctagccagcgagcacggagcttccccctgaccaggtcccagtcctgtgagacgaagctacttgacgaaaagaccagcaaactctattctatcagcagccaagtgtcatcggctgtcatgaaatccttgctgtgccttccatcttctatctcctgtgcccagactccctgcatccccaaggaaggggcatctccaacatcatcatccaacgaagactcagctgcaaatggttctgctgaaacatctgccttggacacggggttctcgctcaacctttcagagctgagagaatatacagagggtctcacggaagccaaggaagacgatgatggggaccacagttcccttcagtctggtcagtccgttatctccctgctgagctcagaagaattaaaaaaactcatcgaggaggtgaaggttctggatgaagcaacattaaagcaattagacggcatccatgtcaccatcttacacaaggaggaaggtgctggtcttgggttcagcttggcaggaggagcagatctagaaaacaaggtgattacggttcacagagtgtttccaaatgggctggcctcccaggaagggactattcagaagggcaatgaggttctttccatcaacggcaagtctctcaaggggaccacgcaccatgatgccttggccatcctccgccaagctcgagagcccaggcaagctgtgattgtcacaaggaagctgactccagaggccatgcccgacctcaactcctccactgactctgcagcctcagcctctgcagccagtgatgtttctgtagaatctacagcagaggccacagtctgcacggtgacactggagaagatgtcggcagggctgggcttcagcctggaaggagggaagggctccctacacggagacaagcctctcaccattaacaggattttcaaaggagcagcctcagaacaaagtgagacagtccagcctggagatgaaatcttgcagctgggtggcactgccatgcagggcctcacacggtttgaagcctggaacatcatcaaggcactgcctgatggacctgtcacgattgtcatcaggagaaaaagcctccagtccaaggaaaccacagctgctggagactcctaggcaggacatgctgaagccaaagccaataacacacagctaacacacagctcccataaccgctgattctcagggtctctgctgccgccccacccagatgggggaaagcacaggtgggcttcccagtggctgctgcccaggcccagaccttctaggacgccacccagcaaaaggttgttcctaaaataagggcagagtcacacgggggcagctgatacaaattgcagactgtgtaaaaagagagcttaatgataatattgtggtgccacaaataaaatggatttattagaatttcatatgacattcatgcctggcttcgcaaaatgtttcaagtactgtaactgtgtcatgattcacccccaaacagtgacatttatttttctcatgaatctgcaatgtgggcagagattggaatgggcagctcatctctgtcccacttggcatcagctggcgtcatgcaaagtcatgcaaaggctgggaccacgtgagatcattcactcatacatctggccgttgatgttggctgggaactcacctggggctgctggcctgaatgcttataggtggcctctccttgtggcctgggcctcctcacaacatggtgtctggattcccaggatgagcatcccaggatcgcaagagccatgtagaagctgcatcttgtttatacctttgccttggaagttgcatggcatcacctccaccatactccatcagttagagctgacacaaacctgcctgggtttaaggggagaggaaatattgctggggtcatttatgaaaaatacagtttgtcacatgaaacatttgcaaaattgtttttggttggattggagaagtaatcctagggaagggtggtggagccagtaaacagaggagtacaggtgaagcaccaagctcaaagcgtggacaggtgtgccgacagaaggaaccagcgtgtatatgagggtatcaaataaaattgctactacttacctaccacatgccaagcactgtgctcagggctaaacgggcattgcctttagtgatcacagcagcttctacggtgtatggttctgtgccaatgtattgataagagggcacacactgtgtacagtaaatggcttatccagctggtgagtgatgcggcagattgagttttcttctgtgattcggtggagactatcagcccaagatgctttaagtgcacaacattacagggaatgcctgagtgcctggccaaagggatatttggtttggccatctctggatgcctgattgccaagctcaggaccaggcaatgtgactttgcatcagcaacaaccagcatcccttgaccaggcctgggccagagtattggtctcctctcagcccctgatcctgtgaagtaaggatgtgggggaagacctggcaaggacacagatgaaacacaaacaatagtaattctcaggccatcatcagtggagccatgttaatgtaatctgatggcttctccagggtccacaggaagtgaagaatctgtttcccagcagtggactcaaaacccatctgggctcctaaccttcctgtaaacccctttagtggcttcattagagcaggcgttcagctcactgttctattcatctcaaggaataatgggcttagagcagtttctgtcctgctggttaacttgtttggcctattccattctggattttgtcaagcagtagacaagcaattagacaagaacttggaggcaccatttgtatccactttttagacttaatagaaacattgaagatgaacataatctaccaacgaaagacgtgattcaattcaacactcccttcccatgacccaggctgggcaaggaggccacgtgatgtggagggcacattccttgcctgcacaaactcaccatctgtgcacgcagtggccttccctaaaatcagggaattgttttaagtcttatcaagcagccaagggatgaaagagaaggtgggttttcatcaagactggaaggtggggacagggatgagcatggagctggccgtgggcctggggtaccaagagactccttgagagaccaggcaaagcaagtgattgggacagaggttatctgtcccaggttatctgggcatagatgcaggtgagcccatggccctcccagtacctcctgtctctggcctgttttagaaggttctctcctccccaaggagacacaacaactcctagggccactgaagatataactattgcccaggtttctggtctctaggctggggaagtcctctgggtaggaatcagcaagaagatcctaaaacaaaagctcatccatttgcgttccatgatgctgggatttacacttgaggcttagctttgctctgccaacttcttcagagctgacacaggatgaaggcaatgccatcctcaaacactgcaggcatcacagctaacaattgtgaagtcgtcttaactcaccataaaaaggaatccactcccaggcagccctacttctttgctttgcccagcattttactgattcatacattatctcacttgtgccaacactcaagaagcaggctacactgacactggtattcctgcctccatattttctttaaaagacaaatcaaagcagatatattaagtgactgttcaagagcacacttggcccaagtggcagagcttggactggatgcatgttttccagctcctcatccagggctctgaccagtttaacctgatgcagtcacgtggaggagcagtgcaggcacagtatgtcccataggcccagtgagatgcattcttggttggctggccttccacttggctacacagggatgtacaaggcgatcccatcttgataagaccaccacctcagagtatggagctcagagagggcaggcatgaagtttccttggctggtgcacctagaattggctgaactcatgagaagttgatatagaacagtgcttgccacagagcggggactcggtaagcacttaatgaatgaatgaattctaagtcaatccaagagtctgatgatttcttgaaaagggtgttagctaaaggatcttaggcatgactgtagaatttgtagttgcaatagaacagagaaagaggaagctttctgtctccttaacactgagctgtcatgttttaaagcttgctcacatcttggcacatttaagagacagtcaccccaggactcaaaaatagggaagtaacagtaacgcaggggaaacgttttctgtttggaggagcaaaggctgagaacactgtgaaaacattttgcgcgcacaatagtaacctgggtaaatgcagcgtgaagggattttagtcacacgtggtctttcttacaaggaaggtggtgggggtgcagatgaggttgctagagaatgttagaggatccctctctggattggagatagggaaagaaagttgcacggctgctgaggccccttctaggtggcaaggctgtgctccctggttctgatgatgtgcctgggtggacatggcccctgtgagtttgtacagtcttgcagcaggatctagaggggggatttccagccagggctgctagacggaggcctactcttccatctttcctgatggcaggatggcctggccagggcctggaagacagagacctcctgcctccgcctcagtaagacgacaaggaaaggcaaatgcccaagggaaagaaaaggaaggctcttctccccagagttccccatgcagacatgagtgcgtgctcagttcagaatcacttctgagaactcatccctaatgctgcagatttgggctggaacagattcacactgtctggtttcaccgaggacatgaaactccaccttgcggggataaagagagaaaaacaaattcatcaaatggaagacacattgaaagtgtttttccttaatgcttatcctgtttttaaaccattatttccaagttgacaccttttttaaggaaaaataaatattttgcggcattaaagccataaaaaaaaaaaaaaaaaaaaaaaaaaaaaaaa

SEQ ID NO: 177 - DNA, 2110 bp, Homo sapiens
  NM_004740
cagaggcctgatgaagactctgagttatgttattgtaccaagaaaggggcttttccccttgggccatcctaaaacaggtcttcccagcctctccttcctgagcctccatttcttgttagtagatgatggcatggtcaaagctgaaaattctcacaggcctcagggagccatcattaggggaaagggttcagatctgggagtatttgacagggaaagcgcctcctcctcagaaggaatctttttaggaatcactcgcctaagagagcccctaccctagacaagttgtactcaccagacctccagtctccaagccaatgtggttcacggactgcctgcttcagagccttctggggaaggactgaaaatgcagaattactgggttgtacccaaaagccagcgattcaaggtttctaagaggaagggcccaggaacctgcatcagattcttatgcacgcttgagtccgagactcactgctctcagcatccctttcctcggagctttaaaagcttccagagtctccttcctcttagcattttgagtatcttctaagtaagccaaggaaggtttttacttaacaatgtttggcttttggggagtgctctgacgcagctttgtgggcctccagaaccaaaggcgtgcgtgcttaatggtaggagtgtcatttgtccctaaccttattcctcggtccctgcagccccaccagctactggaagtcccttgcccctgatcggtcagatgatgagcacgaccctctcgacaacacctccagaccgcgatactcccacagttatctgagtgacagcgacacagaggccaagctgacggagactaacgcatagcccaggggagtggttggcagccctctcaccccagggcctgtggctgcctgggcacctctcccaggaagtggtggggcaccggtctcccccacccgactgctgatctgcatgggaaacaccctgaccttcttctgtcaggggcactttccaggctatgggtgtctgatgtctccacgtggaagaggtgggggaaagaggagtttctgaagagaactttttgctcctctgtctcaaaatgccagactcttggcttctaccctgtgtcaccgtgggcagtggcaggtggcctggcactgcatggagccagcacgttgacctccctctcagctccctgctcagggacggtggacaggttgcctactgggacactctaggttgctgggtccatggggaggattgggggaggagaagcagtgccttccctctcgtgtggggtgggggctctctcttcttggtgcctgctgtctttctactttttaatttaaatacccaacctctccatcacagctgcatccctgagagtgggagggggctgtagtggtagctggggctcccaagaacgactcgggaatgtcatctccatcttcacccttcagagagcagtcctttctctgtgcagctggagacgctggtgaggagagccgggtccaggttcttaagaatgaggtgcggaggggctctccggtgctgctgggctgggttgagcaagcctacgcagacaagtgtgtgtgtggaccatccgcacctccagcccccaccccaccctctttgtctcagcgtgttatgtgcaatgacctatttaaggtaaacccattccaactacagcagttcagggctgatccaagcactgcctccctcctgctctgtccaggtggtctggaccataaactcaacttgagagggaaggcttggggttgaggacttgtgatcagaaaaactgaagatggaagttttggccggtgctcattagacatgagtcctcactctgtgtcctgagcccgtgtcattcttccaacctccctgcccccacacacttatcccagacacaacaccatgtggtctggaggtcccagcccccaccctaaaaaggttatccctgagaactccaccagacttgggagcccaagtgcagtgcctggtgctgctcccatctgccgccccccttctctcctgcaattggtttgtactcactgggctgtgctctcccctgtttacccgatgtatggaaataaaggcccttttcctcctgaaaaaaaaaaaaaaaaaaaaa

SEQ ID NO: 178 - DNA, 2412 bp, Homo sapiens
  NM_005658
caggccaggactccacaaggctggtcccctgccctggagcaacttaaacaggccctctggccagcctggaaccctgagatggcctccagctcaggcagcagtcctcgcccggcccctgatgagaatgagtttccctttgggtgccctcccaccgtctgccaggacccaaaggagcccagggctctctgctgtgcaggctgtctctctgagaacccgaggaatggcgaggatcagatctgccccaaatgcagaggggaagacctccagtctataagcccaggaagccgtcttcgaactcaggagaaggctcaccccgaggtggctgaggctggaattgggtgcccctttgcaggtgtcggctgctccttcaagggaagcccacagtctgtgcaagagcatgaggtcacctcccagacctcccacctaaacctgctgttggggttcatgaaacagtggaaggcccggctgggctgtggcctggagtctgggcccatggccctggagcagaacctgtcagacctgcagctgcaggcagccgtggaagtggcgggggacctggaggtcgattgctaccgggcaccctgctccgagagccaggaggagctggccctgcagcacttcatgaaggagaagcttctggctgagctggaggggaagctgcgtgtgtttgagaacattgttgctgtcctcaacaaggaggtggaggcctcccacctggccctggccacctctatccaccagagccagctggaccgtgagcgcatcctgagcttggagcagagggtggtggagcttcagcagaccctggcccagaaagaccaggccctgggcaagctggagcagagcttgcgcctcatggaggaggcctccttcgatggcactttcctgtggaagatcaccaatgtcaccaggcggtgccatgagtcggcctgtggcaggaccgtcagcctcttctccccagccttctacactgccaagtatggctacaagttgtgcctgcggctgtacctgaatggagatggcactggaaagagaacccatctgtcgctcttcatcgtgatcatgagaggggagtatgatgcgctgctgccgtggcctttccggaacaaggtcaccttcatgctgctggaccagaacaaccgtgagcacgccattgacgccttccggcctgacctaagctcagcgtccttccagaggccccagagtgaaaccaacgtggccagtggatgcccactcttcttccccctcagcaaactgcagtcacccaagcacgcctacgtgaaggacgacacaatgttcctcaagtgcattgtggagaccagcacttagggtgggcggggctcctgagggagctccaactcagaagggagctagccagaggactgtgatgccctgcccttggcacccaagacctcagggcacaaagatgggtgaaggctggcatgatccaagcaagactgaggggtcgacttcgggctggccatctggttaggatggcaggacgtgggctgggcccacaaaggcaaagggtccagaaggagacaggcagagctgctcccctctgcacggaccatgcgacactgggaggccagtgagccactccggccccgaatgttgaggtggactctcaccaaatgagaagaaaatggaaccaggcttggaaccgtaggacccaagcagagaagctctcgggctaggaagatctctgcagggccgccagggagacctggacacaggcctgctctctttttctccagggtcagaaacaggaccgggtggaagggatggggtgccagtttgaatgcagtctgtccaggctcgtcattggaggtgaacaagcaaacccagagggctccactaggacttcaaattgggggttggatttgaagacttttaagtttccttccagcccagaaagtctctcattctaggcctcctggcccaggtgagtcctagagctacaggggttctggaaacattcaggagcttcctgtcctcccagctcctcactcaccttcagtaacccccactggactgacctggtccacagggcacctgccaccctgggcctggcagctcagcttccccaacacgcaggagcacacccagcccccacatcctgtgcctccatcagctaaacaccacgtcacttcatgcaggtgaaacccagtcactgtgagctcccaggtgcagccagaggcacctcaagaagaagaggggcataaactttcctcttcctgcctagaggccccacctttggtgctttccagaatcccgtaacacctgattaactgaggcatccacttctttcagcagactgatcaggacctccaagccactgagcaatgtataaccccaaagaaataatttttagaatctctttcgaagttttcctaaaaaaaaaaaaaaaaa

SEQ ID NO: 179 - DNA, 810 bp, Homo sapiens
  NM_006273
agcagaggggctgagaccaaaccagaaacctccaattctcatgtggaagcccatgccctcaccctccaacatgaaagcctctgcagcacttctgtgtctgctgctcacagcagctgctttcagcccccaggggcttgctcagccagttgggattaatacttcaactacctgctgctacagatttatcaataagaaaatccctaagcagaggctggagagctacagaaggaccaccagtagccactgtccccgggaagctgtaatcttcaagaccaaactggacaaggagatctgtgctgaccccacacagaagtgggtccaggactttatgaagcacctggacaagaaaacccaaactccaaagctttgaacattcatgactgaactaaaaacaagccatgacttgagaaacaaataatttgtataccctgtcctttctcagagtggttctgagattattttaatctaattctaaggaatatgagctttatgtaataatgtgaatcatggtttttcttagtagattttaaaagttattaatattttaatttaatcttccatggattttggtgggttttgaacataaagccttggatgtatatgtcatctcagtgctgtaaaaactgtgggatgctcctcccttctctacctcatgggggtattgtataagtccttgcaagaatcagtgcaaagatttgctttaattgttaagatatgatgtccctatggaagcatattgttattatataattacatatttgcatatgtatgactcccaaattttcacataaaatagatttttgtaaaaaa

SEQ ID NO: 180 - DNA, 1216 bp, Homo sapiens
  NM_006419
ttcggcacttgggagaagatgtttgaaaaaactgactctgctaatgagcctggactcagagctcaagtctgaactctacctccagacagaatgaagttcatctcgacatctctgcttctcatgctgctggtcagcagcctctctccagtccaaggtgttctggaggtctattacacaagcttgaggtgtagatgtgtccaagagagctcagtctttatccctagacgcttcattgatcgaattcaaatcttgccccgtgggaatggttgtccaagaaaagaaatcatagtctggaagaagaacaagtcaattgtgtgtgtggaccctcaagctgaatggatacaaagaatgatggaagtattgagaaaaagaagttcttcaactctaccagttccagtgtttaagagaaagattccctgatgctgatatttccactaagaacacctgcattcttcccttatccctgctctggattttagttttgtgcttagttaaatcttttccagggagaaagaacttccccatacaaataaggcatgaggactatgtgaaaaataaccttgcaggagctgatggggcaaactcaagcttcttcactcacagcaccctatatacacttggagtttgcattcttattcatcagggaggaaagtttctttgaaaatagttattcagttataagtaatacaggattattttgattatatacttgttgtttaatgtttaaaatttcttagaaaacaatggaatgagaatttaagcctcaaatttgaacatgtggcttgaattaagaagaaaattatggcatatattaaaagcaggcttctatgaaagactcaaaaagctgcctgggaggcagatggaacttgagcctgtcaagaggcaaaggaatccatgtagtagatatcctctgcttaaaaactcactacggaggagaattaagtcctacttttaaagaatttctttataaaatttactgtctaagattaatagcattcgaagatccccagacttcatagaatactcagggaaagcatttaaagggtgatgtacacatgtatcctttcacacatttgccttgacaaacttctttcactcacatctttttcactgactttttttgtgggggcggggccggggggactctggtatctaattctttaatgattcctataaatctaatgacattcaataaagttgagcaaacattttactt

SEQ ID NO: 181 - DNA, 1496 bp, Homo sapiens
  NM_006682
cgcactccctgctgggtgagcagcactgtaaagatgaagctggctaactggtactggctgagctcagctgttcttgccacttacggttttttggttgtggcaaacaatgaaacagaggaaattaaagatgaaagagcaaaggatgtctgcccagtgagactagaaagcagagggaaatgcgaagaggcaggggagtgcccctaccaggtaagcctgccccccttgactattcagctcccgaagcaattcagcaggatcgaggaggtgttcaaagaagtccaaaacctcaaggaaatcgtaaatagtctaaagaaatcttgccaagactgcaagctgcaggctgatgacaacggagacccaggcagaaacggactgttgttacccagtacaggagccccgggagaggttggtgataacagagttagagaattagagagtgaggttaacaagctgtcctctgagctaaagaatgccaaagaggagatcaatgtacttcatggtcgcctggagaagctgaatcttgtaaatatgaacaacatagaaaattatgttgacagcaaagtggcaaatctaacatttgttgtcaatagtttggatggcaaatgttcaaagtgtcccagccaagaacaaatacagtcacgtccagttcaacatctaatatataaagattgctctgactactacgcaataggcaaaagaagcagtgagacctacagagttacacctgatcccaaaaatagtagctttgaagtttactgtgacatggagaccatggggggaggctggacagtgctgcaggcacgtctcgatgggagcaccaacttcaccagaacatggcaagactacaaagcaggctttggaaacctcagaagggaattttggctggggaacgataaaattcatcttctgaccaagagtaaggaaatgattctgagaatagatcttgaagactttaatggtgtcgaactatatgccttgtatgatcagttttatgtggctaatgagtttctcaaatatcgtttacacgttggtaactataatggcacagctggagatgcattacgtttcaacaaacattacaaccacgatctgaagtttttcaccactccagataaagacaatgatcgatatccttctgggaactgtgggctgtactacagttcaggctggtggtttgatgcatgtctttctgcaaacttaaatggcaaatattatcaccaaaaatacagaggtgtccgtaatgggattttctggggtacctggcctggtgtaagtgaggcacaccctggtggctacaagtcctccttcaaagaggctaagatgatgatcagacccaagcactttaagccataaatcactctgttcattcctccaggtattcgttatctaatagggcaattaattccttcagcactttagaatatgccttgtttcatatttttcatagctaaaaaatgtttgacatcctttgagatattttattactaaaatctgcc

SEQ ID NO: 182 - DNA, 737 bp, Homo sapiens
  NM_021057
tacccacctcaggtagcctagtgatatttgcaaaatcccaatggcccggtccttttctttactgatggccgtgctggtactcagctacaaatccatctgctctctgggctgtgatctgcctcagacccacagcctgcgtaataggagggccttgatactcctggcacaaatgggaagaatctctcctttctcctgcttgaaggacagacatgaattcagattcccggaggaggagtttgatggccaccagttccagaagactcaagccatctctgtcctccatgagatgatccagcagaccttcaatctcttcagcacagaggactcatctgctgcttgggaacagagcctcctagaaaaattttccactgaactttaccagcaactgaatgacctggaagcatgtgtgatacaggaggttggggtggaagagactcccctgatgaatgaggacttcatcctggctgtgaggaaatacttccaaagaatcactctttatctaacagagaagaaatacagcccttgtgcctgggaggttgtcagagcagaaatcatgagatccttctctttttcaacaaacttgaaaaaaggattaaggaggaaggattgaaaactggttcatcatggaaatgattctcattgactaatgcatcatctcacactttcatgagttcttccatttcaaagactcacttctataaccaccacaagttgaatcaaaatttccaaatgttttc

SEQ ID NO: 183 - DNA, 1869 bp, Homo sapiens
  NM_021972
gctccgcggcgccggctgcgaagttgagcgaaaagtttgaggccggagggagcgaggccggggagtccgctccagcggggcgctccagtccctcagacgtgggctgagcttgggacgagctgcgttccgccccaggccactgtagggaacggcggtggcgcctccccagcaaaccggaccgactgggtccagccgccgcagggaatgacaccggtgctcctacagccacggctccgggcggggaaggcgagccccacagccggccctgcgacgccctcctgggcagcaccgataaggagctgaaggcaggagccgccgccacgggcagcgcccccacagcgccagggaccccctggcagcgggagccgcgggtcgaggttatggatccagtggtcggttgcggacgtggcctctttggttttgttttctcagcgggcggcccccggggcgtgctcccgcggccctgccgcgtgctggtgctgctgaacccgcgcggcggcaagggcaaggccttgcagctcttccggagtcacgtgcagccccttttggctgaggctgaaatctccttcacgctgatgctcactgagcggcggaaccacgcgcgggagctggtgcggtcggaggagctgggccgctgggacgctctggtggtcatgtctggagacgggctgatgcacgaggtggtgaacgggctcatggagcggcctgactgggagaccgccatccagaagcccctgtgtagcctcccagcaggctctggcaacgcgctggcagcttccttgaaccattatgctggctatgagcaggtcaccaatgaagacctcctgaccaactgcacgctattgctgtgccgccggctgctgtcacccatgaacctgctgtctctgcacacggcttcggggctgcgcctcttctctgtgctcagcctggcctggggcttcattgctgatgtggacctagagagtgagaagtatcggcgtctgggggagatgcgcttcactctgggcaccttcctgcgtctggcagccctgcgcacctaccgcggccgactggcctacctccctgtaggaagagtgggttccaagacacctgcctcccccgttgtggtccagcagggcccggtagatgcacaccttgtgccactggaggagccagtgccctctcactggacagtggtgcccgacgaggactttgtgctagtcctggcactgctgcactcgcacctgggcagtgagatgtttgctgcacccatgggccgctgtgcagctggcgtcatgcatctgttctacgtgcgggcgggagtgtctcgtgccatgctgctgcgcctcttcctggccatggagaagggcaggcatatggagtatgaatgcccctacttggtatatgtgcccgtggtcgccttccgcttggagcccaaggatgggaaaggtgtgtttgcagtggatggggaattgatggttagcgaggccgtgcagggccaggtgcacccaaactacttctggatggtcagcggttgcgtggagcccccgcccagctggaagccccagcagatgccaccgccagaagagcccttatgacccctgggccgcgctgtgccttagtgtctacttgcaggacccttcctccttccctagggctgcagagcctgtccacagctcctgtgggggtggaggagactcctctggagaagggtgagaaggtggaggctatgctttggggggacaggccagaatgaagtcctgggtcaggagcccagctggctgggcccagctgcctatgtaaggccttctagtttgttctgagacccccaccccacgaaccaaatccaaataaagtgacattcccagcctgaaaaaaaaaaaaaaaaaa

SEQ ID NO: 184 - DNA, 4000 bp, Homo sapiens
  NM_022740
cggccgcttttttctcaagatggcagattcccactgaggctgagggggccgagctcgcgcgccgcgttcccttctccgttgccatgaaccgcggacaccccggccccgatggcccccgtgtacgaaggtatggcctcacatgtgcaagttttctcccctcacacccttcaatcaagtgccttctgtagtgtgaagaaactaaaagtagagccaagttccaactgggacatgactgggtacggctcccacagcaaagtgtacagccagagcaagaacataccaccttctcagccagcctccacaaccgtcagcacctccttgccggtcccaaacccaagcctaccttacgagcagaccatcgtcttcccaggaagcaccgggcacatcgtggtcacctcagcaagcagcacttctgtcaccgggcaagtcctcggcggaccacacaacctaatgcgtcgaagcactgtgagcctccttgatacctaccaaaaatgtggactcaagcgtaagagcgaggagatcgagaacacaagcagcgtgcagatcatcgaggagcatccacccatgattcagaataatgcaagcggggccactgtcgccactgccaccacgtctactgccacctccaaaaacagcggctccaacagcgagggcgactatcagctggtgcagcatgaggtgctgtgctccatgaccaacacctacgaggtcttagagttcttgggccgagggacgtttggacaagtggtcaagtgctggaaacggggcaccaatgagatcgtagccatcaagatcctgaagaaccgcccatcctatgcccgacaaggtcagattgaagtgagcatcctggcccggttgagcacggagagtgccgatgactataacttcgtccgggcctacgaatgcttccagcacaagaaccacacgtgcttggtcttcgagatgttggagcagaacctctatgactttctgaagcaaaacaagtttagccccttgcccctcaaatacattcgcccagttctccagcaggtagccacagccctgatgaaactcaaaagcctaggtcttatccacgctgacctcaaaccagaaaacatcatgctggtggatccatctagacaaccatacagagtcaaggtcatcgactttggttcagccagccacgtctccaaggctgtgtgctccacctacttgcagtccagatattacagggcccctgagatcatccttggtttaccattttgtgaggcaattgacatgtggtccctgggctgtgttattgcagaattgttcctgggttggccgttatatccaggagcttcggagtatgatcagattcggtatatttcacaaacacagggtttgcctgctgaatatttattaagcgccgggacaaagacaactaggtttttcaaccgtgacacggactcaccatatcctttgtggagactgaagacaccagatgaccatgaagcagagacagggattaagtcaaaagaagcaagaaagtacattttcaactgtttagatgatatggcccaggtgaacatgacgacagatttggaagggagcgacatgttggtagaaaaggctgaccggcgggagttcattgacctgttgaagaagatgctgaccattgatgctgacaagagaatcactccaatcgaaaccctgaaccatccctttgtcaccatgacacacttactcgattttccccacagcacacacgtcaaatcatgtttccagaacatggagatctgcaagcgtcgggtgaatatgtatgacacggtgaaccagagcaaaacccctttcatcacgcacgtggcccccagcacgtccaccaacctgaccatgacctttaacaaccagctgaccactgtccacaaccaggctccctcctctaccagtgccactatttccttagccaatcccgaagtctccatactaaactacccatctacactctaccagccctcagcggcatccatggctgcagtggcccagcggagcatgcccctgcagacaggaacagcccagatttgtgcccggcctgacccgttccagcaagctctcatcgtgtgtccccccggcttccaaggcttgcaggcctctccctctaagcacgctggctactcggtgcgaatggaaaatgcagttcccatcgtcactcaagccccaggagctcagcctcttcagatccaaccaggtctgcttgcccagcaggcttggccaagtgggacccagcagatcctgcttcccccagcatggcagcaactgactggagtggccacccacacatcagtgcagcatgccaccgtgattcccgagaccatggcaggcacccagcagctggcggactggagaaatacgcatgctcacggaagccattataatcccatcatgcagcagcctgcactattgaccggtcatgtgacccttccagcagcacagcccttaaatgtgggtgtggcccacgtgatgcggcagcagccaaccagcaccacctcctcccggaagagtaagcagcaccagtcatctgtgagaaatgtctccacctgtgaggtgtcctcctctcaggccatcagctccccacagcgatccaagcgtgtcaaggagaacacacctccccgctgtgccatggtgcacagtagcccggcctgcagcacctcggtcacctgtgggtggggcgacgtggcctccagcaccacccgggaacggcagcggcagacaattgtcattcccgacactcccagccccacggtcagcgtcatcaccatcagcagtgacacggacgaggaggaggaacagaaacacgcccccaccagcactgtctccaagcaaagaaaaaacgtcatcagctgtgtcacagtccacgactccccctactccgactcctccagcaacaccagcccctactccgtgcagcagcgtgctgggcacaacaatgccaatgcctttgacaccaaggggagcctggagaatcactgcacggggaacccccgaaccatcatcgtgccacccctgaaaacccaggccagcgaagtattggtggagtgtgatagcctggtgccagtcaacaccagtcaccactcgtcctcctacaagtccaagtcctccagcaacgtgacctccaccagcggtcactcttcagggagctcatctggagccatcacctaccggcagcagcggccgggcccccacttccagcagcagcagccactcaatctcagccaggctcagcagcacatcaccacggaccgcactgggagccaccgaaggcagcaggcctacatcactcccaccatggcccaggctccgtactccttcccgcacaacagccccagccacggcactgtgcacccgcatctggctgcagccgctgccgctgcccacctccccacccagccccacctctacacctacactgcgccggcggccctgggctccaccggcaccgtggcccacctggtggcctcgcaaggctctgcgcgccacaccgtgcagcacactgcctacccagccagcatcgtccaccaggtccccgtgagcatgggcccccgggtcctgccctcgcccaccatccacccgagtcagtatccagcccaatttgcccaccagacctacatcagcgcctcgccagcctccaccgtctacactggatacccactgagccccgccaaggtcaaccagtacccttacatataaacactggaggggagggagggagggagggagggagagaatggcccgagggaggagggagagaaggagggaggcgctcctgggaccgtgggcgctggccttttatactgaagatgccgcacacaaacaatgcaaacggggcaggggcggggggggggggggcagagggcagggggacgggtcgggacaccagtgaaacttgaaccgggaagtgggaggacgtagagcagagaagagaacatttttaaaaggaagggattaaagagggtgggaaatctatggtttttattttaaaaaa

SEQ ID NO: 185 - DNA, 1433 bp, Homo sapiens
  NM_000597
attcggggcgagggaggaggaagaagcggaggaggcggctcccgctcgcagggccgtgcacctgcccgcccgcccgctcgctcgctcgcccgccgcgccgcgctgccgaccgccagcatgctgccgagagtgggctgccccgcgctgccgctgccgccgccgccgctgctgccgctgctgccgctgctgctgctgctactgggcgcgagtggcggcggcggcggggcgcgcgcggaggtgctgttccgctgcccgccctgcacacccgagcgcctggccgcctgcgggcccccgccggttgcgccgcccgccgcggtggccgcagtggccggaggcgcccgcatgccatgcgcggagctcgtccgggagccgggctgcggctgctgctcggtgtgcgcccggctggagggcgaggcgtgcggcgtctacaccccgcgctgcggccaggggctgcgctgctatccccacccgggctccgagctgcccctgcaggcgctggtcatgggcgagggcacttgtgagaagcgccgggacgccgagtatggcgccagcccggagcaggttgcagacaatggcgatgaccactcagaaggaggcctggtggagaaccacgtggacagcaccatgaacatgttgggcgggggaggcagtgctggccggaagcccctcaagtcgggtatgaaggagctggccgtgttccgggagaaggtcactgagcagcaccggcagatgggcaagggtggcaagcatcaccttggcctggaggagcccaagaagctgcgaccaccccctgccaggactccctgccaacaggaactggaccaggtcctggagcggatctccaccatgcgccttccggatgagcggggccctctggagcacctctactccctgcacatccccaactgtgacaagcatggcctgtacaacctcaaacagtgcaagatgtctctgaacgggcagcgtggggagtgctggtgtgtgaaccccaacaccgggaagctgatccagggagcccccaccatccggggggaccccgagtgtcatctcttctacaatgagcagcaggaggcttgcggggtgcacacccagcggatgcagtagaccgcagccagccggtgcctggcgcccctgccccccgcccctctccaaacaccggcagaaaacggagagtgcttgggtggtgggtgctggaggattttccagttctgacacacgtatttatatttggaaagagaccagcaccgagctcggcacctccccggcctctctcttcccagctgcagatgccacacctgctccttcttgctttccccgggggaggaagggggttgtggtcggggagctggggtacaggtttggggagggggaagagaaatttttatttttgaacccctgtgtcccttttgcataagattaaaggaaggaaaagt

SEQ ID NO: 186 - DNA, 2130 bp, Homo sapiens
  NM_000678
tcctgccggccgctcgttctgtgcccccggcccggccaccgacggccgcgcgttgagatgactttccgcgatctcctgagcgtcagtttcgagggaccccgcccggacagcagcgcagggggctccagcgcgggcggcggcgggggcagcgcgggcggcgcggccccctcggagggcccggcggtgggcggcgtgccggggggcgcgggcggcggcggcggcgtggtgggcgcaggcagcggcgaggacaaccggagctccgcgggggagccggggagcgcgggcgcgggcggcgacgtgaatggcacggcggccgtcgggggactggtggtgagcgcgcagggcgtgggcgtgggcgtcttcctggcagccttcatccttatggccgtggcaggtaacctgcttgtcatcctctcagtggcctgcaaccgccacctgcagaccgtcaccaactatttcatcgtgaacctggccgtggccgacctgctgctgagcgccaccgtactgcccttctcggccaccatggaggttctgggcttctgggcctttggccgcgccttctgcgacgtatgggccgccgtggacgtgctgtgctgcacggcctccatcctcagcctctgcaccatctccgtggaccggtacgtgggcgtgcgccactcactcaagtacccagccatcatgaccgagcgcaaggcggccgccatcctggccctgctctgggtcgtagccctggtggtgtccgtagggcccctgctgggctggaaggagcccgtgccccctgacgagcgcttctgcggtatcaccgaggaggcgggctacgctgtcttctcctccgtgtgctccttctacctgcccatggcggtcatcgtggtcatgtactgccgcgtgtacgtggtcgcgcgcagcaccacgcgcagcctcgaggcaggcgtcaagcgcgagcgaggcaaggcctccgaggtggtgctgcgcatccactgtcgcggcgcggccacgggcgccgacggggcgcacggcatgcgcagcgccaagggccacaccttccgcagctcgctctccgtgcgcctgctcaagttctcccgtgagaagaaagcggccaagactctggccatcgtcgtgggtgtcttcgtgctctgctggttccctttcttctttgtcctgccgctcggctccttgttcccgcagctgaagccatcggagggcgtcttcaaggtcatcttctggctcggctacttcaacagctgcgtgaacccgctcatctacccctgttccagccgcgagttcaagcgcgccttcctccgtctcctgcgctgccagtgccgtcgtcgccggcgccgccgccctctctggcgtgtctacggccaccactggcgggcctccaccagcggcctgcgccaggactgcgccccgagttcgggcgacgcgccccccggagcgccgctggccctcaccgcgctccccgaccccgaccccgaacccccaggcacgcccgagatgcaggctccggtcgccagccgtcgaaagccacccagcgccttccgcgagtggaggctgctggggccgttccggagacccacgacccagctgcgcgccaaagtctccagcctgtcgcacaagatccgcgccgggggcgcgcagcgcgcagaggcagcgtgcgcccagcgctcagaggtggaggctgtgtccctaggcgtcccacacgaggtggccgagggcgccacctgccaggcctacgaattggccgactacagcaacctacgggagaccgatatttaaggaccccagagctaggccgcggagtgtgctgggcttgggggtaagggggaccagagaggcgggctggtgttctaagagcccccgtgcaaatcggagacccggaaactgatcagggcagctgctctgtgacatccctgaggaactgggcagagcttgaggctggagcccttgaaaggtgaaaagtagtggggccccctgctggactcaggtgcccagaactcttttcttagaagggagaggctgcgggctccgtggggccttttgctcccaatccctatttgagaaacactgccccatcctccatgccctgaaccctgagtagacagccccaagcatggccaggaaggcctgccc

SEQ ID NO: 187 - DNA, 1617 bp, Homo sapiens
  NM_000823
agcagccaaggcttactgaggctggtggagggagccactgctgggctcaccatggaccgccggatgtggggggcccacgtcttctgcgtgttgagcccgttaccgaccgtattgggccacatgcacccagaatgtgacttcatcacccagctgagagaggatgagagtgcctgtctacaagcagcagaggagatgcccaacaccaccctgggctgccctgcgacctgggatgggctgctgtgctggccaacggcaggctctggcgagtgggtcaccctcccctgcccggatttcttctctcacttcagctcagagtcaggggctgtgaaacgggattgtactatcactggctggtctgagccctttccaccttaccctgtggcctgccctgtgcctctggagctgctggctgaggaggaatcttacttctccacagtgaagattatctacaccgtgggccatagcatctctattgtagccctcttcgtggccatcaccatcctggttgctctcaggaggctccactgcccccggaactacgtccacacccagctgttcaccacttttatcctcaaggcgggacgtgtgttcctgaaggatgctgcccttttccacagcgacgacactgaccactgcagcttctccactgttctatgcaaggtctctgtggccgcctcccatttcgccaccatgaccaacttcagctggctgttggcagaagccgtctacctgaactgcctcctggcctccacctcccccagctcaaggagagccttctggtggctggttctcgctggctgggggctgcccgtgctcttcactggcacgtgggtgagctgcaaactggccttcgaggacatcgcgtgctgggacctggacgacacctccccctactggtggatcatcaaagggcccattgtcctctcggtcggggtgaactttgggctttttctcaatattatccgcatcctggtgaggaaactggagccagctcagggcagcctccatacccagtctcagtattggcgtctctccaagtcgacacttttcctgatcccactctttggaattcactacatcatcttcaacttcctgccagacaatgctggcctgggcatccgcctccccctggagctgggactgggttccttccagggcttcattgttgccatcctctactgcttcctcaaccaagaggtgaggactgagatctcacggaagtggcatggccatgaccctgagcttctgccagcctggaggacccgtgctaagtggaccacgccttcccgctcggcggcaaaggtgctgacatctatgtgctaggctgcctcatcacgccactggagtccacacttgaatttgggcagctaccacgggtctgccatgctctggaggagcaagggggccacatccccaccccagctgttacccagcccggggcaggtgcagcccttcctccctgtctctgcatctgactctcttttgaggtccctgtatgtctacctctgacttctgtggtccctctgtgtctgctctcatccattcctcttactggggcctggggctctagcccaaggctcagaggagccaataaacctgtaaatgaaaaaaaaaaaa

SEQ ID NO: 188 - DNA, 1225 bp, Homo sapiens
  NM_001288
gcggctctctgatccagcccgggagaggaccgagctggaggagctgggtgtggggtgcgttgggctggtggggaggcctagtttgggtgcaagtaggtctgattgagcttgtgttgtgctgaagggacagccctgggtctaggggagagagtccctgagtgtgagacccgccttccccggtcccagcccctcccagttcccccagggacggccacttcctggtccccgacgcaaccatggctgaagaacaaccgcaggtcgaattgttcgtgaaggctggcagtgatggggccaagattgggaactgcccattctcccagagactgttcatggtactgtggctcaagggagtcaccttcaatgttaccaccgttgacaccaaaaggcggaccgagacagtgcagaagctgtgcccaggggggcagctcccattcctgctgtatggcactgaagtgcacacagacaccaacaagattgaggaatttctggaggcagtgctgtgccctcccaggtaccccaagctggcagctctgaaccctgagtccaacacagctgggctggacatatttgccaaattttctgcctacatcaagaattcaaacccagcactcaatgacaatctggagaagggactcctgaaagccctgaaggttttagacaattacttaacatcccccctcccagaagaagtggatgaaaccagtgctgaagatgaaggtgtctctcagaggaagtttttggatggcaacgagctcaccctggctgactgcaacctgttgccaaagttacacatagtacaggtggtgtgtaagaagtaccggggattcaccatccccgaggccttccggggagtgcatcggtacttgagcaatgcctacgcccgggaagaattcgcttccacctgtccagatgatgaggagatcgagctcgcctatgagcaagtggcaaaggccctcaaataagcccctcctgggactccctcaaccccctccattttctccacaaaggccctggtggtttccacattgctacccaatggacacactccaaaatggccagtgggcagggaatcctggagcacttgttccgggatggtgtggtggaagaggggatgagggaaagaaatggggggcctgggtcagatttttattgtggggtggggtgagtaggacaacatatttcagtaataaaatacagaataaaaatcaagtgtttttaaaaaaaa

SEQ ID NO: 189 - DNA, 2565 bp, Homo sapiens
  NM_001710
ccgggaaagtgatgtgggtaggacaggcggggcgagccgcaggtgccagaacacagattgtataaaaggctgggggctggtggggagcaggggaagggaatgtgaccaggtctaggtctggagtttcagcttggacactgagccaagcagacaagcaaagcaagccaggacacaccatcctgccccaggcccagcttctctcctgccttccaacgccatggggagcaatctcagcccccaactctgcctgatgccctttatcttgggcctcttgtctggaggtgtgaccaccactccatggtctttggcccagccccagggatcctgctctctggagggggtagagatcaaaggcggctccttccgacttctccaagagggccaggcactggagtacgtgtgtccttctggcttctacccgtaccctgtgcagacacgtacctgcagatctacggggtcctggagcaccctgaagactcaagaccaaaagactgtcaggaaggcagagtgcagagcaatccactgtccaagaccacacgacttcgagaacggggaatactggccccggtctccctactacaatgtgagtgatgagatctctttccactgctatgacggttacactctccggggctctgccaatcgcacctgccaagtgaatggccggtggagtgggcagacagcgatctgtgacaacggagcggggtactgctccaacccgggcatccccattggcacaaggaaggtgggcagccagtaccgccttgaagacagcgtcacctaccactgcagccgggggcttaccctgcgtggctcccagcggcgaacgtgtcaggaaggtggctcttggagcgggacggagccttcctgccaagactccttcatgtacgacacccctcaagaggtggccgaagctttcctgtcttccctgacagagaccatagaaggagtcgatgctgaggatgggcacggcccaggggaacaacagaagcggaagatcgtcctggacccttcaggctccatgaacatctacctggtgctagatggatcagacagcattggggccagcaacttcacaggagccaaaaagtgtctagtcaacttaattgagaaggtggcaagttatggtgtgaagccaagatatggtctagtgacatatgccacataccccaaaatttgggtcaaagtgtctgaagcagacagcagtaatgcagactgggtcacgaagcagctcaatgaaatcaattatgaagaccacaagttgaagtcagggactaacaccaagaaggccctccaggcagtgtacagcatgatgagctggccagatgacgtccctcctgaaggctggaaccgcacccgccatgtcatcatcctcatgactgatggattgcacaacatgggcggggacccaattactgtcattgatgagatccgggacttgctatacattggcaaggatcgcaaaaacccaagggaggattatctggatgtctatgtgtttggggtcgggcctttggtgaaccaagtgaacatcaatgctttggcttccaagaaagacaatgagcaacatgtgttcaaagtcaaggatatggaaaacctggaagatgttttctaccaaatgatcgatgaaagccagtctctgagtctctgtggcatggtttgggaacacaggaagggtaccgattaccacaagcaaccatggcaggccaagatctcagtcattcgcccttcaaagggacacgagagctgtatgggggctgtggtgtctgagtactttgtgctgacagcagcacattgtttcactgtggatgacaaggaacactcaatcaaggtcagcgtaggaggggagaagcgggacctggagatagaagtagtcctatttcaccccaactacaacattaatgggaaaaaagaagcaggaattcctgaattttatgactatgacgttgccctgatcaagctcaagaataagctgaaatatggccagactatcaggcccatttgtctcccctgcaccgagggaacaactcgagctttgaggcttcctccaactaccacttgccagcaacaaaaggaagagctgctccctgcacaggatatcaaagctctgtttgtgtctgaggaggagaaaaagctgactcggaaggaggtctacatcaagaatggggataagaaaggcagctgtgagagagatgctcaatatgccccaggctatgacaaagtcaaggacatctcagaggtggtcacccctcggttcctttgtactggaggagtgagtccctatgctgaccccaatacttgcagaggtgattctggcggccccttgatagttcacaagagaagtcgtttcattcaagttggtgtaatcagctggggagtagtggatgtctgcaaaaaccagaagcggcaaaagcaggtacctgctcacgcccgagactttcacatcaacctctttcaagtgctgccctggctgaaggagaaactccaagatgaggatttgggttttctataaggggtttcctgctggacaggggcgtgggattgaattaaaacagctgcgacaac

SEQ ID NO: 190 - DNA, 1550 bp, Homo sapiens
  NM_002133
tcaacgcctgcctcccctcgagcgtcctcagcgcagccgccgcccgcggagccagcacgaacgagcccagcaccggccggatggagcgtccgcaacccgacagcatgccccaggatttgtcagaggccctgaaggaggccaccaaggaggtgcacacccaggcagagaatgctgagttcatgaggaactttcagaagggccaggtgacccgagacggcttcaagctggtgatggcctccctgtaccacatctatgtggccctggaggaggagattgagcgcaacaaggagagcccagtcttcgcccctgtctacttcccagaagagctgcaccgcaaggctgccctggagcaggacctggccttctggtacgggccccgctggcaggaggtcatcccctacacaccagccatgcagcgctatgtgaagcggctccacgaggtggggcgcacagagcccgagctgctggtggcccacgcctacacccgctacctgggtgacctgtctgggggccaggtgctcaaaaagattgcccagaaagccctggacctgcccagctctggcgagggcctggccttcttcaccttccccaacattgccagtgccaccaagttcaagcagctctaccgctcccgcatgaactccctggagatgactcccgcagtcaggcagagggtgatagaagaggccaagactgcgttcctgctcaacatccagctctttgaggagttgcaggagctgctgacccatgacaccaaggaccagagcccctcacgggcaccagggcttcgccagcgggccagcaacaaagtgcaagattctgcccccgtggagactcccagagggaagcccccactcaacacccgctcccaggctccgcttctccgatgggtccttacactcagctttctggtggcgacagttgctgtagggctttatgccatgtgaatgcaggcatgctggctcccagggccatgaactttgtccggtggaaggccttctttctagagagggaattctcttggctggcttccttaccgtgggcactgaaggctttcagggcctccagccctctcactgtgtccctctctctggaaaggaggaaggagcctatggcatcttccccaacgaaaagcacatccaggcaatggcctaaacttcagagggggcgaaggggtcagccctgcccttcagcatcctcagttcctgcagcagagcctggaagacaccctaatgtggcagctgtctcaaacctccaaaagccctgagtttcaagtatccttgttgacacggccatgaccactttccccgtgggccatggcaatttttacacaaacctgaaaagatgttgtgtcttgtgtttttgtcttatttttgttggagccactctgttcctggctcagcctcaaatgcagtatttttgttgtgttctgttgtttttatagcagggttggggtggtttttgagccatgcgtgggtggggagggaggtgtttaacggcactgtggccttggtctaacttttgtgtgaaataataaacaacattgtctg

SEQ ID NO: 191 - DNA, 1084 bp, Homo sapiens
  NM_003327
cgaggatgtgcgtgggggctcggcggctgggccgcgggccgtgtgcggctctgctcctcctgggcctggggctgagcaccgtgacggggctccactgtgtcggggacacctaccccagcaacgaccggtgctgccacgagtgcaggccaggcaacgggatggtgagccgctgcagccgctcccagaacacggtgtgccgtccgtgcgggccgggcttctacaacgacgtggtcagctccaagccgtgcaagccctgcacgtggtgtaacctcagaagtgggagtgagcggaagcagctgtgcacggccacacaggacacagtctgccgctgccgggcgggcacccagcccctggacagctacaagcctggagttgactgtgccccctgccctccagggcacttctccccaggcgacaaccaggcctgcaagccctggaccaactgcaccttggctgggaagcacaccctgcagccggccagcaatagctcggacgcaatctgtgaggacagggaccccccagccacgcagccccaggagacccagggccccccggccaggcccatcactgtccagcccactgaagcctggcccagaacctcacagggaccctccacccggcccgtggaggtccccgggggccgtgcggttgccgccatcctgggcctgggcctggtgctggggctgctgggccccctggccatcctgctggccctgtacctgctccggagggaccagaggctgccccccgatgcccacaagccccctgggggaggcagtttccggacccccatccaagaggagcaggccgacgcccactccaccctggccaagatctgacctgggcccaccaaggtggacgctgggccccgccaggctggagcccggagggtctgctgggcgagcagggcaggtgcaggccgcctgccccgccacgctcctgggccaactctgcaccgttctaggtgccgatggctgcctccggctctctgcttacgtatgccatgcatacctcctgccccgcgggaccacaataaaaaccttggcagacgggagtctccgaccggcaaaaaaaaaaaaaaaaa

SEQ ID NO: 192 - DNA, 2051 bp, Homo sapiens
  NM_006018
cgccactttgctggagcattcactaggcgaggcgctccatcggactcactagccgcactcatgaatcggcaccatctgcaggatcactttctggaaatagacaagaagaactgctgtgtgttccgagatgacttcattgccaaggtgttgccgccggtgttggggctggagtttatctttgggcttctgggcaatggccttgccctgtggattttctgtttccacctcaagtcctggaaatccagccggattttcctgttcaacctggcagtagctgactttctactgatcatctgcctgccgttcgtgatggactactatgtgcggcgttcagactggaactttggggacatcccttgccggctggtgctcttcatgtttgccatgaaccgccagggcagcatcatcttcctcacggtggtggcggtagacaggtatttccgggtggtccatccccaccacgccctgaacaagatctccaattggacagcagccatcatctcttgccttctgtggggcatcactgttggcctaacagtccacctcctgaagaagaagttgctgatccagaatggccctgcaaatgtgtgcatcagcttcagcatctgccataccttccggtggcacgaagctatgttcctcctggagttcctcctgcccctgggcatcatcctgttctgctcagccagaattatctggagcctgcggcagagacaaatggaccggcatgccaagatcaagagagccatcaccttcatcatggtggtggccatcgtctttgtcatctgcttccttcccagcgtggttgtgcggatccgcatcttctggctcctgcacacttcgggcacgcagaattgtgaagtgtaccgctcggtggacctggcgttctttatcactctcagcttcacctacatgaacagcatgctggaccccgtggtgtactacttctccagcccatcctttcccaacttcttctccactttgatcaaccgctgcctccagaggaagatgacaggtgagccagataataaccgcagcacgagcgtcgagctcacaggggaccccaacaaaaccagaggcgctccagaggcgttaatggccaactccggtgagccatggagcccctcttatctgggcccaacctcaaataaccattccaagaagggacattgtcaccaagaaccagcatctctggagaaacagttgggctgttgcatcgagtaatgtcactggactcggcctaaggtttcctggaacttccagattcagagaatctgatttagggaaactgtggcagatgagtgggagactggttgcaaggtgtgaccacaggaatcctggaggaacagagagtaaagcttctaggcatctgaaacttgcttcatctctgacgctcgcaggactgaagatgggcaaattgtaggcgtttctgctgagcagagttggagccagagatctacttgtgacttgttggccttcttcccacatctgcctcagactggggggggctcagctcctcgggtgatatctagcctgcttgtgagctctagcagggataaggagagctgagattggagggaattgtgttgctcctggaggaagcccaggcatcattaaacaagccagtaggtcacctggcttccgtggaccaattcatctttcagacaagctttagagaaatggactcagggaagagactcacatgctttggttagtatctgtgtttccggtgggtgtaataggggattagccccagaagggactgagctaaacagtgttattatgggaaaggaaatggcattgctgctttcaaccagcgactaatgcaatccattcctctcttgtttatagtaatctaagggttgagcagttaaaacggcttcaggatagaaagctgtttcccacctgtttcgttttaccattaaaagggaaacgtgcctctgccccacgggtagagggggtgcacgttcctcctggttccttcgcttgtgtttctgtacttaccaaaaatctaccacttcaataaattttgataggagacaaaaaaaaaa

SEQ ID NO: 193 - DNA, 5725 bp, Homo sapiens
  NM_007289
cctggaggagggctctggaagtcacgtcaggttggctcttcaggttcatttccatagttccctgcggcctctgccttggggagttatgttttgttaccgagatccgcgctaccagattgcaccggggctgatttgggggctgggaatttgccattctgctgtacagacactgatttttttttcttctttttaaaaagcaaggtttgttttcattttggattttaggtgatgggcaagtcagaaagtcagatggatataactgatatcaacactccaaagccaaagaagaaacagcgatggactcgactggagatcagcctctcggtccttgtcctgctcctcaccatcatagctgtgagaatgatcgcactctatgcaacctacgatgatggtatttgcaagtcatcagactgcataaaatcagctgctcgactgatccaaaacatggatgccaccactgagccttgtagagactttttcaaatatgcttgcggaggctggttgaaacgtaatgtcattcccgagaccagctcccgttacggcaactttgacattttaagagatgaactagaagtcgttttgaaagatgtccttcaagaacccaaaactgaagatatagtagcagtgcagaaagcaaaagcattgtacaggtcttgtataaatgaatctgctattgatagcagaggtggagaacctctactcaaactgttaccagacatatatgggtggccagtagcaacagaaaactgggagcaaaaatatggtgcttcttggacagctgaaaaagctattgcacaactgaattctaaatatgggaaaaaagtccttattaatttgtttgttggcactgatgataagaattctgtgaatcatgtaattcatattgaccaacctcgacttggcctcccttctagagattactatgaatgcactggaatctataaagaggcttgtacagcatatgtggattttatgatttctgtggccagattgattcgtcaggaagaaagattgcccatcgatgaaaaccagcttgctttggaaatgaataaagttatggaattggaaaaagaaattgccaatgctacggctaaacctgaagatcgaaatgatccaatgcttctgtataacaagatgagattggcccagatccaaaataacttttcactagagatcaatgggaagccattcagctggttgaatttcacaaatgaaatcatgtcaactgtgaatattagtattacaaatgaggaagatgtggttgtttatgctccagaatatttaaccaaacttaagcccattcttaccaaatattctgccagagatcttcaaaatttaatgtcctggagattcataatggatcttgtaagcagcctcagccgaacctacaaggagtccagaaatgctttccgcaaggccctttatggtacaacctcagaaacagcaacttggagacgttgtgcaaactatgtcaatgggaatatggaaaatgctgtggggaggctttatgtggaagcagcatttgctggagagagtaaacatgtggtcgaggatttgattgcacagatccgagaagtttttattcagactttagatgacctcacttggatggatgccgagacaaaaaagagagctgaagaaaaggccttagcaattaaagaaaggatcggctatcctgatgacattgtttcaaatgataacaaactgaataatgagtacctcgagttgaactacaaagaagatgaatacttcgagaacataattcaaaatttgaaattcagccaaagtaaacaactgaagaagctccgagaaaaggtggacaaagatgagtggataagtggagcagctgtagtcaatgcattttactcttcaggaagaaatcagatagtcttcccagccggcattctgcagccccccttctttagtgcccagcagtccaactcattgaactatgggggcatcggcatggtcataggacacgaaatcacccatggcttcgatgacaatggcagaaactttaacaaagatggagacctcgttgactggtggactcaacagtctgcaagtaactttaaggagcaatcccagtgcatggtgtatcagtatggaaacttttcctgggacctggcaggtggacagcaccttaatggaattaatacactgggagaaaacattgctgataatggaggtcttggtcaagcatacagagcctatcagaattatattaaaaagaatggcgaagaaaaattacttcctggacttgacctaaatcacaaacaactatttttcttgaactttgcacaggtgtggtgtggaacctataggccagagtatgcggttaactccattaaaacagatgtgcacagtccaggcaatttcaggattattgggactttgcagaactctgcagagttttcagaagcctttcactgccgcaagaattcatacatgaatccagaaaagaagtgccgggtttggtgatcttcaaaagaagcattgcagcccttggctagacttgccaacaccacagaaatggggaattctctaatcgaaagaaaatgggccctaggggtcactgtactgacttgagggtgattaacagagagggcaccatcacaatacagataacattaggttgtcctagaaagggtgtggagggaggaagggggtctaaggtctatcaagtcaatcatttctcactgtgtacataatgcttaatttctaaagataatattactgtttatttctgtttctcatatggtctaccagtttgctgatgtccctagaaaacaatgcaaaacctttgaggtagaccaggatttctaatcaaaagggaaaagaagatgttgaagaatagagttaggcaccagaagaagagtaggtgacactatagtttaaaacacattgcctaactactagtttttacttttatttgcaacatttacagtccttcaaaatccttccaaagaattcttatacacattggggccttggagcttacatagttttaaactcatttttgccatacatcagttattcattctgtgatcatttattttaagcactcttaaagcaaaaaatgaatgtctaaaattgttttttgttgtacctgctttgactgatgctgagattcttcaggcttcctgcaattttctaagcaatttcttgctctatctctcaaaacttggtatttttcagagatttatataaatgtaaaaataataatttttatatttaattattaactacatttatgagtaactattattataggtaatcaatgaatattgaagtttcagcttaaaataaacagttgtgaaccaagatctataaagcgatatacagatgaaaatttgagactatttaaacttataaatcatattgatgaaaagatttaagcacaaactttagggtaaaaattgcgattggacagttgtctagagatatatatacttgtggttttcaaattggactttcaaaattaaatctgtccctgagagtgtctctgataaaagggcaaatctgcacctatgtagctctgcatctcctgtcttttcaggtttgtcatcagatggaaatattttgataataaattgaaattgtgaactcattgctccctaagactgtgacaactgtctaactttagaagtgcatttctgaatagaaatgggaggcctctgatggaccttctagaattataagtcacaaagagttctggaaaagaactgtttactgcttgataggaattcatcttttgaggcttctgttcctctcttttcctgttgtattgactattttcgttcattacttgattaagattttacaaaagaggagcacttccaaaattcttatttttcctaacaaaagatgaaagcagggaatttctatctaaatgatgagtattagttccctgtctcttgaaaaatgcccatttgcctttaaaaaaaaaagttacagaaatactataacatatgtacataaattgcataaagcataagtatacagttcaataaacttaactttaactgaacaatggccctgtagccagcacctgtaagaaacagagcagtaccagcgctctaaaagcacctccttgtcactttattactcccagaacaacaactatcctgacttctaatatcattcactagctttgcctggttttgtcttttatgcagatagaatcaatcagtatgtattcttttgtgcctggcttctttctctcagccttacatttgtgagattcctctgtattgtgctgattgtggatcttttcattctcattgcagaataatgttctattgtgggacttattacaatttgttcatcctattgttgatgggcacttgagaactttccattttggcgctattacaaatagtgcaactatgaatgtactgcatgttaccatcttacttgagcctttaatggacttatttcttcaaatccttccaaaaattattataagcattgaaattatagtttcaagccaactgtggatacccttaccctttcctcctttatcacaaccaccgttacaagtatacttatatttccctaaaatacatttaaaacttacctaagtgacatttgtagttggagtaataggagcttccagctctaataaaacagctgtctctaacttattttatttccatcatgtcagagcaggtgaagagccagaagtgaagagtgactagtacaaattataaaaagccactagactcttcactgttagctttttaaaacattaggctcccatccctatggaggaacaactctccagtgcctggatcccctctgtctacaaatataagattttctgggcctaaaggatagatcaaagtcaaaaatagcaatgcctccctatccctcacacatccagacatcatgaattttacatggtactcttgttgagttctatagagccttctgatgtctctaaagcactaccgattctttggagttgtcacatcagataagacatatctctaattccatccataaatccagttctactatggctgagttctggtcaaagaaagaaagtttagaagctgagacacaaagggttgggagctgatgaaactcacaaatgatggtaggaagaagctctcgacaatacccgttggcaaggagtctgcctccatgctgcagtgttcgagtggattgtaggtgcaagatggaaaggattgtaggtgcaagctgtccagagaaaagagtccttgttccagccctattctgccactcctgacagggtgaccttgggtatttgcaatattcctttgggcctctgcttctctcacctaaaaaaagagaattagattatattggtggttctcagcaagagaaggagtatgtgtccaatgctgccttcccatgaatctgtctcccagttatgaatcagtgggcaggataaactgaaaactcccatttaagtgtctgaatcgagtgagacaaaattttagtccaaataacaagtaccaaagttttatcaagtttgggtctgtgctgctgttactgttaaccatttaagtggggcaaaaccttgctaattttctcaaaagcatttatcattcttgttgccacagctggagctctcaaactaaaagacatttgttattttggaaagaagaaagactctattctcaaagtttcctaatcagaaatttttatcagtttccagtctcaaaaatacaaaataaaaacaaacgtttttaatact

SEQ ID NO: 194 - DNA, 2010 bp, Homo sapiens
  NM_012068
attcattccctgtcctcggatcacagtctcttctcactacagtgtcgccgcctctgcctgcgtagccccggccatggctctgtagcctcgacccctttgtgcccccggcccgtctccgcgctcaccacgcctgcgctctccgctcccaccttctttcttcagccgaggccgccgccgcctctccttgctgcagccatggagtcttccactttcgccttggtgcctgtcttcgcccacctgagcatcctccagagcctcgtgccagctgctggtgcagcctctcctgttgccatcagtgcccagcacctgtgctacagccatgtcactcctggcgaccctggggctggagctggacagggccctgctcccagctagtgggctgggatggctcgtagactatgggaaactccccccggcccctgcccccctggctccctatgaggtccttgggggagccctggagggcgggcttccagtggggggagagcccctggcaggtgatggcttctctgactggatgactgagcgagttgatttcacagctctcctccctctggagcctcccctaccccccggcaccctcccccaaccttccccaaccccacctgacctggaagctatggcctccctcctcaagaaggagctggaacagatggaagacttcttcctagatgccccgcccctcccaccaccctccccgccgccactaccaccaccaccactaccaccagccccctccctccccctgtccctcccctcctttgacctcccccagccccctgtcttggatactctggacttgctggccatctactgccgcaacgaggccgggcaggaggaagtggggatgccgcctctgcccccgccacagcagccccctcctccttctccacctcaaccttctcgcctggccccctacccacatcctgccaccacccgaggggaccgcaagcaaaagaagagagaccagaacaagtcggcggctctgaggtaccgccagcggaagcgggcagagggtgaggccctggagggcgagtgccaggggctggaggcacggaatcgcgagctgaaggaacgggcagagtccgtggagcgcgagatccagtacgtcaaggacctgctcatcgaggtttacaaggcccggagccagaggacccgtagctgctagaagggcaggggtgtggcttctgggggctggtcttcagctctggcgccttcatccccctgcctctaccttcattccaaacccctctcggccgggtgcagtggcttatgcttgtaatcccagcactttgggaggccaaggcaggaggatcgtttgaggccaggaggtcaataccagcctgggcaacatagtaagaccctgtctctattaaaaaaaaaaaatcaacccttcttccccaccaaaccacccaactcctctctactcttatccttttatcctctgtctctgcttatcacctctcttgcgtatttctggatctccttccctcctttctcgtccaaatcatgaaatgtttggccttagtcaatgtctatgcccgtcacataacagccgaggcaccgaggcccacagggaagcagctgggagcttggaaacctggtctcttgaatttcaaacctggtttcttacaggtggttgtctggggtgggtggagtggcgacaggatagagctgaaggactatgcaaatgaggaagtaagtcagggcgggctttgagaaggggacccatatcctacaggcaaaaagcaggctaggtgaccttgggacactacgctaagggagggaggctaaaggcggccaggtttgcagtgcgggaagatgagcaggccagtgggaggaggggcagggcagggctgtagttggtgactgggtgttcattttagctctaagaaaaaaaatcagtgtttcgtgaaggtgttggagaggggctgtgtctgggtgagggatggcggggtactgatttttttgggaggttatgagcaaaaataaaacgaaacatttcctctg

SEQ ID NO: 195 - DNA, 3282 bp, Homo sapiens
  NM_014211
gggacagggctgaggatgaggagaaccctggggacccagaagaccgtgccttgcccggaagtcctgcctgtaggcctgaaggacttgccctaacagagcctcaacaactacctggtgattcctacttcagccccttggtgtgagcagcttctcaacatgaactacagcctccacttggccttcgtgtgtctgagtctcttcactgagaggatgtgcatccaggggagtcagttcaacgtcgaggtcggcagaagtgacaagctttccctgcctggctttgagaacctcacagcaggatataacaaatttctcaggcccaattttggtggagaacccgtacagatagcgctgactctggacattgcaagtatctctagcatttcagagagtaacatggactacacagccaccatatacctccgacagcgctggatggaccagcggctggtgtttgaaggcaacaagagcttcactctggatgcccgcctcgtggagttcctctgggtgccagatacttacattgtggagtccaagaagtccttcctccatgaagtcactgtgggaaacaggctcatccgcctcttctccaatggcacggtcctgtatgccctcagaatcacgacaactgttgcatgtaacatggatctgtctaaataccccatggacacacagacatgcaagttgcagctggaaagctggggctatgatggaaatgatgtggagttcacctggctgagagggaacgactctgtgcgtggactggaacacctgcggcttgctcagtacaccatagagcggtatttcaccttagtcaccagatcgcagcaggagacaggaaattacactagattggtcttacagtttgagcttcggaggaatgttctgtatttcattttggaaacctacgttccttccactttcctggtggtgttgtcctgggtttcattttggatctctctcgattcagtccctgcaagaacctgcattggagtgacgaccgtgttatcaatgaccacactgatgatcgggtcccgcacttctcttcccaacaccaactgcttcatcaaggccatcgatgtgtacctggggatctgctttagctttgtgtttggggccttgctagaatatgcagttgctcactacagttccttacagcagatggcagccaaagatagggggacaacaaaggaagtagaagaagtcagtattactaatatcatcaacagctccatctccagctttaaacggaagatcagctttgccagcattgaaatttccagcgacaacgttgactacagtgacttgacaatgaaaaccagcgacaagttcaagtttgtcttccgagaaaagatgggcaggattgttgattatttcacaattcaaaaccccagtaatgttgatcactattccaaactactgtttcctttgatttttatgctagccaatgtattttactgggcatactacatgtatttttgagtcaatgttaaatttcttgcatgccataggtcttcaacaggacaagataatgatgtaaatggtattttaggccaagtgtgcacccacatccaatggtgctacaagtgactgaaataatatttgagtctttctgctcaaagaatgaagctccaaccattgttctaagctgtgtagaagtcctagcattataggatcttgtaatagaaacatcagtccattcctctttcatcttaatcaaggacattcccatggagcccaagattacaaatgtactcagggctgtttattcggtggctccctggtttgcatttacctcatataaagaatgggaaggagaccattgggtaaccctcaagtgtcagaagttgtttctaaagtaactatacatgttttttactaaatctctgcagtgcttataaaatacattgttgcctatttagggagtaacattttctagtttttgtttctggttaaaatgaaatatgggcttatgtcaattcattggaagtcaatgcactaactcaataccaagatgagtttttaaataatgaatattatttaataccacaacagaattatccccaatttccaataagtcctatcattgaaaattcaaatataagtgaagaaaaaattagtagatcaacaatctaaacaaatccctcggttctaagatacaatggattccccatactggaaggactctgaggctttattcccccactatgcatatcttatcattttattattatacacacatccatcctaaactatactaaagcccttttcccatgcatggatggaaatggaagatttttttgtaacttgttctagaagtcttaatatgggctgttgccatgaaggcttgcagaattgagtccattttctagctgcctttattcacatagtgatggggtactaaaagtactgggttgactcagagagtcgctgtcattctgtcattgctgctactctaacactgagcaacactctcccagtggcagatcccctgtatcattccaagaggagcattcatccctttgctctaatgatcaggaatgatgcttattagaaaacaaactgcttgacccaggaacaagtggcttagcttaagtaaacttggctttgctcagatccctgatccttccagctggtctgctctgagtggcttatcccgcatgagcaggagcgtgctggccctgagtactgaactttctgagtaacaatgagacacgttacagaacctatgttcaggttgcgggtgagctgccctctccaaatccagccagagatgcacattcctcggccagtctcagccaacagtaccaaaagtgatttttgagtgtgccagggtaaaggcttccagttcagcctcagttattttagacaatctcgccatctttaatttcttagcttcctgttctaataaatgcacggctttacctttcctgtcagaaataaaccaaggctctaaaagatgatttcccttctgtaactccctagagccacaggttctcattccttttcccattatacttctcacaattcagtttctatgagtttgatcacctgatttttttaacaaaatatttctaacgggaatgggtgggagtgctggtgaaaagagatgaaatgtggttgtatgagccaatcatatttgtgattttttaaaaaaagtttaaaaggaaatatctgttctgaaaccccacttaagcattgtttttatataaaaacaatgataaagatgtgaactgtgaaataaatataccatattagctacccaccaaaaaaaaaaaaaaaaaa

SEQ ID NO: 196 - DNA, 5386 bp, Homo sapiens
  NM_015318
tgaattcctgtcggagagcgaggacggtgctggcaagaacgagaagagtgacaagagtaccagtgtgaagcgcaggctgagctgcctccgcagtcgagtgaccaggcagaaggagaaggggaagagcccagcacatctgaaggacaagggccaggatgcacgagagaggcgggagtgtgtcaatgggcaccagctgttgcaagggaccttctccggcccctccagctgccccctgtgtggcaaacctttcttgagctcagcctcgctcaaggagcacccccggggcaccctcctgtccgatggcagcccggccctgtccaggaatgtcggtatgacggtctctcagaaagggggtccccagccaacaccgagcccggctggccctgggacgcaactcggaccaatcacaggagagatggatgaagccgattctgcgtttttaaaatttaagcagacagctgatgactctctgtcccttacatctccaaacaccgagtccatttttgtagaagatccctacaccgcctcgctgaggagtgagattgagtcagacggccacgagtttgaagctgagtcctggagcctcgccgtggatgcagcctacgccaagaagcaaaagagggaggtggtgaaaagacaagatgtcctttatgagctgatgcagacagaggtgcaccacgtgcggacgctcaagatcatgctgaaggtgtactccagggccctgcaggaggagctgcagttcagcagcaaggccattggccgcctcttcccatgcgctgacgacctgctggagacgcacagccacttcctcgctcggctcaaggagcgccgccaggagtccctggaggagggcagtgaccggaattatgtcatccagaaaatcggcgacctcctggttcagcagttttcaggtgaaaatggggagagaatgaaagaaaagtacggtgtgttttgtagtggccacaatgaagctgttagtcattacaagttgctgcttcagcaaaacaagaaatttcaaaacttgatcaagaaaattggcaacttctccatcgtgcggcggcttggcgtgcaggagtgcattctcctggttacacaacgcataaccaaatacccagtgctggtggagcgcatcatccagaacacggaagctggcactgaggactatgaagacctgacccaggccttgaacctcatcaaagatatcatctcacaagtggacgccaaggtcagtgagtgtgagaagggccagcgcctcagggagatcgcagggaagatggacctgaagtcttccagcaaactcaagaacgggctcaccttccgcaaggaagacatgcttcagcggcagctccacctggagggcatgctatgctggaagaccacatcagggcgcttgaaagatatcctggctatcctgctgaccgacgtacttttgctgctacaagaaaaagatcagaaatacgtctttgcttctgtggactcaaagccacccgtcatctcgttacaaaagctcatcgtgagggaagtggccaacgaggagaaagcgatgtttctgatcagcgcctccttgcaagggccggagatgtatgaaatctacacgagctccaaagaggacaggaacgcctggatggcccacatccaaagggctgtggagagctgccctgacgaggaggaggggcccttcagcctgcccgaagaggaaaggaaggtggtcgaggcccgcgccacgagactccgggactttcaagagcggttgagcatgaaagaccagctgatcgcacagagcctcctagagaaacagcagatctacctggagatggccgagatgggcggcctcgaagacctgccccagccccgaggcctattccgtggaggggacccatccgagaccctgcagggggagctaattctcaagtcggccatgagcgagatcgagggcatccagagcctgatctgcaggcagctgggcagcgccaacggccaggcggaagacggaggcagctccacaggcccgcccaggagggctgagaccttcgcgggctacgactgcacaaacagccccaccaagaatggcagtttcaagaagaaagtcagcagcactgaccccaggccccgagactggcgaggccccccaaacagcccggacttgaagctcagtgacagtgacattcctgggagctctgaggaatcgccgcaggtggtggaggcgccaggcacggaatccgatccccgtctgcccaccgtcctggagtcggagcttgtccagcggatccagacactgtcccagctgctcctgaaccttcaggcggtaatcgcccaccaggacagctatgtggagacgcagcgggctgccatccaggagcgggagaagcagttccggctgcagtcgacgcgtgggaacctgctgctggagcaggagcggcaacgcaacttcgagaagcagcgggaggagcgcgcggccctggagaagctgcagagccagctgcggcacgagcagcagcgctgggagcgcgagcgccagtggcagcaccaggagctggagcgtgcgggcgcgcggctgcaggagcgcgagggcgaggcgcggcagctacgcgagcggctggagcaggagcgggccgagctggagcgccagcgccaggcctaccagcacgacctggagcggctgcgcgaggcccagcgtgccgtggagcgcgagcgggagcgcctggagctgctgcgccgcctcaagaagcagaacaccgcgccaggcgcgctgccgcccgacacactggccgaggcccagcccccaagccaccctcccagcttcaacggggaagggctggagggccctcgtgtgagcatgctgccatccggcgtggggccagagtacgcagagcgccccgaggtggctcgccgggacagcgcccccaccgagaaccggctggccaagagcgatgtgcccatccagctgctcagcgccaccaaccagttccagaggcaggcggccgtgcagcagcagatccccaccaagctggcggcctccaccaagggtggcaaggacaagggcggcaagagcaggggctctcagcgctgggagagctcagcgtccttcgacctgaagcagcagctgctgctcaacaagctcatggggaaagatgagagcacctcacggaaccgccgctcgctgagccctatcctgcccggcagacacagtcctgcgcccccaccagaccctggcttccccgccccgagcccaccgccagctgacagcccctccgagggcttctctctcaaggccgggggcacagccctcctgcccgggcccccagctccctcgccactgccggccacaccactcagcgccaaggaggacgccagcaaagaagacgtcatcttcttctaaaagggccgtgactcaaggaaagtttttaatggaaagttgagccagaactaaaccagggagctgtctgaaatcatagcaccccatccgggtggcggggagatcaactccgagctgtttttccgaggcagtgaggaacggtgccggctctgcacggagctgaggacaggacagaccttgctttgagaaggagctgccggccggggccacgctccacagccgccgcgcgacagtggagccaagggttagggcaccaggaggggccaggtggcgtcggcagcatctgtccccagaatcaggcagaatccacttcccaaacagagccccacgcaggttcaccatgaacctcagggtcagggaatgagccaggcacgggggcatgggcagagagggccacggggcagggcccactgagggaacatcagtggccctccagtcaggttctgtgggtttggaagcccatcgtgaaaggggctgacctttgcccctttttacttggcattggttttgaaaccagctgtttcccaaactctgcttcccaagggcaaccgttgctgttcacacgctcagcctgtctgggggagcgggcctctagcttcagccagggcgggtacacaccctgggcacagggtcctcagcccccgggaaatgagctcccagggctggcgtcccaccttccaggtgggggctggcacatcacagactgtcgagagcgccatgtcccagggcatgcagaggatgcacctagagacgttgcagcaagtggacaagtggccgctgtgcgggcccctcgcttgtagtgagctgttgcagcttacggtccgttccctggaggggtggaggaaggaggtgttgggcagcatcaaaggtgctgggacatcccagggtggtgagatccatccacgatccagctccggtggagaaagggcccatgtcaagccttgttctgcaccccaagcattggtggtaggactgggtcctggctgatcgtccttgttcccagtggggtacatgtgagcccctgccagggccaagtccttctcccgaacccagggtcctgggaactgcagatcccggggggattcagcccttctcccactgtgctggcagaggcactcctgtgacgctgaatacagtgaacagggacattcccgccactcggggacagatgggcacaagggaggggaaactccatcaggaagtgctcccctgggcagaggcgcccactgggtgctgtgggctcaggagggggcggggcaggagctggtgccaaccgggaaccagagccccacagccatacagcccattggtgacaaggtcctgagaacacagtggccaggtgtccccaggctcctggcccctccgacgacctcaactctgcccagcccggtccctggccatcagcgacgctgtccgccccccgtcagatcccatgtgtgccatgtttatcatcagtgttttgtatttttgtactgagtatcggagcactttacagaagctgactgtacattcctgttctgttgtgaagagaacattcccagaccctggcaccctcctgagccggcgtgtgccggtccagccctccgagatgccacaattccttggatgggggagaagttcaaggaatttctgctcggccacgcggtgggaaccccgcgtccccgccatgtggcagaggggtctcagtcgtgctaggcatcgggcggcagcgccgacagcccttccctcgccagtgcccctcggccactcctgggttggagcccgattttatttgtaaagttgacagtcgagcaaatgttcctattttcgtgggatctgcacacgtctttgtcagttgtggtcatgatcttagtcacctgctaattatttttacaatgattacaacatttcctcactgcgggatatttctgacccgctttagaacttaagacctgattctagcaataaacgtgtccgagatgagcggtgaaaaaaaaaaaaaaaaaaaaaaaaaaaaaaaaaaaaaaaaaa

SEQ ID NO: 197 - DNA, 1337 bp, Homo sapiens
  NM_015645
gaattcggctcgagagggagcgaaccaggactggggtgacggcagggcagggggcgcctggccggggagaagcgcgggggctggagcaccaccaactggagggtccggagtagcgagcgccccgaaggaggccatcggggagccgggaggggggactgcgagaggaccccggcgtccgggctcccggtgccagcgctatgaggccactcctcgtcctgctgctcctgggcctggcggccggctcgcccccactggacgacaacaagatccccagcctctgcccggggcaccccggccttccaggcacgccgggccaccatggcagccagggcttgccgggccgcgatggccgcgacggccgcgacggcgcgcccggggctccgggagagaaaggcgagggcgggaggccgggactgccgggacctcgaggggaccccgggccgcgaggagaggcgggacccgcggggcccaccgggcctgccggggagtgctcggtgcctccgcgatccgccttcagcgccaagcgctccgagagccgggtgcctccgccgtctgacgcacccttgcccttcgaccgcgtgctggtgaacgagcagggacattacgacgccgtcaccggcaagttcacctgccaggtgcctggggtctactacttcgccgtccatgccaccgtctaccgggccagcctgcagtttgatctggtgaagaatggcgaatccattgcctctttcttccagtttttcggggggtggcccaagccagcctcgctctcggggggggccatggtgaggctggagcctgaggaccaagtgtgggtgcaggtgggtgtgggtgactacattggcatctatgccagcatcaagacagacagcaccttctccggatttctggtgtactccgactggcacagctccccagtctttgcttagtgcccactgcaaagtgagctcatgctctcactcctagaaggagggtgtgaggctgacaaccaggtcatccaggagggctggcccccctggaatattgtgaatgactagggaggtggggtagagcactctccgtcctgctgctggcaaggaatgggaacagtggctgtctgcgatcaggtctggcagcatggggcagtggctggatttctgcccaagaccagaggagtgtgctgtgctggcaagtgtaagtcccccagttgctctggtccaggagcccacggtggggtgctctcttcctggtcctctgcttctctggatcctccccaccccctcctgctcctggggccggcccttttctcagagatcactcaataaacctaagaaccctcaaaaaaaaaaaaaaaaaaa

SEQ ID NO: 198 - DNA, 948 bp, Homo sapiens
  NM_018643
attgtggtgccttgtagctgtcccgggagccctcagcagcagttggagctggtgcacaggaaggatgaggaagaccaggctctgggggctgctgtggatgctctttgtctcagaactccgagctgcaactaaattaactgaggaaaagtatgaactgaaagaggggcagaccctggatgtgaaatgtgactacacgctagagaagtttgccagcagccagaaagcttggcagataataagggacggagagatgcccaagaccctggcatgcacagagaggccttcaaagaattcccatccagtccaagtggggaggatcatactagaagactaccatgatcatggtttactgcgcgtccgaatggtcaaccttcaagtggaagattctggactgtatcagtgtgtgatctaccagcctcccaaggagcctcacatgctgttcgatcgcatccgcttggtggtgaccaagggtttttcagggacccctggctccaatgagaattctacccagaatgtgtataagattcctcctaccaccactaaggccttgtgcccactctataccagccccagaactgtgacccaagctccacccaagtcaactgccgatgtctccactcctgactctgaaatcaaccttacaaatgtgacagatatcatcagggttccggtgttcaacattgtcattctcctggctggtggattcctgagtaagagcctggtcttctctgtcctgtttgctgtcacgctgaggtcatttgtaccctaggcccacgaacccacgagaatgtcctctgacttccagccacatccatctggcagttgtgccaagggaggagggaggaggtaaaaggcagggagttaataacatgaattaaatctgtaatcaccagctatttctaaagtcagcgtctcaccttaaaaaaaaaaaaaaaaaaaaaaaaaaaa

SEQ ID NO: 199 - DNA, 1349 bp, Homo sapiens
  NM_019846
cctgatcgaacagcctcacttgtgttgctgtcagtgccagtagggcaggcaggaatgcagcagagaggactcgccatcgtggccttggctgtctgtgcggccctacatgcctcagaagccatacttcccattgcctccagctgttgcacggaggtttcacatcatatttccagaaggctcctggaaagagtgaatatgtgtcgcatccagagagctgatggggattgtgacttggctgctgtcatccttcatgtcaagcgcagaagaatctgtgtcagcccgcacaaccatactgttaagcagtggatgaaagtgcaagctgccaagaaaaatggtaaaggaaatgtttgccacaggaagaaacaccatggcaagaggaacagtaacagggcacatcaggggaaacacgaaacatacggccataaaactccttattagagagtctacagataaatctacagagacaattcctcaagtggacttggccatgattggttagtctcactctatcacacaggctggagggcagtggcaggatctcagttcaccccaacctttgcctcacgggttcaagggattctcgtgcctcagccttccaagtagctgggattacaggtgtgcgccagtacgcctggctagttttagtattttttgttacagacggggtttcaccatgttggctgggctggtctcaaacttctgatctcaagtgatccatctgccttggcctcccaaagtgctgggattacaggcatgagccactgcacctggcctacgtattaggctttatgtaagatgtacatagtactccatactcctttccttttcctcctattgaaactgttcctataaactttataaaattaatcagagaagaagagagggtgagaaacataaataatccaagcttgcaccacattcagcattaatcactaggtcactttgcttgctgaccccttcctcatagttgtttggctattgcttcagaattatgtggagtctgttacaagattatagttccccttaactgctctatagataacagcttgaacattatgaaatgctaagttttcccttcgagacattctttcaggtcctgcatactgatgaaactactgatgtcagctggtctgaaggaccctaccagaagctaaatcatcaaagaatgcaatttccatatcctaatgattcaatctcccttaccctgaccaatcagtggcccaaattttccagccccttgcctcccagaaccccagcccagaactcttcagagatttaagaatctcctcctacctcctgactcagcaccatgtaatcattaaactctctgctgcaaaaaaaaaaaaaaa

SEQ ID NO: 200 - DNA, 3079 bp, Homo sapiens
  NM_021132
gggccctacagagggtccgccatgttccccggcggcgccgccgcttggctctggtagccgccgcccccgcccccaaccccgcccggcccagagcctagccgagccccgggcccagcatggccgccccggagccggcccgggctgcaccgcccccacccccgcccccgccgccccctcccggggctgaccgcgtcgtcaaagctgtccctttccccccaacacatcgcttgacatctgaagaagtatttgatttggatgggatacccagggttgatgttctgaagaaccacttggtgaaagaaggtcgagtagatgaagaaattgcgcttagaattatcaatgagggtgctgccatccttcggagagagaaaaccatgatagaagtagaagctccaatcacagtgtgtggtgacatccatggccaattttttgatctgatgaaactttttgaagtaggaggatcacctgctaatacacgatacctttttcttggcgattatgtggacagaggttattttagtatagagtgtgtcttatatttatgggttctgaagattctatacccaagcacattatttcttctgagaggcaaccatgaatgcagacaccttactgaatattttacctttaagcaggaatgtaaaattaagtattcggaaagagtctatgaagcttgtatggaagcttttgatagtttgcctcttgctgcacttttaaaccaacagtttctttgtgttcatggtggactttcaccagaaatacacacactggatgatattaggagattagatagattcaaagagccacctgcatttggaccaatgtgtgacttgttatggtccgatccttctgaagattttggaaatgaaaaatcacaggaacattttagtcacaatacagttcgaggatgttcttatttttataactatccagcagtgtgtgaatttttgcaaaacaataatttgttatcgattattagagctcatgaagctcaagatgcaggctatagaatgtacagaaaaagtcaaactacagggttcccttcattaataacaattttttcggcacctaattacttagatgtctacaataataaagctgctgtattaaagtatgaaaataatgtgatgaatattcgacagtttaactgttctccacatccttactggttgcctaattttatggatgtcttcacgtggtctttaccgtttgttggagaaaaagtgacagaaatgttggtaaatgttctgagtatttgctctgatgatgaactaatgactgaaggtgaagaccagtttgatggttcagctgcagcccggaaagaaatcataagaaacaaaattcgagcaattggcaagatggcaagagtcttctctgttctcagggaggagagtgaaagtgtgctgacactcaagggcctgactcccacagggatgttgcctagtggagtgttagctggaggacggcagaccctgcaaagtgccacagttgaggctattgaggctgaaaaagcaatacgaggattctctccaccacatagaatctgcagttttgaagaggcaaagggtttggataggatcaatgagagaatgccacctcggaaagatgctgtacagcaagatggtttcaattctctgaacaccgcacatgccactgagaaccacgggacgggcaaccatactgcccagtgacccactacttcccagggactctcacatctcgggccccaaatggacagatcacccgaggagctggaggggtcggccaagctgactgtaaatttcacagtctctctgaagaaaccattgtgcttctgagaccctagcccccttcctggatggaggcttgagggccctgggacatgtgctatctgataagattgggtcatcgctgccaaggtggagagcagtgagcaaggggcttggggcaatttccagtggagggcatccacacctccattttatgcttgtggttcacacatttaagtttacaaatcagatttcttttccccttcagtagaattagattttgtttttcaatcatgatttcaaatgcaatcctaagagctaatgtggacttttctttttccatgaaatgtctttaaaggatgaattagcatggtcttaaaatacatttctgaggttactagctgtattttgaattgtgagcaaaatgccgagaaacccagttggcatttatacaaaatgttgacctcaggtctatagttcttaaatgtggctaattctgtaacatagtcttggtattttttaattatgaatgcatatcctatttccaggcaggctctcttacttgaacacaaatccaaaaactaatttagagtcttttttgcccagatcttttaagacttacaccccagagatttaagaagaaaacctctaaatttcaaaattatgaagaattacagaattactcatttaaggtactttaaaagaagtttgtacattgtcaaagtaaattttaattcaaatcatgtctgtaaaacttgacgtattttgtgtatgcatgttttcattttgcaaatatttaatatatagacctatgatgtacaggtacgacatgtataggttacctagatgttatgagaaattttagtttattgtgagtactcaagttgcttagagagccaccagggtgatttgctgctggctttctatcatttttatgttttaatgcaaaggaaattttaaaatgttctggaagtgtttttgattaagcaatgcagcctagaagcaatggttctgttcaatcattcagatgttagtggaagcataaaagtcaagactgcatgttgaaacctttcttttgatagttactgaactgcttggttaaactaaatggaaccatgtgctaatttttcacaattattgacctgtattgattgccactgtagtttggtatttccctttactttggtggcctgcttccctcatgccctggaatacaactcagagctccaggcagcggaaccatctattgttttgtttgccagaaagtgcaccctgtatggtctcctgtctaagttggaaatattatgcatgtgcaggactattcgagtatt

SEQ ID NO: 201 - DNA, 1736 bp, Homo sapiens
  NM_032721
aaaatttgctgattaaatgaatgtgggtgtgtttgagagggatcctagacagccaagccttctggcatgaaacgctgagaagatgggagtgtctgctggcagagatgaaagtgagcaggggtgagcgcagccactgcccaacgcaaaccgtgaagaagcttctggaagagcagaggcgccgccagcagcagcagcccgacgctggcggggtgcagggacaatttctccctcccccagagcagcccctgaccccatctgtgaatgaggctgtgactggccaccctcccttcccagcacactcggagactgtgggttctggacctagcagcctgggctttccagactgggaccccaacacgcatgctgcctacactgacagcccctactcttgccctgcttctgctgccgaaaatttcctgcctcctgacttctacccaccctcggacccagggcagccgtgcccatttccccagggcatggaggctggaccctggagagtttctgcacccccttcaggacccccacagttccccgctgtggtccctggaccatcgctggaggtggcccgagctcacatgctggctttggggccacagcagctgctggcccaggatgaggagggggacacgctccttcacctgtttgcggctcgggggctgcgctgggcggcatatgctgcggctgaggtgctccaggtgtaccggcgtcttgacattcgtgagcataagggcaagacccctctcctggtggcggctgctgccaaccagcccctgattgtggaggatctgttgaacctgggagcagagcccaatgccgctgaccatcagggacgttcggtcttgcacgtggccgctacctacgggctcccaggagttctcttggctgtgcttaactctggggtccaggttgacctggaagccagagacttcgagggcctcaccccgctccacacggccatcctggcccttaacgttgctatgcgcccttccgacctctgtccccgggtgctgagcacacaggcccgagacaggctggattgtgtccacatgttgctgcaaatgggtgctaatcacaccagccaggagatcaagagcaacaagacagttctgcacttggccgtgcaggctgccaaccccactctggttcagctgctgctggagctgccccggggagacctgcggacctttgtcaacatgaaggcccacgggaacacagccctccacatggcggctgccctgccccctgggccggcccaggaggccatcgtgcggcacctgttggcagctggggcggaccccacactgcgcaacctggagaatgagcagcccgttcacctgctgcggcccgggccgggccctgaggggctccggcagctgttgaagaggagccgtgtggcgccgccaggcctgtcctcttaggactcaaacccagaccctggactgattttccagtccccaccgtcctgcgggacagccagcgtatgctaatgttgcaaacccatgataatgtatgtggaatatcctgccattggggttttacattaaaaccccagaatggctgcagaggggtgaacaggccccaatatttggggtgctgtgatacccctcttctacccacaaggagccctcttgatgatttctgtgaaatcgaggccccttgattgtttctgtgaaacaccctgcacccctagtcctttccccactgagatctttcgggttctctcccctaactcagct

SEQ ID NO: 202 - DNA, 3401 bp, Homo sapiens
  NM_147180
gggtacgagccccgcccgagctcaccgcccctgctcccctctccgaccctttgagccgtggccgttgccagatgtccacaatgggaaacgaggccagttacccggcggagatgtgctcccactttgacaatgatgaaattaaaaggctgggcaggaggtttaagaagttggacttggacaaatcagggtctctgagcgtggaggagttcatgtccctgccggagctgcgccacaacccgttggtgcggcgagtgatcgacgtcttcgacaccgacggtgatggagaagtggacttcaaggaattcatcctggggacctcccagttcagcgtcaagggcgacgaggagcagaagttgaggtttgcgttcagcatttacgacatggataaagatggctacatttccaacggggagctcttccaggtgctgaagatgatggtgggcaacaacctgacggactggcagctccagcagctggtcgacaaaaccatcatcatcctggacaaggatggcgatgggaagatatcctttgaggaattcagtgctgtggtcagagacctggagatccacaagaagctggtcctcatcgtatgagcctttttcttacaagcaccacccaacaacttctgctttcttccctatctctttcaagatttgctcaagacgtccaactgtctctctgacttatctggaagtatttctttttgtgaagccatatgtcctaacaggagcttcatcaccaactcagtgctattaattctccttctctgaatgactcagggtaccctatagggggaagagcaagtcaaatgagcatagtggggaaagaaaaggaaatggcttttataaacatcttttactttgttttgattcaaagaccaaactagaactttaaaagttcaaaaataagaaagtatacatttttgctgttatttctcatcattttgtatatgggaggaaatttataatttgcatgggtgttaggtgaactgttttcatttgcttgtgttcagatatcttgccagattgttaacttcctattgtagcaacagggacaaatatatttgtctttgctgggcatctcgtaatcacttttcttaggggacagaatcccatcttttccttcggcagattgcagccccattccccacaatgcatccagaaatcgctgtgcatgtttgaggggtaggagttcatttggcctcctcctgacttgttgctccagctcctgaacagaaactagcttcagggctcttataggaaatgctaagcctggactaagtgcccagctcagccatcatcctcattagcagtgggtttccagggttggcagcccacaccagcattaatttcaatgaaggggacctccagctaaatagggcaagcaaagtgttctcccagaaagtgctctcacctccgacactggtccctacccccaggtggagacaatgagacatttagttagttgtcctacggaggagaggcagtagagtcaggctgcagtggaatctccctaggatgccaaagtggcattatgtctgttgctcacagatgcagagactgacaattgtgtctccacagcagaggtggatgctagctacaccagctatgctgattttgaaaatcagatctgaggcctagaagagaaactgagagctctctcattccaggaagccttttccctagtggaacattcagtgcagaagtcctcacacactaacagatctccaccattgggtgagccggagcctgtggttccaggggagatccagaatgcatgtgtctcttcccacgcatttatgtcatgttggtagctttagatcagccatggtgagaaaagaacaaaagcttttagttgtttttgttttgttttggagaatttgtttaccagtaaatacatcactgcctgtaccccaaatgttaccagctccctgaggtgtcccacatactattgtgagttctcagagcatgaactgtcctcagaagagcagggctaggacttgtcccagcatctgtgcctccataccaatcctctttctcacagagaaccacttcccatataagatgcttaaggctctcaaaacagcagaacaatgaaacatactctccctacacttgcttagccaagagataccactcaggtaacttttttcaggacatggaagatctgtttcaaggagatttactgctattttatttggaagaagctggcaactggtcttgaccaaaatagaaaaaaaaaaaaaaagtccacaaatttaatcacttgtagggaacccatctatcaaggtaccctaccatatacttttgtatttaatagattacttagaaaccacaaaaataggaatccttaccccttcaattcctgttcaaccctaaaaactgtgataaacgctcccaaccctgtggtgatcagggttatgtaatgttcaaagattcagacacacctgggtttggattcagttgcaactgggttgttatcacactcacttctttttagctgtgtgagcatgaataatttacttagcctctctgtgtctttccacctaattaaagaggatccaccccagggttattttgaggattcaaaaaggcatgcaacacacctggagcacaattccactttcattcaactaattcccttcccttcccccttcttccccttctacaagatcaatatgtaaaggagacatgaggcttactggttgcttttgaacacttacttagttcttagctacacccactctaaaattaactggacattagtgtacagcccatgtccaagcccagagagaaaacaatgggaacaatttcaaggtcctcaccactccttcatttgcagaggggacaacagactttctgaccagagaactggagaatttttaaaacaaaatctctcattccagcccaacctatttaactttttgtggaggaattttacatggaggaagtgagcacatgtcatgctagccaagaggacattattgtcattaaagagaggcattatttatacaccctgcaatgtgcacattaaaatatggaaattttaaaattatgaccaagggcttgaaacatattggattacatgctcacatttaacaaagagaggaaatgtgtttcagtttctggagtggctggaatttacaagctaattgttcaataaatctactcaagatagttacctaaggctttgtggcaatgaccttgaactgagagcctgtatctggatttagcacttgaaagatctaactggatatttgggttaaaagaatcacatttattcccaaatcggaatgctttgtttttcctgtcagttaattgccagttgccaacaaatctagttctatacagtttcttgggatgatgataataaacatttattgagcaaaaaaaaaaaaaaaa

SEQ ID NO: 203 - DNA, 362 bp, Homo sapiens
  misc_feature (276)..(276): n is a, c, g or t.
agggctcggcgtggcagggatgatggcaggctcagcttgggtgcaggtgtggaagcaaccactgggggccggggcaggacctccagatttggcaccgggaactgcacatccatggcagggctgggctttgctgcctcctttgctgtggctgcgtcgtcctctgggggtgcagggaagggctcacttctccggggtggcgtccgactgatgctgttcttgggctggaagatctcgttgtagtcctcggcgttctggatctccgcgcgggactccgtncggtccccgggtcatcctcgctgctgggggctgcggcgcttngctctctgagttcttgcttcaacctctgaggcttacttncaagg

SEQ ID NO: 204 - DNA, 513 bp, Homo sapiens
  misc_feature (27)..(27): n is a, c, g or t.
ggtctgcttctgatgcttagattacangtgcagagttcatccacgttgtgacttgcatcaatactccttttcatggctggataatactcagtttgatggatgtaccatattttgtttatccattcatcagttgatgggcacttggattattttctactttttgactgttgtgaataatgctgcagtgaatatttgtgtacaggtttttgtataaacacgtttttatttctcttggctgtatacctaggtgatgggaattgctgggattataagggtaactttttttgttgttttttgaggatgggagtcttgcttgtcaccagggctgggagtgcagtgccgaganctcggctcactgggaccctccatctcccngggttcaagccgattcccctgcctcagcctcccaagaggctgggnctgcaggnacggaccaccatgtcccggcaatttttngatttaggnggagatgggggttcaccatgttggccggatgggctcaactcctgcctc

SEQ ID NO: 205 - DNA, 336 bp, Homo sapiens
  misc_feature (322)..(322): n is a, c, g or t.
ttttttttttctttatatgtattatatttttcattaataaataggttttcttcattaaacacagaacatataacagattgaaacactccccccctccccccaattccaaagacaagagtctataaaacaaatgccagctgtactaccctaagggcagaaaaagtctggtgacccccacccagccctgcccctgcagcaccaccacccccacactctgcaagagaagggggtctggggcttctcccttggaccctgggggacttaggtgagaagcatgtgaatgtatgatgtcacctctccatgaggcatgggctatgcaaangattgnggtttcct

SEQ ID NO: 206 - DNA, 475 bp, Homo sapiens
  misc_feature (444)..(444): n is a, c, g or t.
ttttttttttttataatttaattttcataacagtatttttatttgtgaaatttttttacaatttgctttttttttggtatgttctacatttttttccatgtactaagtatcgtgggaagttccaacctcatacttatgctgcttttctacttgctaatattggatgcttcttgccaggctccttaaattgtgctgtaacctgggaagaaaccttcctactctccacaaaccctgaataatcctgagagtttaaacctcacctaattattctctatcatttagaaactagaggggtggaggactgagagagggacagtataaaattcgaaaccagtgatgcattggctgaaaaacactgacatgaattgtgagagcaaagaaaccacagctggttttgcaggtgcagtccaggtgggccttggcagctgatgttgaattgtacgnttgtagccntatattttctgggctacagctg

SEQ ID NO: 207 - DNA, 418 bp, Homo sapiens
  misc_feature (178)..(178): n is a, c, g or t.
ttttttttttttgagattttgtttgtttttatttggggagaatgaaggaggagggagattttagactgaatcattctagagtatttgacgactacagctcctctctctttgtactacggagaccctgcttatagcccccaacaggaaatcctcatctgcggttgccagacagccagantatttccacctgtgccagaacaagacagaagaccccaggnagattcttcctgggggacaggtntcaagcagacccaatcccccatacagagantttcttaggatgaaaacagcctcaggcccttaggacccagcctctgggtctctcttctggggcccacaccaggcacaacacctggggagaaggccccaagcnttgcacaagtaccaaacccacccgggggnacagcaccaccttnct

SEQ ID NO: 208 - DNA, 469 bp, Homo sapiens
  misc_feature (199)..(199): n is a, c, g or t.
ttttttttattttcataagttgatatttgtattctcttaataccttacttattttatatatctttacatataaagcatattaatagaatttgtggccaatattactagttattacatcctttttatacactaatcattgctgaaacagtcttttatgcactgtatcattaatcgtcagagccatgttaagtatgtaggntagcagttatgttctatttactttcttattttggggggtttatttatataggatgtataatttatttatttatataggcaaggatggggggtcttgnctctgtctcccagtctaggaggtataggtgggccgcgatctcaggctcaccgcagccctcaaccctcccangggcccaaggcgggatccctccctctcttcaggcctctcaagtagggctnggggtctaacntaggggccnggtacccanccaacccccgggttaactnttaa

SEQ ID NO: 209 - DNA, 482 bp, Homo sapiens
  misc_feature (393)..(393): n is a, c, g or t.
ttttttttttttttggtgtctaatacttttacttcccagcagcataataccactttggtcaggttgaaccactgcacaattttgcagctctttcagaaaaataatgaaaacaattggaaagacatgatgtacatgcaaatggtgagaaaatgatactattgtccaatcctttcccgtaaatgtaaaatatccttagttcatctccaaactgtgtatttattataagcccttacatcagggattttgtttctttcagtttttgtagcaccaaatagacacagcggctaacaagaaataaatctgaaaagtcactgaaatatttatcacatgtcaggaaattttctggtctgtacctttaaccatgttcctgggcttcacatattcttcttaagngtaagctataacataaggttgagttcccacccgtgtttggctaccgntgtggtttgtttacctggataatactttgttaatatcctttg

SEQ ID NO: 210 - DNA, 389 bp, Homo sapiens
  misc_feature (191)..(191): n is a, c, g or t.
ttttttttctcaaggtatttcttttgtaaaggagccagatttggcaactagactgaaaatcttccaaaattctgtacaaatgtgtaatatacaaatatacacaaggcttttgccaagaaaagacatcacaacaaatgacagaggtggctttggtgtcacataccacctacagcatctcaaacccctaggantgtgaggggctgatggaccacaacataggctacacatgtaaacacgctacattacactatgtacactggagtataaacaatccactgtcctctccggtctgttcacaaggccctctggcggggangggaagggccatttcaccggggcaacccacattaagggggaacttctttaaggtggggaggggagaaccctgc

SEQ ID NO: 211 - DNA, 312 bp, Homo sapiens
  misc_feature (269)..(269): n is a, c, g or t.
acattggatttctaattttaagttgaagatatatataacagaaattctatatttgcttcccctaccccatagattgtctcagacaccctaccctggagacaccaagtccagaaggtgacttatgctagtaatccacagatgagggataagggggaccagctgcagagcttggtggtatcagcaaacatgccaaaggaggatcaaaatgatcagatagagatgaaggaaaagaccagctgggtatttgatcagatttagtagagaaaggngaaaggatttcaaaatggttccaggtttttactccaaaaaaca

SEQ ID NO: 212 - DNA, 435 bp, Homo sapiens
  misc_feature (10)..(11): n is a, c, g or t.
tttttttttnntttttttttttttnatttacaaaagaaaggtttaattggacttacagttccacatggctggagagggttcacaatcatggtggaaggcaggaggagcaagtcacatcttacgtgaatggtggcaggcaaaacaaaagagcttttacagagaaactcccgttttttaaaatcatcagntctcatgagacccattcactatcatgagaacagcataggaaagacccatccccatgattcagtcatttcctaccagggtccctcccacatgtgaganttntggggagctacaaggatgagttttggggtaggggacacagngccaaaccatnttcattccacccctggggcccctcccagttttcatatctttcacattttcaaaaccagttcatggcctttccccaacagttcccccaaagttntc

SEQ ID NO: 213 - DNA, 487 bp, Homo sapiens
  misc_feature (464)..(464): n is a, c, g or t.
tttttttttgcatatctcaaatttaagtttatttaaaaattacaaaaatgtctccactcaagttgtccttgaaaaaaatttagaagcagcaacaatcatcttggttagttacaagcagtctagtgcaatttataaaatcgagcaaaatttggatgtgtgtcttatccctctgtagacacgggacccttttttgaggtctctttctttagggtctggtagcatcatgactgggacactttccaaagccttcttcctttggactctatgtaaaataattctatagacccctgtgattgaggctgcgagcatctctcccttgggttatttcgaatatggctcttctgtaatgcccaaatgttctaaagtgcttttggacctcattttcttcttccttggggagtgctgggtttccgacaaatgtttggggatccagttgggaaaggttccaaaggtgtaggggtttnggcacccctngcatcccttcntt

SEQ ID NO: 214 - DNA, 393 bp, Homo sapiens
  misc_feature (328)..(328): n is a, c, g or t.
ttttttttttttttcggtttgttgttcagtgtttattaagtagattcagtcttatatatgatatacagattcaaaaggaaaaggattccacatacttatgaaatcagtgcatttagcaagtaataccatggagataacagctcgattaacagctcattggtcttttgttaagtgagggagagagggagtcggcctgtgctagtcattccacaaacaaaacagaatttagtcacatcacgtagaggaagacacattctaagatttaaaatattaagccacatttactgggtaaaactcactatataggaccacaaataatttttatagngggccttgaggagggagtcctcacctgcttagnagggccncgttagttgcatggggcctntctgt

SEQ ID NO: 215 - DNA, 411 bp, Homo sapiens
  misc_feature (8)..(8): n is a, c, g or t.
tttttttntntttaattaattagaaagtaggctgggcacggtggctcatgcctataatcccagcacttngggaggccnaggatctcctctctggtggatcanttnagggcaggagttaagagaccatcctggccaacatgatgaaaccctgtctctactaaaaatacaaaaagtagctgggcgtggtggcatactcttacaatcccagctacttgggaggctgaggcaggagaatcacttgaacctaggaagcagaggttgcagtgggccaagntcacaccactatactctagcctgggcgaacagaggtggggaaaaaagtaggacccctgtcctatattcaggtttttcttcacatatatgaacccatcttaaattcttacgttgtttaaaggtagtnttaggttaatt

SEQ ID NO: 216 - DNA, 500 bp, Homo sapiens
  misc_feature (271)..(271): n is a, c, g or t.
ttttttttgagacagagtctctctgtcacccaggctggagtgcagtggtgtgatctcagctcactgcaacttctgccttccaggttcaagcaattctcccacctcagcctcccaagtagctgggatcacaggtgtgcaccaccacatccagctaatttttatatctttagtagagacagggtttcaccatgtggcccaggctggtctcgaactcaacctccagtgaaccacctgcctcagcctcccaaagtgctgggattacaggcgtganacncgttgcccagccaagatttggggaattaagaaaggaaatacaaacacaactgaggccaaaggaaagantctgacttgattaacattcactggcatttcaggcattccaaaggaatttccatttatttttaacaacttttgtnattttcttttttaaattacagggcagggcaaaggaggttaacaattttttttaatnggttgnttaacnggcacgtgtttanttt

SEQ ID NO: 217 - DNA, 352 bp, Homo sapiens
  misc_feature (295)..(295): n is a, c, g or t.
tttttttttttaacagtgcagtgatttattgaccagactttgcagcaagaacacagcgaaggtggggcccgtacaatccagcctggcagagggtctggcccccttagagcagaatctggggaccccagtatatttccctcacagccccccaaagtccagcctcaccctgctccaggcccctcctgaagtgaggggcagcagggggaccggagtcctggaggggctggaaggcaggtggtgcccagagcggggctggcaccgggtgcatgcctgccccggtagccagcaggaggtnattcgtncgggggcagtnggggcntgcaggcgggcancaggnttcaccacacggaac

SEQ ID NO: 218 - DNA, 438 bp, Homo sapiens
  misc_feature (407)..(407): n is a, c, g or t.
tttttttttgaaattctcataattttaatgatcaatagcttctggtgggctctggatggtacagttaaacaatagacttaaagacctcccccaaagcacgtccacaccccctcggcagcgtctgcctccatcagcattccactgcccagtcggacttccacagcctcagacccccctcttgggttatggaaatccacatcttagtgtaatgagctgaaaaaccctgggtacacccgtgaatctgttattttttaaaaataacaaatactaagccaaacacctgcccagggtctgctgcctcacagagtgtgggcgatctgaatactctgttgtctcttgtgggccgggtggttttggcttcggtccctgttctactgaaatcatacgggcataggagttcgtaaatnttctttacacagtacaccaggggcgggccag

SEQ ID NO: 219 - DNA, 490 bp, Homo sapiens
  misc_feature (4)..(4): n is a, c, g or t.
tcanagtaaatggccnncaattttttaacgaaatctcactactgcaaatgcattgttgtcctagctaatgaatgcatagagtattgcctgcaaaataataattgagattctatttttaagaagcttagaacagtacatggtgcatagcaaagactctgtgtatgtgaagccagattttaaaatatggtaacaagtgtctgaaaatatgtggctcaatttgtctcccggttacttttccctctccccctttaaaatgtagaggaaggagaagaagagataagaggtttgtgagtgaagacaagggccctttaaggcctggggaagactaacgccatagggntctccctctgccttaaaaggcacagggantcttagtgggggaaaaagaagtgggtgataaatagccagtccntgtgcccgggatttccaagtcagtgcgtgccnggggtcccactgcgggtcacgngcactctgggnctctntttgnaat

SEQ ID NO: 220 - DNA, 399 bp, Homo sapiens
  misc_feature (10)..(10): n is a, c, g or t.
cgcccacggnccgnctgatgtgggccgactcgttcttgccgttgatgagctgcagcccgaggatgccgtctggggcgtangcaggggcgttccccgagatctcgcgctcgccgaccagccacacgtacccggagcccgtcatgttcagcatcgcgnctgncngttatacagtggcagcatcgtcctcgctggcagaaaggatgatgacccgggcctccagctctttcgcctccatcagcagggccgtcacgttcttggtccctgggtcaaactgcagcaccttctctgccttggactcacgctcctccagcagcgtctccaggcgtttctgagccgcccggccctcgtggtcgtcgctgancagcaggatgatgtggttncagctgtagaacacggatc

SEQ ID NO: 221 - DNA, 425 bp, Homo sapiens
  misc_feature (322)..(322): n is a, c, g or t.
tattcacacaaggggttgcccaaatataattttgctttgactattgagatctagtgaaagtggggtatatgaattctaattgcaaatatccaggctcagaggcccagcaggactttctaacacaatcttttagcggaagttagaaatggtatatagcaggagagtcagatttgagaagcatatgtagattcgaagctgggattacaggtgtaagccactgcaccctgcctattcttataatcatatatttatatttcaaatgggattttaactgggttatttaatagtttaattaggataaagtaattcagggcggggtgtnggtggctcacgcctgtaaatcccagcactttnggcagggctgagggcagggtgggttaccccgagggtccggnagtttcgnggacccagccccaacccaacgt

SEQ ID NO: 222 - DNA, 399 bp, Homo sapiens
  misc_feature (175)..(175): n is a, c, g or t.
gcgtgtgtgtaaccttgaactcctaggctcaagtgatcctcccaccttagcctctcaagtagctgggtctacaggtgtgtaccaccatgtctggctaatttattaattttttttgtagagacagggtctcactatgttgcccaggctggtcttgaattcctgggcttcaagtgancctaatgcctcagcctcctaaagctctgggactacaggcatgagctatcatgcccagccagtactaaataatttttaacaaaagantaaatcattattttttatataaggtttctgtaaggggggctacaggatttattatacttttctgacatccaaagntttcaaatttggttatatttttccngatatatggagggcccaaaatacttttttaataacctt

SEQ ID NO: 223 - DNA, 385 bp, Homo sapiens
  misc_feature (28)..(28): n is a, c, g or t.
agataggaaatgaactctattatatatncctttttaatcttttttaaaaaattatactttnctgggatacatgtgcagaatgcataggtttattacacaggtacacacgtgccattgtagtttgctgcacccatcaatccgtcatctacattaggtgtttctcccaatgctatccctcctgtacccctcaccccctaaaaggccctggtttgtgatgtttccctccctgtgtccatgtgttctcattgttcagcttccacttatgagtgagaacatgtgggtgtttggttttctatccttgtgatagtttgctgaggaatgatgggtttccaggcttcatccgngtcccggcaaaggaccatggaactcatccntttttgtgggc

SEQ ID NO: 224 - DNA, 344 bp, Homo sapiens
  R97095
ataaactatcttgcatttaatcaatcattacaaaaagtattcatgccacatacaactttgtaacacccaggtgcgtgaccatctataaaaggggggaaacccaacctctatcccttttccacgtgggcaagccaagggttctgagggctcatccacagggctcgccggcctggcctcctgctgcgtcccgggatgtggaccactgacccagaggcttctgagtcctgagcacagataagggctcctttcaggcctttccttgagctgcacgtgaacctgtgtggggcagggcagcgtttgcagggcgtgtttacggggcagggcagcgttttgcagggcgtgtt

SEQ ID NO: 225 - DNA, 428 bp, Homo sapiens
  misc_feature (420)..(420): n is a, c, g or t.
ggagtggccagtttattctcttaagagagaaaatgaggggattaatggactgtagttctggacaaggtggaaaactcttaaagtggaagtattggggcgaatctctgacaggctaggatggtgcagtcagtcccttcacccagaaatcagtagaatgttagcagttcagactcaaaccttgtgaaaaacaggtggtggaaaggaaatccctcacagcaactggcaccataatcaagacaatgtttgcagaattaatggagttacctgctttcagccccaggtcatagctattgtctgccctgctggatatgtgataattaatttgtgattcatgttgtctttaaaatgggggttcactcatctccagtaggaatttaaggtccacagtgaaagtttgttcccccccttccccaaaggggntaaacctt

SEQ ID NO: 226 - DNA, 397 bp, Homo sapiens
  misc_feature (371)..(371): n is a, c, g or t.
gggtgctgtaaaataaatgcaactttacagaatatggtaaaagtggaggatagacaaagcacattaaatattcagctactatttacaatttctcccacaattacatgttaaaacatactttttttttcaagttgctgtttaaatctaggcaaaagtgcttaaaaaaatctttcactatttacagttaaaaccagaccacataaatgctcaggacatcttacaacgaccctccgccagtgcttccaaacagatacagtaaacacccagcaggccggttctcagctcttactcaagggcacccgggaagctcagggaacttgcctataggcgggtcatctggattggtttaaaccatggatttcatggaaggnccccattatctggaattaacctggct

SEQ ID NO: 227 - DNA, 423 bp, Homo sapiens
  misc_feature (373)..(373): n is a, c, g or t.
gtttattcagaaggttacgttctcagcctttattccagggcccaattttcaagacaagccttcgcgggacaggaggagtcctctgaagatagcagtgcatgagcagagaaacacacatggcttggtctcgccgcccagcagctgattccgaggaggggctgagaggggccaatttcacccttccctagcaagactcggaggaggttcttcctacacaagaagcccatctgaccaccagggcttcctggcagagagtggtgctagatgtcagccaggaactccccagaccaggtattcttccaggctcaggcataaagaaccaggaggcttttattttctattaatcaacctagttgggaagcagagcccattntaaaggcagagaggtattgattacaggcttaaagaaggaaaacgctgctt

SEQ ID NO: 228 - DNA, 408 bp, Homo sapiens
  misc_feature (343)..(343): n is a, c, g or t.
cagggttttagttgtctagtctggtattgaactcctggcttcaagcaatcctcctgcctcggcctcccaaagtgttgggattataggcatgagcaccatgcccagactaaacacattatttaaagagaagtaaactggcatcttttccttgaagagtaacgaaagctgggatttatgaagcattttgattgagctaacttttaagtatgtgccagtatacttcactatcaaaacgaagaccagaagagaattctagcccaaatcttttaatatagcatttcaaattaaactgtggggaatttaatcacattttccctccctatcaggaatgacagtgttgggncacaacatggcacaaagaggtcccaggtaatatttttgcaggangtacttcaagaggggaggcaa

SEQ ID NO: 229 - DNA, 394 bp, Homo sapiens
  T97352
atgctgaatcttctatggcacaacttgaacaaactctaaaactgaataattgctgaaacaatatgtttctatactagccactttaaattagaaaagtctctttaagctacaggtttatggttctaaaaagttccatgcactgcccagagctatttgtaaattaaatcgtttaaaataaagctctttaatactctataattaggtcttgtctctctgaacatgactttactgtaaatgctccttcttgactctcctattgtcccaaggattcctgaggaaggaaaggcagccggggggattaattcaacattattggctggcttcaggtggcactaaatcggctttaaggaggcaggcaaggactctggtggctctctggccccacaggttcccg

SEQ ID NO: 230 - DNA, 491 bp, Homo sapiens
  W15233
tttttttttttaaggccaaaggtgcaacatcaccgattttagtagcaaatgccttgaaatattcatttttgtaaatcactcttacatttggaaagtcaggcatgcagatcttacagcggaactggaagtaaactttctcttacatttctgggcatatgactactgaactagaaaaacacttactgaacctctttggcaaacatttatcaagacaattttggacacaaatgcttgtttagaaattttgctcaggaaggtgagtcagttccttaagaatgctggtttgtttcagcatatcctgaaatctaagtctgggataccaagagtccctttaactaaatatcatttaggagttttttaatggggtgggttaccttggtggtacagccaataaaactggaccagtccagttacaggccccacagtaatggcataagaatactgacttattctccgtttaaagaaagccctctggcaagataccaggtcat

SEQ ID NO: 231 - DNA, 436 bp, Homo sapiens
  misc_feature (1)..(1): n is a, c, g or t.
ngcgaaaccataggcagccagacacagagactatactgtttctcatttcatcagccttgggacttgaacagcaggtcactccatttgaaaactagcagacgtaataaatagaaagaggcatagaaagcaagctgaccacagattgggctgtagcacccagccatttctgtctgtcttggaaagggccaaggggaatgccctngtatactgccattgtatcaaatgttttgggcacctttttcagggcatttatgaaagaaaaaaagaaaagggagggattaaggnctggataagccttttcctgcctgagtacctgtttcatgatgtttctttataagtcatcatgactggcaaaataatctttaaggttgcttggcagtgtnccaaaaagtagaccaatagaccctgtancccgttggagaaaatttttaaancc

SEQ ID NO: 232 - DNA, 594 bp, Homo sapiens
  misc_feature (86)..(92): n is a, c, g or t.
ttttatgattgtaaaaattttattctcaataacaattccactaaaattatacaaagtacattcaatacttaagaatggcaaaggtnnnnnnnggaggagaggaagccagtttgnnctggaagcatcaacagttgactccaacaaaggaagcccagctggntggacggaagttgggggtggaggtccggttgtaaaaaataaaaaaggggatcggtaaaaaaaggccacccacaaggaagcagagtgagcgtgcatgttcctnggggagggcaggaggctcagctgacatgctgctgctgggcactggagctcaggcnggctccgagtttggtggggggcangtttttatgctttgaaatactgcacaactttgttgggggcagctccgccagcacagcttttggccaaggtctcttttgctgcgttgcggaactctcgaaagggaacgaactgcacaatatcgcgggctgcctcctcccccgtgtgggaacgccaatgcggcttgtccccatccaggaactccatngcagcganttccgcattgcccccgtgccgaatntttggccccgaggccnaatttcccaanagtgaatc

SEQ ID NO: 233 - DNA, 473 bp, Homo sapiens
  misc_feature (323)..(323): n is a, c, g or t.
atacttaataattttattacatgtacacacaaaatcatagcaaaatatgatatattttataggatttttttgtcttttcatgatgccatcgaatatttgcaaatgcctaaatggaaccctttcttcattctccacacaagtccaaaaacacaaacacacacgcacacacatacacctatacacatgcatccttttaaccaaaggtaatactcactggtgttaaaactaacagctctactggacaatggcttcatggacattttagaggcagtcttaagtctgctgtgggcacaggcatgtcggatatgatttccctgcagggntgccctctcgggtggctgtggggggatncctgcnggcccctcngcctgccagcgcttganctcagnctcacagtgagccgagatgaacaggtgcnctcttctctggcggcggggggcacgttgaaggtcggntccctggagaaaacgttc

SEQ ID NO: 234 - DNA, 435 bp, Homo sapiens
  misc_feature (156)..(156): n is a, c, g or t.
ttttactttgaaattactttaatttagaaatagaaaacatcttgaaaggaaaaaaaaaaacccacaaaacatacaggcaaatttgtattctgtgagtttctcacaccctcacacttgttcaccaatctctaagatgaaaaactctcttccaaaatngtattcagccaccaacaatgatgttattaataggaatatgggatcaatttcacaaagaagccaatgaatcccattatagcaaatcctattgctgttgccatggaatcttctggaattcttttctatcaggtttagtgcatcttttaaccagccgaatggagtcccttacaaactggccggacttggntcaacaaactgcattacctgatccatggactggcctacccaaccggacacctaaaatggccaggggacacgtagcactggaggcctcgtgcc

SEQ ID NO: 235 - DNA, 1048 bp, Homo sapiens
  X00457
aggagctggggccatcaaggcggaccatgtgtcaacttatgccgcgtttgtacagacgcatagaccaacaggggagtttatgtttgaatttgatgaagatgagatgttctatgtggatctggacaagaaggagaccgtctggcatctggaggagtttggccaagccttttcctttgaggctcagggcgggctggctaacattgctatattgaacaacaacttgaataccttgatccagcgttccaaccacactcaggccaccaacgatccccctgaggtgaccgtgtttcccaaggagcctgtggagctgggccagcccaacaccctcatctgccacattgacaagttcttcccaccagtgctcaacgtcacgtggctgtgcaacggggagctggtcactgagggtgtcgctgagagcctcttcctgcccagaacagattacagcttccacaagttccattacctgacctttgtgccctcagcagaggacttctatgactgcagggtggagcactggggcttggaccagccgctcctcaagcactgggaggcccaagagccaatccagatgcctgagacaacggagactgtgctctgtgccctgggcctggtgctgggcctagtcggcttcatcgtgggcaccgtcctcatcataaagtctctgcgttctggccatgacccccgggcccaggggaccctgtgaaatactgtaaaggtgacaaaatatctgaacagaagaggacttaggagagatctgaactccagctgccctacaaactccatctcagcttttcttctcacttcatgtgaaaactactccagtggctgactgaattgctgacccttcaagctctgtccttatccattacctcaaagcagtcattccttagtaaagtttccaacaaatagaaattaatgacactttggtagcactaatatggagattatcctttcattgagccttttatcctctgttctcctttgaagagcccctcactgtcaccttcccgagaataccctaagaccaataaatacttcagtatttcag

SEQ ID NO: 236 - DNA, 1169 bp, Homo sapiens
  misc_feature (189)..(190): n is a, c, g or t.
gaattcctttgagactaactgaaatttctgacagccccacactctataactttattgtaaattttctgccaaaaatgatgctttcctatacactcctaatacaagtataaatatattatttaatctagtcttaggttgatttaaaattttgaaaattcactccaaacatatgctctgtaaccgtatggnnaccaatgagaagtgtactctttcaaggtaaatctgtgctgccctggtctgacctgggactctggggatactgcgcccgtgtgctgagttactgagacgagccagccctgcagctgtgctcagcctgccccatcccctgctgatttgcctgttcctagagcacagccccctgccctgaagacttcttataggctggtcacacccggtgcaggagtcagccccagtcaggacacagcaaggacgtgagggcccccactcagctcctggggctcctggggctcctggggctctggctgccaggtaaggaaggagaacactaggattatactcggtcagtgtgctcagtactgtcaggaacttcagggaagtcctctgataacatgattaattgcaagaatatttgtttttatgtttctaacttcaggtgccagatgtgacatccagatgacccagtctccatcctccctgtctgcatctgtaggaggcagagtcaccatcacttgccgggcgagtcagggcattagcaataatttaaattggtatcagcagaaaccagggaaaactcctaagctcctgatctatgctgcatccagtctgcaaagtgggattccctctcggttcagtgacagtggatctgggacagattacactctcaccatcagcagcctgcagcctgaagattttgcagcttattactgtcaacagagtgacagtacccctcccacagtgttacaagtcataacataaaccccaaggaagcagatgtgtgaggctgggctgccccagtgctccttctggtgcctctatctgctgagggaagttctcaaactcagtcaggtttggaaagtcatcgggagattttcctagaggaggccagggaggttcctctgaaccctaagcctctttcgccctcatccccagcagaaaagacgtgacaatgcctgtcctgactgaataaagaagagagataagtccagctg

SEQ ID NO: 237 - DNA, 2602 bp, Homo sapiens
  XM_001754
gcctcggcggcccccctcaccgcgctcccggcgcccctcccgtcagttcgccagctgccagccccgggaccttttcatctcttcccttttggccggaggagccgagttcagatccgccactccgcacccgagactgacacactgaactccacttcctcctcttaaatttatttctacttaatagccactcgtctctttttttccccatctcattgctccaagaatttttttcttcttactcgccaaagtcagggttccctctgcccgtcccgtattaatatttccacttttggaactactggccttttctttttaaaggaattcaagcaggatacgtttttctgttgggcattgactagattgtttgcaaaagtttcgcatcaaaaacaacaacaacaaaaaaccaaacaactctccttgatctatactttgagaattgttgatttcttttttttattctgacttttaaaaacaacttttttttccacttttttaaaaaatgcactactgtgtgctgagcgcttttctgatcctgcatctggtcacggtcgcgctcagcctgtctacctgcagcacactcgatatggaccagttcatgcgcaagaggatcgaggcgatccgcgggcagatcctgagcaagctgaagctcaccagtcccccagaagactatcctgagcccgaggaagtccccccggaggtgatttccatctacaacagcaccagggacttgctccaggagaaggcgagccggagggcggccgcctgcgagcgcgagaggagcgacgaagagtactacgccaaggaggtttacaaaatagacatgccgcccttcttcccctccgaaactgtctgcccagttgttacaacaccctctggctcagtgggcagcttgtgctccagacagtcccaggtgctctgtgggtaccttgatgccatcccgcccactttctacagaccctacttcagaattgttcgatttgacgtctcagcaatggagaagaatgcttccaatttggtgaaagcagagttcagagtctttcgtttgcagaacccaaaagccagagtgcctgaacaacggattgagctatatcagattctcaagtccaaagatttaacatctccaacccagcgctacatcgacagcaaagttgtgaaaacaagagcagaaggcgaatggctctccttcgatgtaactgatgctgttcatgaatggcttcaccataaagacaggaacctgggatttaaaataagcttacactgtccctgctgcacttttgtaccatctaataattacatcatcccaaataaaagtgaagaactagaagcaagatttgcaggtattgatggcacctccacatataccagtggtgatcagaaaactataaagtccactaggaaaaaaaacagtgggaagaccccacatctcctgctaatgttattgccctcctacagacttgagtcacaacagaccaaccggcggaagaagcgtgctttggatgcggcctattgctttagaaatgtgcaggataattgctgcctacgtccactttacattgatttcaagagggatctagggtggaaatggatacacgaacccaaagggtacaatgccaacttctgtgctggagcatgcccgtatttatggagttcagacactcagcacagcagggtcctgagcttatataataccataaatccagaagcatctgcttctccttgctgcgtgtcccaagatttagaacctctaaccattctctactacattggcaaaacacccaagattgaacagctttctaatatgattgtaaagtcttgcaaatgcagctaaaattcttggaaaagtggcaagaccaaaatgacaatgatgatgataatgatgatgacgacgacaacgatgatgcttgtaacaagaaaacataagagagccttggttcatcagtgttaaaaaatttttgaaaaggcggtactagttcagacactttggaagtttgtgttctgtttgttaaaactggcatctgacacaaaaaaagttgaaggccttattctacatttcacctactttgtaagtgagagagacaagaagcaaattttttttaaagaaaaaaataaacactggaagaatttattagtgttaattatgtgaacaacgacaacaacaacaacaacaacaaacaggaaaatcccattaagtggagttgctgtacgtaccgttcctatcccgcgcctcacttgatttttctgtattgctatgcaataggcacccttcccattcttactcttagagttaacagtgagttatttattgtgtgttactatataatgaacgtttcattgcccttggaaaataaaacaggtgtataaagtggagaccaaatactttgccagaaactcatggatggcttaaggaacttgaactcaaacgagccagaaaaaaagaggtcatattaatgggatgaaaacccaagtgagttattatatgaccgagaaagtctgcattaagataaagaccctgaaaacacatgttatgtatcagctgcctaaggaagcttcttgtaaggtccaaaaactaaaaagactgttaataaaagaaactttcagtcag

SEQ ID NO: 238 - DNA, 5722 bp, Homo sapiens
  XM_007189
gcagccgccacattcaacaggcagcagcgcagcgggcgcgccgctggggagagcaagcggcccgcggcgtccgtccgtccttccgtccgcggccctgtcagctggagcgcggcgcaggctctgccccggcccggcggctctggccggccgtccagtccgtgcggcggaccccgaggagcctcgatgtggatggccccgcgaagttaagttctgggctcgcgcttccactccgccgcgccttcctcccagtttccgtccgctcgccgcaccggcttcgttcccccaaatctcggaccgtcccttcgcgccccctccccgtccgcccccagtgctgcgttctccccctcttggctctcctgcggctgggggaggggcgggggtcaccatggccgaggcgcctcaggtggtggagatcgacccggacttcgagccgctgccccggccgcgctcgtgcacctggccgctgcccaggccggagtttagccagtccaactcggccacctccagcccggcgccgtcgggcagcgcggctgccaaccccgacgccgcggcgggcctgccctcggcctcggctgccgctgtcagcgccgacttcatgagcaacctgagcttgctggaggagagcgaggacttcccgcaggcgcccggctccgtggcggcggcggtggcggcggcggccgccgcggccgccaccggggggctgtgcggggacttccagggcccggaggcgggctgcctgcacccagcgccaccgcagcccccgccgcccgggccgctgtcgcagcacccgccggtgccccccgccgccgctgggccgctcgcggggcagccgcgcaagagcagctcgtcccgccgcaacgcgtggggcaacctgtcctacgccgacctcatcaccaaggccatcgagagctcggcggagaagcggctcacgctgtcgcagatctacgagtggatggtcaagagcgtgccctacttcaaggataagggtgacagcaacagctcggcgggctggaagaattcaattcgtcataatctgtccctacacagcaagttcattcgtgtgcagaatgaaggaactggaaaaagttcttggtggatgctcaatccagagggtggcaagagcgggaaatctcctaggagaagagctgcatccatggacaacaacagtaaatttgctaagagccgaagccgagctgccaagaagaaagcatctctccagtctggccaggagggtgctggggacagccctggatcacagttttccaaatggcctgcaagccctggctctcacagcaatgatgactttgataactggagtacatttcgccctcgaactagctcaaatgctagtactattagtgggagactctcacccattatgaccgaacaggatgatcttggagaaggggatgtgcattctatggtgtacccgccatctgccgcaaagatggcctctactttacccagtctgtctgagataagcaatcccgaaaacatggaaaatcttttggataatctcaaccttctctcatcaccaacatcattaactgtttcgacccagtcctcacctggcaccatgatgcagcagacgccgtgctactcgtttgcgccaccaaacaccagtttgaattcacccagcccaaactaccaaaaatatacatatggccaatccagcatgagccctttgccccagatgcctatacaaacacttcaggacaataagtcgagttatggaggtatgagtcagtataactgtgcgcctggactcttgaaggagttgctgacttctgactctcctccccataatgacattatgacaccagttgatcctggggtagcccagcccaacagccgggttctgggccagaacgtcatgatgggccctaattcggtcatgtcaacctatggcagccaggcatctcataacaaaatgatgaatcccagctcccatacccaccctggacatgctcagcagacatctgcagttaacgggcgtcccctgccccacacggtaagcaccatgccccacacctcgggtatgaaccgcctgacccaagtgaagacacctgtacaagtgcctctgccccaccccatgcagatgagtgccctggggggctactcctccgtgagcagctgcaatggctatggcagaatgggccttctccaccaggagaagctcccaagtgacttggatggcatgttcattgagcgcttagactgtgacatggaatccatcattcggaatgacctcatggatggagatacattggattttaactttgacaatgtgttgcccaaccaaagcttcccacacagtgtcaagacaacgacacatagctgggtgtcaggctgagggttagtgagcaggttacacttaaaagtacttcagattgtctgacagcaggaactgagagaagcagtccaaagatgtctttcaccaactcccttttagttttcttggttaaaaaaaaaaacaaaaaaaaaaaccctccttttttcctttcgtcagacttggcagcaaagacatttttcctgtacaggatgtttgcccaatgtgtgcaggttatgtgctgctgtagataaggactgtgccattggaaatttcattacaatgaagtgccaaactcactacaccatataattgcagaaaagattttcagatcctggtgtgctttcaagttttgtatataagcagtagatacagattgtatttgtgtgtgtttttggtttttctaaatatccaattggtccaaggaaagtttatactctttttgtaatactgtgatgggcctcatgtcttgataagttaaacttttgtttgtactacctgttttctgcggaactgacggatcacaaagaactgaatctccattctgcatctccattgaacagccttggacctgttcacgttgccacagaattcacatgagaaccaagtagcctgttatcaatctgctaaattaatggacttgttaaacttttggaaaaaaaaagattaaatgccagctttgtacaggtcttttctatttttttttgtttattttgttatttgcaaatttgtacaaacatttaaatggttctaatttccagataaatgatttttgatgttattgttgggacttaagaacatttttggaatagatattgaactgtaataatgttttcttaaaactagagtctactttgttacatagtcagcttgtaaattttgtggaaccacaggtatttggggcagcattcataattttcattttgtattctaactggattagtactaattttatacatgcttaactggtttgtacactttgggatgctacttagtgatgtttctgactaatcttaaatcattgtaattagtacttgcatattcaacgtttcaggccctggttgggcaggaaagtgatgtatagttatggacactttgcgtttcttatttaggataacttaatatgtttttatgtatgtattttaaagaaatttcatctgcttctactgaactatgcgtactgcatagcatcaagtcttctctagagacctctgtagtcctgggaggcctcataatgtttgtagatcagaaaagggagatctgcatctaaagcaatggtcctttgtcaaacgagggattttgatccacttcaccattttgagttgagctttagcaaaagtttcccctcataattctttgctcttgtttcagtccaggtggaggttggttttgtagttctgccttgaggaattatgtcaacactcatacttcatctcattctcccttctgccctgcagattagattacttagcacactgtggaagtttaagtggaaggagggaatttaaaaatgggacttgagtggtttgtagaatttgtgttcataagttcagatgggtagcaaatggaatagaacttacttaaaaattggggagatttatttgaaaaccagctgtaagttgtgcattgagattatgttaaaagccttggcttaagaatttgaaaatttctttagcctgtagcaacctaaactgtaattcctatcattatgttttattactttccaattacctgtaactgacagaccaaattaattggctttgtgtcctatttagtccatcagtattttcaagtcatgtggaaagcccaaagtcatcacaatgaagagaacaggtgcacagcactgttcctcttgtgttcttgagaaggatctaatttttctgtatatagcccacatcacacttgctttgtcttgtatgttaattgcatcttcattggcttggtatttcctaaatgtttaacaagaacacaagtgttcctgataagatttcctacagtaagccagctctattgtaagcttcccactgtgatgatcatttttttgaagattcattgaacagccaccactctatcatcctcattttggggcagtccaagacatagctggttttagaaacccaagttcctctaagcacagcctcccgggtatgtaactgaacttggtgccaaagtacttgtgtactaatttctattactacgtactgtcactttcctcccgtgccattactgcatcataatacaaggaacctcagagcccccatttgttcattaaagaggcaactacagccaaaatcactgttaaaatcttactacttcatggagtagctcttaggaaaatatatcttcctcctgagtctgggtaattatacctctcccaagcccccattgtgtgttgaaatcctgtcatgaatccttggtagctctctgagaacagtgaagtccagggaaaggcatctggtctgtctggaaagcaaacattatgtggcctctggtagtttttttcctgtaagaatactgactttctggagtaatgagtatatatcagttattgtacatgattgctttgtgaaatgtgcaaatgatatcacctatgcagccttgtttgatttattttctctggtttgtactgttattaaaagcatattgtattatagagctattcagatattttaaatataaagatgtattgtttccgtaatatagacgtatggaatatatttaggtaatagatgtattacttggaaagttctgctttgacaaactgacaaagtctaaatgagcacatgtatcccagtgagcagtaaatcaatggaacatcccaagaagaggataaggatgcttaaaatggaaatcattctccaacgatatacaaattggacttgttcaactgctggatatatgctaccaataaccccagccccaacttaaaattcttacattcaagctcctaagagttcttaatttataactaattttaaaagagaagtttcttttctggttttagtttgggaataatcattcattaaaaaaaatgtattgtggtttatgcgaacagaccaacctggcattacagttggcctctccttgaggtgggcacagcctggcagtgtggccaggggtggccatgtaagtcccatcaggacgtagtcatgcctcctgcatttcgctacccgagtttagtaacagtgcagattccacgttcttgttccgatactctgagaagtgcctgatgttgatgtacttacagacacaagaacaatctttgctataattgtataaagccataaatgtacataaattatgtttaaatggcttggtgtctttcttttctaattatgcagaataagctctttattaggaattttttgtgaagctattaaatacttgagttaa

SEQ ID NO: 239 - DNA, 2857 bp, Homo sapiens
  XM_012039
ctgctcctgcgcggcagctgctttagaaggtctcgagcctcctgtaccttcccagggatgaaccgggccttccctctggaaggcgagggttcgggccacagtgagcgagggccagggcggtgggcgcgcgcagagggaaaccggatcagttgagagagaatcaagagtagcggatgaggcgcttgtggggcgcggcccggaagccctcgggcgcgggctgggagaaggagtgggcggaggcgccgcaggaggctcccggggcctggtcgggccggctgggccccgggcgcagtggaagaaagggacgggcggtgcccggttgggcgtcctggccagctcaccttgccctggcggctcgccccgcccggcacttgggaggagcagggcagggcccgcggcctttgcattctgggaccgcccccttccattcccgggccagcggcgagcggcagcgacggctggagccgcagctacagcatgagagccggtgccgctcctccacgcctgcggacgcgtggcgagcggaggcagcgctgcctgttcgcgccatgggggcaccgtggggctcgccgacggcggcggcgggcgggcggcgcgggtggcgccgaggccgggggctgccatggaccgtctgtgtgctggcggccgccggcttgacgtgtacggcgctgatcacctacgcttgctgggggcagctgccgccgctgccctgggcgtcgccaaccccgtcgcgaccggtgggcgtgctgctgtggtgggagcccttcggggggcgcgatagcgccccgaggccgccccctgactgccggctgcgcttcaacatcagcggctgccgcctgctcaccgaccgcgcgtcctacggagaggctcaggccgtgcttttccaccaccgcgacctcgtgaaggggccccccgactggcccccgccctggggcatccaggcgcacactgccgaggaggtggatctgcgcgtgttggactacgaggaggcagcggcggcggcagaagccctggcgacctccagccccaggcccccgggccagcgctgggtttggatgaacttcgagtcgccctcgcactccccggggctgcgaagcctggcaagtaacctcttcaactggacgctctcctaccgggcggactcggacgtctttgtgccttatggctacctctaccccagaagccaccccggcgacccgccctcaggcctggccccgccactgtccaggaaacaggggctggtggcatgggtggtgagccactgggacgagcgccaggcccgggtccgctactaccaccaactgagccaacatgtgaccgtggacgtgttcggccggggcgggccggggcagccggtgcccgaaattgggctcctgcacacagtggcccgctacaagttctacctggctttcgagaactcgcagcacctggattatatcaccgagaagctctggcgcaacgcgttgctcgctggggcggtgccggtggtgctgggcccagaccgtgccaactacgagcgctttgtgccccgcggcgccttcatccacgtggacgacttcccaagtgcctcctccctggcctcgtacctgcttttcctcgaccgcaaccccgcggtctatcgccgctacttccactggcgccggagctacgctgtccacatcacctccttctgggacgagccttggtgccgggtgtgccaggctgtacagagggctggggaccggcccaagagcatacggaacttggccagctggttcgagcggtgaagccgcgctcccctggaagcgacccaggggaggccaagttgtcagctttttgatcctctactgtgcatctccttgactgccgcatcatgggagtaagttcttcaaacacccatttttgctctatgggaaaaaaacgatttaccaattaatattactcagcacagagatgggggcccggtttccatattttttgcacagctagcaattgggctccctttgctgctgatgggcatcattgtttaggggtgaaggagggggttcttcctcaccttgtaaccagtgcagaaatgaaatagcttagcggcaagaagccgttgaggcggtttcctgaatttccccatctgccacaggccatatttgtggcccgtgcagcttccaaatctcatacacaactgttcccgattcacgtttttctggaccaaggtgaagcaaatttgtggttgtagaaggagccttgttggtggagagtggaaggactgtggctgcaggtgggactttgttgtttggattcctcacagccttggctcctgagaaaggtgaggagggcagtccaagaggggccgctgacttctttcacaagtactatctgttcccctgtcctgtgaatggaagcaaagtgctggattgtccttggaggaaacttaagatgaatacatgcgtgtacctcactttacataagaaatgtattcctgaaaagctgcatttaaatcaagtcccaaattcattgacttaggggagttcagtatttaatgaaaccctatggagaatttatccctttacaatgtgaatagtcatctcctaatttgtttcttctgtctttatgtttttctataacctggattttttaaatcatattaaaattacagatgtgaaaataaagcagaagcaacctttttccctcttcccagaaaaccagtctgtgtttacagacagaagagaaggaagccatagtgtcacttccacacaattatttatttcatgtctttactggacctgaaatttaaactgcaatgccagtcctgcaggagtgctggcattaccctctgcagaacagtgaaaggtattgcactacattatggaatcatgcaaaa

SEQ ID NO: 240 - DNA, 1269 bp, Homo sapiens
  XM_012608
aggggacaggctgcagccggtgcagttacacgttttcctccaaggagcctcggacgttgtcacgggtttggggtcggggacagagcggtgaccatggccaggctggcgttgtctcctgtgcccagccactggatggtggcgttgctgctgctgctctcagctgagccagtaccagcagccagatcggaggaccggtaccggaatcccaaaggtagtgcttgttcgcggatctggcagagcccacgtttcatagccaggaaacggggcttcacggtgaaaatgcactgctacatgaacagcgcctccggcaatgtgagctggctctggaagcaggagatggacgagaatccccagcagctgaagctggaaaagggccgcatggaagagtcccagaacgaatctctcgccaccctcaccatccaaggcatccggtttgaggacaatggcatctacttctgtcagcagaagtgcaacaacacctcggaggtctaccagggctgcggcacagagctgcgagtcatgggattcagcaccttggcacagctgaagcagaggaacacgctgaaggatggtatcatcatgatccagacgctgctgatcatcctcttcatcatcgtgcctatcttcctgctgctggacaaggatgacagcaaggctggcatggaggaagatcacacctacgagggcctggacattgaccagacagccacctatgaggacatagtgacgctgcggacaggggaagtgaagtggtctgtaggtgagcacccaggccaggagtgagagccaggtcgccccatgacctgggtgcaggctccctggcctcagtgactgcttcggagctgcctggctcatggcccaacccctttcctggaccccccagctggcctctgaagctggcccaccagagctgccatttgtctccagcccctggtccccagctcttgccaaagggcctggagtagaaggacaacagggcagcaacttggagggagttctctggggatggacgggacccagccttctgggggtgctatgaggtgatccgtccccacacatgggatgggggaggcagagactggtccagagcccgcaaatggactcggagccgagggcctcccagcagagcttgggaagggccatggacccaactgggccccagaagagccacaggaacatcattcctctcccgcaaccactcccaccccagggaggccctggcctccagtgccttcccccgtggaataaacggtgtgtcctgagaaacca

SEQ ID NO: 241 - DNA, 1642 bp, Homo sapiens
  XM_027216
tgtgataagttattcaacttatgaaattcaagttacatgtgaattctgccaggcaatacaaggacctgtggaatatgagtgatgacaaaccctttctatgtactgcgcctggatgtggccagcgttttaccaacgaggatcatttggctgtccataaacataaacatgagatgacactgaaatttggtccagcacgtaatgacagtgtcattgtggctgatcagaccccaacaccaacaagattcttgaaaaactgtgaagaagtgggtttgtttaatgagttggcgagtccatttgagaatgaattcaagaaagcttcagaagatgacattaaaaaaatgcctctagatttatcccctcttgcaacacctatcataagaagcaaaattgaggagccttctgttgtagaaacaactcaccaggatagtcctttacctcacccagagtctactaccagtgatgagaaggaagtaccattggcacaaactgcacagcccacatcagctattgttcgtccagcatcattacaggttcccaatgtgctgcttacaagttctgactcaagtgtaattattcagcaggcagtaccttcaccaacctcaagtactgtaatcacccaggcaccatcctctaacaggccaattgtccctgtaccaggcccatttcctcttctgttacatcttcctaatggacaaaccatgcctgttgctattcctgcatcaattacaagttctaatgtgcatgttccagctgcagtcccactcgttcgaccagtcaccatggtgcctagtgttccaggaatcccaggtccttcctctccccaaccagtacagtcagaagcaaaaatgagattaaaagctgctttgacccagcaacatcctccagttaccaatggtgatactgtcaaaggtcatggtagcggattggttaggactcagtcagaggaatctcgaccgcagtcattacaacagccagccacatccactacagaaactccggcttctccagctcacacaactccacagacccaaagtacaagtggtcgtcggagaagagcagctaacgaagatcctgatgaaaaaaggagaaagtttttagagcgaaatagagcagcagcttcaagatgccgacaaaaaaggaaagtctgggttcagtctttagagaagaaagctgaagacttgagttcattaaatggtcagctgcagagtgaagtcaccctgctgagaaatgaagtggcacagctgaaacagcttcttctggctcataaagattgccctgtaaccgccatgcagaagaaatctggctatcatactgctgataaagatgatagttcagaagacatttcagtgccgagtagtccacatacagaagctatacagcatagttcggtcagcacatccaatggagtcagttcaacctccaaggcagaagctgtagccacttcagtcctcacccagatggcggaccagagtacagagcctgctctttcacagatcgttatggctccttcctcccagtcacagccctcaggaagttgattaaaaacctgcagtacaacagttttagatactcattagtgacttcaaagggaaatcaaggaaagaccagtttccatttatgcgaaatctgtggttgtaaattt

SEQ ID NO: 242 - DNA, 3022 bp, Homo sapiens
  XM_043467
gaacagcaaagggtagagcagacctgcgccaggggcgcacaacggccgtgtccacctcccggccccaagatggtgcttcccacaggcagccacgcgtagcagccagagacagctccagacatgtggctcttcttcgggatcactggattgctgacggcagccctctcaggtcatccatctccagccccacctgaccagctcaacaccagcagtgcagagagtgagctctgggagccaggggagcggcttccggtccgtctgacaaacgggagcagcagctgcagcgggacggtggaggtgcggctcgaggcgtcctgggagcccgcgtgcggggcgctctgggacagccgcgccgccgaggccgtgtgccgagcactgggctgcggcggggcggaggccgcctctcagctcgccccgccgacccctgagctgccgcccccgcctgcagccgggaacaccagcgtagcagctaatgccactctggccggggcgcccgccctcctgtgcagcggcgccgagtggcggctctgcgaggtggtggagcacgcgtgccgcagcgacgggaggcgggcccgtgtcacctgtgcagagaaccgcgcgctgcgcctggtggacggtggcggcgcctgcgccggccgcgtggagatgctggagcatggcgagtggggatcagtgtgcgatgacacttgggacctggaggacgcccacgtggtgtgcaggcaactgggctgcggctgggcagtccaggccctgcccggcttgcacttcacgcccggccgcgggcctatccaccgggaccaggtgaactgctcgggggccgaagcttacctgtgggactgcccggggctgccaggacagcactactgcggccacaaagaggacgcgggcgcggtgtgctcagagcaccagtcctggcgcctgacagggggcgctgaccgctgcgaggggcaggtggaggtacacttccgaggggtctggaacacagtgtgtgacagtgagtggtacccatcggaggccaaggtgctctgccagtccttgggctgtggaactgcggttgagaggcccaaggggctgccccactccttgtccggcaggatgtactactcatgcaatggggaggagctcaccctctccaactgctcctggcggttcaacaactccaacctctgcagccagtcgctggcagccagggtcctctgctcagcttcccggagtttgcacaatctgtccactcccgaagtccctgcaagtgttcagacagtcactatagaatcttctgtgacagtgaaaatagagaacaaggaatctcgggagctaatgctcctcatcccctccatcgttctgggaattctcctccttggctccctcatcttcatagccttcatcctcttgagaattaaaggaaaatatgccctccccgtaatggtgaaccaccagcacctacccaccaccatcccggcagggagcaatagctatcaaccggtccccatcaccatccccaaagaaggtccaggccccgccccctgaggactcagactctggctcggactcagactatgagcactatgacttcagcgcccagcctcctgtggccctgaccaccttctacaattcccagcggcatcgggtcacagatgaggaggtccagcaaagcaggttccagatgccacccttggaggaaggacttgaagagttgcatgcctcccacatcccaactgccaaccctggacactgcattacagacccgccatccctgggccctcagtatcacccgaggagcaacagtgagtcgagcacctcttcaggggaggattactgcaatagtcccaaaagcaagctgcctccatggaacccccaggtgttttcttcagagaggagttccttcctggagcagcccccaaacttggagctggccggcacccagccagccttttcagggtcccccagccctcagcctgactccaccgacaacgatgactacgatgacatcagcgcagcctaggccggggccagccgaggctcctggggtggctctgaccctctggcctcctgctctacctactccctttcccctttcccaccctcccagctcacctccccatggagctgagaggcctcccttggagagatggaaggaaacgttataccttgtacccctcggtctccatccatcaagccaaacctgctgccacagccctcccccggccccagatagcagccccagggaggatgctgcctccaagaggtgtgagccctctgtctcggggatgaacaagcagagtctgggctacctcttgacagctggtggaggggagttggggagctggactggatgactctggaggccccttccaaacctcaagtgtccggcgctttgattgcctgagtttctgacacttcagggcccagaggtcctgcgaggggcagaactggacccccatgccagtgctgctgcaggagggcccatatactagggtctgctgagctgttgtcactgatcggtgggcgctgggggggtagggtagcacaccagctgtcccaggctttgctccgggcggtaactgcacttgggcagggaatatagccttcctgggcacaactagctgacaatgacaggttgactgtgtacccccaaccaaggagctggggcccaaggccagtcctgccccagagacactccaagtccgccaggggcacagaccagttctgcagtgactgtccctggacaatgggtctttattctgagtttcctatggtttacaaagagggccccagcccagccccaccacagatcccagagataggggcccagtctccatgggggcaaggagcatagagatgttttccaggaaggggctcagaagctgcactaggccccgagtccccatgtgtctccttgaattgatgaggatgctcctgggagggatgcgtgactatgtggtgttgcacccggggctgcaaacgtctccgtgcagcccccagagagaggcccatgggctcagaccaggctttgttgtcctgc

SEQ ID NO: 243 - DNA, 2470 bp, Homo sapiens
  XM_043864
gacgagccgagccgagccaagcggagctgggccactgtgcacgcccggagggtcctggcggcgcccccgctcctgcgcgcactctcggcgccggacacgagcactgcctgccgggaacaggctggggggaggtgcgggggcttggcccacttggtggaagaacagctttggggattttttttttttcattgtcggatacaggcatttcaaagggaaaccgttgaaatgcataacctgcaaacactgcctcctaaaccaccaaaacctactactgtagccaacaacggtatgaataacaatatgtccttacaagatgctgaatggtactggggagatatctcgagggaagaagtgaatgaaaaacttcgagatacagcagacgggacctttttggtacgagatgcgtctactaaaatgcatggtgattatactcttacactaaggaaagggggaaataacaaattaatcaaaatatttcatcgagatgggaaatatggcttctctgacccattaaccttcagttctgtggttgaattaataaaccactaccggaatgaatctctagctcagtataatcccaaattggatgtgaaattactttatccagtatccaaataccaacaggatcaagttgtcaaagaagataatattgaagctgtagggaaaaaattacatgaatataacactcagtttcaagaaaaaagtcgagaatatgatagattatatgaagaatatacccgcacatcccaggaaatccaaatgaaaaggacagctattgaagcatttaatgaaaccataaaaatatttgaagaacagtgccagacccaagagcggtacagcaaagaatacatagaaaagtttaaacgtgaaggcaatgagaaagaaatacaaaggattatgcataattatgataagttgaagtctcgaatcagtgaaattattgacagtagaagaagattggaagaagacttgaagaagcaggcagctgagtatcgagaaattgacaaacgtatgaacagcattaaaccagaccttatccagctgagaaagacgagagaccaatacttgatgtggttgactcaaaaaggtgttcggcaaaagaagttgaacgagtggttgggcaatgaaaacactgaagaccaatattcactggtggaagatgatgaagatttgccccatcatgatgagaagacatggaatgttggaagcagcaaccgaaacaaagctgaaaacctgttgcgagggaagcgagatggcacttttcttgtccgggagagcagtaaacagggctgctatgcctgctctgtagtggtggacggcgaagtaaagcattgtgtcataaacaaaacagcaactggctatggctttgccgagccctataacttgtacagctctctgaaagaactggtgctacattaccaacacacctcccttgtgcagcacaacgactccctcaatgtcacactagcctacccagtatatgcacagcagaggcgatgaagcgcttactctttgatccttctcctgaagttcagccaccctgaggcctctggaaagcaaagggctcctctccagtctgatctgtgaattgagctgcagaaacgaagccatctttctttggatgggactagagctttctttcacaaaaaagaagtaggggaagacatgcagcctaaggctgtatgatgaccacacgttcctaagctggagtgcttatcccttctttttctttttttctttggtttaatttaaagccacaaccacatacaacacaaagagaaaaagaaatgcaaaaatctctgcgtgcagggacaaagaggcctttaaccatggtgcttgttaatgctttctgaagctttaccagctgaaagttgggactctggagagcggaggagagagaggcagaagaaccctggcctgagaaggtttggtccagcctggtttagcctggatgttgctgtgcacggtggacccagacacatcgcactgtggattatttcattttgtaacaaatgaacgatatgtagcagaaaggcacgtccactcacaagggacgctttgggagaatgtcagttcatgtatgttcagaagaaattctgtcatagaaagtgccagaaagtgtttaacttgtcaaaaaacaaaaacccagcaacagaaaaatggagtttggaaaacaggacttaaaatgacattcagtatataaaatatgtacataatattggatgactaactatcaaatagatggatttgtatcaataccaaatagcttctgttttgttttgctgaaggctaaattcacagcgctatgcaattcttaattttcattaagttgttatttcagttttaaatgtaccttcagaataagcttccccaccccagtttttgttgcttgaaaatattgttgtcccggatttttgttaatattcatttttgttatccttttttaaaagtaaatgtacaggatgccagt

SEQ ID NO: 244 - DNA, 2088 bp, Homo sapiens
  XM_048792
gggcagtcgtaggagactctgaaaaagcaaataaatcaatgttaaatcagaaatgtgaatgtagtaaggggctgaagagacaggggaagagaatacatgggaaaatattgaaaaggacagagtgatcaaaaagagcagggacatgggagcattgggcagcacactgggagccatttcactttatgctcttattgtatgattgagaaaaaaatgtccttagtggttaagtggcttttcaatgccacatcagacttgttccatagcagttgaattaggggaaggtgaataagttggaggttggtgacaaggagagaagctggaacagagaggagagtcagaaccagagggaaatgagggactgagtaggcatctcagggtttttgaaggagtggattttctttgttgcagtcaggggaggtttgtctgttggctgcagaaagaagtcagaatagagatatcgtggggtaggtttgtttggaacagaaatcaaagaccaatttttctgagagaaggaaataacatctgcaaatgatatgctgtttttgctacttccattgttagctgttctcccaggtgatggcaatgcagacgggctcaaggagcctctctccttccatgtcacctggatcgcatccttttacaaccattcctggaaacaaaatctggtctcaggttggctgagtgatttgcagactcatacctgggacagcaattccagcaccatcgttttcctgtgcccctggtccaggggaaacttcagcaatgaggagtggaaggaactggaaacattattccgtatacgcaccattcggtcatttgagggaattcgtagatacgcccatgaattgcagtttgaatatccttttgagatacaggtgacaggaggctgtgagctgcactctggaaaggtctcaggaagcttcttgcagttagcttatcaaggatcagactttgtgagcttccagaacaattcatggttgccatatccagtggctgggaatatggccaagcatttctgcaaagtgctcaatcagaatcagcatgaaaatgacataacacacaatcttctcagtgacacctgcccacgtttcatcttgggtcttcttgatgcaggaaaggcacatctccagcggcaagtgaagcccgaggcctggctgtcccatggccccagtcctggccctggccatctgcagcttgtgtgccatgtctcaggattctacccaaagcccgtgtgggtgatgtggatgcggggtgagcaggagcagcagggcactcagcgaggggacatcttgcccagtgctgatgggacatggtatctccgcgcaaccctggaggtggccgctggggaggcagctgacctgtcctgtcgggtgaagcacagcagtctagagggccaggacatcgtcctctactgggagcatcacagttccgtgggcttcatcatcttggcggtgatagtgcctttacttcttctgataggtcttgcgctttggttcaggaaacgctgtttctgttaagacacaccatgagcctcctcgtcacccttctccttttggggtgagagaccagcagcccaagggctccagacacacctgaacacatcgtgatgatgacgtcctctcaactctctttgtaaaaattttgttatttttgcttgtttctgattaatgattgtttgtcaatataagctcaatttaattttgcaggatttgttgttctgacctgggttctgggacttttaaattcaaattttatctccagatggaatggggtcctagcaacctccacatgttcaactattaatggatcatcaggcctgttttagatatcccttactccagagggccttccctgacttacaagtgggaagcagtctcttcctggtctgaactcccgccacattttagccgtactttgctaactgtgctcctcacttcctcttcttcattgcagttatttagatcccccctttccttctaatttttcagctccttcaatgcaaagtacatgtatttttaatatatgcatccctggtgaaggatcttgcctgcatgaaacatgttctcaataaaactctgtgttgaattt

SEQ ID NO: 245 - DNA, 2139 bp, Homo sapiens
  XM_049959
agacaggggtagtgcgaggccgggcacagccttcctgtgtggttttaccgcccagagagcgtcatggacctggggaaaccaatgaaaagcgtgctggtggtggctctccttgtcattttccaggtatgcctgtgtcaagatgaggtcacggacgattacatcggagacaacaccacagtggactacactttgttcgagtctttgtgctccaagaaggacgtgcggaactttaaagcctggttcctccctatcatgtactccatcatttgtttcgtgggcctactgggcaatgggctggtcgtgttgacctatatctatttcaagaggctcaagaccatgaccgatacctacctgctcaacctggcggtggcagacatcctcttcctcctgacccttcccttctgggcctacagcgcggccaagtcctgggtcttcggtgtccacttttgcaagctcatctttgccatctacaagatgagcttcttcagtggcatgctcctacttctttgcatcagcattgaccgctacgtggccatcgtccaggctgtctcagctcaccgccaccgtgcccgcgtccttctcatcagcaagctgtcctgtgtgggcatctggatactagccacagtgctctccatcccagagctcctgtacagtgacctccagaggagcagcagtgagcaagcgatgcgatgctctctcatcacagagcatgtggaggcctttatcaccatccaggtggcccagatggtgatcggctttctggtccccctgctggccatgagcttctgttaccttgtcatcatccgcaccctgctccaggcacgcaactttgagcgcaacaaggccatcaaggtgatcatcgctgtggtcgtggtcttcatagtcttccagctgccctacaatggggtggtcctggcccagacggtggccaacttcaacatcaccagtagcacctgtgagctcagtaagcaactcaacatcgcctacgacgtcacctacagcctggcctgcgtccgctgctgcgtcaaccctttcttgtacgccttcatcggcgtcaagttccgcaacgatctcttcaagctcttcaaggacctgggctgcctcagccaggagcagctccggcagtggtcttcctgtcggcacatccggcgctcctccatgagtgtggaggccgagaccaccaccaccttctccccataggcgactcttctgcctggactagagggacctctcccagggtccctggggtggggatagggagcagatgcaatgactcaggacatccccccgccaaaagctgctcagggaaaagcagctctcccctcagagtgcaagcccctgctccagaagatagcttcaccccaatcccagctacctcaaccaatgccaaaaaaagacagggctgataagctaacaccagacagacaacactgggaaacagaggctattgtcccctaaaccaaaaactgaaagtgaaagtccagaaactgttcccacctgctggagtgaaggggccaaggagggtgagtgcaaggggcgtgggagtggcctgaagagtcctctgaatgaaccttctggcctcccacagactcaaatgctcagaccagctcttccgaaaaccaggccttatctccaagaccagagatagtggggagacttcttggcttggtgaggaaaagcggacatcagctggtcaaacaaactctctgaacccctccctccatcgttttcttcactgtcctccaagccagcgggaatggcagctgccacgccgccctaaaagcacactcatcccctcacttgccgcgtcgccctcccaggctctcaacaggggagagtgtggtgtttcctgcaggccaggccagctgcctccgcgtgatcaaagccacactctgggctccagagtggggatgacatgcactcagctcttggctccactgggatgggaggagaggacaagggaaatgtcaggggcggggagggtgacagtggccgcccaaggcccacgagcttgttctttgttctttgtcacagggactgaaaacctctcctcatgttctgctttcgattcgttaagagagcaacattttacccacacacagataaagttttcccttgaggaaacaacagctttaaaag

SEQ ID NO: 246 - DNA, 1307 bp, Homo sapiens
  XM_051535
gtaagtctgctggcctccgccatcttagtaaagtaacagtcccatgaaacaaagatgcagtcgggcactcactggagagttctgggcctctgcctcttatcagttggcgtttgggggcaagatggtaatgaagaaatgggtggtattacacagacaccatataaagtctccatctctggaaccacagtaatattgacatgccctcagtatcctggatctgaaatactatggcaacacaatgataaaaacataggcggtgatgaggatgataaaaacataggcagtgatgaggatcacctgtcactgaaggaattttcagaattggagcaaagtggttattatgtctgctaccccagaggaagcaaaccagaagatgcgaacttttatctctacctgagggcaagagtgtgtgagaactgcatggagatggatgtgatgtcggtggccacaattgtcatagtggacatctgcatcactgggggcttgctgctgctggtttactactggagcaagaatagaaaggccaaggccaagcctgtgacacgaggagcgggtgctggcggcaggcaaaggggacaaaacaaggagaggccaccacctgttcccaacccagactatgagcccatccggaaaggccagcgggacctgtattctggcctgaatcagagacgcatctgaccctctggagaacactgcctcccgctggcccaggtctcctctccagtccccctgcgactccctgtttcctgggctagtcttggaccccacgagagagaatcgttcctcagcctcatggtgaactcgcgccctccagcctgatcccccgctccctcctccctgccttctctgctggtacccagtcctaaaatattgctgcttcctcttcctttgaagcatcatcagtagtcacaccctcacagctggcctgccctcttgccaggatatttatttgtgctattcactcccttccctttggatgtaacttctccgttcagttccctccttttcttgcatgtaagttgtcccccatcccaaagtattccatctacttttctatcgccgtccccttttgcagccctctctggggatggactgggtaaatgttgacagaggccctgccccgttcacagatcctggccctgagccagccctgtgctcctccctcccccaacactccctaccaaccccctaatcccctactccctccaccccccctccactgtaggccactggatggtcatttgcatctccgtaaatgtgctctgctcctcagctgagagagaaaaaaataaactgtatttggctgc

SEQ ID NO: 247 - DNA, 1078 bp, Homo sapiens
  NM_005368
gcagcctcaaaccccagctgttggggccaggacacccagtgagcccatacttgctctttttgtcttcttcagactgcgccatggggctcagcgacggggaatggcagttggtgctgaacgtctgggggaaggtggaggctgacatcccaggccatgggcaggaagtcctcatcaggctctttaagggtcacccagagactctggagaagtttgacaagttcaagcacctgaagtcagaggacgagatgaaggcgtctgaggacttaaagaagcatggtgccaccgtgctcaccgccctgggtggcatccttaagaagaaggggcatcatgaggcagagattaagcccctggcacagtcgcatgccaccaagcacaagatccccgtgaagtacctggagttcatctcggaatgcatcatccaggttctgcagagcaagcatcccggggactttggtgctgatgcccagggggccatgaacaaggccctggagctgttccggaaggacatggcctccaactacaaggagctgggcttccagggctaggcccctgccgctcccacccccacccatctgggccccgggttcaagagagagcggggtctgatctcgtgtagccatatagagtttgcttctgagtgtctgctttgtttagtagaggtgggcaggaggagctgaggggctggggctggggtgttgaagttggctttgcatgcccagcgatgcgcctccctgtgggatgtcatcaccctgggaaccgggagtggcccttggctcactgtgttctgcatggtttggatctgaattaattgtcctttcttctaaatcccaaccgaacttcttccaacctccaaactggctgtaaccccaaatccaagccattaactacacctgacagtagcaattgtctgattaatcactggccccttgaagacagcagaatgtccctttgcaatgaggaggagatctgggctgggcgggccagctggggaagcatttgactatctggaacttgtgtgtgcctcctcaggtatggcagtgactcacctggttttaataaaacaacctgcaacatctca